SEQUENCE LISTING (240 sequences)

SEQ ID NO: 1 - protein, 8 aa, synthetic construct
GFTFSNYG

SEQ ID NO: 2 - protein, 8 aa, synthetic construct
GYTFTSYW

SEQ ID NO: 3 - protein, 8 aa, synthetic construct
GFSLSRYS

SEQ ID NO: 4 - protein, 8 aa, synthetic construct
ISSGGGDT

SEQ ID NO: 5 - protein, 8 aa, synthetic construct
IHPSDSET

SEQ ID NO: 6 - protein, 7 aa, synthetic construct
IWGGGYT

SEQ ID NO: 7 - protein, 13 aa, synthetic construct
ARDYGGTWDYFDY

SEQ ID NO: 8 - protein, 15 aa, synthetic construct
AREEGQLGHGGAMDY

SEQ ID NO: 9 - protein, 11 aa, synthetic construct
ARYIDSSGYDY

SEQ ID NO: 10 - protein, 6 aa, synthetic construct
QDISKY

SEQ ID NO: 11 - protein, 6 aa, synthetic construct
QTVNDD

SEQ ID NO: 12 - protein, 5 aa, synthetic construct
SSVSY

SEQ ID NO: 13 - protein, 6 aa, synthetic construct
ENIYSY

SEQ ID NO: 14 - protein, 9 aa, synthetic construct
QQGDTFPWT

SEQ ID NO: 15 - protein, 9 aa, synthetic construct
QQDYSSPYT

SEQ ID NO: 16 - protein, 9 aa, synthetic construct
QQWSSNPLT

SEQ ID NO: 17 - protein, 9 aa, synthetic construct
QHHYGTPYT

SEQ ID NO: 18 - protein, 120 aa, synthetic construct
EVKLVESGGGLVKPGASLKLSCAASGFTFSNYGMSWVRQTSDKRLEWVASISSGGGDTYYPDNVKGRFTISRENAKNTLYLQMSSLNSEDTALYYCARDYGGTWDYFDYWGQGTTLTVSS

SEQ ID NO: 19 - protein, 122 aa, synthetic construct
QVQLQQPGAELVRPGVSVKLSCKASGYTFTSYWMNWVKQRPGQGLEWIGMIHPSDSETRLNQKFKDKAILTVDKSSSTAYMQLSSPTSEDSAVYYCAREEGQLGHGGAMDYWGQGTSVTVSS

SEQ ID NO: 20 - protein, 117 aa, synthetic construct
QVQLKESGPGLVAPSQSLSITCTVSGFSLSRYSVHWVRQPPGKGLEWLGMIWGGGYTDYNSALKSRLSISKDNSKSQVFLKMNSLQTDDTAMYYCARYIDSSGYDYWGQGTTLTVSS

SEQ ID NO: 21 - protein, 107 aa, synthetic construct
DIQMTQTTSSLSASLGDRVTISCRASQDISKYLNWYQQKPDGTVKLLIYYTSRLHSGVPSRFSGSGSGTDYSLTISNLEQEDIATYFCQQGDTFPWTFGGGTKLEIK

SEQ ID NO: 22 - protein, 107 aa, synthetic construct
SIVMTQTPKFLLVSAGDRVTITCKASQTVNDDVAWYQQKPGQSPKLLIYYVSNRHTGVPDRFTGSGYGTDFTFTISTVQAEDLAVYFCQQDYSSPYTFGGGTKLEIK

SEQ ID NO: 23 - protein, 106 aa, synthetic construct
QIVLTQSPAIMSASPGEKVTMTCSASSSVSYMHWYQQKSGTSPKRWIYDTSKLASGVPARFSGSGSGTSYSLTISSMEAEDAATYYCQQWSSNPLTFGAGTKLELK

SEQ ID NO: 24 - protein, 107 aa, synthetic construct
DIQMTQSPASLSASVGETVTITCRASENIYSYLAWYQQKQGKSPQLLVYNAKTLAEGVPSRFSGSGSGTQFSLKINSLQPEDFGSYYCQHHYGTPYTFGGGTKLEIK

SEQ ID NO: 25 - protein, 15 aa, synthetic construct
GGGGSGGGGSGGGGS

SEQ ID NO: 26 - protein, 242 aa, synthetic construct
EVKLVESGGGLVKPGASLKLSCAASGFTFSNYGMSWVRQTSDKRLEWVASISSGGGDTYYPDNVKGRFTISRENAKNTLYLQMSSLNSEDTALYYCARDYGGTWDYFDYWGQGTTLTVSSGGGGSGGGGSGGGGSDIQMTQTTSSLSASLGDRVTISCRASQDISKYLNWYQQKPDGTVKLLIYYTSRLHSGVPSRFSGSGSGTDYSLTISNLEQEDIATYFCQQGDTFPWTFGGGTKLEIK

SEQ ID NO: 27 - protein, 244 aa, synthetic construct
QVQLQQPGAELVRPGVSVKLSCKASGYTFTSYWMNWVKQRPGQGLEWIGMIHPSDSETRLNQKFKDKAILTVDKSSSTAYMQLSSPTSEDSAVYYCAREEGQLGHGGAMDYWGQGTSVTVSSGGGGSGGGGSGGGGSDIQMTQTTSSLSASLGDRVTISCRASQDISKYLNWYQQKPDGTVKLLIYYTSRLHSGVPSRFSGSGSGTDYSLTISNLEQEDIATYFCQQGDTFPWTFGGGTKLEIK

SEQ ID NO: 28 - protein, 239 aa, synthetic construct
QVQLKESGPGLVAPSQSLSITCTVSGFSLSRYSVHWVRQPPGKGLEWLGMIWGGGYTDYNSALKSRLSISKDNSKSQVFLKMNSLQTDDTAMYYCARYIDSSGYDYWGQGTTLTVSSGGGGSGGGGSGGGGSSIVMTQTPKFLLVSAGDRVTITCKASQTVNDDVAWYQQKPGQSPKLLIYYVSNRHTGVPDRFTGSGYGTDFTFTISTVQAEDLAVYFCQQDYSSPYTFGGGTKLEIK

SEQ ID NO: 29 - protein, 238 aa, synthetic construct
QVQLKESGPGLVAPSQSLSITCTVSGFSLSRYSVHWVRQPPGKGLEWLGMIWGGGYTDYNSALKSRLSISKDNSKSQVFLKMNSLQTDDTAMYYCARYIDSSGYDYWGQGTTLTVSSGGGGSGGGGSGGGGSQIVLTQSPAIMSASPGEKVTMTCSASSSVSYMHWYQQKSGTSPKRWIYDTSKLASGVPARFSGSGSGTSYSLTISSMEAEDAATYYCQQWSSNPLTFGAGTKLELK

SEQ ID NO: 30 - protein, 239 aa, synthetic construct
QVQLKESGPGLVAPSQSLSITCTVSGFSLSRYSVHWVRQPPGKGLEWLGMIWGGGYTDYNSALKSRLSISKDNSKSQVFLKMNSLQTDDTAMYYCARYIDSSGYDYWGQGTTLTVSSGGGGSGGGGSGGGGSDIQMTQSPASLSASVGETVTITCRASENIYSYLAWYQQKQGKSPQLLVYNAKTLAEGVPSRFSGSGSGTQFSLKINSLQPEDFGSYYCQHHYGTPYTFGGGTKLEIK

SEQ ID NO: 31 - DNA, 792 bp, synthetic construct
  source 1..792: mol_type = other DNA
atggccctcccggtaacggctctgctgcttccactcgcactgctcttgcatgctgccagaccagaagtgaagctggtggagtctgggggaggcttagtgaagcctggagcgtctctgaaactctcctgtgcagcctctggattcactttcagtaactatggcatgtcttgggttcgccagacttcagacaagaggctggagtgggtcgcatccattagtagtggtggtggtgacacctactatccagacaatgtaaagggccgattcaccatctccagagagaatgccaagaacaccctgtacctgcaaatgagtagtctgaactctgaggacacggccttgtattactgtgcaagagactatggtggtacttgggactactttgactactggggccaaggcaccactctcacagtctcctcaggtggaggtggatcaggtggaggtggatctggtggaggtggatctgatatccagatgacacagactacatcctccctgtctgcctctctgggagacagagtcaccatcagttgcagggcaagtcaggacattagcaagtatttaaactggtatcagcagaaaccagatggaactgttaaactcctgatctactacacatcaagattacactcaggagtcccatcgaggttcagtggcagtgggtctggaacagattattctctcaccattagcaacctggagcaagaagatattgccacttacttttgccaacagggtgatacgtttccgtggacgttcggtggaggcaccaagctggaaatcaaacgg

SEQ ID NO: 32 - DNA, 798 bp, synthetic construct
  source 1..798: mol_type = other DNA
atggccctcccggtaacggctctgctgcttccactcgcactgctcttgcatgctgccagaccacaggtccaactgcagcagcctggggctgagctggtgaggcctggagtttcagtgaagctgtcctgcaaggcttctggctacaccttcaccagctactggatgaactgggtgaagcagaggcctggacaaggccttgagtggattggcatgattcatccttccgatagtgaaactaggttaaatcagaagttcaaggacaaggccatattgactgtagacaaatcctccagcacagcctacatgcaactcagcagcccgacatctgaggactctgcggtctattactgtgcaagagaagagggacagctcgggcacggcggtgctatggactactggggtcaaggaacctcagtcaccgtctcctcaggtggaggtggatcaggtggaggtggatctggtggaggtggatctgatatccagatgacacagactacatcctccctgtctgcctctctgggagacagagtcaccatcagttgcagggcaagtcaggacattagcaagtatttaaactggtatcagcagaaaccagatggaactgttaaactcctgatctactacacatcaagattacactcaggagtcccatcgaggttcagtggcagtgggtctggaacagattattctctcaccattagcaacctggagcaagaagatattgccacttacttttgccaacagggtgatacgtttccgtggacgttcggtggaggcaccaagctggaaatcaaacgg

SEQ ID NO: 33 - DNA, 783 bp, synthetic construct
  source 1..783: mol_type = other DNA
atggccctcccggtaacggctctgctgcttccactcgcactgctcttgcatgctgccagaccacaggtgcagctgaaggagtcaggacctggcctggtggcaccctcacagagcctgtccatcacatgcacggtctctgggttctcattatccagatatagtgtacactgggttcgccagcctccaggaaagggtctggagtggctgggaatgatatggggtggtggatacacagactataattcagctctcaaatccagactgagcatcagcaaggacaactccaagagccaagttttcttaaaaatgaacagtctgcaaactgatgacacagccatgtactactgtgccagatatatagacagctcgggctacgactactggggccaaggcaccactctcacagtctcctcaggtggaggtggatcaggtggaggtggatctggtggaggtggatctagtattgtgatgacccagactcccaaattcctgcttgtatcagcaggagacagggttaccataacctgcaaggccagtcagactgtgaatgatgatgtagcttggtatcaacagaagccaggacagtctcctaaattgctgatatattatgtatccaatcgccacactggagtccctgatcgcttcactggcagtggatatgggacggatttcactttcaccatcagcactgtgcaggctgaagacctggcagtttatttctgtcagcaggattatagctctccgtacacgttcggaggggggaccaagctggaaataaaacgg

SEQ ID NO: 34 - DNA, 780 bp, synthetic construct
  source 1..780: mol_type = other DNA
atggccctcccggtaacggctctgctgcttccactcgcactgctcttgcatgctgccagaccacaggtgcagctgaaggagtcaggacctggcctggtggcaccctcacagagcctgtccatcacatgcacggtctctgggttctcattatccagatatagtgtacactgggttcgccagcctccaggaaagggtctggagtggctgggaatgatatggggtggtggatacacagactataattcagctctcaaatccagactgagcatcagcaaggacaactccaagagccaagttttcttaaaaatgaacagtctgcaaactgatgacacagccatgtactactgtgccagatatatagacagctcgggctacgactactggggccaaggcaccactctcacagtctcctcaggtggaggtggatcaggtggaggtggatctggtggaggtggatctcaaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccactcacgttcggtgctgggaccaagctggagctgaaacgg

SEQ ID NO: 35 - DNA, 783 bp, synthetic construct
  source 1..783: mol_type = other DNA
atggccctcccggtaacggctctgctgcttccactcgcactgctcttgcatgctgccagaccacaggtgcagctgaaggagtcaggacctggcctggtggcaccctcacagagcctgtccatcacatgcacggtctctgggttctcattatccagatatagtgtacactgggttcgccagcctccaggaaagggtctggagtggctgggaatgatatggggtggtggatacacagactataattcagctctcaaatccagactgagcatcagcaaggacaactccaagagccaagttttcttaaaaatgaacagtctgcaaactgatgacacagccatgtactactgtgccagatatatagacagctcgggctacgactactggggccaaggcaccactctcacagtctcctcaggtggaggtggatcaggtggaggtggatctggtggaggtggatctgacatccagatgactcagtctccagcctccctatctgcatctgtgggagaaactgtcaccatcacatgtcgagcaagtgagaatatttacagttatttagcatggtatcagcagaaacagggaaaatctcctcagctcctggtctataatgcaaaaaccttagcagaaggtgtgccatcaaggttcagtggcagtggatcaggcacacagttttctctgaagatcaacagtctgcagcctgaagattttgggagttattactgtcaacatcattatggtactccgtacacgttcggaggggggaccaagctggaaataaaacgg

SEQ ID NO: 36 - protein, 8 aa, synthetic construct
GYTFTDYN

SEQ ID NO: 37 - protein, 8 aa, synthetic construct
INPNNGGT

SEQ ID NO: 38 - protein, 14 aa, synthetic construct
ARKGYGGNYDYFDY

SEQ ID NO: 39 - protein, 6 aa, synthetic construct
QSIGTS

SEQ ID NO: 40 - protein, 9 aa, synthetic construct
QQSNSWPYT

SEQ ID NO: 41 - protein, 8 aa, synthetic construct
GFNIKDTY

SEQ ID NO: 42 - protein, 10 aa, synthetic construct
GFSLSTYGMG

SEQ ID NO: 43 - protein, 8 aa, synthetic construct
IDPANGNT

SEQ ID NO: 44 - protein, 7 aa, synthetic construct
IYWDDDK

SEQ ID NO: 45 - protein, 11 aa, synthetic construct
ALYYYGGSLDY

SEQ ID NO: 46 - protein, 14 aa, synthetic construct
AQSLIYDGYYGFAY

SEQ ID NO: 47 - protein, 12 aa, synthetic construct
QSLLYSGNQKNY

SEQ ID NO: 48 - protein, 9 aa, synthetic construct
QQYYSYPRT

SEQ ID NO: 49 - protein, 8 aa, synthetic construct
GYTFTYYG

SEQ ID NO: 50 - protein, 8 aa, synthetic construct
INTYSGVP

SEQ ID NO: 51 - protein, 14 aa, synthetic construct
ARWIYYSDLYGMDY

SEQ ID NO: 52 - protein, 11 aa, synthetic construct
QSIVHSNGDTY

SEQ ID NO: 53 - protein, 9 aa, synthetic construct
FQGSHVPWT

SEQ ID NO: 54 - protein, 15 aa, synthetic construct
GGGGSGGGGSGGGGS

SEQ ID NO: 55 - protein, 121 aa, synthetic construct
EVQLQQSGPELVKPGSSVKISCKASGYTFTDYNMDWVKQSHGKSLEWIGTINPNNGGTSYNQKFKGKATLTVDKSSSTAYMELRSLTSEDSAVYYCARKGYGGNYDYFDYWGQGTTLTVSS

SEQ ID NO: 56 - protein, 118 aa, synthetic construct
EVQLQQSGAELVKPGASVKLSCTASGFNIKDTYMHWVKQRPEQGLEWIGRIDPANGNTIYASKFQGKATITADTSSNTAYMQLSSLTSGDTAVYYCALYYYGGSLDYWGQGTTLTVSS

SEQ ID NO: 57 - protein, 122 aa, synthetic construct
QVTLKESGPGILQPSQTLSLTCSFSGFSLSTYGMGVSWIRQPSGKGLEWLAHIYWDDDKRYNPSLKSRLTISKDTSNNQVFLKITSVDTADTATYYCAQSLIYDGYYGFAYWGQGTLVTVSA

SEQ ID NO: 58 - protein, 121 aa, synthetic construct
QIQLVQSGPELKKPGETVKISCKASGYTFTYYGMNWVKQAPGKGLEWMGWINTYSGVPTYADDFKGRFAFSLETSVSTAYLQINNLKNEDTATYFCARWIYYSDLYGMDYWGQGTSVTVSS

SEQ ID NO: 59 - protein, 120 aa, synthetic construct
QVQLQQSGAELAKPGASVKMSCKASGYTFSSYWMHWLKQRPGQGLEWIGYINPSSGYTNYNQKFKDKATLTADKSSSTAYMQLSSLTSEDSAVYYCARDGNYDHWYFDVWGTGTTVTVSS

SEQ ID NO: 60 - protein, 119 aa, synthetic construct
QVQLQQPGAELVRPGASVKMSCKASGYTLTTYLMDWVKQRLGQGFEWIGNINPNSGSSNYNEKFKGKAKLTVDKSSSTAYMQLSSLTSEDSAVYYCAIRHYGGSLFDYWGQGTTLTVSS

SEQ ID NO: 61 - protein, 107 aa, synthetic construct
DILLTQSPAILSVSPGERVSFSCRASQSIGTSIHWYQQRTNGSPRLLIKYASESISGIPSRFSGSGSGTDFTLSINSVESEDIADYYCQQSNSWPYTFGGGTKLEIK

SEQ ID NO: 62 - protein, 113 aa, synthetic construct
DIVMSQSPSSLAVSVGERVTMSCKSSQSLLYSGNQKNYLAWYQQKPGQSPKLLIYWASTRESGVPDRFTGSGSGTDFTLTISSVKAEDLAVYYCQQYYSYPRTFGGGTKLEIK

SEQ ID NO: 63 - protein, 112 aa, synthetic construct
DVLMTQSPLSLPVSLGDQASISCRSSQSIVHSNGDTYLEWYLQKPGQSPKLLIYKVSNRFSGVPDRFSGSGSGTDFTLKISRVEAEDLGVYHCFQGSHVPWTFGGGTKLEIK

SEQ ID NO: 64 - protein, 106 aa, synthetic construct
DIKMTQSPSSMYASLGERVTITCKASQDINSYLSWFQQKPGKSPKTLIYRANRLVDGVPSRFSGSGSGQDYSLTISSLEYEDMGIYYCLQYDELLTFGAGTKLELK

SEQ ID NO: 65 - protein, 112 aa, synthetic construct
DIVMSQSPSSLAVSAGERVTMSCRSSQSLLNSRTRKNYLAWYQQKPGQSPKLLIYWASTRESGVPDRFSGSGSGTDFTLTISSVQAEDLAVYYCEQSYNLFTFGSGTKLEIK

SEQ ID NO: 66 - protein, 243 aa, synthetic construct
EVQLQQSGPELVKPGSSVKISCKASGYTFTDYNMDWVKQSHGKSLEWIGTINPNNGGTSYNQKFKGKATLTVDKSSSTAYMELRSLTSEDSAVYYCARKGYGGNYDYFDYWGQGTTLTVSSGGGGSGGGGSGGGGSDILLTQSPAILSVSPGERVSFSCRASQSIGTSIHWYQQRTNGSPRLLIKYASESISGIPSRFSGSGSGTDFTLSINSVESEDIADYYCQQSNSWPYTFGGGTKLEIK

SEQ ID NO: 67 - protein, 246 aa, synthetic construct
EVQLQQSGAELVKPGASVKLSCTASGFNIKDTYMHWVKQRPEQGLEWIGRIDPANGNTIYASKFQGKATITADTSSNTAYMQLSSLTSGDTAVYYCALYYYGGSLDYWGQGTTLTVSSGGGGSGGGGSGGGGSDIVMSQSPSSLAVSVGERVTMSCKSSQSLLYSGNQKNYLAWYQQKPGQSPKLLIYWASTRESGVPDRFTGSGSGTDFTLTISSVKAEDLAVYYCQQYYSYPRTFGGGTKLEIK

SEQ ID NO: 68 - protein, 250 aa, synthetic construct
QVTLKESGPGILQPSQTLSLTCSFSGFSLSTYGMGVSWIRQPSGKGLEWLAHIYWDDDKRYNPSLKSRLTISKDTSNNQVFLKITSVDTADTATYYCAQSLIYDGYYGFAYWGQGTLVTVSAGGGGSGGGGSGGGGSDIVMSQSPSSLAVSVGERVTMSCKSSQSLLYSGNQKNYLAWYQQKPGQSPKLLIYWASTRESGVPDRFTGSGSGTDFTLTISSVKAEDLAVYYCQQYYSYPRTFGGGTKLEIK

SEQ ID NO: 69 - protein, 248 aa, synthetic construct
QIQLVQSGPELKKPGETVKISCKASGYTFTYYGMNWVKQAPGKGLEWMGWINTYSGVPTYADDFKGRFAFSLETSVSTAYLQINNLKNEDTATYFCARWIYYSDLYGMDYWGQGTSVTVSSGGGGSGGGGSGGGGSDVLMTQSPLSLPVSLGDQASISCRSSQSIVHSNGDTYLEWYLQKPGQSPKLLIYKVSNRFSGVPDRFSGSGSGTDFTLKISRVEAEDLGVYHCFQGSHVPWTFGGGTKLEIK

SEQ ID NO: 70 - protein, 241 aa, synthetic construct
QVQLQQSGAELAKPGASVKMSCKASGYTFSSYWMHWLKQRPGQGLEWIGYINPSSGYTNYNQKFKDKATLTADKSSSTAYMQLSSLTSEDSAVYYCARDGNYDHWYFDVWGTGTTVTVSSGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDELLTFGAGTKLELK

SEQ ID NO: 71 - protein, 247 aa, synthetic construct
QVQLQQSGAELAKPGASVKMSCKASGYTFSSYWMHWLKQRPGQGLEWIGYINPSSGYTNYNQKFKDKATLTADKSSSTAYMQLSSLTSEDSAVYYCARDGNYDHWYFDVWGTGTTVTVSSGGGGSGGGGSGGGGSDIVMSQSPSSLAVSAGERVTMSCRSSQSLLNSRTRKNYLAWYQQKPGQSPKLLIYWASTRESGVPDRFSGSGSGTDFTLTISSVQAEDLAVYYCEQSYNLFTFGSGTKLEIK

SEQ ID NO: 72 - protein, 240 aa, synthetic construct
QVQLQQPGAELVRPGASVKMSCKASGYTLTTYLMDWVKQRLGQGFEWIGNINPNSGSSNYNEKFKGKAKLTVDKSSSTAYMQLSSLTSEDSAVYYCAIRHYGGSLFDYWGQGTTLTVSSGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDELLTFGAGTKLELK

SEQ ID NO: 73 - protein, 246 aa, synthetic construct
QVQLQQPGAELVRPGASVKMSCKASGYTLTTYLMDWVKQRLGQGFEWIGNINPNSGSSNYNEKFKGKAKLTVDKSSSTAYMQLSSLTSEDSAVYYCAIRHYGGSLFDYWGQGTTLTVSSGGGGSGGGGSGGGGSDIVMSQSPSSLAVSAGERVTMSCRSSQSLLNSRTRKNYLAWYQQKPGQSPKLLIYWASTRESGVPDRFSGSGSGTDFTLTISSVQAEDLAVYYCEQSYNLFTFGSGTKLEIK

SEQ ID NO: 74 - DNA, 806 bp, synthetic construct
  source 1..806: mol_type = other DNA
ccatggccctcccggtaacggctctgctgcttccactcgcactgctcttgcatgctgccagaccagaggtccagctgcaacagtctggacctgagctggtgaagcctgggtcttcagtgaagatatcctgcaaagcttctggatacacattcactgactacaacatggactgggtgaagcagagtcatggaaagagccttgagtggattggaactattaatcctaacaatggtggtactagctacaaccagaagttcaagggcaaggccacattgactgtagacaagtcctccagcacagcctacatggagctccgcagcctgacatctgaagactctgcagtctattactgtgcaagaaagggctatggtggtaactacgactactttgactactggggccaaggcaccactctcacagtctcctcaggtggaggtggatcaggtggaggtggatctggtggaggtggatctgacatcttgctgactcagtctccagccatcctgtctgtgagtccaggagaaagagtcagtttctcctgcagggccagtcagagcattggcacaagcatacactggtatcagcaaagaacaaatggttctccaaggcttctcataaagtatgcttctgagtctatctctgggttcccttccaggtttagtggcagtggatcagggacagattttactcttagcatcaacagtgtggagtctgaagatattgcagattattactgtcaacaaagtaatagctggccgtacacgttcggaggggggaccaagctggaaataaaacgggcggccgca

SEQ ID NO: 75 - DNA, 804 bp, synthetic construct
  source 1..804: mol_type = other DNA
atggccctcccggtaacggctctgctgcttccactcgcactgctcttgcatgctgccagaccagaggttcagctgcagcagtctggggcagagcttgtgaagccaggggcctcagtcaagttgtcctgcacagcttctggcttcaacattaaagacacctatatgcactgggtgaagcagaggcctgaacagggcctggagtggattggaaggattgatcctgcgaatggtaatactatatatgcctcaaagttccagggcaaggccactataacagcagacacatcatccaacacagcctacatgcagctcagcagcctgacatctggggacactgccgtctattactgtgctctttattactatggtggtagccttgactactggggccaaggcaccactctcacagtctcctcaggtggaggtggatcaggtggaggtggatctggtggaggtggatctgacattgtgatgtcacagtctccatcctccctagctgtgtcagttggagagagggttactatgagctgcaagtccagtcagagccttttatatagtggcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatcctcggacgttcggtggaggcaccaagctggaaatcaaacgg

SEQ ID NO: 76 - protein, 816 aa, synthetic construct
ATGGCCCTCCCGGTAACGGCTCTGCTGCTTCCACTCGCACTGCTCTTGCATGCTGCCAGACCACAGGTTACTCTGAAAGAGTCTGGCCCTGGGATATTGCAGCCCTCCCAGACCCTCAGTCTGACTTGTTCTTTCTCTGGGTTTTCACTGAGCACTTATGGTATGGGTGTGAGCTGGATTCGTCAGCCTTCAGGAAAGGGTCTGGAGTGGCTGGCACACATTTACTGGGATGATGACAAGCGCTATAACCCATCCCTGAAGAGCCGGCTCACAATCTCCAAGGATACCTCCAACAACCAGGTATTCCTCAAGATCACCAGTGTGGACACTGCAGATACTGCCACATACTACTGTGCTCAAAGCCTGATCTATGATGGTTACTACGGGTTTGCCTACTGGGGCCAAGGGACTCTGGTCACTGTCTCTGCAGGTGGAGGTGGATCAGGTGGAGGTGGATCTGGTGGAGGTGGATCTGACATTGTGATGTCACAGTCTCCATCCTCCCTAGCTGTGTCAGTTGGAGAGAGGGTTACTATGAGCTGCAAGTCCAGTCAGAGCCTTTTATATAGTGGCAATCAAAAGAACTACTTGGCCTGGTACCAGCAGAAACCAGGGCAGTCTCCTAAACTGCTGATTTACTGGGCATCCACTAGGGAATCTGGGGTCCCTGATCGCTTCACAGGCAGTGGATCTGGGACAGATTTCACTCTCACCATCAGCAGTGTGAAGGCTGAAGACCTGGCAGTTTATTACTGTCAGCAATATTATAGCTATCCTCGGACGTTCGGTGGAGGCACCAAGCTGGAAATCAAACGG

SEQ ID NO: 77 - protein, 810 aa, synthetic construct
ATGGCCCTCCCGGTAACGGCTCTGCTGCTTCCACTCGCACTGCTCTTGCATGCTGCCAGACCACAGATCCAGTTGGTGCAATCTGGACCTGAGCTGAAGAAGCCTGGAGAGACAGTCAAGATCTCCTGCAAGGCTTCTGGGTATACCTTCACATACTATGGAATGAACTGGGTGAAGCAGGCTCCAGGAAAGGGTTTAGAGTGGATGGGCTGGATAAACACCTACTCTGGAGTGCCAACATATGCTGATGACTTCAAGGGACGGTTTGCCTTCTCTTTGGAAACCTCTGTCAGCACTGCCTATTTGCAGATCAACAACCTCAAAAATGAGGACACGGCTACATATTTTTGTGCAAGATGGATCTACTATAGTGACCTCTATGGTATGGACTACTGGGGTCAAGGAACCTCAGTCACCGTCTCCTCAGGTGGAGGTGGATCAGGTGGAGGTGGATCTGGTGGAGGTGGATCTGATGTTTTGATGACCCAAAGTCCACTCTCCCTGCCTGTCAGTCTTGGAGATCAAGCCTCCATCTCTTGTAGATCTAGTCAGAGTATTGTACATAGTAATGGAGACACGTATTTAGAATGGTATTTGCAGAAACCAGGCCAGTCTCCAAAGCTCCTGATCTACAAAGTTTCTAACCGATTTTCTGGGGTCCCAGACAGGTTCAGTGGCAGTGGATCAGGGACAGATTTCACACTCAAGATCAGCAGAGTGGAGGCTGAGGATCTGGGAGTTTATCACTGCTTTCAAGGTTCACATGTTCCGTGGACGTTCGGTGGAGGCACCAAGCTGGAAATCAAACGG

SEQ ID NO: 78 - protein, 789 aa, synthetic construct
ATGGCCCTCCCGGTAACGGCTCTGCTGCTTCCACTCGCACTGCTCTTGCATGCTGCCAGACCACAGGTCCAGCTGCAGCAGTCTGGGGCTGAACTGGCAAAACCTGGGGCCTCAGTGAAGATGTCCTGCAAGGCTTCTGGCTACACCTTTTCTAGCTACTGGATGCACTGGCTAAAACAGAGGCCTGGACAGGGTCTGGAGTGGATTGGATACATTAATCCTAGCAGTGGTTATACTAACTACAATCAGAAGTTCAAGGACAAGGCCACATTGACTGCAGACAAATCCTCCAGCACAGCCTACATGCAACTGAGCAGCCTGACATCTGAGGACTCTGCAGTCTATTACTGTGCAAGAGATGGTAACTATGACCACTGGTACTTCGATGTCTGGGGCACAGGGACCACGGTCACCGTCTCCTCAGGTGGAGGTGGATCAGGTGGAGGTGGATCTGGTGGAGGTGGATCTGACATCAAGATGACCCAGTCTCCATCTTCCATGTATGCATCTCTAGGAGAGAGAGTCACTATCACTTGCAAGGCGAGTCAGGACATTAATAGCTATTTAAGCTGGTTCCAGCAGAAACCAGGGAAATCTCCTAAGACCCTGATCTATCGTGCAAACAGATTGGTAGATGGGGTCCCATCAAGGTTCAGTGGCAGTGGATCTGGGCAAGATTATTCTCTCACCATCAGCAGCCTGGAGTATGAAGATATGGGAATTTATTATTGTCTACAGTATGATGAGTTGCTCACGTTCGGTGCTGGGACCAAGCTGGAGCTGAAACGG

SEQ ID NO: 79 - protein, 807 aa, synthetic construct
ATGGCCCTCCCGGTAACGGCTCTGCTGCTTCCACTCGCACTGCTCTTGCATGCTGCCAGACCACAGGTCCAGCTGCAGCAGTCTGGGGCTGAACTGGCAAAACCTGGGGCCTCAGTGAAGATGTCCTGCAAGGCTTCTGGCTACACCTTTTCTAGCTACTGGATGCACTGGCTAAAACAGAGGCCTGGACAGGGTCTGGAGTGGATTGGATACATTAATCCTAGCAGTGGTTATACTAACTACAATCAGAAGTTCAAGGACAAGGCCACATTGACTGCAGACAAATCCTCCAGCACAGCCTACATGCAACTGAGCAGCCTGACATCTGAGGACTCTGCAGTCTATTACTGTGCAAGAGATGGTAACTATGACCACTGGTACTTCGATGTCTGGGGCACAGGGACCACGGTCACCGTCTCCTCAGGTGGAGGTGGATCAGGTGGAGGTGGATCTGGTGGAGGTGGATCTGACATTGTGATGTCACAGTCTCCATCCTCCCTGGCTGTGTCAGCAGGAGAGAGGGTCACTATGAGCTGCAGATCCAGTCAGAGTCTGCTCAACAGTAGAACCCGAAAGAACTACTTGGCTTGGTACCAGCAGAAACCAGGGCAGTCTCCTAAGCTGCTGATCTACTGGGCATCCACTAGGGAATCTGGGGTCCCTGATCGCTTCTCAGGCAGTGGATCTGGGACAGATTTCACTCTCACCATCAGCAGTGTGCAGGCTGAAGACCTGGCAGTTTATTACTGCGAGCAATCTTATAATCTATTCACGTTCGGCTCGGGGACAAAGTTGGAAATAAAACGG

SEQ ID NO: 80 - protein, 786 aa, synthetic construct
ATGGCCCTCCCGGTAACGGCTCTGCTGCTTCCACTCGCACTGCTCTTGCATGCTGCCAGACCACAGGTTCAACTGCAGCAGCCTGGGGCTGAGCTGGTGAGGCCTGGGGCTTCAGTGAAGATGTCCTGCAAGGCTTCTGGCTACACCCTCACCACCTACTTGATGGACTGGGTAAAACAGAGGCTTGGACAAGGCTTTGAGTGGATTGGAAATATTAATCCTAATAGTGGTAGTAGTAACTACAATGAGAAGTTCAAGGGCAAGGCCAAGCTGACTGTAGACAAATCCTCCAGCACAGCCTACATGCAACTCAGCAGCCTGACATCTGAGGACTCTGCGGTCTATTACTGTGCAATACGGCACTATGGTGGTAGTCTCTTTGACTACTGGGGCCAAGGCACCACTCTCACAGTCTCCTCAGGTGGAGGTGGATCAGGTGGAGGTGGATCTGGTGGAGGTGGATCTGACATCAAGATGACCCAGTCTCCATCTTCCATGTATGCATCTCTAGGAGAGAGAGTCACTATCACTTGCAAGGCGAGTCAGGACATTAATAGCTATTTAAGCTGGTTCCAGCAGAAACCAGGGAAATCTCCTAAGACCCTGATCTATCGTGCAAACAGATTGGTAGATGGGGTCCCATCAAGGTTCAGTGGCAGTGGATCTGGGCAAGATTATTCTCTCACCATCAGCAGCCTGGAGTATGAAGATATGGGAATTTATTATTGTCTACAGTATGATGAGTTGCTCACGTTCGGTGCTGGGACCAAGCTGGAGCTGAAACGG

SEQ ID NO: 81 - protein, 804 aa, synthetic construct
ATGGCCCTCCCGGTAACGGCTCTGCTGCTTCCACTCGCACTGCTCTTGCATGCTGCCAGACCACAGGTTCAACTGCAGCAGCCTGGGGCTGAGCTGGTGAGGCCTGGGGCTTCAGTGAAGATGTCCTGCAAGGCTTCTGGCTACACCCTCACCACCTACTTGATGGACTGGGTAAAACAGAGGCTTGGACAAGGCTTTGAGTGGATTGGAAATATTAATCCTAATAGTGGTAGTAGTAACTACAATGAGAAGTTCAAGGGCAAGGCCAAGCTGACTGTAGACAAATCCTCCAGCACAGCCTACATGCAACTCAGCAGCCTGACATCTGAGGACTCTGCGGTCTATTACTGTGCAATACGGCACTATGGTGGTAGTCTCTTTGACTACTGGGGCCAAGGCACCACTCTCACAGTCTCCTCAGGTGGAGGTGGATCAGGTGGAGGTGGATCTGGTGGAGGTGGATCTGACATTGTGATGTCACAGTCTCCATCCTCCCTGGCTGTGTCAGCAGGAGAGAGGGTCACTATGAGCTGCAGATCCAGTCAGAGTCTGCTCAACAGTAGAACCCGAAAGAACTACTTGGCTTGGTACCAGCAGAAACCAGGGCAGTCTCCTAAGCTGCTGATCTACTGGGCATCCACTAGGGAATCTGGGGTCCCTGATCGCTTCTCAGGCAGTGGATCTGGGACAGATTTCACTCTCACCATCAGCAGTGTGCAGGCTGAAGACCTGGCAGTTTATTACTGCGAGCAATCTTATAATCTATTCACGTTCGGCTCGGGGACAAAGTTGGAAATAAAACGG

SEQ ID NO: 82 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 83 - protein, 42 aa, synthetic construct
KRGRKKLLYIFKQPFMRPVQTTQEEDGCSCRFPEEEEGGCEL

SEQ ID NO: 84 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 154 aa, synthetic construct
KRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 8 aa, synthetic construct
GFDIKDTY

SEQ ID NO: 87 - protein, 5 aa, synthetic construct
TYAMY

SEQ ID NO: 88 - protein, 4 aa, synthetic construct
TFWM

SEQ ID NO: 89 - protein, 5 aa, synthetic construct
TFWMQ

SEQ ID NO: 90 - protein, 8 aa, synthetic construct
IDPANGDT

SEQ ID NO: 91 - protein, 22 aa, synthetic construct
RIRSKVNNYATYYADSVKDRFT

SEQ ID NO: 92 - protein, 20 aa, synthetic construct
TIYPGDDDTRYTQKFKGRAT

SEQ ID NO: 93 - protein, 7 aa, synthetic construct
ARRGGLS

SEQ ID NO: 94 - protein, 5 aa, synthetic construct
DPMDY

SEQ ID NO: 95 - protein, 12 aa, synthetic construct
SGYERGPYYFDS

SEQ ID NO: 96 - protein, 10 aa, synthetic construct
SGYERGPYYF

SEQ ID NO: 97 - protein, 6 aa, synthetic construct
GNIHNY

SEQ ID NO: 98 - protein, 16 aa, synthetic construct
GSSKSLLHSNGNTYLY

SEQ ID NO: 99 - protein, 17 aa, synthetic construct
KSSQSLLCRSNQKNYLA

SEQ ID NO: 100 - protein, 17 aa, synthetic construct
KSSQSLLYRSNQKNYLA

SEQ ID NO: 101 - protein, 7 aa, synthetic construct
RVSNLAS

SEQ ID NO: 102 - protein, 7 aa, synthetic construct
WASTRES

SEQ ID NO: 103 - protein, 9 aa, synthetic construct
QHFWSTPWT

SEQ ID NO: 104 - protein, 9 aa, synthetic construct
MQHLEYPYT

SEQ ID NO: 105 - protein, 9 aa, synthetic construct
QQYYSYPLT

SEQ ID NO: 106 - protein, 114 aa, synthetic construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSS

SEQ ID NO: 107 - protein, 116 aa, synthetic construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 108 - protein, 116 aa, synthetic construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 109 - protein, 121 aa, synthetic construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 110 - protein, 121 aa, synthetic construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 111 - protein, 121 aa, synthetic construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 112 - protein, 107 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 113 - protein, 112 aa, synthetic construct
GNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 114 - protein, 112 aa, synthetic construct
GNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 115 - protein, 112 aa, synthetic construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 116 - protein, 112 aa, synthetic construct; identical to SEQ ID NO: 115

SEQ ID NO: 117 - protein, 113 aa, synthetic construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 118 - protein, 113 aa, synthetic construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 119 - protein, 236 aa, synthetic construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 120 - protein, 241 aa, synthetic construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 121 - protein, 241 aa, synthetic construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSGNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 122 - protein, 241 aa, synthetic construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 123 - protein, 242 aa, synthetic construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 124 - protein, 242 aa, synthetic construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 125 - protein, 238 aa, unidentified
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 126 - protein, 243 aa, synthetic construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 127 - protein, 243 aa, synthetic construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSGNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 128 - protein, 243 aa, synthetic construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 129 - protein, 244 aa, synthetic construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 130 - protein, 244 aa, synthetic construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 131 - protein, 238 aa, synthetic construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 132 - protein, 243 aa, synthetic construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 133 - protein, 243 aa, synthetic construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSGNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 134 - protein, 243 aa, synthetic construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 135 - protein, 244 aa, synthetic construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 136 - protein, 244 aa, synthetic construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 137 - protein, 243 aa, synthetic construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 138 - protein, 248 aa, synthetic construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 139 - protein, 248 aa, synthetic construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 140 - protein, 248 aa, synthetic construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 141 - protein, 249 aa, synthetic construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 142 - protein, 249 aa, synthetic construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 143 - protein, 243 aa, synthetic construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 144 - protein, 248 aa, synthetic construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 145 - protein, 248 aa, synthetic construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 146 - protein, 248 aa, synthetic construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 147 - protein, 248 aa, synthetic construct; identical to SEQ ID NO: 146

SEQ ID NO: 148 - protein, 249 aa, synthetic construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 149 - protein, 249 aa, synthetic construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 150 - protein, 243 aa, synthetic construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 151 - protein, 248 aa, synthetic construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 152 - protein, 248 aa, synthetic construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 153 - protein, 248 aa, synthetic construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 154 - protein, 248 aa, synthetic construct; identical to SEQ ID NO: 153

SEQ ID NO: 155 - protein, 249 aa, synthetic construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 156 - protein, 249 aa, synthetic construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 157 - protein, 236 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSEVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSS

SEQ ID NO: 158 - protein, 238 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSEVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 159 - protein, 238 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSEVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 160 - protein, 243 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSQVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 161 - protein, 243 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSDVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 162 - protein, 243 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSQVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 163 - protein, 241 aa, synthetic construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSEVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSS

SEQ ID NO: 164 - protein, 243 aa, synthetic construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSEVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 165 - protein, 243 aa, synthetic construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSEVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 166 - protein, 248 aa, synthetic construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSQVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 167 - protein, 248 aa, synthetic construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSDVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 168 - protein, 248 aa, synthetic construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSQVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 169 - protein, 242 aa, synthetic construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSS

SEQ ID NO: 170 - protein, 244 aa, synthetic construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 171 - protein, 244 aa, synthetic construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 172 - protein, 249 aa, synthetic construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSQVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 173 - protein, 249 aa, synthetic construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSDVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 174 - protein, 249 aa, synthetic construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSQVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 175 - protein, 242 aa, synthetic construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSS

SEQ ID NO: 176 - protein, 244 aa, synthetic construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 177 - protein, 244 aa, synthetic construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 178 - protein, 249 aa, synthetic construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSQVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 179 - protein, 249 aa, synthetic construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSDVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 180 - protein, 249 aa, synthetic construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSQVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 181 - protein, 8 aa, synthetic construct
GFTFSSFA

SEQ ID NO: 182 - protein, 5 aa, synthetic construct
SFAVS

SEQ ID NO: 183 - protein, 5 aa, synthetic construct
SHDMS

SEQ ID NO: 184 - protein, 8 aa, synthetic construct
ISSGGAYT

SEQ ID NO: 185 - protein, 20 aa, synthetic construct
TISSGGAYTFYKDSVKGRFT

SEQ ID NO: 186 - protein, 20 aa, synthetic construct
YISGGGTNIYYSDTVKGRFT

SEQ ID NO: 187 - protein, 16 aa, synthetic construct
ARHSGYDGYYLYAMDY

SEQ ID NO: 188 - protein, 14 aa, synthetic construct
HSGYDGYYLYAMDY

SEQ ID NO: 189 - protein, 12 aa, synthetic construct
PNYNYGGSWFAY

SEQ ID NO: 190 - protein, 5 aa, synthetic construct
SSVHY

SEQ ID NO: 191 - protein, 9 aa, synthetic construct
ASSSVHYMH

SEQ ID NO: 192 - protein, 10 aa, synthetic construct
SASSSVHYMH

SEQ ID NO: 193 - protein, 7 aa, synthetic construct
DTSKLAS

SEQ ID NO: 194 - protein, 9 aa, synthetic construct
QQWTSNPPT

SEQ ID NO: 195 - protein, 123 aa, synthetic construct
ELILVESGGGLVKPGGSLKLSCAVSGFTFSSFAMSWVRQTPEKRLEWVATISSGGAYTFYKDSVKGRFTISRDNAKNTLYLQMSSLRSEDSAMYYCARHSGYDGYYLYAMDYWGQGTSVTVSS

SEQ ID NO: 196 - protein, 127 aa, synthetic construct
GVQCELILVESGGGLVKPGGSLKLSCAVSGFTFSSFAVSWVRQTPEKRLEWVATISSGGAYTFYKDSVKGRFTISRDNAKNTLYLQMSSLRSEDSAMYYCARHSGYDGYYLYAMDYWGQGTSVTVSS

SEQ ID NO: 197 - protein, 121 aa, synthetic construct
EVQLEESGGGLVQPGGSLKVSCAVSGLAFSSHDMSWVRQTPEKRLEWVAYISGGGTNIYYSDTVKGRFTISRDNAKNTLYLQMSSLKSEDTAIYYCARPNYNYGGSWFAYWGQGTLVTVSA

SEQ ID NO: 198 - protein, 106 aa, synthetic construct
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIK

SEQ ID NO: 199 - protein, 244 aa, synthetic construct
ELILVESGGGLVKPGGSLKLSCAVSGFTFSSFAMSWVRQTPEKRLEWVATISSGGAYTFYKDSVKGRFTISRDNAKNTLYLQMSSLRSEDSAMYYCARHSGYDGYYLYAMDYWGQGTSVTVSSGGGGSGGGGSGGGGSQIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIK

SEQ ID NO: 200 - protein, 248 aa, synthetic construct
GVQCELILVESGGGLVKPGGSLKLSCAVSGFTFSSFAVSWVRQTPEKRLEWVATISSGGAYTFYKDSVKGRFTISRDNAKNTLYLQMSSLRSEDSAMYYCARHSGYDGYYLYAMDYWGQGTSVTVSSGGGGSGGGGSGGGGSQIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIK

SEQ ID NO: 201 - protein, 242 aa, synthetic construct
EVQLEESGGGLVQPGGSLKVSCAVSGLAFSSHDMSWVRQTPEKRLEWVAYISGGGTNIYYSDTVKGRFTISRDNAKNTLYLQMSSLKSEDTAIYYCARPNYNYGGSWFAYWGQGTLVTVSAGGGGSGGGGSGGGGSQIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIK

SEQ ID NO: 202 - protein, 244 aa, synthetic construct
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIKGGGGSGGGGSGGGGSELILVESGGGLVKPGGSLKLSCAVSGFTFSSFAMSWVRQTPEKRLEWVATISSGGAYTFYKDSVKGRFTISRDNAKNTLYLQMSSLRSEDSAMYYCARHSGYDGYYLYAMDYWGQGTSVTVSS

SEQ ID NO: 203 - protein, 248 aa, synthetic construct
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIKGGGGSGGGGSGGGGSGVQCELILVESGGGLVKPGGSLKLSCAVSGFTFSSFAVSWVRQTPEKRLEWVATISSGGAYTFYKDSVKGRFTISRDNAKNTLYLQMSSLRSEDSAMYYCARHSGYDGYYLYAMDYWGQGTSVTVSS

SEQ ID NO: 204 - protein, 242 aa, synthetic construct
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIKGGGGSGGGGSGGGGSEVQLEESGGGLVQPGGSLKVSCAVSGLAFSSHDMSWVRQTPEKRLEWVAYISGGGTNIYYSDTVKGRFTISRDNAKNTLYLQMSSLKSEDTAIYYCARPNYNYGGSWFAYWGQGTLVTVSA

SEQ ID NO: 205 - protein, 13 aa, synthetic construct
KASGGTFSSYAIS

SEQ ID NO: 206 - protein, 17 aa, synthetic construct
GIIPIFGTANYAQKFQG

SEQ ID NO: 207 - protein, 19 aa, synthetic construct
AREEGPYCSSTSCYGAFDI

SEQ ID NO: 208 - protein, 11 aa, synthetic construct
QGDSLRSYFAS

SEQ ID NO: 209 - protein, 8 aa, synthetic construct
YARNDRPA

SEQ ID NO: 210 - protein, 11 aa, synthetic construct
AAWDDSLNGYL

SEQ ID NO: 211 - protein, 117 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTV

SEQ ID NO: 212 - protein, 126 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREEGPYCSSTSCYGAFDIWGQGTLVTVSS

SEQ ID NO: 213 - protein, 109 aa, synthetic construct
LLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVA

SEQ ID NO: 214 - protein, 101 aa, synthetic construct
QSVLTQDPAVSVALGQTVKITCQGDSLRSYFASWYQQKPGQAPTLVMYGVPDRFSGSKSGTSASLAISGLQSEDEADYYCAAWDDSLNGYLFGAGTKLTVL

SEQ ID NO: 215 - protein, 242 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREEGPYCSSTSCYGAFDIWGQGTLVTVSSGGGGSGGGGSGGGGSQSVLTQDPAVSVALGQTVKITCQGDSLRSYFASWYQQKPGQAPTLVMYGVPDRFSGSKSGTSASLAISGLQSEDEADYYCAAWDDSLNGYLFGAGTKLTVL

SEQ ID NO: 216 - protein, 242 aa, synthetic construct
QSVLTQDPAVSVALGQTVKITCQGDSLRSYFASWYQQKPGQAPTLVMYGVPDRFSGSKSGTSASLAISGLQSEDEADYYCAAWDDSLNGYLFGAGTKLTVLGGGGSGGGGSGGGGSEVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREEGPYCSSTSCYGAFDIWGQGTLVTVSS

SEQ ID NO: 217 - protein, 5 aa, synthetic construct
NYGMN

SEQ ID NO: 218 - protein, 5 aa, synthetic construct
GYAMS

SEQ ID NO: 219 - protein, 17 aa, synthetic construct
  SITE 12: note = Xaa is Ala or Val
  SITE 13: note = Xaa is Gly orAsp
WINTYTGEPTYXXDFKG

SEQ ID NO: 220 - protein, 17 aa, synthetic construct
TISSGGTYIYYPDSVKG

SEQ ID NO: 221 - protein, 9 aa, synthetic construct
TGTTAILNG

SEQ ID NO: 222 - protein, 9 aa, synthetic construct
SGDGYWYYA

SEQ ID NO: 223 - protein, 10 aa, synthetic construct
  SITE 10: note = Xaa is Gly or Ala
DNYGXXYDYX

SEQ ID NO: 224 - protein, 10 aa, synthetic construct
  SITE 6: note = Xaa is Val or Il
  SITE 9: note = Xaa is Met or Ile
  SITE 10: note = Xaa is His or Tyr
SASSSXSYXX

SEQ ID NO: 225 - protein, 15 aa, synthetic construct
RASKSVSTSGYSYMH

SEQ ID NO: 226 - protein, 7 aa, synthetic construct
  SITE 1: note = Xaa is Ser or Gly
XTSNLAS

SEQ ID NO: 227 - protein, 7 aa, synthetic construct
LASNLES

SEQ ID NO: 228 - protein, 9 aa, synthetic construct
  SITE 5: note = Xaa is Ser or Asn
  SITE 8: note = Xaa is Ser or Phe
QQRSXYPXT

SEQ ID NO: 229 - protein, 9 aa, synthetic construct
QHSRELPFT

SEQ ID NO: 230 - protein, 116 aa, synthetic construct
VQLQESGGGLVQPGGSMKLSCVASGFTFSNYWMNWVRQSPEKGLEWVAEIRLKSNNYATHYAESVKGRFTISRDDSKSSVYLQMNNLRAEDTGIYYCTGVGQFAYWGQGTTVTVSS

SEQ ID NO: 231 - protein, 106 aa, synthetic construct
DIELTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKL

SEQ ID NO: 232 - protein, 106 aa, synthetic construct; identical to SEQ ID NO: 231

SEQ ID NO: 233 - protein, 121 aa, synthetic construct
GGGGSVQLQESGGGLVQPGGSMKLSCVASGFTFSNYWMNWVRQSPEKGLEWVAEIRLKSNNYATHYAESVKGRFTISRDDSKSSVYLQMNNLRAEDTGIYYCTGVGQFAYWGQGTTVTVSS

SEQ ID NO: 234 - protein, 237 aa, synthetic construct
VQLQESGGGLVQPGGSMKLSCVASGFTFSNYWMNWVRQSPEKGLEWVAEIRLKSNNYATHYAESVKGRFTISRDDSKSSVYLQMNNLRAEDTGIYYCTGVGQFAYWGQGTTVTVSSGGGGSGGGGSGGGGSDIELTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKL

SEQ ID NO: 235 - protein, 243 aa, synthetic construct
EIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKGGGGSGGGGSGGGGSEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSS

SEQ ID NO: 236 - protein, 69 aa, synthetic construct; identical to SEQ ID NO: 82

SEQ ID NO: 237 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 238 - protein, 183 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 239 - protein, 138 aa, synthetic construct
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 240 - protein, 207 aa, synthetic construct
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSTTTPAPRPPTPAPTIASQPLSLRPEACRPAAGGAVHTRGLDFACDIYIWAPLAGTCGVLLLSLVITLYC

(19) United States
(12) Patent Application Publication
(10) Pub. No.: US 2025/0302878 A1
(43) Pub. Date: Oct. 2, 2025

(54) NKG2D EXPRESSING CAR-T CELLS

(71) Applicant: H. LEE MOFFITT CANCER CENTER AND RESEARCH INSTITUTE INC., Tampa, FL (US)

(72) Inventor: Marco DAVILA, Tampa, FL (US)

(21) Appl. No.: 18/866,630

(22) PCT Filed: Jun. 14, 2023
(86) PCT No.: PCT/US2023/068414

§ 371 Date: Nov. 18, 2024

Related U.S. Application Data
(60) Provisional application No. 63/366,359, filed on Jun. 14, 2022.
(60) Provisional application No. 63/366,862, filed on Jun. 23, 2022.
(60) Provisional application No. 63/383,993, filed on Nov. 16, 2022.

(51) Int. Cl. A61K 35/17; A61K 40/11; A61K 40/15; A61K 40/31; A61P 35/00; C07K 14/54; C07K 14/705; C07K 16/28; C07K 16/30; C12N 15/85

(52) U.S. Cl. CPC A61K 35/17; A61K 40/11; A61K 40/15; A61K 40/31; A61P 35/00; C07K 14/5434; C07K 14/5443; C07K 14/7056; C07K 16/2803; C07K 16/2818; C07K 16/2827; C07K 16/2851; C07K 16/2863; C07K 16/2866; C07K 16/2896; C07K 16/3092; C12N 15/85; A61K 2239/13; A61K 2239/55

(57) ABSTRACT

Disclosed herein are immune effector cells that are expanded and enriched for NKG2D expression and genetically modified to express chimeric antigen receptor (CAR) polypeptides that can be used with adoptive cell transfer to target and kill CD33-expressing and/or CD123-expressing cancers. In some embodiments, the immune effector cells are gamma-delta (γδ) T cells, Natural Killer (NK) cells, or a combination thereof.

### CROSS-REFERENCE TO RELATED APPLICATIONS

This application claims benefit of U.S. Provisional Application No. 63/366,359, filed Jun. 14, 2022, U.S. Provisional Application No. 63/366,862, filed Jun. 23, 2022, U.S. Provisional Application No. 63/383,993, filed Nov. 16, 2022, which are hereby incorporated herein by reference in their entireties.

### SEQUENCE LISTING

This application contains a sequence listing filed in ST.26 format entitled “320803_2900_Sequence_Listing” created on Jun. 14, 2023, having 248,396 bytes. The content of the sequence listing is incorporated herein in its entirety.

### BACKGROUND

Surgery, radiation therapy, and chemotherapy have been the standard accepted approaches for treatment of cancers including leukemia, solid tumors, and metastases. Immunotherapy (sometimes called biological therapy, biotherapy, or biological response modifier therapy), which uses the body's immune system, either directly or indirectly, to shrink or eradicate cancer has been studied for many years as an adjunct to conventional cancer therapy. It is believed that the human immune system is an untapped resource for cancer therapy and that effective treatment can be developed once the components of the immune system are properly harnessed.

### SUMMARY

Disclosed herein are immune effector cells that are expanded and enriched for NKG2D expression and genetically modified to express chimeric antigen receptor (CAR) polypeptides that can be used with adoptive cell transfer to target and kill CD33-expressing cancers, CD123-expressing cancers, CD99-expressing cancers, CLEC12A-expressing cancers, EGFR-expressing cancers, MUC1-expressing cancers, or a combination thereof.

In some embodiments, the immune effector cells are gamma-delta (γδ) T cells, Natural Killer (NK) cells, or a combination thereof. In some embodiments, at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the immune effector cells express detectable levels of NKG2D.

In some embodiments, the immune effector cells are γδ T cells that have been expanded with artificial antigen presenting cells (aAPCs). In some embodiments, the aAPCs contain on their surface anti-CD3 single chain antibodies, anti-CD28 single chain antibodies (scFv), and optionally anti-41BBL antibodies. In some embodiments, the aAPCs also contain on their surface a heparin binding domain (HBD). Anti-CD3 and Anti-CD28 scFvs bind and activate expanding T cells ex vivo, while the Heparin Binding Domain binds the viral vector, thereby bringing the T cells into close proximity with virus for effective gene transfer. This is a less costly, renewable, modifiable, and efficacious alternative to coated beads and RetroNectin® for gene transfer. Methods for producing and using aAPCs to generate CAR-T cells can be found in Shrestha B, et al. J Immunotherapy 2020 43(3):79-88, which is hereby incorporated by reference for this teaching.

Also disclosed is a dual CAR T cell using the disclosed NKG2D expressing immune effector cells. In some cases, the dual CAR T cell expresses a CD33-specific CAR and a CD123-specific CAR. In these embodiments, one CAR can include only the CD3ζ domain and the other CAR can include only the co-stimulatory domain(s). In these embodiments, dual CAR T cell activation would require co-expression of both targets on the target cell. Therefore, in some embodiments, the cell exhibits an anti-tumor immunity when both the antigen binding domain of a first CAR binds to CD33 and the antigen binding domain of a second CAR binds to CD123. In these embodiments, each of the first and second CAR polypeptides can have incomplete endodomains.

In some embodiments, the immune effector cells are genetically modified to express at least two CAR polypeptides that can be used with adoptive cell transfer to target cancers co-expressing CD99 and/or CLEC12A.

Also disclosed are bi-specific CAR-T cells that contain CAR polypeptides that can bind EGFR/MUC1-expressing cells. Therefore, also disclosed is an NKG2D expressing immune effector cell genetically modified to express an anti-EGFR CAR binding agent and an anti-MUC1 binding agent.

Also disclosed herein is a bi-specific CAR polypeptide that includes an EGFR antigen binding domain, a MUC1 antigen binding domain, a transmembrane domain, an intracellular signaling domain, and a co-stimulatory signaling region. In some embodiments, the EGFR antigen binding domain is a single-chain variable fragment (scFv) of an antibody comprising a variable heavy (V_{H}) domain and a variable light (V_{L}) domain, and wherein the MUC1 antigen binding domain is a scFv comprising a V_{H} domain and a V_{L} domain.

In some embodiments, the NKG2D-expressing immune effector cell is further genetically modified to express a membrane-bound IL-15 molecule. For example, the membrane-bound IL-15 molecule can have the amino acid sequence SEQ ID NO:91.

In some embodiments, the NKG2D-expressing immune effector cell is further genetically modified to express a membrane-bound IL-21 molecule. For example, the membrane-bound IL-21 molecule can have the amino acid sequence SEQ ID NO:93.

Also disclosed herein is an expression vector comprising a gene encoding the CAR polypeptide, a gene encoding the membrane-bound IL-15 molecule, a gene encoding the membrane-bound IL-21 molecule, or a combination thereof, operably linked to a single expression control sequence or separate expression control sequences. In some embodiments, the gene encoding the CAR polypeptide, the gene encoding the membrane-bound IL-15 molecule, the gene encoding the membrane-bound IL-21 molecule, or combination thereof are separated by a nucleic acid sequence encoding a self-cleaving peptide, such as P2A.

In some embodiments, the cell further comprises a molecular suicide switch system to remove the transferred cell population. For example, the nucleic acid encoding the CAR polypeptide can be part of an expression cassette that also includes an accessory gene. For example, in some embodiments, the accessory gene is a truncated EGFR gene (EGFRt). An EGFRt may be used as a non-immunogenic selection tool (e.g., immunomagnetic selection using biotinylated cetuximab in combination with anti-biotin microbeads for enrichment of T cells that have been lentivirally transduced with EGFRt-containing constructs), tracking marker (e.g., flow cytometric analysis for tracking T cell engraftment), or a suicide gene (e.g., via Cetuximab/Erbitux® mediated antibody dependent cellular cytotoxicity (ADCC) pathways). An example of a truncated EGFR (EGFRt) gene that may be used in accordance with the embodiments described herein is described in International Application No. PCT/US2010/055329, the subject matter of which is hereby incorporated by reference as if fully set forth herein. In other embodiments, the accessory gene is a truncated CD19 gene (CD19t). In other embodiments, the accessory gene is an inducible caspase 9 gene.

Also disclosed is a method of providing an anti-tumor immunity in a subject with a CD33-expressing and/or CD123-expressing cancer that involves administering to the subject an effective amount of an NKG2D expressing immune effector cell genetically modified with a disclosed CD33-specific CAR and/or CD123-specific CAR. In some cases, the cancer can be any CD123-expressing malignancy. In some cases, the cancer comprises Acute Myeloid Leukemia (AML), blastic plasmocytoid dendritic cell neoplasm, hairy cell leukemia, and Acute Lymphoblastic Leukemia.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B show K-562 CD3/CD137L/CD28/IL15RA aAPC characterization. FIG. 1A shows a schema of K-562 CD3/CD137L/CD28/IL15RA aAPC. FIG. 1B shows post-sort analysis of aAPC. Flow cytometry plots and histograms of aAPCs and FMO controls.

FIGS. 2A to 2F show co-culture of K-562 aAPC enhances γδ T cell expansion and memory phenotype. FIG. 2A shows an experimental timeline. At day 0, 1×10⁶ γδ T cells were added to 1×10⁸ irradiated aAPCs. At days 7, 10, and 14, a portion of cells were removed, counted, and phenotypic markers analyzed by flow cytometry. FIG. 2B shows flow cytometry gating strategy for γδ T cells. FIG. 2C shows co-culture of γδ T cells with aAPCs results in 2429-fold expansion. FIG. 2D shows CD16+ γδ T cell counts expand between days 0 and 10. FIG. 2E shows CD56+ γδ T cells counts increase between days 7 and 10. FIG. 2F shows percentages of γδ T cell memory phenotypes at indicated days. Data representative of 4 independent, healthy donors.

FIGS. 3A to 3D show 10:1 aAPC:γδ T cell ratio is optimal for expansion. FIG. 3A shows an experimental timeline. PBMCs were cultured with zol and IL-2 at day −7. Zol enriched γδ T cells were co-cultured with irradiated aAPCs at 1:0, 1:1, 5:1, 10:1, and 50:1 aAPC:γδ T cell ratios. A portion of cells were collected at days 7 and 10 for enumeration and phenotyping by flow cytometry. FIG. 3B shows γδ T cell fold change and cell counts are highest at a 10:1 aAPC:γδ T cell ratio at days 7 and 10. FIG. 3C shows CD16+ γδ T cell fold change and counts are similar at either a 10:1 or 50:1 aAPC:γδ T cell ratio. FIG. 3D shows CD56+ γδ T cells have the highest fold change and count at a 10:1 aAPC:γδ T cell ratio at days 7 and 10. Data is from a healthy donor.

FIGS. 4A to 41 show co-culture of zol enriched γδ T cells with K-562 aAPCs enhances expansion and memory phenotype. FIG. 4A shows flow cytometry gating strategy for γδ T cells. At days 7 and 10, a portion of cells were removed, counted, and phenotypic markers analyzed by flow cytometry. FIG. 4B shows co-culture of γδ T cells with aAPCs results in an average expansion of 633-fold. FIG. 4C shows γδ T cell absolute numbers increase with aAPC co-culture. FIG. 4D shows CD16+ γδ T fold change increases after 10 days of aAPC co-culture. FIG. 4E shows CD56+ γδ T fold change expands with aAPC co-culture. FIGS. 4F and 4G show percentage of γδ T cells that are PD1+ (FIG. 4F) or LAG3+ (FIG. 4G). FIG. 4H shows NKG2D percentage and number of γδ T cells expressing NKG2D increases after 10 days of aAPC co-culture. FIG. 4I shows percentages of γδ T cell memory phenotypes at indicated days. At day 0 γδ T cells were added to irradiated aAPCs at a 10:1 aAPC:γδ T cell ratio. Data shows 3 independent, healthy donors.

FIGS. 5A and 5B show γδ T cells maintain cytotoxic function after expansion with aAPCs. FIG. 5A shows donor 1 γδ T cell cytotoxicity. FIG. 5B shows donor 2 γδ T cell cytotoxicity. Target CHO cells were co-cultured with γδ T cells at a 10:1 ET ratio in triplicate. Cytotoxicity was measured by an xCelligence RTCA assay.

FIGS. 6A to 6C show aAPC:γδ T cell ratios of 10:1 or higher facilitate similar expansion of γδ T cells. Irradiated aAPCs were co-cultured with zol enriched γδ T cells at 0:1, 10:1, 50:1 and 100:1 aAPC:γδ T cell ratios. At day 10 γδ T cells were enumerated and phenotyped by flow cytometry. FIG. 6A shows γδ T cell fold change and cell counts are similar at all aAPC:γδ T cell ratios. FIG. 6B shows CD16+ γδ T cell fold change and counts are similar at all aAPC:γδ T cell ratios. FIG. 6C shows CD56+ γδ T cells have similar fold change and count at all aAPC:γδ T cell ratios. Data is from a healthy donor.

FIG. 7 is a schematic of gamma delta CAR T cell enrichment and CAR transduction at timepoints A, B, and C.

FIG. 8A shows live dead staining of cells at indicated days. FIG. 8B shows live cells stained for CD19 and CD14. Live cells that were double negative for CD19 and CD14 were possible gamma delta cells.

FIG. 9A shows live CD14− CD19− cells stained for TCRab and TCRgd. FIG. 9B shows live CD14− CD19− TCRgd+ cells stained for CD3 and CD45. Cells which were double positive for CD3 and CD45 were considered true gamma delta cells for further figures.

FIG. 10A shows gamma delta T cell percentage. FIG. 10B shows absolute counts of gamma delta T cells. FIG. 10C shows fold increase of gamma delta T cells from day 7.

FIG. 11 shows percentage of GFP (CAR) positive gamma delta T cells.

FIG. 12 is a schematic of an experimental design.

FIG. 13 shows CAR cytotoxicity against CD33 expressing targets. 10:1 effector:target ratio. Lower the line equals more killing.

FIG. 14 shows CAR cytotoxicity against CD123 expressing targets. 10:1 effector:target ratio. Lower the line equals more killing.

FIGS. 15A to 15H show cytokine secretion from gamma delta CAR T cells.

FIGS. 16A to 16D show immune phenotype of gamma delta CAR T cells. CM=central memory. EM=effector memory. EMRA=effector memory RA (most exhausted).

FIGS. 17A and 17B show immune phenotype after stimulation with CD33 targets (FIG. 17A) or CD123 targets (FIG. 17B). FIG. 17C shows NKG2D expression on gamma delta CAR T cells.

FIG. 18 is a schematic of a NK cell expansion and transduction protocol. NK cells were isolated from healthy donor PBMC and cultured with 30Gy-irradiated aAPC (K562 cells expressing 4-1BBL, IL-15RA, anti-CD28 scFv and ProteinL (aAPC:NK 2:1 ratio) in the presence of IL-15 5 ng/ml (Day 0). After 6 days, NK cells were transduced with SFG retrovirus containing hCD33BBz CAR with different anti-CD33 scFvs sequences (6A11-HC1 LC, 6A11-HC2 LC, 27A3-HC1 LC1, 27A3-HC1 LC2 or 27A3-HC1 LC3) or mock transduced (UT). Between day 14 and 21, CAR-NK cell were harvest and characterized by flow cytometry and functional assays.

FIG. 19A shows NK cells obtained after expansion were characterized by flow cytometry. FIG. 2A shows representative plots for UT cells: NK cells, gated on live cells based on their expression of CD56 and lack of CD3, represented more than 97% of the product after 14 days. NK cells highly expressed CD16 and NKG2D with variable levels of NKG2A and low PD-1. FIG. 19B shows transduction efficiency estimated by flow cytometry after staining with biotinylated Protein L followed by fluorophore-labeled streptavidin. Percentage of Protein L positive cells was calculated after gating on CD3-CD56+ live cells. At least 37% of the NK cells expressed CD33BBz CAR on the surface, with different expression levels for each anti-CD33 scFv construct.

FIG. 20 shows NK cells counted every week by flow cytometry using CountBright absolute counting beads. Co-culture with aAPC resulted in a fold increase of around 2000 for UT and CD33BBz CAR-NK cells on D21.

FIGS. 21A and 21B show cytotoxicity evaluated by xCelligence real-time cell analysis (RTCA) using CHO (FIG. 21A) or CHO-CD33 (FIG. 21B) target cells at 3:1 E:T ratio. CD33BBz CAR-NK cells expressing 6A11-HC1 LC scFv showed the highest cytotoxicity against CHO-CD33 cell line. FIG. 21C shows cytotoxicity evaluated by a luminescence assay using MV4-11 AML cell line (expressing luciferase) at 1:3 E:T ratio. CD33BBz CAR-NK cells expressing 6A11-HC1 LC scFv showed the highest cytotoxicity.

FIG. 22 shows IFN-γ production by CD33 CAR-NK cells evaluated in the supernatant of a co-culture with CHO or CHO-CD33 target cells at 1:1 E:T ratio by a Simple Plex assay on ELLA platform. CD33BBz CAR-NK cells expressing 6A11-HC1 LC scFv showed the highest IFN-γ production against CHO-CD33 cell line.

FIG. 23 illustrates an experiment to study the ability of CD33 CAR-NK cells expressing membrane bound IL-15 (mb-IL15) to survive and kill tumor cells in vivo and to compare the activity of CAR-NK cells expressing mb-IL15 vs mb-IL15+membrane bound IL-21 (mb-IL-21).

FIG. 24 shows tumors 7, 14, 21, and 28 days after treatment with UT, CD33 CAR-NK cells, CD33 CAR-NK cells with mb-IL5, and CD33 CAR-NK cells with mb-IL5 and mb-IL21.

FIGS. 25A and 25B show BLI (FIG. 25A) and body weight (FIG. 25B) 7, 14, 21, and 28 days after treatment with UT, CD33 CAR-NK cells, CD33 CAR-NK cells with mb-IL5, and CD33 CAR-NK cells with mb-IL5 and mb-IL21.

FIGS. 26A and 26B show number of NK cells per microliter one week (FIG. 26A) or 7 to 21 days (FIG. 26B) after treatment with UT, CD33 CAR-NK cells, CD33 CAR-NK cells with mb-IL5, and CD33 CAR-NK cells with mb-IL5 and mb-IL21.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class. In addition to intact immunoglobulin molecules, also included in the term “antibodies” are fragments or polymers of those immunoglobulin molecules, and human or humanized versions of immunoglobulin molecules that selectively bind the target antigen.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)2, scFv, Fv, dsFv diabody, Fc, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “antigen binding site” refers to a region of an antibody that specifically binds an epitope on an antigen.

The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “chimeric molecule” refers to a single molecule created by joining two or more molecules that exist separately in their native state. The single, chimeric molecule has the desired functionality of all of its constituent molecules. One type of chimeric molecules is a fusion protein.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (for example IgA, IgD, IgE, IgG, IgM and IgY).

The term “epitope” refers to the region of an antigen to which an antibody binds preferentially and specifically. A monoclonal antibody binds preferentially to a single specific epitope of a molecule that can be molecularly defined. In the present invention, multiple epitopes can be recognized by a multispecific antibody.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “Fab fragment” refers to a fragment of an antibody comprising an antigen-binding site generated by cleavage of the antibody with the enzyme papain, which cuts at the hinge region N-terminally to the inter-H-chain disulfide bond and generates two Fab fragments from one antibody molecule.

The term “F(ab′)2 fragment” refers to a fragment of an antibody containing two antigen-binding sites, generated by cleavage of the antibody molecule with the enzyme pepsin which cuts at the hinge region C-terminally to the inter-H-chain disulfide bond. The term “Fc fragment” refers to the fragment of an antibody comprising the constant domain of its heavy chain.

The term “Fv fragment” refers to the fragment of an antibody comprising the variable domains of its heavy chain and light chain.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “multivalent antibody” refers to an antibody or engineered antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “peptide,” “protein,” and “polypeptide” are used interchangeably to refer to a natural or synthetic molecule comprising two or more amino acids linked by the carboxyl group of one amino acid to the alpha amino group of another.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “protein domain” refers to a portion of a protein, portions of a protein, or an entire protein showing structural integrity; this determination may be based on amino acid composition of a portion of a protein, portions of a protein, or the entire protein.

The term “single chain variable fragment or scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

A “spacer” as used herein refers to a peptide that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “transformation” and “transfection” mean the introduction of a nucleic acid, e.g., an expression vector, into a recipient cell including introduction of a nucleic acid to the chromosomal DNA of said cell.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “variant” refers to an amino acid or peptide sequence having conservative amino acid substitutions, non-conservative amino acid substitutions (i.e. a degenerate variant), substitutions within the wobble position of each codon (i.e. DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

The term “vector” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element).

### Chimeric Antigen Receptors (CAR)

The disclosed CARs are generally made up of three domains: an ectodomain, a transmembrane domain, and an endodomain. The ectodomain comprises the antigen-binding region and is responsible for antigen recognition. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain a signaling domain (ISD) and a co-stimulatory signaling region (CSR).

CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45). Disclosed herein is a CD33-specific chimeric antigen receptor (CAR) and/or CD123-specific CAR that can be that can be expressed in the disclosed NKG2D expressing immune effector cells to enhance antitumor activity against CD33-specific and/or CD123-specific CARs.

### CD33 CAR

The anti-CD33 binding agent is in some embodiments an antibody fragment that specifically binds CD33. For example, the antigen binding domain can be a Fab or a single-chain variable fragment (scFv) of an antibody that specifically binds CD33. The anti-CD33 binding agent is in some embodiments an aptamer that specifically binds CD33. For example, the anti-CD33 binding agent can be a peptide aptamer selected from a random sequence pool based on its ability to bind CD33. The anti-CD33 binding agent can also be a natural ligand of CD33, or a variant and/or fragment thereof capable of binding CD33.

In some embodiments, the anti-CD33 region of the disclosed antibody or CAR is derived from hybridoma 27A3, 33G3, 36C2, 6A11, 35D5, 38G5, or combinations thereof. In some embodiments, the anti-CD33 region (e.g. scFv) can comprise a variable heavy (V_{H}) domain having CDR1, CDR2 and CDR3 sequences and a variable light (V_{L}) domain having CDR1, CDR2 and CDR3 sequences.

For example, in some embodiments, the CDR1 sequence of the V_{H} domain comprises the amino acid sequence GFTFSNYG (SEQ ID NO:1), GYTFTSYW (SEQ ID NO: 2), or GFSLSRYS (SEQ ID NO:3), wherein the CDR2 sequence of the V_{H} domain comprises the amino acid sequence ISSGGGDT (SEQ ID NO:4), IHPSDSET (SEQ ID NO: 5), or IWGGGYT (SEQ ID NO:6), wherein the CDR3 sequence of the V_{H} domain comprises the amino acid sequence ARDYGGTWDYFDY (SEQ ID NO:7), AREEGQLGHGGAMDY (SEQ ID NO:8), or ARYIDSSGYDY (SEQ ID NO:9), wherein the CDR1 sequence of the V_{L} comprises the amino acid sequence QDISKY (SEQ ID NO: 10), QTVNDD (SEQ ID NO:11), SSVSY (SEQ ID NO:12), or ENIYSY (SEQ ID NO: 13), wherein the CDR2 sequence of the V_{L} domain comprises the amino acid sequence YTS, YVS, DTS, or NAK, wherein the CDR3 sequence of the V_{L} domain comprises the amino acid sequence QQGDTFPWT (SEQ ID NO: 14), QQDYSSPYT (SEQ ID NO:15), QQWSSNPLT (SEQ ID NO:16), or QHHYGTPYT (SEQ ID NO:17), or any combination thereof.

Therefore, in some embodiments, the anti-CD33 scFv V_{H} domain comprises the amino acid sequence

(SEQ ID NO: 18)
EVKLVESGGGLVKPGASLKLSCAASGFTFSNYGMSWVRQTSDKRLEWVA
SISSGGGDTYYPDNVKGRFTISRENAKNTLYLQMSSLNSEDTALYYCAR
DYGGTWDYFDYWGQGTTLTVSS,
(SEQ ID NO: 19)
QVQLQQPGAELVRPGVSVKLSCKASGYTFTSYWMNWVKQRPGQGLEWIG
MIHPSDSETRLNQKFKDKAILTVDKSSSTAYMQLSSPTSEDSAVYYCAR
EEGQLGHGGAMDYWGQGTSVTVSS,
or
(SEQ ID NO: 20)
QVQLKESGPGLVAPSQSLSITCTVSGFSLSRYSVHWVRQPPGKGLEWLG
MIWGGGYTDYNSALKSRLSISKDNSKSQVFLKMNSLQTDDTAMYYCARY
IDSSGYDYWGQGTTLTVSS.

In some embodiments, the anti-CD33 scFv V_{L} domain comprises the amino acid sequence

(SEQ ID NO: 21)
DIQMTQTTSSLSASLGDRVTISCRASQDISKYLNWYQQKPDGTVKLLIYYTSRLHSGVP
SRFSGSGSGTDYSLTISNLEQEDIATYFCQQGDTFPWTFGGGTKLEIK,
(SEQ ID NO: 22)
SIVMTQTPKFLLVSAGDRVTITCKASQTVNDDVAWYQQKPGQSPKLLIYYVSNRHTGVP
DRFTGSGYGTDFTFTISTVQAEDLAVYFCQQDYSSPYTFGGGTKLEIK,
(SEQ ID NO: 23)
QIVLTQSPAIMSASPGEKVTMTCSASSSVSYMHWYQQKSGTSPKRWIYDTSKLASGVP
ARFSGSGSGTSYSLTISSMEAEDAATYYCQQWSSNPLTFGAGTKLELK,
or
(SEQ ID NO: 24)
DIQMTQSPASLSASVGETVTITCRASENIYSYLAWYQQKQGKSPQLLVYNAKTLAEGVP
SRFSGSGSGTQFSLKINSLQPEDFGSYYCQHHYGTPYTFGGGTKLEIK.

The heavy and light chains are preferably separated by a linker. Suitable linkers for scFv antibodies are known in the art. In some embodiments, the linker comprises the amino acid sequence GGGGSGGGGSGGGGS (SEQ ID NO:25).

In some embodiments, the anti-CD33 scFv comprises the amino acid sequence:

(SEQ ID NO: 26, 6A11HC1_LC)
EVKLVESGGGLVKPGASLKLSCAASGFTFSNYGMSWVRQTSDKRLEWVASISSGGGD
TYYPDNVKGRFTISRENAKNTLYLQMSSLNSEDTALYYCARDYGGTWDYFDYWGQGT
TLTVSSGGGGSGGGGSGGGGSDIQMTQTTSSLSASLGDRVTISCRASQDISKYLNWY
QQKPDGTVKLLIYYTSRLHSGVPSRFSGSGSGTDYSLTISNLEQEDIATYFCQQGDTFP
WTFGGGTKLEIK.

In some embodiments, the anti-CD33 scFv comprises the amino acid sequence:

(SEQ ID NO: 27, 6A11HC2_LC)
QVQLQQPGAELVRPGVSVKLSCKASGYTFTSYWMNWVKQRPGQGLEWIGMIHPSDS
ETRLNQKFKDKAILTVDKSSSTAYMQLSSPTSEDSAVYYCAREEGQLGHGGAMDYWG
QGTSVTVSSGGGGSGGGGSGGGGSDIQMTQTTSSLSASLGDRVTISCRASQDISKYL
NWYQQKPDGTVKLLIYYTSRLHSGVPSRFSGSGSGTDYSLTISNLEQEDIATYFCQQG
DTFPWTFGGGTKLEIK.

In some embodiments, the anti-CD33 scFv comprises the amino acid sequence:

(SEQ ID NO: 28, 27A3HC_LC1)
QVQLKESGPGLVAPSQSLSITCTVSGFSLSRYSVHWVRQPPGKGLEWLGMIWGGGYT
DYNSALKSRLSISKDNSKSQVFLKMNSLQTDDTAMYYCARYIDSSGYDYWGQGTTLTV
SSGGGGSGGGGSGGGGSSIVMTQTPKFLLVSAGDRVTITCKASQTVNDDVAWYQQK
PGQSPKLLIYYVSNRHTGVPDRFTGSGYGTDFTFTISTVQAEDLAVYFCQQDYSSPYTF
GGGTKLEIK.

In some embodiments, the anti-CD33 scFv comprises the amino acid sequence:

(SEQ ID NO: 29, 27A3HC_LC2)
QVQLKESGPGLVAPSQSLSITCTVSGFSLSRYSVHWVRQPPGKGLEWLGMIWGGGYT
DYNSALKSRLSISKDNSKSQVFLKMNSLQTDDTAMYYCARYIDSSGYDYWGQGTTLTV
SSGGGGSGGGGSGGGGSQIVLTQSPAIMSASPGEKVTMTCSASSSVSYMHWYQQKS
GTSPKRWIYDTSKLASGVPARFSGSGSGTSYSLTISSMEAEDAATYYCQQWSSNPLTF
GAGTKLELK.

In some embodiments, the anti-CD33 scFv comprises the amino acid sequence:

(SEQ ID NO: 30, 27A3HC_LC3)
QVQLKESGPGLVAPSQSLSITCTVSGFSLSRYSVHWVRQPPGKGLEWLGMIWGGGYT
DYNSALKSRLSISKDNSKSQVFLKMNSLQTDDTAMYYCARYIDSSGYDYWGQGTTLTV
SSGGGGSGGGGSGGGGSDIQMTQSPASLSASVGETVTITCRASENIYSYLAWYQQKQ
GKSPQLLVYNAKTLAEGVPSRFSGSGSGTQFSLKINSLQPEDFGSYYCQHHYGTPYTF
GGGTKLEIK.

In some embodiments, the anti-CD33 scFv is encoded by the nucleic acid sequence:

(SEQ ID NO: 31, 6A11HC1_LC)
ATGGCCCTCCCGGTAACGGCTCTGCTGCTTCCACTCGCACTGCTCTTGCATGCTGC
CAGACCAGAAGTGAAGCTGGTGGAGTCTGGGGGAGGCTTAGTGAAGCCTGGAGC
GTCTCTGAAACTCTCCTGTGCAGCCTCTGGATTCACTTTCAGTAACTATGGCATGTC
TTGGGTTCGCCAGACTTCAGACAAGAGGCTGGAGTGGGTCGCATCCATTAGTAGT
GGTGGTGGTGACACCTACTATCCAGACAATGTAAAGGGCCGATTCACCATCTCCAG
AGAGAATGCCAAGAACACCCTGTACCTGCAAATGAGTAGTCTGAACTCTGAGGACA
CGGCCTTGTATTACTGTGCAAGAGACTATGGTGGTACTTGGGACTACTTTGACTACT
GGGGCCAAGGCACCACTCTCACAGTCTCCTCAGGTGGAGGTGGATCAGGTGGAG
GTGGATCTGGTGGAGGTGGATCTGATATCCAGATGACACAGACTACATCCTCCCTG
TCTGCCTCTCTGGGAGACAGAGTCACCATCAGTTGCAGGGCAAGTCAGGACATTAG
CAAGTATTTAAACTGGTATCAGCAGAAACCAGATGGAACTGTTAAACTCCTGATCTA
CTACACATCAAGATTACACTCAGGAGTCCCATCGAGGTTCAGTGGCAGTGGGTCTG
GAACAGATTATTCTCTCACCATTAGCAACCTGGAGCAAGAAGATATTGCCACTTACT
TTTGCCAACAGGGTGATACGTTTCCGTGGACGTTCGGTGGAGGCACCAAGCTGGA
AATCAAACGG.

In some embodiments, the anti-CD33 scFv is encoded by the nucleic acid sequence:

(SEQ ID NO: 32, 6A11HC2_LC)
ATGGCCCTCCCGGTAACGGCTCTGCTGCTTCCACTCGCACTGCTCTTGCATGCTGC
CAGACCACAGGTCCAACTGCAGCAGCCTGGGGCTGAGCTGGTGAGGCCTGGAGTT
TCAGTGAAGCTGTCCTGCAAGGCTTCTGGCTACACCTTCACCAGCTACTGGATGAA
CTGGGTGAAGCAGAGGCCTGGACAAGGCCTTGAGTGGATTGGCATGATTCATCCT
TCCGATAGTGAAACTAGGTTAAATCAGAAGTTCAAGGACAAGGCCATATTGACTGTA
GACAAATCCTCCAGCACAGCCTACATGCAACTCAGCAGCCCGACATCTGAGGACTC
TGCGGTCTATTACTGTGCAAGAGAAGAGGGACAGCTCGGGCACGGCGGTGCTATG
GACTACTGGGGTCAAGGAACCTCAGTCACCGTCTCCTCAGGTGGAGGTGGATCAG
GTGGAGGTGGATCTGGTGGAGGTGGATCTGATATCCAGATGACACAGACTACATC
CTCCCTGTCTGCCTCTCTGGGAGACAGAGTCACCATCAGTTGCAGGGCAAGTCAG
GACATTAGCAAGTATTTAAACTGGTATCAGCAGAAACCAGATGGAACTGTTAAACTC
CTGATCTACTACACATCAAGATTACACTCAGGAGTCCCATCGAGGTTCAGTGGCAG
TGGGTCTGGAACAGATTATTCTCTCACCATTAGCAACCTGGAGCAAGAAGATATTG
CCACTTACTTTTGCCAACAGGGTGATACGTTTCCGTGGACGTTCGGTGGAGGCACC
AAGCTGGAAATCAAACGG.

In some embodiments, the anti-CD33 scFv is encoded by the nucleic acid sequence:

(SEQ ID NO: 33, 27A3HC_LC1)
ATGGCCCTCCCGGTAACGGCTCTGCTGCTTCCACTCGCACTGCTCTTGCATGCTGC
CAGACCACAGGTGCAGCTGAAGGAGTCAGGACCTGGCCTGGTGGCACCCTCACAG
AGCCTGTCCATCACATGCACGGTCTCTGGGTTCTCATTATCCAGATATAGTGTACAC
TGGGTTCGCCAGCCTCCAGGAAAGGGTCTGGAGTGGCTGGGAATGATATGGGGTG
GTGGATACACAGACTATAATTCAGCTCTCAAATCCAGACTGAGCATCAGCAAGGAC
AACTCCAAGAGCCAAGTTTTCTTAAAAATGAACAGTCTGCAAACTGATGACACAGCC
ATGTACTACTGTGCCAGATATATAGACAGCTCGGGCTACGACTACTGGGGCCAAGG
CACCACTCTCACAGTCTCCTCAGGTGGAGGTGGATCAGGTGGAGGTGGATCTGGT
GGAGGTGGATCTAGTATTGTGATGACCCAGACTCCCAAATTCCTGCTTGTATCAGC
AGGAGACAGGGTTACCATAACCTGCAAGGCCAGTCAGACTGTGAATGATGATGTAG
CTTGGTATCAACAGAAGCCAGGACAGTCTCCTAAATTGCTGATATATTATGTATCCA
ATCGCCACACTGGAGTCCCTGATCGCTTCACTGGCAGTGGATATGGGACGGATTTC
ACTTTCACCATCAGCACTGTGCAGGCTGAAGACCTGGCAGTTTATTTCTGTCAGCA
GGATTATAGCTCTCCGTACACGTTCGGAGGGGGGACCAAGCTGGAAATAAAACGG.

In some embodiments, the anti-CD33 scFv is encoded by the nucleic acid sequence:

(SEQ ID NO: 34, 27A3HC_LC2)
ATGGCCCTCCCGGTAACGGCTCTGCTGCTTCCACTCGCACTGCTCTTGCATGCTGC
CAGACCACAGGTGCAGCTGAAGGAGTCAGGACCTGGCCTGGTGGCACCCTCACAG
AGCCTGTCCATCACATGCACGGTCTCTGGGTTCTCATTATCCAGATATAGTGTACAC
TGGGTTCGCCAGCCTCCAGGAAAGGGTCTGGAGTGGCTGGGAATGATATGGGGTG
GTGGATACACAGACTATAATTCAGCTCTCAAATCCAGACTGAGCATCAGCAAGGAC
AACTCCAAGAGCCAAGTTTTCTTAAAAATGAACAGTCTGCAAACTGATGACACAGCC
ATGTACTACTGTGCCAGATATATAGACAGCTCGGGCTACGACTACTGGGGCCAAGG
CACCACTCTCACAGTCTCCTCAGGTGGAGGTGGATCAGGTGGAGGTGGATCTGGT
GGAGGTGGATCTCAAATTGTTCTCACCCAGTCTCCAGCAATCATGTCTGCATCTCC
AGGGGAGAAGGTCACCATGACCTGCAGTGCCAGCTCAAGTGTAAGTTACATGCACT
GGTACCAGCAGAAGTCAGGCACCTCCCCCAAAAGATGGATTTATGACACATCCAAA
CTGGCTTCTGGAGTCCCTGCTCGCTTCAGTGGCAGTGGGTCTGGGACCTCTTACTC
TCTCACAATCAGCAGCATGGAGGCTGAAGATGCTGCCACTTATTACTGCCAGCAGT
GGAGTAGTAACCCACTCACGTTCGGTGCTGGGACCAAGCTGGAGCTGAAACGG.

In some embodiments, the anti-CD33 scFv is encoded by the nucleic acid sequence:

(SEQ ID NO: 35, 27A3HC_LC3)
ATGGCCCTCCCGGTAACGGCTCTGCTGCTTCCACTCGCACTGCTCTTGCATGCTGC
CAGACCACAGGTGCAGCTGAAGGAGTCAGGACCTGGCCTGGTGGCACCCTCACAG
AGCCTGTCCATCACATGCACGGTCTCTGGGTTCTCATTATCCAGATATAGTGTACAC
TGGGTTCGCCAGCCTCCAGGAAAGGGTCTGGAGTGGCTGGGAATGATATGGGGTG
GTGGATACACAGACTATAATTCAGCTCTCAAATCCAGACTGAGCATCAGCAAGGAC
AACTCCAAGAGCCAAGTTTTCTTAAAAATGAACAGTCTGCAAACTGATGACACAGCC
ATGTACTACTGTGCCAGATATATAGACAGCTCGGGCTACGACTACTGGGGCCAAGG
CACCACTCTCACAGTCTCCTCAGGTGGAGGTGGATCAGGTGGAGGTGGATCTGGT
GGAGGTGGATCTGACATCCAGATGACTCAGTCTCCAGCCTCCCTATCTGCATCTGT
GGGAGAAACTGTCACCATCACATGTCGAGCAAGTGAGAATATTTACAGTTATTTAGC
ATGGTATCAGCAGAAACAGGGAAAATCTCCTCAGCTCCTGGTCTATAATGCAAAAA
CCTTAGCAGAAGGTGTGCCATCAAGGTTCAGTGGCAGTGGATCAGGCACACAGTTT
TCTCTGAAGATCAACAGTCTGCAGCCTGAAGATTTTGGGAGTTATTACTGTCAACAT
CATTATGGTACTCCGTACACGTTCGGAGGGGGGACCAAGCTGGAAATAAAACGG.

### CD123 CAR

The anti-CD123 binding agent is in some embodiments an antibody fragment that specifically binds CD123. For example, the antigen binding domain can be a Fab or a single-chain variable fragment (scFv) of an antibody that specifically binds CD123. The anti-CD123 binding agent is in some embodiments an aptamer that specifically binds CD123. For example, the anti-CD123 binding agent can be a peptide aptamer selected from a random sequence pool based on its ability to bind CD123. The anti-CD123 binding agent can also be a natural ligand of CD123, or a variant and/or fragment thereof capable of binding CD123.

In some embodiments, the anti-CD123 scFv is derived from hybridoma 3F5, 4E10, 12H5, 15A12, 17E7, 12H11, or combinations thereof. In some embodiments, the anti-CD123 scFv can comprise a variable heavy (V_{H}) domain having CDR1, CDR2 and CDR3 sequences and a variable light (V_{L}) domain having CDR1, CDR2 and CDR3 sequences.

For example, in some embodiments, the CDR1 sequence of the V_{H} domain comprises the amino acid sequence GYTFTDYN (SEQ ID NO:36), CDR2 sequence of the V_{H} domain comprises the amino acid sequence INPNNGGT (SEQ ID NO:37), CDR3 sequence of the V_{H} domain comprises the amino acid sequence ARKGYGGNYDYFDY (SEQ ID NO:38), CDR1 sequence of the V_{L} comprises the amino acid sequence QSIGTS (SEQ ID NO:39), CDR2 sequence of the V_{L} domain comprises the amino acid sequence YAS, and CDR3 sequence of the V_{L} domain comprises the amino acid sequence QQSNSWPYT (SEQ ID NO:40).

In some embodiments, the CDR1 sequence of the V_{H} domain comprises the amino acid sequence GFNIKDTY (SEQ ID NO:41) or GFSLSTYGMG (SEQ ID NO:42), the CDR2 sequence of the V_{H} domain comprises the amino acid sequence IDPANGNT (SEQ ID NO:43) or IYWDDDK (SEQ ID NO:44), the CDR3 sequence of the V_{H} domain comprises the amino acid sequence ALYYYGGSLDY (SEQ ID NO:45) or AQSLIYDGYYGFAY (SEQ ID NO:46), the CDR1 sequence of the V_{L} comprises the amino acid sequence QSLLYSGNQKNY (SEQ ID NO:47), the CDR2 sequence of the V_{L} domain comprises the amino acid sequence WAS, and the CDR3 sequence of the V_{L} domain comprises the amino acid sequence QQYYSYPRT (SEQ ID NO:48).

In some embodiments, the CDR1 sequence of the V_{H} domain comprises the amino acid sequence GYTFTYYG (SEQ ID NO:49), the CDR2 sequence of the V_{H} domain comprises the amino acid sequence INTYSGVP (SEQ ID NO:50), the CDR3 sequence of the V_{H} domain comprises the amino acid sequence ARWIYYSDLYGMDY (SEQ ID NO:51), the CDR1 sequence of the V_{L} comprises the amino acid sequence QSIVHSNGDTY (SEQ ID NO:52), the CDR2 sequence of the V_{L} domain comprises the amino acid sequence KVS, and the CDR3 sequence of the V_{L} domain comprises the amino acid sequence FQGSHVPWT (SEQ ID NO:53).

The heavy and light chains are preferably separated by a linker. Suitable linkers for scFv antibodies are known in the art. In some embodiments, the linker comprises the amino acid sequence GGGGSGGGGSGGGGS (SEQ ID NO:54).

Therefore, in some embodiments, the anti-CD123 scFv V_{H} domain comprises the amino acid sequence:

(SEQ ID NO: 55, 3F5HC1)
EVQLQQSGPELVKPGSSVKISCKASGYTFTDYNMDWVKQSHGKSLEWIGTINPNNGG
TSYNQKFKGKATLTVDKSSSTAYMELRSLTSEDSAVYYCARKGYGGNYDYFDYWGQG
TTLTVSS,
(SEQ ID NO: 56, 12H1HC1)
EVQLQQSGAELVKPGASVKLSCTASGFNIKDTYMHWVKQRPEQGLEWIGRIDPANGN
TIYASKFQGKATITADTSSNTAYMQLSSLTSGDTAVYYCALYYYGGSLDYWGQGTTLTV
SS,
(SEQ ID NO: 57, 12H1HC2)
QVTLKESGPGILQPSQTLSLTCSFSGFSLSTYGMGVSWIRQPSGKGLEWLAHIYWDDD
KRYNPSLKSRLTISKDTSNNQVFLKITSVDTADTATYYCAQSLIYDGYYGFAYWGQGTL
VTVSA,
(SEQ ID NO: 58, 12H2HC1)
QIQLVQSGPELKKPGETVKISCKASGYTFTYYGMNWVKQAPGKGLEWMGWINTYSGV
PTYADDFKGRFAFSLETSVSTAYLQINNLKNEDTATYFCARWIYYSDLYGMDYWGQGT
SVTVSS,
(SEQ ID NO: 59, 15A12HC1)
QVQLQQSGAELAKPGASVKMSCKASGYTFSSYWMHWLKQRPGQGLEWIGYINPSSG
YTNYNQKFKDKATLTADKSSSTAYMQLSSLTSEDSAVYYCARDGNYDHWYFDVWGTG
TTVTVSS,
or
(SEQ ID NO: 60, 15A12HC2)
QVQLQQPGAELVRPGASVKMSCKASGYTLTTYLMDWVKQRLGQGFEWIGNINPNSGS
SNYNEKFKGKAKLTVDKSSSTAYMQLSSLTSEDSAVYYCAIRHYGGSLFDYWGQGTTL
TVSS.

In some embodiments, the anti-CD123 scFv V_{L} domain comprises the amino acid sequence:

(SEQ ID NO: 61, 3F5LC1)
DILLTQSPAILSVSPGERVSFSCRASQSIGTSIHWYQQRTNGSPRLLIKYASESISGIPSR
FSGSGSGTDFTLSINSVESEDIADYYCQQSNSWPYTFGGGTKLEIK,
(SEQ ID NO: 62, 12H1LC1)
DIVMSQSPSSLAVSVGERVTMSCKSSQSLLYSGNQKNYLAWYQQKPGQSPKLLIYWA
STRESGVPDRFTGSGSGTDFTLTISSVKAEDLAVYYCQQYYSYPRTFGGGTKLEIK,
(SEQ ID NO: 63, 12H2LC)
DVLMTQSPLSLPVSLGDQASISCRSSQSIVHSNGDTYLEWYLQKPGQSPKLLIYKVSNR
FSGVPDRFSGSGSGTDFTLKISRVEAEDLGVYHCFQGSHVPWTFGGGTKLEIK,
(SEQ ID NO: 64, 15A12LC1)
DIKMTQSPSSMYASLGERVTITCKASQDINSYLSWFQQKPGKSPKTLIYRANRLVDGVP
SRFSGSGSGQDYSLTISSLEYEDMGIYYCLQYDELLTFGAGTKLELK,
or
(SEQ ID NO: 65, 15A12LC2)
DIVMSQSPSSLAVSAGERVTMSCRSSQSLLNSRTRKNYLAWYQQKPGQSPKLLIYWAS
TRESGVPDRFSGSGSGTDFTLTISSVQAEDLAVYYCEQSYNLFTFGSGTKLEIK.

In some embodiments, the anti-CD123 scFv comprises an amino acid sequence:

(SEQ ID NO: 66 3F5HC1_LC)
EVQLQQSGPELVKPGSSVKISCKASGYTFTDYNMDWVKQSHGKSLEWIGTINPNNGG
TSYNQKFKGKATLTVDKSSSTAYMELRSLTSEDSAVYYCARKGYGGNYDYFDYWGQG
TTLTVSSGGGGSGGGGSGGGGSDILLTQSPAILSVSPGERVSFSCRASQSIGTSIHWY
QQRTNGSPRLLIKYASESISGIPSRFSGSGSGTDFTLSINSVESEDIADYYCQQSNSWP
YTFGGGTKLEIK.

In some embodiments, the anti-CD123 scFv comprises an amino acid sequence:

(SEQ ID NO: 67, 12H1HC1_LC1)
EVQLQQSGAELVKPGASVKLSCTASGFNIKDTYMHWVKQRPEQGLEWIGRIDPANGN
TIYASKFQGKATITADTSSNTAYMQLSSLTSGDTAVYYCALYYYGGSLDYWGQGTTLTV
SSGGGGSGGGGSGGGGSDIVMSQSPSSLAVSVGERVTMSCKSSQSLLYSGNQKNYL
AWYQQKPGQSPKLLIYWASTRESGVPDRFTGSGSGTDFTLTISSVKAEDLAVYYCQQY
YSYPRTFGGGTKLEIK.

In some embodiments, the anti-CD123 scFv comprises an amino acid sequence:

(SEQ ID NO: 68, 12H1HC2_LC1)
QVTLKESGPGILQPSQTLSLTCSFSGFSLSTYGMGVSWIRQPSGKGLEW
LAHIYWDDDKRYNPSLKSRLTISKDTSNNQVFLKITSVDTADTATYYCA
QSLIYDGYYGFAYWGQGTLVTVSAGGGGSGGGGSGGGGSDIVMSQSPSS
LAVSVGERVTMSCKSSQSLLYSGNQKNYLAWYQQKPGQSPKLLIYWAST
RESGVPDRFTGSGSGTDFTLTISSVKAEDLAVYYCQQYYSYPRTFGGGT
KLEIK.

In some embodiments, the anti-CD123 scFv comprises an amino acid sequence:

(SEQ ID NO: 69, 12H2HC1_LC1)
QIQLVQSGPELKKPGETVKISCKASGYTFTYYGMNWVKQAPGKGLEWMG
WINTYSGVPTYADDFKGRFAFSLETSVSTAYLQINNLKNEDTATYFCAR
WIYYSDLYGMDYWGQGTSVTVSSGGGGSGGGGSGGGGSDVLMTQSPLSL
PVSLGDQASISCRSSQSIVHSNGDTYLEWYLQKPGQSPKLLIYKVSNRF
SGVPDRFSGSGSGTDFTLKISRVEAEDLGVYHCFQGSHVPWTFGGGTKL
EIK.

In some embodiments, the anti-CD123 scFv comprises an amino acid sequence:

(SEQ ID NO: 70, 15A12HC1_LC1)
QVQLQQSGAELAKPGASVKMSCKASGYTFSSYWMHWLKQRPGQGLEWIG
YINPSSGYTNYNQKFKDKATLTADKSSSTAYMQLSSLTSEDSAVYYCAR
DGNYDHWYFDVWGTGTTVTVSSGGGGSGGGGSGGGGSDIKMTQSPSSMY
ASLGERVTITCKASQDINSYLSWFQQKPGKSPKTLIYRANRLVDGVPSR
FSGSGSGQDYSLTISSLEYEDMGIYYCLQYDELLTFGAGTKLELK.

In some embodiments, the anti-CD123 scFv comprises an amino acid sequence:

(SEQ ID NO: 71, 15A12HC1_LC2)
QVQLQQSGAELAKPGASVKMSCKASGYTFSSYWMHWLKQRPGQGLEWIG
YINPSSGYTNYNQKFKDKATLTADKSSSTAYMQLSSLTSEDSAVYYCAR
DGNYDHWYFDVWGTGTTVTVSSGGGGSGGGGSGGGGSDIVMSQSPSSLA
VSAGERVTMSCRSSQSLLNSRTRKNYLAWYQQKPGQSPKLLIYWASTRE
SGVPDRFSGSGSGTDFTLTISSVQAEDLAVYYCEQSYNLFTFGSGTKLE
IK.

In some embodiments, the anti-CD123 scFv comprises an amino acid sequence:

(SEQ ID NO: 72, 15A12HC2_LC1)
QVQLQQPGAELVRPGASVKMSCKASGYTLTTYLMDWVKQRLGQGFEWIG
NINPNSGSSNYNEKFKGKAKLTVDKSSSTAYMQLSSLTSEDSAVYYCAI
RHYGGSLFDYWGQGTTLTVSSGGGGSGGGGSGGGGSDIKMTQSPSSMYA
SLGERVTITCKASQDINSYLSWFQQKPGKSPKTLIYRANRLVDGVPSRF
SGSGSGQDYSLTISSLEYEDMGIYYCLQYDELLTFGAGTKLELK.

In some embodiments, the anti-CD123 scFv comprises an amino acid sequence:

(SEQ ID NO: 73, 15A12HC2_LC2)
QVQLQQPGAELVRPGASVKMSCKASGYTLTTYLMDWVKQRLGQGFEWIG
NINPNSGSSNYNEKFKGKAKLTVDKSSSTAYMQLSSLTSEDSAVYYCAI
RHYGGSLFDYWGQGTTLTVSSGGGGSGGGGSGGGGSDIVMSQSPSSLAV
SAGERVTMSCRSSQSLLNSRTRKNYLAWYQQKPGQSPKLLIYWASTRES
GVPDRFSGSGSGTDFTLTISSVQAEDLAVYYCEQSYNLFTFGSGTKLEI
K.

In some embodiments, the anti-CD123 scFv is encoded by the nucleic acid sequence:

(SEQ ID NO: 74, 3F5HC1_LC)
CCATGGCCCTCCCGGTAACGGCTCTGCTGCTTCCACTCGCACTGCTCTT
GCATGCTGCCAGACCAGAGGTCCAGCTGCAACAGTCTGGACCTGAGCTG
GTGAAGCCTGGGTCTTCAGTGAAGATATCCTGCAAAGCTTCTGGATACA
CATTCACTGACTACAACATGGACTGGGTGAAGCAGAGTCATGGAAAGAG
CCTTGAGTGGATTGGAACTATTAATCCTAACAATGGTGGTACTAGCTAC
AACCAGAAGTTCAAGGGCAAGGCCACATTGACTGTAGACAAGTCCTCCA
GCACAGCCTACATGGAGCTCCGCAGCCTGACATCTGAAGACTCTGCAGT
CTATTACTGTGCAAGAAAGGGCTATGGTGGTAACTACGACTACTTTGAC
TACTGGGGCCAAGGCACCACTCTCACAGTCTCCTCAGGTGGAGGTGGAT
CAGGTGGAGGTGGATCTGGTGGAGGTGGATCTGACATCTTGCTGACTCA
GTCTCCAGCCATCCTGTCTGTGAGTCCAGGAGAAAGAGTCAGTTTCTCC
TGCAGGGCCAGTCAGAGCATTGGCACAAGCATACACTGGTATCAGCAAA
GAACAAATGGTTCTCCAAGGCTTCTCATAAAGTATGCTTCTGAGTCTAT
CTCTGGGTTCCCTTCCAGGTTTAGTGGCAGTGGATCAGGGACAGATTTT
ACTCTTAGCATCAACAGTGTGGAGTCTGAAGATATTGCAGATTATTACT
GTCAACAAAGTAATAGCTGGCCGTACACGTTCGGAGGGGGGACCAAGCT
GGAAATAAAACGGGCGGCCGCA.

In some embodiments, the anti-CD123 scFv is encoded by the nucleic acid sequence:

(SEQ ID NO: 75, 12H1HC1_LC1)
ATGGCCCTCCCGGTAACGGCTCTGCTGCTTCCACTCGCACTGCTCTTGC
ATGCTGCCAGACCAGAGGTTCAGCTGCAGCAGTCTGGGGCAGAGCTTGT
GAAGCCAGGGGCCTCAGTCAAGTTGTCCTGCACAGCTTCTGGCTTCAAC
ATTAAAGACACCTATATGCACTGGGTGAAGCAGAGGCCTGAACAGGGCC
TGGAGTGGATTGGAAGGATTGATCCTGCGAATGGTAATACTATATATGC
CTCAAAGTTCCAGGGCAAGGCCACTATAACAGCAGACACATCATCCAAC
ACAGCCTACATGCAGCTCAGCAGCCTGACATCTGGGGACACTGCCGTCT
ATTACTGTGCTCTTTATTACTATGGTGGTAGCCTTGACTACTGGGGCCA
AGGCACCACTCTCACAGTCTCCTCAGGTGGAGGTGGATCAGGTGGAGGT
GGATCTGGTGGAGGTGGATCTGACATTGTGATGTCACAGTCTCCATCCT
CCCTAGCTGTGTCAGTTGGAGAGAGGGTTACTATGAGCTGCAAGTCCAG
TCAGAGCCTTTTATATAGTGGCAATCAAAAGAACTACTTGGCCTGGTAC
CAGCAGAAACCAGGGCAGTCTCCTAAACTGCTGATTTACTGGGCATCCA
CTAGGGAATCTGGGGTCCCTGATCGCTTCACAGGCAGTGGATCTGGGAC
AGATTTCACTCTCACCATCAGCAGTGTGAAGGCTGAAGACCTGGCAGTT
TATTACTGTCAGCAATATTATAGCTATCCTCGGACGTTCGGTGGAGGCA
CCAAGCTGGAAATCAAACGG.

In some embodiments, the anti-CD123 scFv is encoded by the nucleic acid sequence:

(SEQ ID NO: 76, 12H1HC1_LC2)
ATGGCCCTCCCGGTAACGGCTCTGCTGCTTCCACTCGCACTGCTCTTGC
ATGCTGCCAGACCACAGGTTACTCTGAAAGAGTCTGGCCCTGGGATATT
GCAGCCCTCCCAGACCCTCAGTCTGACTTGTTCTTTCTCTGGGTTTTCA
CTGAGCACTTATGGTATGGGTGTGAGCTGGATTCGTCAGCCTTCAGGAA
AGGGTCTGGAGTGGCTGGCACACATTTACTGGGATGATGACAAGCGCTA
TAACCCATCCCTGAAGAGCCGGCTCACAATCTCCAAGGATACCTCCAAC
AACCAGGTATTCCTCAAGATCACCAGTGTGGACACTGCAGATACTGCCA
CATACTACTGTGCTCAAAGCCTGATCTATGATGGTTACTACGGGTTTGC
CTACTGGGGCCAAGGGACTCTGGTCACTGTCTCTGCAGGTGGAGGTGGA
TCAGGTGGAGGTGGATCTGGTGGAGGTGGATCTGACATTGTGATGTCAC
AGTCTCCATCCTCCCTAGCTGTGTCAGTTGGAGAGAGGGTTACTATGAG
CTGCAAGTCCAGTCAGAGCCTTTTATATAGTGGCAATCAAAAGAACTAC
TTGGCCTGGTACCAGCAGAAACCAGGGCAGTCTCCTAAACTGCTGATTT
ACTGGGCATCCACTAGGGAATCTGGGGTCCCTGATCGCTTCACAGGCAG
TGGATCTGGGACAGATTTCACTCTCACCATCAGCAGTGTGAAGGCTGAA
GACCTGGCAGTTTATTACTGTCAGCAATATTATAGCTATCCTCGGACGT
TCGGTGGAGGCACCAAGCTGGAAATCAAACGG.

In some embodiments, the anti-CD123 scFv is encoded by the nucleic acid sequence:

(SEQ ID NO: 77, 12H2HC1_LC1)
ATGGCCCTCCCGGTAACGGCTCTGCTGCTTCCACTCGCACTGCTCTTGC
ATGCTGCCAGACCACAGATCCAGTTGGTGCAATCTGGACCTGAGCTGAA
GAAGCCTGGAGAGACAGTCAAGATCTCCTGCAAGGCTTCTGGGTATACC
TTCACATACTATGGAATGAACTGGGTGAAGCAGGCTCCAGGAAAGGGTT
TAGAGTGGATGGGCTGGATAAACACCTACTCTGGAGTGCCAACATATGC
TGATGACTTCAAGGGACGGTTTGCCTTCTCTTTGGAAACCTCTGTCAGC
ACTGCCTATTTGCAGATCAACAACCTCAAAAATGAGGACACGGCTACAT
ATTTTTGTGCAAGATGGATCTACTATAGTGACCTCTATGGTATGGACTA
CTGGGGTCAAGGAACCTCAGTCACCGTCTCCTCAGGTGGAGGTGGATCA
GGTGGAGGTGGATCTGGTGGAGGTGGATCTGATGTTTTGATGACCCAAA
ACATAGTAATGGAGACACGTATTTAGAATGGTATTTGCAGAAACCAGGC
CGTCCACTCTCCCTGCCTGTCAGTCTTGGAGATCAAGCCTCCATCTCTT
GTAGATCTAGTCAGAGTATTGTAGTCTCCAAAGCTCCTGATCTACAAAG
TTTCTAACCGATTTTCTGGGGTCCCAGACAGGTTCAGTGGCAGTGGATC
AGGGACAGATTTCACACTCAAGATCAGCAGAGTGGAGGCTGAGGATCTG
GGAGTTTATCACTGCTTTCAAGGTTCACATGTTCCGTGGACGTTCGGTG
GAGGCACCAAGCTGGAAATCAAACGG.

In some embodiments, the anti-CD123 scFv is encoded by the nucleic acid sequence:

(SEQ ID NO: 78, 15A12HC1_LC1)
ATGGCCCTCCCGGTAACGGCTCTGCTGCTTCCACTCGCACTGCTCTTGC
ATGCTGCCAGACCACAGGTCCAGCTGCAGCAGTCTGGGGCTGAACTGGC
AAAACCTGGGGCCTCAGTGAAGATGTCCTGCAAGGCTTCTGGCTACACC
TTTTCTAGCTACTGGATGCACTGGCTAAAACAGAGGCCTGGACAGGGTC
TGGAGTGGATTGGATACATTAATCCTAGCAGTGGTTATACTAACTACAA
TCAGAAGTTCAAGGACAAGGCCACATTGACTGCAGACAAATCCTCCAGC
ACAGCCTACATGCAACTGAGCAGCCTGACATCTGAGGACTCTGCAGTCT
ATTACTGTGCAAGAGATGGTAACTATGACCACTGGTACTTCGATGTCTG
GGGCACAGGGACCACGGTCACCGTCTCCTCAGGTGGAGGTGGATCAGGT
GGAGGTGGATCTGGTGGAGGTGGATCTGACATCAAGATGACCCAGTCTC
CATCTTCCATGTATGCATCTCTAGGAGAGAGAGTCACTATCACTTGCAA
GGCGAGTCAGGACATTAATAGCTATTTAAGCTGGTTCCAGCAGAAACCA
GGGAAATCTCCTAAGACCCTGATCTATCGTGCAAACAGATTGGTAGATG
GGGTCCCATCAAGGTTCAGTGGCAGTGGATCTGGGCAAGATTATTCTCT
CACCATCAGCAGCCTGGAGTATGAAGATATGGGAATTTATTATTGTCTA
CAGTATGATGAGTTGCTCACGTTCGGTGCTGGGACCAAGCTGGAGCTGA
AACGG.

In some embodiments, the anti-CD123 scFv is encoded by the nucleic acid sequence:

(SEQ ID NO: 79, 15A12HC1_LC2)
ATGGCCCTCCCGGTAACGGCTCTGCTGCTTCCACTCGCACTGCTCTTGC
ATGCTGCCAGACCACAGGTCCAGCTGCAGCAGTCTGGGGCTGAACTGGC
AAAACCTGGGGCCTCAGTGAAGATGTCCTGCAAGGCTTCTGGCTACACC
TTTTCTAGCTACTGGATGCACTGGCTAAAACAGAGGCCTGGACAGGGTC
TGGAGTGGATTGGATACATTAATCCTAGCAGTGGTTATACTAACTACAA
TCAGAAGTTCAAGGACAAGGCCACATTGACTGCAGACAAATCCTCCAGC
ACAGCCTACATGCAACTGAGCAGCCTGACATCTGAGGACTCTGCAGTCT
ATTACTGTGCAAGAGATGGTAACTATGACCACTGGTACTTCGATGTCTG
GGGCACAGGGACCACGGTCACCGTCTCCTCAGGTGGAGGTGGATCAGGT
GGAGGTGGATCTGGTGGAGGTGGATCTGACATTGTGATGTCACAGTCTC
CATCCTCCCTGGCTGTGTCAGCAGGAGAGAGGGTCACTATGAGCTGCAG
ATCCAGTCAGAGTCTGCTCAACAGTAGAACCCGAAAGAACTACTTGGCT
TGGTACCAGCAGAAACCAGGGCAGTCTCCTAAGCTGCTGATCTACTGGG
CATCCACTAGGGAATCTGGGGTCCCTGATCGCTTCTCAGGCAGTGGATC
TGGGACAGATTTCACTCTCACCATCAGCAGTGTGCAGGCTGAAGACCTG
GCAGTTTATTACTGCGAGCAATCTTATAATCTATTCACGTTCGGCTCGG
GGACAAAGTTGGAAATAAAACGG.

In some embodiments, the anti-CD123 scFv is encoded by the nucleic acid sequence:

(SEQ ID NO: 80, 15A12HC2_LC1)
ATGGCCCTCCCGGTAACGGCTCTGCTGCTTCCACTCGCACTGCTCTTGC
ATGCTGCCAGACCACAGGTTCAACTGCAGCAGCCTGGGGCTGAGCTGGT
GAGGCCTGGGGCTTCAGTGAAGATGTCCTGCAAGGCTTCTGGCTACACC
CTCACCACCTACTTGATGGACTGGGTAAAACAGAGGCTTGGACAAGGCT
TTGAGTGGATTGGAAATATTAATCCTAATAGTGGTAGTAGTAACTACAA
TGAGAAGTTCAAGGGCAAGGCCAAGCTGACTGTAGACAAATCCTCCAGC
ACAGCCTACATGCAACTCAGCAGCCTGACATCTGAGGACTCTGCGGTCT
ATTACTGTGCAATACGGCACTATGGTGGTAGTCTCTTTGACTACTGGGG
CCAAGGCACCACTCTCACAGTCTCCTCAGGTGGAGGTGGATCAGGTGGA
GGTGGATCTGGTGGAGGTGGATCTGACATCAAGATGACCCAGTCTCCAT
CTTCCATGTATGCATCTCTAGGAGAGAGAGTCACTATCACTTGCAAGGC
GAGTCAGGACATTAATAGCTATTTAAGCTGGTTCCAGCAGAAACCAGGG
AAATCTCCTAAGACCCTGATCTATCGTGCAAACAGATTGGTAGATGGGG
TCCCATCAAGGTTCAGTGGCAGTGGATCTGGGCAAGATTATTCTCTCAC
CATCAGCAGCCTGGAGTATGAAGATATGGGAATTTATTATTGTCTACAG
TATGATGAGTTGCTCACGTTCGGTGCTGGGACCAAGCTGGAGCTGAAAC
GG.

In some embodiments, the anti-CD123 scFv is encoded by the nucleic acid sequence:

(SEQ ID NO: 81, 15A12HC2_LC2)
ATGGCCCTCCCGGTAACGGCTCTGCTGCTTCCACTCGCACTGCTCTTGC
ATGCTGCCAGACCACAGGTTCAACTGCAGCAGCCTGGGGCTGAGCTGGT
GAGGCCTGGGGCTTCAGTGAAGATGTCCTGCAAGGCTTCTGGCTACACC
CTCACCACCTACTTGATGGACTGGGTAAAACAGAGGCTTGGACAAGGCT
TTGAGTGGATTGGAAATATTAATCCTAATAGTGGTAGTAGTAACTACAA
TGAGAAGTTCAAGGGCAAGGCCAAGCTGACTGTAGACAAATCCTCCAGC
ACAGCCTACATGCAACTCAGCAGCCTGACATCTGAGGACTCTGCGGTCT
ATTACTGTGCAATACGGCACTATGGTGGTAGTCTCTTTGACTACTGGGG
CCAAGGCACCACTCTCACAGTCTCCTCAGGTGGAGGTGGATCAGGTGGA
GGTGGATCTGGTGGAGGTGGATCTGACATTGTGATGTCACAGTCTCCAT
CCTCCCTGGCTGTGTCAGCAGGAGAGAGGGTCACTATGAGCTGCAGATC
CAGTCAGAGTCTGCTCAACAGTAGAACCCGAAAGAACTACTTGGCTTGG
TACCAGCAGAAACCAGGGCAGTCTCCTAAGCTGCTGATCTACTGGGCAT
CCACTAGGGAATCTGGGGTCCCTGATCGCTTCTCAGGCAGTGGATCTGG
GACAGATTTCACTCTCACCATCAGCAGTGTGCAGGCTGAAGACCTGGCA
GTTTATTACTGCGAGCAATCTTATAATCTATTCACGTTCGGCTCGGGGA
CAAAGTTGGAAATAAAACGG.

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

In some embodiments, the CAR comprises a CD8 hinge and transmembrane domain having the amino acid sequence:

(SEQ ID NO: 82)
TTTPAPRPPTPAPTIASQPLSLRPEACRPAAGGAVHTRGLDFACDIYIW
APLAGTCGVLLLSLVITLYC.

In some embodiments, the CSR comprises a 41BB domain having the amino acid sequence:

(SEQ ID NO: 83)
KRGRKKLLYIFKQPFMRPVQTTQEEDGCSCRFPEEEEGGCEL.

In some embodiments, the CSR comprises a CD3z domain having the amino acid sequence:

(SEQ ID NO: 84)
RVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

In some embodiments, the CSR comprises the amino acid sequence:

(SEQ ID NO: 85)
KRGRKKLLYIFKQPFMRPVQTTQEEDGCSCRFPEEEEGGCELRVKFSRS
ADAPAYKQGQNQLYNELNLGRREEYDVLDKRRGRDPEMGGKPRRKNPQE
GLYNELQKDKMAEAYSEIGMKGERRRGKGHDGLYQGLSTATKDTYDALH
MQALPPR.

Also disclosed is dual CAR T cell containing the disclosed CD33-specific CAR, and at least one other CAR with a different ligand binding target. In these embodiments, one CAR can include only the CD3ζ domain and the other CAR can include only the co-stimulatory domain(s). In these embodiments, dual CAR T cell activation would require co-expression of both targets on the target cell.

Therefore, in some embodiments, the disclosed CD33-specific CAR polypeptide contains an incomplete endodomain. For example, the CAR polypeptide can contain only an intracellular signaling domain or a co-stimulatory domain, but not both. In these embodiments, the immune effector cell is not activated unless it and a second CAR polypeptide (or endogenous T-cell receptor) that contains the missing domain both bind their respective targets. Therefore, in some embodiments, the CAR polypeptide contains a CD3 zeta (CD3ζ) signaling domain but does not contain a costimulatory signaling region (CSR). In other embodiments, the CAR polypeptide contains the cytoplasmic domain of CD28, 4-1BB, or a combination thereof, but does not contain a CD3 zeta (CD3ζ) signaling domain (SD).

In some embodiments, the intracellular signaling domain is a CD3 zeta (CD3ζ) signaling domain. In some embodiments, the costimulatory signaling region comprises the cytoplasmic domain of CD28, 4-1BB, or a combination thereof. In some cases, the costimulatory signaling region contains 1, 2, 3, or 4 cytoplasmic domains of one or more intracellular signaling and/or costimulatory molecules. In some embodiments, the co-stimulatory signaling region contains one or more mutations in the cytoplasmic domains of CD28 and/or 4-1BB that enhance signaling.

In some embodiments, the disclosed CARs comprise a costimulatory signaling region comprising a mutated form of the cytoplasmic domain of CD28 with altered phosphorylation at Y206 and/or Y218. In some embodiments, the disclosed CAR comprises an attenuating mutation at Y206, which will reduce the activity of the CAR. In some embodiments, the disclosed CAR comprises an attenuating mutation at Y218, which will reduce expression of the CAR. Any amino acid residue, such as alanine or phenylalanine, can be substituted for the tyrosine to achieve attenuation. In some embodiments, the tyrosine at Y206 and/or Y218 is substituted with a phosphomimetic residue. In some embodiments, the disclosed CAR substitution of Y206 with a phosphomimetic residue, which will increase the activity of the CAR. In some embodiments, the disclosed CAR comprises substitution of Y218 with a phosphomimetic residue, which will increase expression of the CAR. For example, the phosphomimetic residue can be phosphotyrosine. In some embodiments, a CAR may contain a combination of phosphomimetic amino acids and substitution(s) with non-phosphorylatable amino acids in different residues of the same CAR. For instance, a CAR may contain an alanine or phenylalanine substitution in Y209 and/or Y191 PLUS a phosphomimetic substitution in Y206 and/or Y218.

In some embodiments, the disclosed CARs comprises one or more 41BB domains with mutations that enhance binding to specific TRAF proteins, such as TRAF1, TRAF2, TRAF3, TRAF4, TRAF5, TRAF6, or any combination thereof. In some cases, the 41BB mutation enhances TRAF1- and/or TRAF2-dependent proliferation and survival of the T-cell, e.g. through NF-kB. In some cases, the 41BB mutation enhances TRAF3-dependent antitumor efficacy, e.g. through IRF7/INFβ.

Also as disclosed herein, TRAF proteins can in some cases enhance CAR T cell function independent of NFκB and 41BB. For example, TRAF proteins can in some cases enhance CD28 co-stimulation in T cells. Therefore, also disclosed herein are immune effector cells co-expressing CARs with one or more TRAF proteins, such as TRAF1, TRAF2, TRAF3, TRAF4, TRAF5, TRAF6, or any combination thereof. In some cases, the CAR is any CAR that targets a tumor antigen. For example, first-generation CARs typically had the intracellular domain from the CD32 chain, while second-generation CARs added intracellular signaling domains from various costimulatory protein receptors (e.g., CD28, 41BB, ICOS) to the endodomain of the CAR to provide additional signals to the T cell. In some cases, the CAR is the disclosed CAR with enhanced 41BB activation.

Additional CAR constructs are described, for example, in Fresnak A D, et al. Engineered T cells: the promise and challenges of cancer immunotherapy. Nat Rev Cancer. 2016 Aug. 23; 16(9):566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or sCAR.

TRUCKs (T cells redirected for universal cytokine killing) co-express a chimeric antigen receptor (CAR) and an antitumor cytokine. Cytokine expression may be constitutive or induced by T cell activation. Targeted by CAR specificity, localized production of pro-inflammatory cytokines recruits endogenous immune cells to tumor sites and may potentiate an antitumor response.

Universal, allogeneic CAR T cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds to a tumor ligand, thereby enhancing tumor homing.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

Marked CAR T cells express a CAR plus a tumor epitope to which an existing monoclonal antibody agent binds. In the setting of intolerable adverse effects, administration of the monoclonal antibody clears the CAR T cells and alleviates symptoms with no additional off-tumor effects.

A tandem CAR (TanCAR) T cell expresses a single CAR consisting of two linked single-chain variable fragments (scFvs) that have different affinities fused to intracellular co-stimulatory domain(s) and a CD33 domain. TanCAR T cell activation is achieved only when target cells co-express both targets.

A dual CAR T cell expresses two separate CARs with different ligand binding targets; one CAR includes only the CD3ζ domain and the other CAR includes only the co-stimulatory domain(s). Dual CAR T cell activation requires co-expression of both targets on the tumor.

A safety CAR (sCAR) consists of an extracellular scFv fused to an intracellular inhibitory domain. SCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3Z, CD3δ, CD3γ, CD3ε, CD32 (Fc gamma RIIa), DAP10, DAP12, CD79a, CD79b, FcγRIγ, FcγRIIIγ, FcεRIβ (FCERIB), and FcεRIγ (FCERIG).

In particular embodiments, the intracellular signaling domain is derived from CD3 zeta (CD3Z) (TCR zeta, GenBank accno. BAG36664.1). T-cell surface glycoprotein CD3 zeta (CD32) chain, also known as T-cell receptor T3 zeta chain or CD247 (Cluster of Differentiation 247), is a protein that in humans is encoded by the CD247 gene.

First-generation CARs typically had the intracellular domain from the CD3ζ chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. Preclinical studies have indicated that the second generation of CAR designs improves the antitumor activity of T cells. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g., anti-CD33 scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

Tables 1, 2, and 3 below provide some example combinations of CD33- or CD123-binding region (“ABD”), co-stimulatory signaling regions, and intracellular signaling domain that can occur in the disclosed CARs.

TABLE 1
First Generation CARs

| ScFv | Signal Domain |
|---|---|
| ABD | CD8 |
| ABD | CD3ζ |
| ABD | CD3δ |
| ABD | CD3γ |
| ABD | CD3ε |
| ABD | FcγRI-γ |
| ABD | FcγRIII-γ |
| ABD | FcεRIβ |
| ABD | FcεRIγ |
| ABD | DAP10 |
| ABD | DAP12 |
| ABD | CD32 |
| ABD | CD79a |

TABLE 2
Second Generation CARs

| | Co-stimulatory | Signal | | Co-stimulatory | Signal |
|---|---|---|---|---|---|
| ScFv | Signal | Domain | ScFv | Signal | Domain |
| ABD | CD28 | CD8 | CD33 | CD80 | FcεRIβ |
| ABD | CD28 | CD3ζ | CD33 | CD80 | FcεRIγ |
| ABD | CD28 | CD3δ | CD33 | CD80 | DAP10 |
| ABD | CD28 | CD3γ | CD33 | CD80 | DAP12 |
| ABD | CD28 | CD3ε | CD33 | CD80 | CD32 |
| ABD | CD28 | FcγRI-γ | CD33 | CD80 | CD79a |
| ABD | CD28 | FcγRIII-γ | CD33 | CD80 | CD79b |
| ABD | CD28 | FcεRIβ | CD33 | CD86 | CD8 |
| ABD | CD28 | FcεRIγ | CD33 | CD86 | CD3ζ |
| ABD | CD28 | DAP10 | CD33 | CD86 | CD3δ |
| ABD | CD28 | DAP12 | CD33 | CD86 | CD3γ |
| ABD | CD28 | CD32 | CD33 | CD86 | CD3ε |
| ABD | CD28 | CD79a | CD33 | CD86 | FcγRI-γ |
| ABD | CD28 | CD79b | CD33 | CD86 | FcγRIII-γ |
| ABD | CD8 | CD8 | CD33 | CD86 | FcεRIβ |
| ABD | CD8 | CD3ζ | CD33 | CD86 | FcεRIγ |
| ABD | CD8 | CD3δ | CD33 | CD86 | DAP10 |
| ABD | CD8 | CD3γ | CD33 | CD86 | DAP12 |
| ABD | CD8 | CD3ε | CD33 | CD86 | CD32 |
| ABD | CD8 | FcγRI-γ | CD33 | CD86 | CD79a |
| ABD | CD8 | FcγRIII-γ | CD33 | CD86 | CD79b |
| ABD | CD8 | FcεRIβ | CD33 | OX40 | CD8 |
| ABD | CD8 | FcεRIγ | CD33 | OX40 | CD3ζ |
| ABD | CD8 | DAP10 | CD33 | OX40 | CD3δ |
| ABD | CD8 | DAP12 | CD33 | OX40 | CD3γ |
| ABD | CD8 | CD32 | CD33 | OX40 | CD3ε |
| ABD | CD8 | CD79a | CD33 | OX40 | FcγRI-γ |
| ABD | CD8 | CD79b | CD33 | OX40 | FcγRIII-γ |
| ABD | CD4 | CD8 | CD33 | OX40 | FcεRIβ |
| ABD | CD4 | CD3ζ | CD33 | OX40 | FcεRIγ |
| ABD | CD4 | CD3δ | CD33 | OX40 | DAP10 |
| ABD | CD4 | CD3γ | CD33 | OX40 | DAP12 |
| ABD | CD4 | CD3ε | CD33 | OX40 | CD32 |
| ABD | CD4 | FcγRI-γ | CD33 | OX40 | CD79a |
| ABD | CD4 | FcγRIII-γ | CD33 | OX40 | CD79b |
| ABD | CD4 | FcεRIβ | CD33 | DAP10 | CD8 |
| ABD | CD4 | FcεRIγ | CD33 | DAP10 | CD3ζ |
| ABD | CD4 | DAP10 | CD33 | DAP10 | CD3δ |
| ABD | CD4 | DAP12 | CD33 | DAP10 | CD3γ |
| ABD | CD4 | CD32 | CD33 | DAP10 | CD3ε |
| ABD | CD4 | CD79a | CD33 | DAP10 | FcγRI-γ |
| ABD | CD4 | CD79b | CD33 | DAP10 | FcγRIII-γ |
| ABD | b2c | CD8 | CD33 | DAP10 | FcεRIβ |
| ABD | b2c | CD3ζ | CD33 | DAP10 | FcεRIγ |
| ABD | b2c | CD3δ | CD33 | DAP10 | DAP10 |
| ABD | b2c | CD3γ | CD33 | DAP10 | DAP12 |
| ABD | b2c | CD3ε | CD33 | DAP10 | CD32 |
| ABD | b2c | FcγRI-γ | CD33 | DAP10 | CD79a |
| ABD | b2c | FcγRIII-γ | CD33 | DAP10 | CD79b |
| ABD | b2c | FcεRIβ | CD33 | DAP12 | CD8 |
| ABD | b2c | FcεRIγ | CD33 | DAP12 | CD3ζ |
| ABD | b2c | DAP10 | CD33 | DAP12 | CD3δ |
| ABD | b2c | DAP12 | CD33 | DAP12 | CD3γ |
| ABD | b2c | CD32 | CD33 | DAP12 | CD3ε |
| ABD | b2c | CD79a | CD33 | DAP12 | FcγRI-γ |
| ABD | b2c | CD79b | CD33 | DAP12 | FcγRIII-γ |
| ABD | CD137/41BB | CD8 | CD33 | DAP12 | FcεRIβ |
| ABD | CD137/41BB | CD3ζ | CD33 | DAP12 | FcεRIγ |
| ABD | CD137/41BB | CD3δ | CD33 | DAP12 | DAP10 |
| ABD | CD137/41BB | CD3γ | CD33 | DAP12 | DAP12 |
| ABD | CD137/41BB | CD3ε | CD33 | DAP12 | CD32 |
| ABD | CD137/41BB | FcγRI-γ | CD33 | DAP12 | CD79a |
| ABD | CD137/41BB | FcγRIII-γ | CD33 | DAP12 | CD79b |
| ABD | CD137/41BB | FcεRIβ | CD33 | MyD88 | CD8 |
| ABD | CD137/41BB | FcεRIγ | CD33 | MyD88 | CD3ζ |
| ABD | CD137/41BB | DAP10 | CD33 | MyD88 | CD3δ |
| ABD | CD137/41BB | DAP12 | CD33 | MyD88 | CD3γ |
| ABD | CD137/41BB | CD32 | CD33 | MyD88 | CD3ε |
| ABD | CD137/41BB | CD79a | CD33 | MyD88 | FcγRI-γ |
| ABD | CD137/41BB | CD79b | CD33 | MyD88 | FcγRIII-γ |
| ABD | ICOS | CD8 | CD33 | MyD88 | FcεRIβ |
| ABD | ICOS | CD3ζ | CD33 | MyD88 | FcεRIγ |
| ABD | ICOS | CD3δ | CD33 | MyD88 | DAP10 |
| ABD | ICOS | CD3γ | CD33 | MyD88 | DAP12 |
| ABD | ICOS | CD3ε | CD33 | MyD88 | CD32 |
| ABD | ICOS | FcγRI-γ | CD33 | MyD88 | CD79a |
| ABD | ICOS | FcγRIII-γ | CD33 | MyD88 | CD79b |
| ABD | ICOS | FcεRIβ | CD33 | CD7 | CD8 |
| ABD | ICOS | FcεRIγ | CD33 | CD7 | CD3ζ |
| ABD | ICOS | DAP10 | CD33 | CD7 | CD3δ |
| ABD | ICOS | DAP12 | CD33 | CD7 | CD3γ |
| ABD | ICOS | CD32 | CD33 | CD7 | CD3ε |
| ABD | ICOS | CD79a | CD33 | CD7 | FcγRI-γ |
| ABD | ICOS | CD79b | CD33 | CD7 | FcγRIII-γ |
| ABD | CD27 | CD8 | CD33 | CD7 | FcεRIβ |
| ABD | CD27 | CD3ζ | CD33 | CD7 | FcεRIγ |
| ABD | CD27 | CD3δ | CD33 | CD7 | DAP10 |
| ABD | CD27 | CD3γ | CD33 | CD7 | DAP12 |
| ABD | CD27 | CD3ε | CD33 | CD7 | CD32 |
| ABD | CD27 | FcγRI-γ | CD33 | CD7 | CD79a |
| ABD | CD27 | FcγRIII-γ | CD33 | CD7 | CD79b |
| ABD | CD27 | FcεRIβ | CD33 | BTNL3 | CD8 |
| ABD | CD27 | FcεRIγ | CD33 | BTNL3 | CD3ζ |
| ABD | CD27 | DAP10 | CD33 | BTNL3 | CD3δ |
| ABD | CD27 | DAP12 | CD33 | BTNL3 | CD3γ |
| ABD | CD27 | CD32 | CD33 | BTNL3 | CD3ε |
| ABD | CD27 | CD79a | CD33 | BTNL3 | FcγRI-γ |
| ABD | CD27 | CD79b | CD33 | BTNL3 | FcγRIII-γ |
| ABD | CD28δ | CD8 | CD33 | BTNL3 | FcεRIβ |
| ABD | CD28δ | CD3ζ | CD33 | BTNL3 | FcεRIγ |
| ABD | CD28δ | CD3δ | CD33 | BTNL3 | DAP10 |
| ABD | CD28δ | CD3γ | CD33 | BTNL3 | DAP12 |
| ABD | CD28δ | CD3ε | CD33 | BTNL3 | CD32 |
| ABD | CD28δ | FcγRI-γ | CD33 | BTNL3 | CD79a |
| ABD | CD28δ | FcγRIII-γ | CD33 | BTNL3 | CD79b |
| ABD | CD28δ | FcεRIβ | CD33 | NKG2D | CD8 |
| ABD | CD28δ | FcεRIγ | CD33 | NKG2D | CD3ζ |
| ABD | CD28δ | DAP10 | CD33 | NKG2D | CD3δ |
| ABD | CD28δ | DAP12 | CD33 | NKG2D | CD3γ |
| ABD | CD28δ | CD32 | CD33 | NKG2D | CD3ε |
| ABD | CD28δ | CD79a | CD33 | NKG2D | FcγRI-γ |
| ABD | CD28δ | CD79b | CD33 | NKG2D | FcγRIII-γ |
| ABD | CD80 | CD8 | CD33 | NKG2D | FcεRIβ |
| ABD | CD80 | CD3ζ | CD33 | NKG2D | FcεRIγ |
| ABD | CD80 | CD3δ | CD33 | NKG2D | DAP10 |
| ABD | CD80 | CD3γ | CD33 | NKG2D | DAP12 |
| ABD | CD80 | CD3ε | CD33 | NKG2D | CD32 |
| ABD | CD80 | FcγRI-γ | CD33 | NKG2D | CD79a |
| ABD | CD80 | FcγRIII-γ | CD33 | NKG2D | CD79b |

TABLE 3
Third Generation CARs

| | Co-stimulatory | Co-stimulatory | Signal |
|---|---|---|---|
| ScFv | Signal | Signal | Domain |
| ABD | CD28 | CD28 | CD8 |
| ABD | CD28 | CD28 | CD3ζ |
| ABD | CD28 | CD28 | CD3δ |
| ABD | CD28 | CD28 | CD3γ |
| ABD | CD28 | CD28 | CD3ε |
| ABD | CD28 | CD28 | FcγRI-γ |
| ABD | CD28 | CD28 | FcγRIII-γ |
| ABD | CD28 | CD28 | FcεRIβ |
| ABD | CD28 | CD28 | FcεRIγ |
| ABD | CD28 | CD28 | DAP10 |
| ABD | CD28 | CD28 | DAP12 |
| ABD | CD28 | CD28 | CD32 |
| ABD | CD28 | CD28 | CD79a |
| ABD | CD28 | CD28 | CD79b |
| ABD | CD28 | CD8 | CD8 |
| ABD | CD28 | CD8 | CD3ζ |
| ABD | CD28 | CD8 | CD3δ |
| ABD | CD28 | CD8 | CD3γ |
| ABD | CD28 | CD8 | CD3ε |
| ABD | CD28 | CD8 | FcγRI-γ |
| ABD | CD28 | CD8 | FcγRIII-γ |
| ABD | CD28 | CD8 | FcεRIβ |
| ABD | CD28 | CD8 | FcεRIγ |
| ABD | CD28 | CD8 | DAP10 |
| ABD | CD28 | CD8 | DAP12 |
| ABD | CD28 | CD8 | CD32 |
| ABD | CD28 | CD8 | CD79a |
| ABD | CD28 | CD8 | CD79b |
| ABD | CD28 | CD4 | CD8 |
| ABD | CD28 | CD4 | CD3ζ |
| ABD | CD28 | CD4 | CD3δ |
| ABD | CD28 | CD4 | CD3γ |
| ABD | CD28 | CD4 | CD3ε |
| ABD | CD28 | CD4 | FcγRI-γ |
| ABD | CD28 | CD4 | FcγRIII-γ |
| ABD | CD28 | CD4 | FcεRIβ |
| ABD | CD28 | CD4 | FcεRIγ |
| ABD | CD28 | CD4 | DAP10 |
| ABD | CD28 | CD4 | DAP12 |
| ABD | CD28 | CD4 | CD32 |
| ABD | CD28 | CD4 | CD79a |
| ABD | CD28 | CD4 | CD79b |
| ABD | CD28 | b2c | CD8 |
| ABD | CD28 | b2c | CD3ζ |
| ABD | CD28 | b2c | CD3δ |
| ABD | CD28 | b2c | CD3γ |
| ABD | CD28 | b2c | CD3ε |
| ABD | CD28 | b2c | FcγRI-γ |
| ABD | CD28 | b2c | FcγRIII-γ |
| ABD | CD28 | b2c | FcεRIβ |
| ABD | CD28 | b2c | FcεRIγ |
| ABD | CD28 | b2c | DAP10 |
| ABD | CD28 | b2c | DAP12 |
| ABD | CD28 | b2c | CD32 |
| ABD | CD28 | b2c | CD79a |
| ABD | CD28 | b2c | CD79b |
| ABD | CD28 | CD137/41BB | CD8 |
| ABD | CD28 | CD137/41BB | CD3ζ |
| ABD | CD28 | CD137/41BB | CD3δ |
| ABD | CD28 | CD137/41BB | CD3γ |
| ABD | CD28 | CD137/41BB | CD3ε |
| ABD | CD28 | CD137/41BB | FcγRI-γ |
| ABD | CD28 | CD137/41BB | FcγRIII-γ |
| ABD | CD28 | CD137/41BB | FcεRIβ |
| ABD | CD28 | CD137/41BB | FcεRIγ |
| ABD | CD28 | CD137/41BB | DAP10 |
| ABD | CD28 | CD137/41BB | DAP12 |
| ABD | CD28 | CD137/41BB | CD32 |
| ABD | CD28 | CD137/41BB | CD79a |
| ABD | CD28 | CD137/41BB | CD79b |
| ABD | CD28 | ICOS | CD8 |
| ABD | CD28 | ICOS | CD3ζ |
| ABD | CD28 | ICOS | CD3δ |
| ABD | CD28 | ICOS | CD3γ |
| ABD | CD28 | ICOS | CD3ε |
| ABD | CD28 | ICOS | FcγRI-γ |
| ABD | CD28 | ICOS | FcγRIII-γ |
| ABD | CD28 | ICOS | FcεRIβ |
| ABD | CD28 | ICOS | FcεRIγ |
| ABD | CD28 | ICOS | DAP10 |
| ABD | CD28 | ICOS | DAP12 |
| ABD | CD28 | ICOS | CD32 |
| ABD | CD28 | ICOS | CD79a |
| ABD | CD28 | ICOS | CD79b |
| ABD | CD28 | CD27 | CD8 |
| ABD | CD28 | CD27 | CD3ζ |
| ABD | CD28 | CD27 | CD3δ |
| ABD | CD28 | CD27 | CD3γ |
| ABD | CD28 | CD27 | CD3ε |
| ABD | CD28 | CD27 | FcγRI-γ |
| ABD | CD28 | CD27 | FcγRIII-γ |
| ABD | CD28 | CD27 | FcεRIβ |
| ABD | CD28 | CD27 | FcεRIγ |
| ABD | CD28 | CD27 | DAP10 |
| ABD | CD28 | CD27 | DAP12 |
| ABD | CD28 | CD27 | CD32 |
| ABD | CD28 | CD27 | CD79a |
| ABD | CD28 | CD27 | CD79b |
| ABD | CD28 | CD28δ | CD8 |
| ABD | CD28 | CD28δ | CD3ζ |
| ABD | CD28 | CD28δ | CD3δ |
| ABD | CD28 | CD28δ | CD3γ |
| ABD | CD28 | CD28δ | CD3ε |
| ABD | CD28 | CD28δ | FcγRI-γ |
| ABD | CD28 | CD28δ | FcγRIII-γ |
| ABD | CD28 | CD28δ | FcεRIß |
| ABD | CD28 | CD28δ | FcεRIγ |
| ABD | CD28 | CD28δ | DAP10 |
| ABD | CD28 | CD28δ | DAP12 |
| ABD | CD28 | CD28δ | CD32 |
| ABD | CD28 | CD28δ | CD79a |
| ABD | CD28 | CD28δ | CD79b |
| ABD | CD28 | CD80 | CD8 |
| ABD | CD28 | CD80 | CD3ζ |
| ABD | CD28 | CD80 | CD3δ |
| ABD | CD28 | CD80 | CD3γ |
| ABD | CD28 | CD80 | CD3ε |
| ABD | CD28 | CD80 | FcγRI-γ |
| ABD | CD28 | CD80 | FcγRIII-γ |
| ABD | CD28 | CD80 | FcεRIß |
| ABD | CD28 | CD80 | FcεRIγ |
| ABD | CD28 | CD80 | DAP10 |
| ABD | CD28 | CD80 | DAP12 |
| ABD | CD28 | CD80 | CD32 |
| ABD | CD28 | CD80 | CD79a |
| ABD | CD28 | CD80 | CD79b |
| ABD | CD28 | CD86 | CD8 |
| ABD | CD28 | CD86 | CD3ζ |
| ABD | CD28 | CD86 | CD3δ |
| ABD | CD28 | CD86 | CD3γ |
| ABD | CD28 | CD86 | CD3ε |
| ABD | CD28 | CD86 | FcγRI-γ |
| ABD | CD28 | CD86 | FcγRIII-γ |
| ABD | CD28 | CD86 | FcεRIβ |
| ABD | CD28 | CD86 | FcεRIγ |
| ABD | CD28 | CD86 | DAP10 |
| ABD | CD28 | CD86 | DAP12 |
| ABD | CD28 | CD86 | CD32 |
| ABD | CD28 | CD86 | CD79a |
| ABD | CD28 | CD86 | CD79b |
| ABD | CD28 | OX40 | CD8 |
| ABD | CD28 | OX40 | CD3ζ |
| ABD | CD28 | OX40 | CD3δ |
| ABD | CD28 | OX40 | CD3γ |
| ABD | CD28 | OX40 | CD3ε |
| ABD | CD28 | OX40 | FcγRI-γ |
| ABD | CD28 | OX40 | FcγRIII-γ |
| ABD | CD28 | OX40 | FcεRIβ |
| ABD | CD28 | OX40 | FcεRIγ |
| ABD | CD28 | OX40 | DAP10 |
| ABD | CD28 | OX40 | DAP12 |
| ABD | CD28 | OX40 | CD32 |
| ABD | CD28 | OX40 | CD79a |
| ABD | CD28 | OX40 | CD79b |
| ABD | CD28 | DAP10 | CD8 |
| ABD | CD28 | DAP10 | CD3ζ |
| ABD | CD28 | DAP10 | CD3δ |
| ABD | CD28 | DAP10 | CD3γ |
| ABD | CD28 | DAP10 | CD3ε |
| ABD | CD28 | DAP10 | FcγRI-γ |
| ABD | CD28 | DAP10 | FcγRIII-γ |
| ABD | CD28 | DAP10 | FcεRIβ |
| ABD | CD28 | DAP10 | FcεRIγ |
| ABD | CD28 | DAP10 | DAP10 |
| ABD | CD28 | DAP10 | DAP12 |
| ABD | CD28 | DAP10 | CD32 |
| ABD | CD28 | DAP10 | CD79a |
| ABD | CD28 | DAP10 | CD79b |
| ABD | CD28 | DAP12 | CD8 |
| ABD | CD28 | DAP12 | CD3ζ |
| ABD | CD28 | DAP12 | CD3δ |
| ABD | CD28 | DAP12 | CD3γ |
| ABD | CD28 | DAP12 | CD3ε |
| ABD | CD28 | DAP12 | FcγRI-γ |
| ABD | CD28 | DAP12 | FcγRIII-γ |
| ABD | CD28 | DAP12 | FcεRIβ |
| ABD | CD28 | DAP12 | FcεRIγ |
| ABD | CD28 | DAP12 | DAP10 |
| ABD | CD28 | DAP12 | DAP12 |
| ABD | CD28 | DAP12 | CD32 |
| ABD | CD28 | DAP12 | CD79a |
| ABD | CD28 | DAP12 | CD79b |
| ABD | CD28 | MyD88 | CD8 |
| ABD | CD28 | MyD88 | CD3ζ |
| ABD | CD28 | MyD88 | CD3δ |
| ABD | CD28 | MyD88 | CD3γ |
| ABD | CD28 | MyD88 | CD3ε |
| ABD | CD28 | MyD88 | FcγRI-γ |
| ABD | CD28 | MyD88 | FcγRIII-γ |
| ABD | CD28 | MyD88 | FcεRIβ |
| ABD | CD28 | MyD88 | FcεRIγ |
| ABD | CD28 | MyD88 | DAP10 |
| ABD | CD28 | MyD88 | DAP12 |
| ABD | CD28 | MyD88 | CD32 |
| ABD | CD28 | MyD88 | CD79a |
| ABD | CD28 | MyD88 | CD79b |
| ABD | CD28 | CD7 | CD8 |
| ABD | CD28 | CD7 | CD3ζ |
| ABD | CD28 | CD7 | CD3δ |
| ABD | CD28 | CD7 | CD3γ |
| ABD | CD28 | CD7 | CD3ε |
| ABD | CD28 | CD7 | FcγRI-γ |
| ABD | CD28 | CD7 | FcγRIII-γ |
| ABD | CD28 | CD7 | FcεRIβ |
| ABD | CD28 | CD7 | FcεRIγ |
| ABD | CD28 | CD7 | DAP10 |
| ABD | CD28 | CD7 | DAP12 |
| ABD | CD28 | CD7 | CD32 |
| ABD | CD28 | CD7 | CD79a |
| ABD | CD28 | CD7 | CD79b |
| ABD | CD28 | BTNL3 | CD8 |
| ABD | CD28 | BTNL3 | CD3ζ |
| ABD | CD28 | BTNL3 | CD3δ |
| ABD | CD28 | BTNL3 | CD3γ |
| ABD | CD28 | BTNL3 | CD3ε |
| ABD | CD28 | BTNL3 | FcγRI-γ |
| ABD | CD28 | BTNL3 | FcγRIII-γ |
| ABD | CD28 | BTNL3 | FcεRIβ |
| ABD | CD28 | BTNL3 | FcεRIγ |
| ABD | CD28 | BTNL3 | DAP10 |
| ABD | CD28 | BTNL3 | DAP12 |
| ABD | CD28 | BTNL3 | CD32 |
| ABD | CD28 | BTNL3 | CD79a |
| ABD | CD28 | BTNL3 | CD79b |
| ABD | CD28 | NKG2D | CD8 |
| ABD | CD28 | NKG2D | CD3ζ |
| ABD | CD28 | NKG2D | CD3δ |
| ABD | CD28 | NKG2D | CD3γ |
| ABD | CD28 | NKG2D | CD3ε |
| ABD | CD28 | NKG2D | FcγRI-γ |
| ABD | CD28 | NKG2D | FcγRIII-γ |
| ABD | CD28 | NKG2D | FcεRIβ |
| ABD | CD28 | NKG2D | FcεRIγ |
| ABD | CD28 | NKG2D | DAP10 |
| ABD | CD28 | NKG2D | DAP12 |
| ABD | CD28 | NKG2D | CD32 |
| ABD | CD28 | NKG2D | CD79a |
| ABD | CD28 | NKG2D | CD79b |
| ABD | CD8 | CD28 | CD8 |
| ABD | CD8 | CD28 | CD3ζ |
| ABD | CD8 | CD28 | CD3δ |
| ABD | CD8 | CD28 | CD3γ |
| ABD | CD8 | CD28 | CD3ε |
| ABD | CD8 | CD28 | FcγRI-γ |
| ABD | CD8 | CD28 | FcγRIII-γ |
| ABD | CD8 | CD28 | FcεRIβ |
| ABD | CD8 | CD28 | FcεRIγ |
| ABD | CD8 | CD28 | DAP10 |
| ABD | CD8 | CD28 | DAP12 |
| ABD | CD8 | CD28 | CD32 |
| ABD | CD8 | CD28 | CD79a |
| ABD | CD8 | CD28 | CD79b |
| ABD | CD8 | CD8 | CD8 |
| ABD | CD8 | CD8 | CD3ζ |
| ABD | CD8 | CD8 | CD3δ |
| ABD | CD8 | CD8 | CD3γ |
| ABD | CD8 | CD8 | CD3ε |
| ABD | CD8 | CD8 | FcγRI-γ |
| ABD | CD8 | CD8 | FcγRIII-γ |
| ABD | CD8 | CD8 | FcεRIβ |
| ABD | CD8 | CD8 | FcεRIγ |
| ABD | CD8 | CD8 | DAP10 |
| ABD | CD8 | CD8 | DAP12 |
| ABD | CD8 | CD8 | CD32 |
| ABD | CD8 | CD8 | CD79a |
| ABD | CD8 | CD8 | CD79b |
| ABD | CD8 | CD4 | CD8 |
| ABD | CD8 | CD4 | CD3ζ |
| ABD | CD8 | CD4 | CD3δ |
| ABD | CD8 | CD4 | CD3γ |
| ABD | CD8 | CD4 | CD3ε |
| ABD | CD8 | CD4 | FcγRI-γ |
| ABD | CD8 | CD4 | FcγRIII-γ |
| ABD | CD8 | CD4 | FcεRIβ |
| ABD | CD8 | CD4 | FcεRIγ |
| ABD | CD8 | CD4 | DAP10 |
| ABD | CD8 | CD4 | DAP12 |
| ABD | CD8 | CD4 | CD32 |
| ABD | CD8 | CD4 | CD79a |
| ABD | CD8 | CD4 | CD79b |
| ABD | CD8 | b2c | CD8 |
| ABD | CD8 | b2c | CD3ζ |
| ABD | CD8 | b2c | CD3δ |
| ABD | CD8 | b2c | CD3γ |
| ABD | CD8 | b2c | CD3ε |
| ABD | CD8 | b2c | FcγRI-γ |
| ABD | CD8 | b2c | FcγRIII-γ |
| ABD | CD8 | b2c | FcεRIβ |
| ABD | CD8 | b2c | FcεRIγ |
| ABD | CD8 | b2c | DAP10 |
| ABD | CD8 | b2c | DAP12 |
| ABD | CD8 | b2c | CD32 |
| ABD | CD8 | b2c | CD79a |
| ABD | CD8 | b2c | CD79b |
| ABD | CD8 | CD137/41BB | CD8 |
| ABD | CD8 | CD137/41BB | CD3ζ |
| ABD | CD8 | CD137/41BB | CD3δ |
| ABD | CD8 | CD137/41BB | CD3γ |
| ABD | CD8 | CD137/41BB | CD3ε |
| ABD | CD8 | CD137/41BB | FcγRI-γ |
| ABD | CD8 | CD137/41BB | FcγRIII-γ |
| ABD | CD8 | CD137/41BB | FcεRIβ |
| ABD | CD8 | CD137/41BB | FcεRIγ |
| ABD | CD8 | CD137/41BB | DAP10 |
| ABD | CD8 | CD137/41BB | DAP12 |
| ABD | CD8 | CD137/41BB | CD32 |
| ABD | CD8 | CD137/41BB | CD79a |
| ABD | CD8 | CD137/41BB | CD79b |
| ABD | CD8 | ICOS | CD8 |
| ABD | CD8 | ICOS | CD3ζ |
| ABD | CD8 | ICOS | CD3δ |
| ABD | CD8 | ICOS | CD3γ |
| ABD | CD8 | ICOS | CD3ε |
| ABD | CD8 | ICOS | FcγRI-γ |
| ABD | CD8 | ICOS | FcγRIII-γ |
| ABD | CD8 | ICOS | FcεRIβ |
| ABD | CD8 | ICOS | FcεRIγ |
| ABD | CD8 | ICOS | DAP10 |
| ABD | CD8 | ICOS | DAP12 |
| ABD | CD8 | ICOS | CD32 |
| ABD | CD8 | ICOS | CD79a |
| ABD | CD8 | ICOS | CD79b |
| ABD | CD8 | CD27 | CD8 |
| ABD | CD8 | CD27 | CD3ζ |
| ABD | CD8 | CD27 | CD3δ |
| ABD | CD8 | CD27 | CD3γ |
| ABD | CD8 | CD27 | CD3ε |
| ABD | CD8 | CD27 | FcγRI-γ |
| ABD | CD8 | CD27 | FcγRIII-γ |
| ABD | CD8 | CD27 | FcεRIß |
| ABD | CD8 | CD27 | FcεRIγ |
| ABD | CD8 | CD27 | DAP10 |
| ABD | CD8 | CD27 | DAP12 |
| ABD | CD8 | CD27 | CD32 |
| ABD | CD8 | CD27 | CD79a |
| ABD | CD8 | CD27 | CD79b |
| ABD | CD8 | CD28δ | CD8 |
| ABD | CD8 | CD28δ | CD3ζ |
| ABD | CD8 | CD28δ | CD3δ |
| ABD | CD8 | CD28δ | CD3γ |
| ABD | CD8 | CD28δ | CD3ε |
| ABD | CD8 | CD28δ | FcγRI-γ |
| ABD | CD8 | CD28δ | FcγRIII-γ |
| ABD | CD8 | CD28δ | FcεRIβ |
| ABD | CD8 | CD28δ | FcεRIγ |
| ABD | CD8 | CD28δ | DAP10 |
| ABD | CD8 | CD28δ | DAP12 |
| ABD | CD8 | CD28δ | CD32 |
| ABD | CD8 | CD28δ | CD79a |
| ABD | CD8 | CD28δ | CD79b |
| ABD | CD8 | CD80 | CD8 |
| ABD | CD8 | CD80 | CD3ζ |
| ABD | CD8 | CD80 | CD3δ |
| ABD | CD8 | CD80 | CD3γ |
| ABD | CD8 | CD80 | CD3ε |
| ABD | CD8 | CD80 | FcγRI-γ |
| ABD | CD8 | CD80 | FcγRIII-γ |
| ABD | CD8 | CD80 | FcεRIβ |
| ABD | CD8 | CD80 | FcεRIγ |
| ABD | CD8 | CD80 | DAP10 |
| ABD | CD8 | CD80 | DAP12 |
| ABD | CD8 | CD80 | CD32 |
| ABD | CD8 | CD80 | CD79a |
| ABD | CD8 | CD80 | CD79b |
| ABD | CD8 | CD86 | CD8 |
| ABD | CD8 | CD86 | CD3ζ |
| ABD | CD8 | CD86 | CD3δ |
| ABD | CD8 | CD86 | CD3γ |
| ABD | CD8 | CD86 | CD3ε |
| ABD | CD8 | CD86 | FcγRI-γ |
| ABD | CD8 | CD86 | FcγRIII-γ |
| ABD | CD8 | CD86 | FcεRIB |
| ABD | CD8 | CD86 | FcεRIγ |
| ABD | CD8 | CD86 | DAP10 |
| ABD | CD8 | CD86 | DAP12 |
| ABD | CD8 | CD86 | CD32 |
| ABD | CD8 | CD86 | CD79a |
| ABD | CD8 | CD86 | CD79b |
| ABD | CD8 | OX40 | CD8 |
| ABD | CD8 | OX40 | CD3ζ |
| ABD | CD8 | OX40 | CD3δ |
| ABD | CD8 | OX40 | CD3γ |
| ABD | CD8 | OX40 | CD3ε |
| ABD | CD8 | OX40 | FcγRI-γ |
| ABD | CD8 | OX40 | FcγRIII-γ |
| ABD | CD8 | OX40 | FcεRIβ |
| ABD | CD8 | OX40 | FcεRIγ |
| ABD | CD8 | OX40 | DAP10 |
| ABD | CD8 | OX40 | DAP12 |
| ABD | CD8 | OX40 | CD32 |
| ABD | CD8 | OX40 | CD79a |
| ABD | CD8 | OX40 | CD79b |
| ABD | CD8 | DAP10 | CD8 |
| ABD | CD8 | DAP10 | CD3ζ |
| ABD | CD8 | DAP10 | CD3δ |
| ABD | CD8 | DAP10 | CD3γ |
| ABD | CD8 | DAP10 | CD3ε |
| ABD | CD8 | DAP10 | FcγRI-γ |
| ABD | CD8 | DAP10 | FcγRIII-γ |
| ABD | CD8 | DAP10 | FcεRIβ |
| ABD | CD8 | DAP10 | FcεRIγ |
| ABD | CD8 | DAP10 | DAP10 |
| ABD | CD8 | DAP10 | DAP12 |
| ABD | CD8 | DAP10 | CD32 |
| ABD | CD8 | DAP10 | CD79a |
| ABD | CD8 | DAP10 | CD79b |
| ABD | CD8 | DAP12 | CD8 |
| ABD | CD8 | DAP12 | CD3ζ |
| ABD | CD8 | DAP12 | CD3δ |
| ABD | CD8 | DAP12 | CD3γ |
| ABD | CD8 | DAP12 | CD3ε |
| ABD | CD8 | DAP12 | FcγRI-γ |
| ABD | CD8 | DAP12 | FcγRIII-γ |
| ABD | CD8 | DAP12 | FcεRIβ |
| ABD | CD8 | DAP12 | FcεRIγ |
| ABD | CD8 | DAP12 | DAP10 |
| ABD | CD8 | DAP12 | DAP12 |
| ABD | CD8 | DAP12 | CD32 |
| ABD | CD8 | DAP12 | CD79a |
| ABD | CD8 | DAP12 | CD79b |
| ABD | CD8 | MyD88 | CD8 |
| ABD | CD8 | MyD88 | CD3ζ |
| ABD | CD8 | MyD88 | CD3δ |
| ABD | CD8 | MyD88 | CD3γ |
| ABD | CD8 | MyD88 | CD3ε |
| ABD | CD8 | MyD88 | FcγRI-γ |
| ABD | CD8 | MyD88 | FcγRIII-γ |
| ABD | CD8 | MyD88 | FcεRIβ |
| ABD | CD8 | MyD88 | FcεRIγ |
| ABD | CD8 | MyD88 | DAP10 |
| ABD | CD8 | MyD88 | DAP12 |
| ABD | CD8 | MyD88 | CD32 |
| ABD | CD8 | MyD88 | CD79a |
| ABD | CD8 | MyD88 | CD79b |
| ABD | CD8 | CD7 | CD8 |
| ABD | CD8 | CD7 | CD3ζ |
| ABD | CD8 | CD7 | CD3δ |
| ABD | CD8 | CD7 | CD3γ |
| ABD | CD8 | CD7 | CD3ε |
| ABD | CD8 | CD7 | FcγRI-γ |
| ABD | CD8 | CD7 | FcγRIII-γ |
| ABD | CD8 | CD7 | FcεRIβ |
| ABD | CD8 | CD7 | FcεRIγ |
| ABD | CD8 | CD7 | DAP10 |
| ABD | CD8 | CD7 | DAP12 |
| ABD | CD8 | CD7 | CD32 |
| ABD | CD8 | CD7 | CD79a |
| ABD | CD8 | CD7 | CD79b |
| ABD | CD8 | BTNL3 | CD8 |
| ABD | CD8 | BTNL3 | CD3ζ |
| ABD | CD8 | BTNL3 | CD3δ |
| ABD | CD8 | BTNL3 | CD3γ |
| ABD | CD8 | BTNL3 | CD3ε |
| ABD | CD8 | BTNL3 | FcγRI-γ |
| ABD | CD8 | BTNL3 | FcγRIII-γ |
| ABD | CD8 | BTNL3 | FcεRIβ |
| ABD | CD8 | BTNL3 | FcεRIγ |
| ABD | CD8 | BTNL3 | DAP10 |
| ABD | CD8 | BTNL3 | DAP12 |
| ABD | CD8 | BTNL3 | CD32 |
| ABD | CD8 | BTNL3 | CD79a |
| ABD | CD8 | BTNL3 | CD79b |
| ABD | CD8 | NKG2D | CD8 |
| ABD | CD8 | NKG2D | CD3ζ |
| ABD | CD8 | NKG2D | CD3δ |
| ABD | CD8 | NKG2D | CD3γ |
| ABD | CD8 | NKG2D | CD3ε |
| ABD | CD8 | NKG2D | FcγRI-γ |
| ABD | CD8 | NKG2D | FcγRIII-γ |
| ABD | CD8 | NKG2D | FcεRIβ |
| ABD | CD8 | NKG2D | FcεRIγ |
| ABD | CD8 | NKG2D | DAP10 |
| ABD | CD8 | NKG2D | DAP12 |
| ABD | CD8 | NKG2D | CD32 |
| ABD | CD8 | NKG2D | CD79a |
| ABD | CD8 | NKG2D | CD79b |
| ABD | CD4 | CD28 | CD8 |
| ABD | CD4 | CD28 | CD3ζ |
| ABD | CD4 | CD28 | CD3δ |
| ABD | CD4 | CD28 | CD3γ |
| ABD | CD4 | CD28 | CD3ε |
| ABD | CD4 | CD28 | FcγRI-γ |
| ABD | CD4 | CD28 | FcγRIII-γ |
| ABD | CD4 | CD28 | FcεRIβ |
| ABD | CD4 | CD28 | FcεRIγ |
| ABD | CD4 | CD28 | DAP10 |
| ABD | CD4 | CD28 | DAP12 |
| ABD | CD4 | CD28 | CD32 |
| ABD | CD4 | CD28 | CD79a |
| ABD | CD4 | CD28 | CD79b |
| ABD | CD4 | CD8 | CD8 |
| ABD | CD4 | CD8 | CD3ζ |
| ABD | CD4 | CD8 | CD3δ |
| ABD | CD4 | CD8 | CD3γ |
| ABD | CD4 | CD8 | CD3ε |
| ABD | CD4 | CD8 | FcγRI-γ |
| ABD | CD4 | CD8 | FcγRIII-γ |
| ABD | CD4 | CD8 | FcεRIβ |
| ABD | CD4 | CD8 | FcεRIγ |
| ABD | CD4 | CD8 | DAP10 |
| ABD | CD4 | CD8 | DAP12 |
| ABD | CD4 | CD8 | CD32 |
| ABD | CD4 | CD8 | CD79a |
| ABD | CD4 | CD8 | CD79b |
| ABD | CD4 | CD4 | CD8 |
| ABD | CD4 | CD4 | CD3ζ |
| ABD | CD4 | CD4 | CD3δ |
| ABD | CD4 | CD4 | CD3γ |
| ABD | CD4 | CD4 | CD3ε |
| ABD | CD4 | CD4 | FcγRI-γ |
| ABD | CD4 | CD4 | FcγRIII-γ |
| ABD | CD4 | CD4 | FcεRIβ |
| ABD | CD4 | CD4 | FcεRIγ |
| ABD | CD4 | CD4 | DAP10 |
| ABD | CD4 | CD4 | DAP12 |
| ABD | CD4 | CD4 | CD32 |
| ABD | CD4 | CD4 | CD79a |
| ABD | CD4 | CD4 | CD79b |
| ABD | CD4 | b2c | CD8 |
| ABD | CD4 | b2c | CD3ζ |
| ABD | CD4 | b2c | CD3δ |
| ABD | CD4 | b2c | CD3γ |
| ABD | CD4 | b2c | CD3ε |
| ABD | CD4 | b2c | FcγRI-γ |
| ABD | CD4 | b2c | FcγRIII-γ |
| ABD | CD4 | b2c | FcεRIβ |
| ABD | CD4 | b2c | FcεRIγ |
| ABD | CD4 | b2c | DAP10 |
| ABD | CD4 | b2c | DAP12 |
| ABD | CD4 | b2c | CD32 |
| ABD | CD4 | b2c | CD79a |
| ABD | CD4 | b2c | CD79b |
| ABD | CD4 | CD137/41BB | CD8 |
| ABD | CD4 | CD137/41BB | CD3ζ |
| ABD | CD4 | CD137/41BB | CD3δ |
| ABD | CD4 | CD137/41BB | CD3γ |
| ABD | CD4 | CD137/41BB | CD3ε |
| ABD | CD4 | CD137/41BB | FcγRI-γ |
| ABD | CD4 | CD137/41BB | FcγRIII-γ |
| ABD | CD4 | CD137/41BB | FcεRIβ |
| ABD | CD4 | CD137/41BB | FcεRIγ |
| ABD | CD4 | CD137/41BB | DAP10 |
| ABD | CD4 | CD137/41BB | DAP12 |
| ABD | CD4 | CD137/41BB | CD32 |
| ABD | CD4 | CD137/41BB | CD79a |
| ABD | CD4 | CD137/41BB | CD79b |
| ABD | CD4 | ICOS | CD8 |
| ABD | CD4 | ICOS | CD3ζ |
| ABD | CD4 | ICOS | CD3δ |
| ABD | CD4 | ICOS | CD3γ |
| ABD | CD4 | ICOS | CD3ε |
| ABD | CD4 | ICOS | FcγRI-γ |
| ABD | CD4 | ICOS | FcγRIII-γ |
| ABD | CD4 | ICOS | FcεRIβ |
| ABD | CD4 | ICOS | FcεRIγ |
| ABD | CD4 | ICOS | DAP10 |
| ABD | CD4 | ICOS | DAP12 |
| ABD | CD4 | ICOS | CD32 |
| ABD | CD4 | ICOS | CD79a |
| ABD | CD4 | ICOS | CD79b |
| ABD | CD4 | CD27 | CD8 |
| ABD | CD4 | CD27 | CD3ζ |
| ABD | CD4 | CD27 | CD3δ |
| ABD | CD4 | CD27 | CD3γ |
| ABD | CD4 | CD27 | CD3ε |
| ABD | CD4 | CD27 | FcγRI-γ |
| ABD | CD4 | CD27 | FcγRIII-γ |
| ABD | CD4 | CD27 | FcεRIβ |
| ABD | CD4 | CD27 | FcεRIγ |
| ABD | CD4 | CD27 | DAP10 |
| ABD | CD4 | CD27 | DAP12 |
| ABD | CD4 | CD27 | CD32 |
| ABD | CD4 | CD27 | CD79a |
| ABD | CD4 | CD27 | CD79b |
| ABD | CD4 | CD28δ | CD8 |
| ABD | CD4 | CD28δ | CD3ζ |
| ABD | CD4 | CD28δ | CD3δ |
| ABD | CD4 | CD28δ | CD3γ |
| ABD | CD4 | CD28δ | CD3ε |
| ABD | CD4 | CD28δ | FcγRI-γ |
| ABD | CD4 | CD28δ | FcγRIII-γ |
| ABD | CD4 | CD28δ | FcεRIβ |
| ABD | CD4 | CD28δ | FcεRIγ |
| ABD | CD4 | CD28δ | DAP10 |
| ABD | CD4 | CD28δ | DAP12 |
| ABD | CD4 | CD28δ | CD32 |
| ABD | CD4 | CD28δ | CD79a |
| ABD | CD4 | CD28δ | CD79b |
| ABD | CD4 | CD80 | CD8 |
| ABD | CD4 | CD80 | CD3ζ |
| ABD | CD4 | CD80 | CD3δ |
| ABD | CD4 | CD80 | CD3γ |
| ABD | CD4 | CD80 | CD3ε |
| ABD | CD4 | CD80 | FcγRI-γ |
| ABD | CD4 | CD80 | FcγRIII-γ |
| ABD | CD4 | CD80 | FcεRIβ |
| ABD | CD4 | CD80 | FcεRIγ |
| ABD | CD4 | CD80 | DAP10 |
| ABD | CD4 | CD80 | DAP12 |
| ABD | CD4 | CD80 | CD32 |
| ABD | CD4 | CD80 | CD79a |
| ABD | CD4 | CD80 | CD79b |
| ABD | CD4 | CD86 | CD8 |
| ABD | CD4 | CD86 | CD3ζ |
| ABD | CD4 | CD86 | CD3δ |
| ABD | CD4 | CD86 | CD3γ |
| ABD | CD4 | CD86 | CD3ε |
| ABD | CD4 | CD86 | FcγRI-γ |
| ABD | CD4 | CD86 | FcγRIII-γ |
| ABD | CD4 | CD86 | FcεRIβ |
| ABD | CD4 | CD86 | FcεRIγ |
| ABD | CD4 | CD86 | DAP10 |
| ABD | CD4 | CD86 | DAP12 |
| ABD | CD4 | CD86 | CD32 |
| ABD | CD4 | CD86 | CD79a |
| ABD | CD4 | CD86 | CD79b |
| ABD | CD4 | OX40 | CD8 |
| ABD | CD4 | OX40 | CD3ζ |
| ABD | CD4 | OX40 | CD3δ |
| ABD | CD4 | OX40 | CD3γ |
| ABD | CD4 | OX40 | CD3ε |
| ABD | CD4 | OX40 | FcγRI-γ |
| ABD | CD4 | OX40 | FcγRIII-γ |
| ABD | CD4 | OX40 | FcεRIβ |
| ABD | CD4 | OX40 | FcεRIγ |
| ABD | CD4 | OX40 | DAP10 |
| ABD | CD4 | OX40 | DAP12 |
| ABD | CD4 | OX40 | CD32 |
| ABD | CD4 | OX40 | CD79a |
| ABD | CD4 | OX40 | CD79b |
| ABD | CD4 | DAP10 | CD8 |
| ABD | CD4 | DAP10 | CD3ζ |
| ABD | CD4 | DAP10 | CD3δ |
| ABD | CD4 | DAP10 | CD3γ |
| ABD | CD4 | DAP10 | CD3ε |
| ABD | CD4 | DAP10 | FcγRI-γ |
| ABD | CD4 | DAP10 | FcγRIII-γ |
| ABD | CD4 | DAP10 | FcεRIβ |
| ABD | CD4 | DAP10 | FcεRIγ |
| ABD | CD4 | DAP10 | DAP10 |
| ABD | CD4 | DAP10 | DAP12 |
| ABD | CD4 | DAP10 | CD32 |
| ABD | CD4 | DAP10 | CD79a |
| ABD | CD4 | DAP10 | CD79b |
| ABD | CD4 | DAP12 | CD8 |
| ABD | CD4 | DAP12 | CD3ζ |
| ABD | CD4 | DAP12 | CD3δ |
| ABD | CD4 | DAP12 | CD3γ |
| ABD | CD4 | DAP12 | CD3ε |
| ABD | CD4 | DAP12 | FcγRI-γ |
| ABD | CD4 | DAP12 | FcγRIII-γ |
| ABD | CD4 | DAP12 | FcεRIβ |
| ABD | CD4 | DAP12 | FcεRIγ |
| ABD | CD4 | DAP12 | DAP10 |
| ABD | CD4 | DAP12 | DAP12 |
| ABD | CD4 | DAP12 | CD32 |
| ABD | CD4 | DAP12 | CD79a |
| ABD | CD4 | DAP12 | CD79b |
| ABD | CD4 | MyD88 | CD8 |
| ABD | CD4 | MyD88 | CD3ζ |
| ABD | CD4 | MyD88 | CD3δ |
| ABD | CD4 | MyD88 | CD3γ |
| ABD | CD4 | MyD88 | CD3ε |
| ABD | CD4 | MyD88 | FcγRI-γ |
| ABD | CD4 | MyD88 | FcγRIII-γ |
| ABD | CD4 | MyD88 | FcεRIβ |
| ABD | CD4 | MyD88 | FcεRIγ |
| ABD | CD4 | MyD88 | DAP10 |
| ABD | CD4 | MyD88 | DAP12 |
| ABD | CD4 | MyD88 | CD32 |
| ABD | CD4 | MyD88 | CD79a |
| ABD | CD4 | MyD88 | CD79b |
| ABD | CD4 | CD7 | CD8 |
| ABD | CD4 | CD7 | CD3ζ |
| ABD | CD4 | CD7 | CD3δ |
| ABD | CD4 | CD7 | CD3γ |
| ABD | CD4 | CD7 | CD3ε |
| ABD | CD4 | CD7 | FcγRI-γ |
| ABD | CD4 | CD7 | FcγRIII-γ |
| ABD | CD4 | CD7 | FcεRIβ |
| ABD | CD4 | CD7 | FcεRIγ |
| ABD | CD4 | CD7 | DAP10 |
| ABD | CD4 | CD7 | DAP12 |
| ABD | CD4 | CD7 | CD32 |
| ABD | CD4 | CD7 | CD79a |
| ABD | CD4 | CD7 | CD79b |
| ABD | CD4 | BTNL3 | CD8 |
| ABD | CD4 | BTNL3 | CD3ζ |
| ABD | CD4 | BTNL3 | CD3δ |
| ABD | CD4 | BTNL3 | CD3γ |
| ABD | CD4 | BTNL3 | CD3ε |
| ABD | CD4 | BTNL3 | FcγRI-γ |
| ABD | CD4 | BTNL3 | FcγRIII-γ |
| ABD | CD4 | BTNL3 | FcεRIβ |
| ABD | CD4 | BTNL3 | FcεRIγ |
| ABD | CD4 | BTNL3 | DAP10 |
| ABD | CD4 | BTNL3 | DAP12 |
| ABD | CD4 | BTNL3 | CD32 |
| ABD | CD4 | BTNL3 | CD79a |
| ABD | CD4 | BTNL3 | CD79b |
| ABD | CD4 | NKG2D | CD8 |
| ABD | CD4 | NKG2D | CD3ζ |
| ABD | CD4 | NKG2D | CD3δ |
| ABD | CD4 | NKG2D | CD3γ |
| ABD | CD4 | NKG2D | CD3ε |
| ABD | CD4 | NKG2D | FcγRI-γ |
| ABD | CD4 | NKG2D | FcγRIII-γ |
| ABD | CD4 | NKG2D | FcεRIβ |
| ABD | CD4 | NKG2D | FcεRIγ |
| ABD | CD4 | NKG2D | DAP10 |
| ABD | CD4 | NKG2D | DAP12 |
| ABD | CD4 | NKG2D | CD32 |
| ABD | CD4 | NKG2D | CD79a |
| ABD | CD4 | NKG2D | CD79b |
| ABD | b2c | CD28 | CD8 |
| ABD | b2c | CD28 | CD3ζ |
| ABD | b2c | CD28 | CD3δ |
| ABD | b2c | CD28 | CD3γ |
| ABD | b2c | CD28 | CD3ε |
| ABD | b2c | CD28 | FcγRI-γ |
| ABD | b2c | CD28 | FcγRIII-γ |
| ABD | b2c | CD28 | FcεRIβ |
| ABD | b2c | CD28 | FcεRIγ |
| ABD | b2c | CD28 | DAP10 |
| ABD | b2c | CD28 | DAP12 |
| ABD | b2c | CD28 | CD32 |
| ABD | b2c | CD28 | CD79a |
| ABD | b2c | CD28 | CD79b |
| ABD | b2c | CD8 | CD8 |
| ABD | b2c | CD8 | CD3ζ |
| ABD | b2c | CD8 | CD3δ |
| ABD | b2c | CD8 | CD3γ |
| ABD | b2c | CD8 | CD3ε |
| ABD | b2c | CD8 | FcγRI-γ |
| ABD | b2c | CD8 | FcγRIII-γ |
| ABD | b2c | CD8 | FcεRIβ |
| ABD | b2c | CD8 | FcεRIγ |
| ABD | b2c | CD8 | DAP10 |
| ABD | b2c | CD8 | DAP12 |
| ABD | b2c | CD8 | CD32 |
| ABD | b2c | CD8 | CD79a |
| ABD | b2c | CD8 | CD79b |
| ABD | b2c | CD4 | CD8 |
| ABD | b2c | CD4 | CD3ζ |
| ABD | b2c | CD4 | CD3δ |
| ABD | b2c | CD4 | CD3γ |
| ABD | b2c | CD4 | CD3ε |
| ABD | b2c | CD4 | FcγRI-γ |
| ABD | b2c | CD4 | FcγRIII-γ |
| ABD | b2c | CD4 | FcεRIβ |
| ABD | b2c | CD4 | FcεRIγ |
| ABD | b2c | CD4 | DAP10 |
| ABD | b2c | CD4 | DAP12 |
| ABD | b2c | CD4 | CD32 |
| ABD | b2c | CD4 | CD79a |
| ABD | b2c | CD4 | CD79b |
| ABD | b2c | b2c | CD8 |
| ABD | b2c | b2c | CD3ζ |
| ABD | b2c | b2c | CD3δ |
| ABD | b2c | b2c | CD3γ |
| ABD | b2c | b2c | CD3ε |
| ABD | b2c | b2c | FcγRI-γ |
| ABD | b2c | b2c | FcγRIII-γ |
| ABD | b2c | b2c | FcεRIβ |
| ABD | b2c | b2c | FcεRIγ |
| ABD | b2c | b2c | DAP10 |
| ABD | b2c | b2c | DAP12 |
| ABD | b2c | b2c | CD32 |
| ABD | b2c | b2c | CD79a |
| ABD | b2c | b2c | CD79b |
| ABD | b2c | CD137/41BB | CD8 |
| ABD | b2c | CD137/41BB | CD3ζ |
| ABD | b2c | CD137/41BB | CD3δ |
| ABD | b2c | CD137/41BB | CD3γ |
| ABD | b2c | CD137/41BB | CD3ε |
| ABD | b2c | CD137/41BB | FcγRI-γ |
| ABD | b2c | CD137/41BB | FcγRIII-γ |
| ABD | b2c | CD137/41BB | FcεRIβ |
| ABD | b2c | CD137/41BB | FcεRIγ |
| ABD | b2c | CD137/41BB | DAP10 |
| ABD | b2c | CD137/41BB | DAP12 |
| ABD | b2c | CD137/41BB | CD32 |
| ABD | b2c | CD137/41BB | CD79a |
| ABD | b2c | CD137/41BB | CD79b |
| ABD | b2c | ICOS | CD8 |
| ABD | b2c | ICOS | CD3ζ |
| ABD | b2c | ICOS | CD3δ |
| ABD | b2c | ICOS | CD3γ |
| ABD | b2c | ICOS | CD3ε |
| ABD | b2c | ICOS | FcγRI-γ |
| ABD | b2c | ICOS | FcγRIII-γ |
| ABD | b2c | ICOS | FcεRIβ |
| ABD | b2c | ICOS | FcεRIγ |
| ABD | b2c | ICOS | DAP10 |
| ABD | b2c | ICOS | DAP12 |
| ABD | b2c | ICOS | CD32 |
| ABD | b2c | ICOS | CD79a |
| ABD | b2c | ICOS | CD79b |
| ABD | b2c | CD27 | CD8 |
| ABD | b2c | CD27 | CD3ζ |
| ABD | b2c | CD27 | CD3δ |
| ABD | b2c | CD27 | CD3γ |
| ABD | b2c | CD27 | CD3ε |
| ABD | b2c | CD27 | FcγRI-γ |
| ABD | b2c | CD27 | FcγRIII-γ |
| ABD | b2c | CD27 | FcεRIβ |
| ABD | b2c | CD27 | FcεRIγ |
| ABD | b2c | CD27 | DAP10 |
| ABD | b2c | CD27 | DAP12 |
| ABD | b2c | CD27 | CD32 |
| ABD | b2c | CD27 | CD79a |
| ABD | b2c | CD27 | CD79b |
| ABD | b2c | CD28δ | CD8 |
| ABD | b2c | CD28δ | CD3ζ |
| ABD | b2c | CD28δ | CD3δ |
| ABD | b2c | CD28δ | CD3γ |
| ABD | b2c | CD28δ | CD3ε |
| ABD | b2c | CD28δ | FcγRI-γ |
| ABD | b2c | CD28δ | FcγRIII-γ |
| ABD | b2c | CD28δ | FcεRIβ |
| ABD | b2c | CD28δ | FcεRIγ |
| ABD | b2c | CD28δ | DAP10 |
| ABD | b2c | CD28δ | DAP12 |
| ABD | b2c | CD28δ | CD32 |
| ABD | b2c | CD28δ | CD79a |
| ABD | b2c | CD28δ | CD79b |
| ABD | b2c | CD80 | CD8 |
| ABD | b2c | CD80 | CD3ζ |
| ABD | b2c | CD80 | CD3δ |
| ABD | b2c | CD80 | CD3γ |
| ABD | b2c | CD80 | CD3ε |
| ABD | b2c | CD80 | FcγRI-γ |
| ABD | b2c | CD80 | FcγRIII-γ |
| ABD | b2c | CD80 | FcεRIβ |
| ABD | b2c | CD80 | FcεRIγ |
| ABD | b2c | CD80 | DAP10 |
| ABD | b2c | CD80 | DAP12 |
| ABD | b2c | CD80 | CD32 |
| ABD | b2c | CD80 | CD79a |
| ABD | b2c | CD80 | CD79b |
| ABD | b2c | CD86 | CD8 |
| ABD | b2c | CD86 | CD3ζ |
| ABD | b2c | CD86 | CD3δ |
| ABD | b2c | CD86 | CD3γ |
| ABD | b2c | CD86 | CD3ε |
| ABD | b2c | CD86 | FcγRI-γ |
| ABD | b2c | CD86 | FcγRIII-γ |
| ABD | b2c | CD86 | FcεRIβ |
| ABD | b2c | CD86 | FcεRIγ |
| ABD | b2c | CD86 | DAP10 |
| ABD | b2c | CD86 | DAP12 |
| ABD | b2c | CD86 | CD32 |
| ABD | b2c | CD86 | CD79a |
| ABD | b2c | CD86 | CD79b |
| ABD | b2c | OX40 | CD8 |
| ABD | b2c | OX40 | CD3ζ |
| ABD | b2c | OX40 | CD3δ |
| ABD | b2c | OX40 | CD3γ |
| ABD | b2c | OX40 | CD3ε |
| ABD | b2c | OX40 | FcγRI-γ |
| ABD | b2c | OX40 | FcγRIII-γ |
| ABD | b2c | OX40 | FcεRIβ |
| ABD | b2c | OX40 | FcεRIγ |
| ABD | b2c | OX40 | DAP10 |
| ABD | b2c | OX40 | DAP12 |
| ABD | b2c | OX40 | CD32 |
| ABD | b2c | OX40 | CD79a |
| ABD | b2c | OX40 | CD79b |
| ABD | b2c | DAP10 | CD8 |
| ABD | b2c | DAP10 | CD3ζ |
| ABD | b2c | DAP10 | CD3δ |
| ABD | b2c | DAP10 | CD3γ |
| ABD | b2c | DAP10 | CD3ε |
| ABD | b2c | DAP10 | FcγRI-γ |
| ABD | b2c | DAP10 | FcγRIII-γ |
| ABD | b2c | DAP10 | FcεRIβ |
| ABD | b2c | DAP10 | FcεRIγ |
| ABD | b2c | DAP10 | DAP10 |
| ABD | b2c | DAP10 | DAP12 |
| ABD | b2c | DAP10 | CD32 |
| ABD | b2c | DAP10 | CD79a |
| ABD | b2c | DAP10 | CD79b |
| ABD | b2c | DAP12 | CD8 |
| ABD | b2c | DAP12 | CD3ζ |
| ABD | b2c | DAP12 | CD3δ |
| ABD | b2c | DAP12 | CD3γ |
| ABD | b2c | DAP12 | CD3ε |
| ABD | b2c | DAP12 | FcγRI-γ |
| ABD | b2c | DAP12 | FcγRIII-γ |
| ABD | b2c | DAP12 | FcεRIβ |
| ABD | b2c | DAP12 | FcεRIγ |
| ABD | b2c | DAP12 | DAP10 |
| ABD | b2c | DAP12 | DAP12 |
| ABD | b2c | DAP12 | CD32 |
| ABD | b2c | DAP12 | CD79a |
| ABD | b2c | DAP12 | CD79b |
| ABD | b2c | MyD88 | CD8 |
| ABD | b2c | MyD88 | CD3ζ |
| ABD | b2c | MyD88 | CD3δ |
| ABD | b2c | MyD88 | CD3γ |
| ABD | b2c | MyD88 | CD3ε |
| ABD | b2c | MyD88 | FcγRI-γ |
| ABD | b2c | MyD88 | FcγRIII-γ |
| ABD | b2c | MyD88 | FcεRIβ |
| ABD | b2c | MyD88 | FcεRIγ |
| ABD | b2c | MyD88 | DAP10 |
| ABD | b2c | MyD88 | DAP12 |
| ABD | b2c | MyD88 | CD32 |
| ABD | b2c | MyD88 | CD79a |
| ABD | b2c | MyD88 | CD79b |
| ABD | b2c | CD7 | CD8 |
| ABD | b2c | CD7 | CD3ζ |
| ABD | b2c | CD7 | CD3δ |
| ABD | b2c | CD7 | CD3γ |
| ABD | b2c | CD7 | CD3ε |
| ABD | b2c | CD7 | FcγRI-γ |
| ABD | b2c | CD7 | FcγRIII-γ |
| ABD | b2c | CD7 | FcεRIβ |
| ABD | b2c | CD7 | FcεRIγ |
| ABD | b2c | CD7 | DAP10 |
| ABD | b2c | CD7 | DAP12 |
| ABD | b2c | CD7 | CD32 |
| ABD | b2c | CD7 | CD79a |
| ABD | b2c | CD7 | CD79b |
| ABD | b2c | BTNL3 | CD8 |
| ABD | b2c | BTNL3 | CD3ζ |
| ABD | b2c | BTNL3 | CD3δ |
| ABD | b2c | BTNL3 | CD3γ |
| ABD | b2c | BTNL3 | CD3ε |
| ABD | b2c | BTNL3 | FcγRI-γ |
| ABD | b2c | BTNL3 | FcγRIII-γ |
| ABD | b2c | BTNL3 | FcεRIβ |
| ABD | b2c | BTNL3 | FcεRIγ |
| ABD | b2c | BTNL3 | DAP10 |
| ABD | b2c | BTNL3 | DAP12 |
| ABD | b2c | BTNL3 | CD32 |
| ABD | b2c | BTNL3 | CD79a |
| ABD | b2c | BTNL3 | CD79b |
| ABD | b2c | NKG2D | CD8 |
| ABD | b2c | NKG2D | CD3ζ |
| ABD | b2c | NKG2D | CD3δ |
| ABD | b2c | NKG2D | CD3γ |
| ABD | b2c | NKG2D | CD3ε |
| ABD | b2c | NKG2D | FcγRI-γ |
| ABD | b2c | NKG2D | FcγRIII-γ |
| ABD | b2c | NKG2D | FcεRIβ |
| ABD | b2c | NKG2D | FcεRIγ |
| ABD | b2c | NKG2D | DAP10 |
| ABD | b2c | NKG2D | DAP12 |
| ABD | b2c | NKG2D | CD32 |
| ABD | b2c | NKG2D | CD79a |
| ABD | b2c | NKG2D | CD79b |
| ABD | CD137/41BB | CD28 | CD8 |
| ABD | CD137/41BB | CD28 | CD3ζ |
| ABD | CD137/41BB | CD28 | CD3δ |
| ABD | CD137/41BB | CD28 | CD3γ |
| ABD | CD137/41BB | CD28 | CD3ε |
| ABD | CD137/41BB | CD28 | FcγRI-γ |
| ABD | CD137/41BB | CD28 | FcγRIII-γ |
| ABD | CD137/41BB | CD28 | FcεRIβ |
| ABD | CD137/41BB | CD28 | FcεRIγ |
| ABD | CD137/41BB | CD28 | DAP10 |
| ABD | CD137/41BB | CD28 | DAP12 |
| ABD | CD137/41BB | CD28 | CD32 |
| ABD | CD137/41BB | CD28 | CD79a |
| ABD | CD137/41BB | CD28 | CD79b |
| ABD | CD137/41BB | CD8 | CD8 |
| ABD | CD137/41BB | CD8 | CD3ζ |
| ABD | CD137/41BB | CD8 | CD3δ |
| ABD | CD137/41BB | CD8 | CD3γ |
| ABD | CD137/41BB | CD8 | CD3ε |
| ABD | CD137/41BB | CD8 | FcγRI-γ |
| ABD | CD137/41BB | CD8 | FcγRIII-γ |
| ABD | CD137/41BB | CD8 | FcεRIβ |
| ABD | CD137/41BB | CD8 | FcεRIγ |
| ABD | CD137/41BB | CD8 | DAP10 |
| ABD | CD137/41BB | CD8 | DAP12 |
| ABD | CD137/41BB | CD8 | CD32 |
| ABD | CD137/41BB | CD8 | CD79a |
| ABD | CD137/41BB | CD8 | CD79b |
| ABD | CD137/41BB | CD4 | CD8 |
| ABD | CD137/41BB | CD4 | CD3ζ |
| ABD | CD137/41BB | CD4 | CD3δ |
| ABD | CD137/41BB | CD4 | CD3γ |
| ABD | CD137/41BB | CD4 | CD3ε |
| ABD | CD137/41BB | CD4 | FcγRI-γ |
| ABD | CD137/41BB | CD4 | FcγRIII-γ |
| ABD | CD137/41BB | CD4 | FcεRIβ |
| ABD | CD137/41BB | CD4 | FcεRIγ |
| ABD | CD137/41BB | CD4 | DAP10 |
| ABD | CD137/41BB | CD4 | DAP12 |
| ABD | CD137/41BB | CD4 | CD32 |
| ABD | CD137/41BB | CD4 | CD79a |
| ABD | CD137/41BB | CD4 | CD79b |
| ABD | CD137/41BB | b2c | CD8 |
| ABD | CD137/41BB | b2c | CD3ζ |
| ABD | CD137/41BB | b2c | CD3δ |
| ABD | CD137/41BB | b2c | CD3γ |
| ABD | CD137/41BB | b2c | CD3ε |
| ABD | CD137/41BB | b2c | FcγRI-γ |
| ABD | CD137/41BB | b2c | FcγRIII-γ |
| ABD | CD137/41BB | b2c | FcεRIβ |
| ABD | CD137/41BB | b2c | FcεRIγ |
| ABD | CD137/41BB | b2c | DAP10 |
| ABD | CD137/41BB | b2c | DAP12 |
| ABD | CD137/41BB | b2c | CD32 |
| ABD | CD137/41BB | b2c | CD79a |
| ABD | CD137/41BB | b2c | CD79b |
| ABD | CD137/41BB | CD137/41BB | CD8 |
| ABD | CD137/41BB | CD137/41BB | CD3ζ |
| ABD | CD137/41BB | CD137/41BB | CD3δ |
| ABD | CD137/41BB | CD137/41BB | CD3γ |
| ABD | CD137/41BB | CD137/41BB | CD3ε |
| ABD | CD137/41BB | CD137/41BB | FcγRI-γ |
| ABD | CD137/41BB | CD137/41BB | FcγRIII-γ |
| ABD | CD137/41BB | CD137/41BB | FcεRIβ |
| ABD | CD137/41BB | CD137/41BB | FcεRIγ |
| ABD | CD137/41BB | CD137/41BB | DAP10 |
| ABD | CD137/41BB | CD137/41BB | DAP12 |
| ABD | CD137/41BB | CD137/41BB | CD32 |
| ABD | CD137/41BB | CD137/41BB | CD79a |
| ABD | CD137/41BB | CD137/41BB | CD79b |
| ABD | CD137/41BB | ICOS | CD8 |
| ABD | CD137/41BB | ICOS | CD3ζ |
| ABD | CD137/41BB | ICOS | CD3δ |
| ABD | CD137/41BB | ICOS | CD3γ |
| ABD | CD137/41BB | ICOS | CD3ε |
| ABD | CD137/41BB | ICOS | FcγRI-γ |
| ABD | CD137/41BB | ICOS | FcγRIII-γ |
| ABD | CD137/41BB | ICOS | FcεRIβ |
| ABD | CD137/41BB | ICOS | FcεRIγ |
| ABD | CD137/41BB | ICOS | DAP10 |
| ABD | CD137/41BB | ICOS | DAP12 |
| ABD | CD137/41BB | ICOS | CD32 |
| ABD | CD137/41BB | ICOS | CD79a |
| ABD | CD137/41BB | ICOS | CD79b |
| ABD | CD137/41BB | CD27 | CD8 |
| ABD | CD137/41BB | CD27 | CD3ζ |
| ABD | CD137/41BB | CD27 | CD3δ |
| ABD | CD137/41BB | CD27 | CD3γ |
| ABD | CD137/41BB | CD27 | CD3ε |
| ABD | CD137/41BB | CD27 | FcγRI-γ |
| ABD | CD137/41BB | CD27 | FcγRIII-γ |
| ABD | CD137/41BB | CD27 | FcεRIβ |
| ABD | CD137/41BB | CD27 | FcεRIγ |
| ABD | CD137/41BB | CD27 | DAP10 |
| ABD | CD137/41BB | CD27 | DAP12 |
| ABD | CD137/41BB | CD27 | CD32 |
| ABD | CD137/41BB | CD27 | CD79a |
| ABD | CD137/41BB | CD27 | CD79b |
| ABD | CD137/41BB | CD28δ | CD8 |
| ABD | CD137/41BB | CD28δ | CD3ζ |
| ABD | CD137/41BB | CD28δ | CD3δ |
| ABD | CD137/41BB | CD28δ | CD3γ |
| ABD | CD137/41BB | CD28δ | CD3ε |
| ABD | CD137/41BB | CD28δ | FcγRI-γ |
| ABD | CD137/41BB | CD28δ | FcγRIII-γ |
| ABD | CD137/41BB | CD28δ | FcεRIβ |
| ABD | CD137/41BB | CD28δ | FcεRIγ |
| ABD | CD137/41BB | CD28δ | DAP10 |
| ABD | CD137/41BB | CD28δ | DAP12 |
| ABD | CD137/41BB | CD28δ | CD32 |
| ABD | CD137/41BB | CD28δ | CD79a |
| ABD | CD137/41BB | CD28δ | CD79b |
| ABD | CD137/41BB | CD80 | CD8 |
| ABD | CD137/41BB | CD80 | CD3ζ |
| ABD | CD137/41BB | CD80 | CD3δ |
| ABD | CD137/41BB | CD80 | CD3γ |
| ABD | CD137/41BB | CD80 | CD3ε |
| ABD | CD137/41BB | CD80 | FcγRI-γ |
| ABD | CD137/41BB | CD80 | FcγRIII-γ |
| ABD | CD137/41BB | CD80 | FcεRIβ |
| ABD | CD137/41BB | CD80 | FcεRIγ |
| ABD | CD137/41BB | CD80 | DAP10 |
| ABD | CD137/41BB | CD80 | DAP12 |
| ABD | CD137/41BB | CD80 | CD32 |
| ABD | CD137/41BB | CD80 | CD79a |
| ABD | CD137/41BB | CD80 | CD79b |
| ABD | CD137/41BB | CD86 | CD8 |
| ABD | CD137/41BB | CD86 | CD3ζ |
| ABD | CD137/41BB | CD86 | CD3δ |
| ABD | CD137/41BB | CD86 | CD3γ |
| ABD | CD137/41BB | CD86 | CD3ε |
| ABD | CD137/41BB | CD86 | FcγRI-γ |
| ABD | CD137/41BB | CD86 | FcγRIII-γ |
| ABD | CD137/41BB | CD86 | FcεRIβ |
| ABD | CD137/41BB | CD86 | FcεRIγ |
| ABD | CD137/41BB | CD86 | DAP10 |
| ABD | CD137/41BB | CD86 | DAP12 |
| ABD | CD137/41BB | CD86 | CD32 |
| ABD | CD137/41BB | CD86 | CD79a |
| ABD | CD137/41BB | CD86 | CD79b |
| ABD | CD137/41BB | OX40 | CD8 |
| ABD | CD137/41BB | OX40 | CD3ζ |
| ABD | CD137/41BB | OX40 | CD3δ |
| ABD | CD137/41BB | OX40 | CD3γ |
| ABD | CD137/41BB | OX40 | CD3ε |
| ABD | CD137/41BB | OX40 | FcγRI-γ |
| ABD | CD137/41BB | OX40 | FcγRIII-γ |
| ABD | CD137/41BB | OX40 | FcεRIβ |
| ABD | CD137/41BB | OX40 | FcεRIγ |
| ABD | CD137/41BB | OX40 | DAP10 |
| ABD | CD137/41BB | OX40 | DAP12 |
| ABD | CD137/41BB | OX40 | CD32 |
| ABD | CD137/41BB | OX40 | CD79a |
| ABD | CD137/41BB | OX40 | CD79b |
| ABD | CD137/41BB | DAP10 | CD8 |
| ABD | CD137/41BB | DAP10 | CD3ζ |
| ABD | CD137/41BB | DAP10 | CD3δ |
| ABD | CD137/41BB | DAP10 | CD3γ |
| ABD | CD137/41BB | DAP10 | CD3ε |
| ABD | CD137/41BB | DAP10 | FcγRI-γ |
| ABD | CD137/41BB | DAP10 | FcγRIII-γ |
| ABD | CD137/41BB | DAP10 | FcεRIβ |
| ABD | CD137/41BB | DAP10 | FcεRIγ |
| ABD | CD137/41BB | DAP10 | DAP10 |
| ABD | CD137/41BB | DAP10 | DAP12 |
| ABD | CD137/41BB | DAP10 | CD32 |
| ABD | CD137/41BB | DAP10 | CD79a |
| ABD | CD137/41BB | DAP10 | CD79b |
| ABD | CD137/41BB | DAP12 | CD8 |
| ABD | CD137/41BB | DAP12 | CD3ζ |
| ABD | CD137/41BB | DAP12 | CD3δ |
| ABD | CD137/41BB | DAP12 | CD3γ |
| ABD | CD137/41BB | DAP12 | CD3ε |
| ABD | CD137/41BB | DAP12 | FcγRI-γ |
| ABD | CD137/41BB | DAP12 | FcγRIII-γ |
| ABD | CD137/41BB | DAP12 | FcεRIβ |
| ABD | CD137/41BB | DAP12 | FcεRIγ |
| ABD | CD137/41BB | DAP12 | DAP10 |
| ABD | CD137/41BB | DAP12 | DAP12 |
| ABD | CD137/41BB | DAP12 | CD32 |
| ABD | CD137/41BB | DAP12 | CD79a |
| ABD | CD137/41BB | DAP12 | CD79b |
| ABD | CD137/41BB | MyD88 | CD8 |
| ABD | CD137/41BB | MyD88 | CD3ζ |
| ABD | CD137/41BB | MyD88 | CD3δ |
| ABD | CD137/41BB | MyD88 | CD3γ |
| ABD | CD137/41BB | MyD88 | CD3ε |
| ABD | CD137/41BB | MyD88 | FcγRI-γ |
| ABD | CD137/41BB | MyD88 | FcγRIII-γ |
| ABD | CD137/41BB | MyD88 | FcεRIβ |
| ABD | CD137/41BB | MyD88 | FcεRIγ |
| ABD | CD137/41BB | MyD88 | DAP10 |
| ABD | CD137/41BB | MyD88 | DAP12 |
| ABD | CD137/41BB | MyD88 | CD32 |
| ABD | CD137/41BB | MyD88 | CD79a |
| ABD | CD137/41BB | MyD88 | CD79b |
| ABD | CD137/41BB | CD7 | CD8 |
| ABD | CD137/41BB | CD7 | CD3ζ |
| ABD | CD137/41BB | CD7 | CD3δ |
| ABD | CD137/41BB | CD7 | CD3γ |
| ABD | CD137/41BB | CD7 | CD3ε |
| ABD | CD137/41BB | CD7 | FcγRI-γ |
| ABD | CD137/41BB | CD7 | FcγRIII-γ |
| ABD | CD137/41BB | CD7 | FcεRIβ |
| ABD | CD137/41BB | CD7 | FcεRIγ |
| ABD | CD137/41BB | CD7 | DAP10 |
| ABD | CD137/41BB | CD7 | DAP12 |
| ABD | CD137/41BB | CD7 | CD32 |
| ABD | CD137/41BB | CD7 | CD79a |
| ABD | CD137/41BB | CD7 | CD79b |
| ABD | CD137/41BB | BTNL3 | CD8 |
| ABD | CD137/41BB | BTNL3 | CD3ζ |
| ABD | CD137/41BB | BTNL3 | CD3δ |
| ABD | CD137/41BB | BTNL3 | CD3γ |
| ABD | CD137/41BB | BTNL3 | CD3ε |
| ABD | CD137/41BB | BTNL3 | FcγRI-γ |
| ABD | CD137/41BB | BTNL3 | FcγRIII-γ |
| ABD | CD137/41BB | BTNL3 | FcεRIβ |
| ABD | CD137/41BB | BTNL3 | FcεRIγ |
| ABD | CD137/41BB | BTNL3 | DAP10 |
| ABD | CD137/41BB | BTNL3 | DAP12 |
| ABD | CD137/41BB | BTNL3 | CD32 |
| ABD | CD137/41BB | BTNL3 | CD79a |
| ABD | CD137/41BB | BTNL3 | CD79b |
| ABD | CD137/41BB | NKG2D | CD8 |
| ABD | CD137/41BB | NKG2D | CD3ζ |
| ABD | CD137/41BB | NKG2D | CD3δ |
| ABD | CD137/41BB | NKG2D | CD3γ |
| ABD | CD137/41BB | NKG2D | CD3ε |
| ABD | CD137/41BB | NKG2D | FcγRI-γ |
| ABD | CD137/41BB | NKG2D | FcγRIII-γ |
| ABD | CD137/41BB | NKG2D | FcεRIβ |
| ABD | CD137/41BB | NKG2D | FcεRIγ |
| ABD | CD137/41BB | NKG2D | DAP10 |
| ABD | CD137/41BB | NKG2D | DAP12 |
| ABD | CD137/41BB | NKG2D | CD32 |
| ABD | CD137/41BB | NKG2D | CD79a |
| ABD | CD137/41BB | NKG2D | CD79b |
| ABD | ICOS | CD28 | CD8 |
| ABD | ICOS | CD28 | CD3ζ |
| ABD | ICOS | CD28 | CD3δ |
| ABD | ICOS | CD28 | CD3γ |
| ABD | ICOS | CD28 | CD3ε |
| ABD | ICOS | CD28 | FcγRI-γ |
| ABD | ICOS | CD28 | FcγRIII-γ |
| ABD | ICOS | CD28 | FcεRIβ |
| ABD | ICOS | CD28 | FcεRIγ |
| ABD | ICOS | CD28 | DAP10 |
| ABD | ICOS | CD28 | DAP12 |
| ABD | ICOS | CD28 | CD32 |
| ABD | ICOS | CD28 | CD79a |
| ABD | ICOS | CD28 | CD79b |
| ABD | ICOS | CD8 | CD8 |
| ABD | ICOS | CD8 | CD3ζ |
| ABD | ICOS | CD8 | CD3δ |
| ABD | ICOS | CD8 | CD3γ |
| ABD | ICOS | CD8 | CD3ε |
| ABD | ICOS | CD8 | FcγRI-γ |
| ABD | ICOS | CD8 | FcγRIII-γ |
| ABD | ICOS | CD8 | FcεRIβ |
| ABD | ICOS | CD8 | FcεRIγ |
| ABD | ICOS | CD8 | DAP10 |
| ABD | ICOS | CD8 | DAP12 |
| ABD | ICOS | CD8 | CD32 |
| ABD | ICOS | CD8 | CD79a |
| ABD | ICOS | CD8 | CD79b |
| ABD | ICOS | CD4 | CD8 |
| ABD | ICOS | CD4 | CD3ζ |
| ABD | ICOS | CD4 | CD3δ |
| ABD | ICOS | CD4 | CD3γ |
| ABD | ICOS | CD4 | CD3ε |
| ABD | ICOS | CD4 | FcγRI-γ |
| ABD | ICOS | CD4 | FcγRIII-γ |
| ABD | ICOS | CD4 | FcεRIβ |
| ABD | ICOS | CD4 | FcεRIγ |
| ABD | ICOS | CD4 | DAP10 |
| ABD | ICOS | CD4 | DAP12 |
| ABD | ICOS | CD4 | CD32 |
| ABD | ICOS | CD4 | CD79a |
| ABD | ICOS | CD4 | CD79b |
| ABD | ICOS | b2c | CD8 |
| ABD | ICOS | b2c | CD3ζ |
| ABD | ICOS | b2c | CD3δ |
| ABD | ICOS | b2c | CD3γ |
| ABD | ICOS | b2c | CD3ε |
| ABD | ICOS | b2c | FcγRI-γ |
| ABD | ICOS | b2c | FcγRIII-γ |
| ABD | ICOS | b2c | FcεRIβ |
| ABD | ICOS | b2c | FcεRIγ |
| ABD | ICOS | b2c | DAP10 |
| ABD | ICOS | b2c | DAP12 |
| ABD | ICOS | b2c | CD32 |
| ABD | ICOS | b2c | CD79a |
| ABD | ICOS | b2c | CD79b |
| ABD | ICOS | CD137/41BB | CD8 |
| ABD | ICOS | CD137/41BB | CD3ζ |
| ABD | ICOS | CD137/41BB | CD3δ |
| ABD | ICOS | CD137/41BB | CD3γ |
| ABD | ICOS | CD137/41BB | CD3ε |
| ABD | ICOS | CD137/41BB | FcγRI-γ |
| ABD | ICOS | CD137/41BB | FcγRIII-γ |
| ABD | ICOS | CD137/41BB | FcεRIβ |
| ABD | ICOS | CD137/41BB | FcεRIγ |
| ABD | ICOS | CD137/41BB | DAP10 |
| ABD | ICOS | CD137/41BB | DAP12 |
| ABD | ICOS | CD137/41BB | CD32 |
| ABD | ICOS | CD137/41BB | CD79a |
| ABD | ICOS | CD137/41BB | CD79b |
| ABD | ICOS | ICOS | CD8 |
| ABD | ICOS | ICOS | CD3ζ |
| ABD | ICOS | ICOS | CD3δ |
| ABD | ICOS | ICOS | CD3γ |
| ABD | ICOS | ICOS | CD3ε |
| ABD | ICOS | ICOS | FcγRI-γ |
| ABD | ICOS | ICOS | FcγRIII-γ |
| ABD | ICOS | ICOS | FcεRIβ |
| ABD | ICOS | ICOS | FcεRIγ |
| ABD | ICOS | ICOS | DAP10 |
| ABD | ICOS | ICOS | DAP12 |
| ABD | ICOS | ICOS | CD32 |
| ABD | ICOS | ICOS | CD79a |
| ABD | ICOS | ICOS | CD79b |
| ABD | ICOS | CD27 | CD8 |
| ABD | ICOS | CD27 | CD3ζ |
| ABD | ICOS | CD27 | CD3δ |
| ABD | ICOS | CD27 | CD3γ |
| ABD | ICOS | CD27 | CD3ε |
| ABD | ICOS | CD27 | FcγRI-γ |
| ABD | ICOS | CD27 | FcγRIII-γ |
| ABD | ICOS | CD27 | FcεRIβ |
| ABD | ICOS | CD27 | FcεRIγ |
| ABD | ICOS | CD27 | DAP10 |
| ABD | ICOS | CD27 | DAP12 |
| ABD | ICOS | CD27 | CD32 |
| ABD | ICOS | CD27 | CD79a |
| ABD | ICOS | CD27 | CD79b |
| ABD | ICOS | CD28δ | CD8 |
| ABD | ICOS | CD28δ | CD3ζ |
| ABD | ICOS | CD28δ | CD3δ |
| ABD | ICOS | CD28δ | CD3γ |
| ABD | ICOS | CD28δ | CD3ε |
| ABD | ICOS | CD28δ | FcγRI-γ |
| ABD | ICOS | CD28δ | FcγRIII-γ |
| ABD | ICOS | CD28δ | FcεRIβ |
| ABD | ICOS | CD28δ | FcεRIγ |
| ABD | ICOS | CD28δ | DAP10 |
| ABD | ICOS | CD28δ | DAP12 |
| ABD | ICOS | CD28δ | CD32 |
| ABD | ICOS | CD28δ | CD79a |
| ABD | ICOS | CD28δ | CD79b |
| ABD | ICOS | CD80 | CD8 |
| ABD | ICOS | CD80 | CD3ζ |
| ABD | ICOS | CD80 | CD3δ |
| ABD | ICOS | CD80 | CD3γ |
| ABD | ICOS | CD80 | CD3ε |
| ABD | ICOS | CD80 | FcγRI-γ |
| ABD | ICOS | CD80 | FcγRIII-γ |
| ABD | ICOS | CD80 | FcεRIβ |
| ABD | ICOS | CD80 | FcεRIγ |
| ABD | ICOS | CD80 | DAP10 |
| ABD | ICOS | CD80 | DAP12 |
| ABD | ICOS | CD80 | CD32 |
| ABD | ICOS | CD80 | CD79a |
| ABD | ICOS | CD80 | CD79b |
| ABD | ICOS | CD86 | CD8 |
| ABD | ICOS | CD86 | CD3ζ |
| ABD | ICOS | CD86 | CD3δ |
| ABD | ICOS | CD86 | CD3γ |
| ABD | ICOS | CD86 | CD3ε |
| ABD | ICOS | CD86 | FcγRI-γ |
| ABD | ICOS | CD86 | FcγRIII-γ |
| ABD | ICOS | CD86 | FcεRIβ |
| ABD | ICOS | CD86 | FcεRIγ |
| ABD | ICOS | CD86 | DAP10 |
| ABD | ICOS | CD86 | DAP12 |
| ABD | ICOS | CD86 | CD32 |
| ABD | ICOS | CD86 | CD79a |
| ABD | ICOS | CD86 | CD79b |
| ABD | ICOS | OX40 | CD8 |
| ABD | ICOS | OX40 | CD3ζ |
| ABD | ICOS | OX40 | CD3δ |
| ABD | ICOS | OX40 | CD3γ |
| ABD | ICOS | OX40 | CD3ε |
| ABD | ICOS | OX40 | FcγRI-γ |
| ABD | ICOS | OX40 | FcγRIII-γ |
| ABD | ICOS | OX40 | FcεRIβ |
| ABD | ICOS | OX40 | FcεRIγ |
| ABD | ICOS | OX40 | DAP10 |
| ABD | ICOS | OX40 | DAP12 |
| ABD | ICOS | OX40 | CD32 |
| ABD | ICOS | OX40 | CD79a |
| ABD | ICOS | OX40 | CD79b |
| ABD | ICOS | DAP10 | CD8 |
| ABD | ICOS | DAP10 | CD3ζ |
| ABD | ICOS | DAP10 | CD3δ |
| ABD | ICOS | DAP10 | CD3γ |
| ABD | ICOS | DAP10 | CD3ε |
| ABD | ICOS | DAP10 | FcγRI-γ |
| ABD | ICOS | DAP10 | FcγRIII-γ |
| ABD | ICOS | DAP10 | FcεRIβ |
| ABD | ICOS | DAP10 | FcεRIγ |
| ABD | ICOS | DAP10 | DAP10 |
| ABD | ICOS | DAP10 | DAP12 |
| ABD | ICOS | DAP10 | CD32 |
| ABD | ICOS | DAP10 | CD79a |
| ABD | ICOS | DAP10 | CD79b |
| ABD | ICOS | DAP12 | CD8 |
| ABD | ICOS | DAP12 | CD3ζ |
| ABD | ICOS | DAP12 | CD3δ |
| ABD | ICOS | DAP12 | CD3γ |
| ABD | ICOS | DAP12 | CD3ε |
| ABD | ICOS | DAP12 | FcγRI-γ |
| ABD | ICOS | DAP12 | FcγRIII-γ |
| ABD | ICOS | DAP12 | FcεRIβ |
| ABD | ICOS | DAP12 | FcεRIγ |
| ABD | ICOS | DAP12 | DAP10 |
| ABD | ICOS | DAP12 | DAP12 |
| ABD | ICOS | DAP12 | CD32 |
| ABD | ICOS | DAP12 | CD79a |
| ABD | ICOS | DAP12 | CD79b |
| ABD | ICOS | MyD88 | CD8 |
| ABD | ICOS | MyD88 | CD3ζ |
| ABD | ICOS | MyD88 | CD3δ |
| ABD | ICOS | MyD88 | CD3γ |
| ABD | ICOS | MyD88 | CD3ε |
| ABD | ICOS | MyD88 | FcγRI-γ |
| ABD | ICOS | MyD88 | FcγRIII-γ |
| ABD | ICOS | MyD88 | FcεRIβ |
| ABD | ICOS | MyD88 | FcεRIγ |
| ABD | ICOS | MyD88 | DAP10 |
| ABD | ICOS | MyD88 | DAP12 |
| ABD | ICOS | MyD88 | CD32 |
| ABD | ICOS | MyD88 | CD79a |
| ABD | ICOS | MyD88 | CD79b |
| ABD | ICOS | CD7 | CD8 |
| ABD | ICOS | CD7 | CD3ζ |
| ABD | ICOS | CD7 | CD3δ |
| ABD | ICOS | CD7 | CD3γ |
| ABD | ICOS | CD7 | CD3ε |
| ABD | ICOS | CD7 | FcγRI-γ |
| ABD | ICOS | CD7 | FcγRIII-γ |
| ABD | ICOS | CD7 | FcεRIβ |
| ABD | ICOS | CD7 | FcεRIγ |
| ABD | ICOS | CD7 | DAP10 |
| ABD | ICOS | CD7 | DAP12 |
| ABD | ICOS | CD7 | CD32 |
| ABD | ICOS | CD7 | CD79a |
| ABD | ICOS | CD7 | CD79b |
| ABD | ICOS | BTNL3 | CD8 |
| ABD | ICOS | BTNL3 | CD3ζ |
| ABD | ICOS | BTNL3 | CD3δ |
| ABD | ICOS | BTNL3 | CD3γ |
| ABD | ICOS | BTNL3 | CD3ε |
| ABD | ICOS | BTNL3 | FcγRI-γ |
| ABD | ICOS | BTNL3 | FcγRIII-γ |
| ABD | ICOS | BTNL3 | FcεRIβ |
| ABD | ICOS | BTNL3 | FcεRIγ |
| ABD | ICOS | BTNL3 | DAP10 |
| ABD | ICOS | BTNL3 | DAP12 |
| ABD | ICOS | BTNL3 | CD32 |
| ABD | ICOS | BTNL3 | CD79a |
| ABD | ICOS | BTNL3 | CD79b |
| ABD | ICOS | NKG2D | CD8 |
| ABD | ICOS | NKG2D | CD3ζ |
| ABD | ICOS | NKG2D | CD3δ |
| ABD | ICOS | NKG2D | CD3γ |
| ABD | ICOS | NKG2D | CD3ε |
| ABD | ICOS | NKG2D | FcγRI-γ |
| ABD | ICOS | NKG2D | FcγRIII-γ |
| ABD | ICOS | NKG2D | FcεRIβ |
| ABD | ICOS | NKG2D | FcεRIγ |
| ABD | ICOS | NKG2D | DAP10 |
| ABD | ICOS | NKG2D | DAP12 |
| ABD | ICOS | NKG2D | CD32 |
| ABD | ICOS | NKG2D | CD79a |
| ABD | ICOS | NKG2D | CD79b |
| ABD | CD27 | CD28 | CD8 |
| ABD | CD27 | CD28 | CD3ζ |
| ABD | CD27 | CD28 | CD3δ |
| ABD | CD27 | CD28 | CD3γ |
| ABD | CD27 | CD28 | CD3ε |
| ABD | CD27 | CD28 | FcγRI-γ |
| ABD | CD27 | CD28 | FcγRIII-γ |
| ABD | CD27 | CD28 | FcεRIβ |
| ABD | CD27 | CD28 | FcεRIγ |
| ABD | CD27 | CD28 | DAP10 |
| ABD | CD27 | CD28 | DAP12 |
| ABD | CD27 | CD28 | CD32 |
| ABD | CD27 | CD28 | CD79a |
| ABD | CD27 | CD28 | CD79b |
| ABD | CD27 | CD8 | CD8 |
| ABD | CD27 | CD8 | CD3ζ |
| ABD | CD27 | CD8 | CD3δ |
| ABD | CD27 | CD8 | CD3γ |
| ABD | CD27 | CD8 | CD3ε |
| ABD | CD27 | CD8 | FcγRI-γ |
| ABD | CD27 | CD8 | FcγRIII-γ |
| ABD | CD27 | CD8 | FcεRIβ |
| ABD | CD27 | CD8 | FcεRIγ |
| ABD | CD27 | CD8 | DAP10 |
| ABD | CD27 | CD8 | DAP12 |
| ABD | CD27 | CD8 | CD32 |
| ABD | CD27 | CD8 | CD79a |
| ABD | CD27 | CD8 | CD79b |
| ABD | CD27 | CD4 | CD8 |
| ABD | CD27 | CD4 | CD3ζ |
| ABD | CD27 | CD4 | CD3δ |
| ABD | CD27 | CD4 | CD3γ |
| ABD | CD27 | CD4 | CD3ε |
| ABD | CD27 | CD4 | FcγRI-γ |
| ABD | CD27 | CD4 | FcγRIII-γ |
| ABD | CD27 | CD4 | FcεRIβ |
| ABD | CD27 | CD4 | FcεRIγ |
| ABD | CD27 | CD4 | DAP10 |
| ABD | CD27 | CD4 | DAP12 |
| ABD | CD27 | CD4 | CD32 |
| ABD | CD27 | CD4 | CD79a |
| ABD | CD27 | CD4 | CD79b |
| ABD | CD27 | b2c | CD8 |
| ABD | CD27 | b2c | CD3ζ |
| ABD | CD27 | b2c | CD3δ |
| ABD | CD27 | b2c | CD3γ |
| ABD | CD27 | b2c | CD3ε |
| ABD | CD27 | b2c | FcγRI-γ |
| ABD | CD27 | b2c | FcγRIII-γ |
| ABD | CD27 | b2c | FcεRIβ |
| ABD | CD27 | b2c | FcεRIγ |
| ABD | CD27 | b2c | DAP10 |
| ABD | CD27 | b2c | DAP12 |
| ABD | CD27 | b2c | CD32 |
| ABD | CD27 | b2c | CD79a |
| ABD | CD27 | b2c | CD79b |
| ABD | CD27 | CD137/41BB | CD8 |
| ABD | CD27 | CD137/41BB | CD3ζ |
| ABD | CD27 | CD137/41BB | CD3δ |
| ABD | CD27 | CD137/41BB | CD3γ |
| ABD | CD27 | CD137/41BB | CD3ε |
| ABD | CD27 | CD137/41BB | FcγRI-γ |
| ABD | CD27 | CD137/41BB | FcγRIII-γ |
| ABD | CD27 | CD137/41BB | FcεRIβ |
| ABD | CD27 | CD137/41BB | FcεRIγ |
| ABD | CD27 | CD137/41BB | DAP10 |
| ABD | CD27 | CD137/41BB | DAP12 |
| ABD | CD27 | CD137/41BB | CD32 |
| ABD | CD27 | CD137/41BB | CD79a |
| ABD | CD27 | CD137/41BB | CD79b |
| ABD | CD27 | ICOS | CD8 |
| ABD | CD27 | ICOS | CD3ζ |
| ABD | CD27 | ICOS | CD3δ |
| ABD | CD27 | ICOS | CD3γ |
| ABD | CD27 | ICOS | CD3ε |
| ABD | CD27 | ICOS | FcγRI-γ |
| ABD | CD27 | ICOS | FcγRIII-γ |
| ABD | CD27 | ICOS | FcεRIβ |
| ABD | CD27 | ICOS | FcεRIγ |
| ABD | CD27 | ICOS | DAP10 |
| ABD | CD27 | ICOS | DAP12 |
| ABD | CD27 | ICOS | CD32 |
| ABD | CD27 | ICOS | CD79a |
| ABD | CD27 | ICOS | CD79b |
| ABD | CD27 | CD27 | CD8 |
| ABD | CD27 | CD27 | CD3ζ |
| ABD | CD27 | CD27 | CD3δ |
| ABD | CD27 | CD27 | CD3γ |
| ABD | CD27 | CD27 | CD3ε |
| ABD | CD27 | CD27 | FcγRI-γ |
| ABD | CD27 | CD27 | FcγRIII-γ |
| ABD | CD27 | CD27 | FcεRIβ |
| ABD | CD27 | CD27 | FcεRIγ |
| ABD | CD27 | CD27 | DAP10 |
| ABD | CD27 | CD27 | DAP12 |
| ABD | CD27 | CD27 | CD32 |
| ABD | CD27 | CD27 | CD79a |
| ABD | CD27 | CD27 | CD79b |
| ABD | CD27 | CD28δ | CD8 |
| ABD | CD27 | CD28δ | CD3ζ |
| ABD | CD27 | CD28δ | CD3δ |
| ABD | CD27 | CD28δ | CD3γ |
| ABD | CD27 | CD28δ | CD3ε |
| ABD | CD27 | CD28δ | FcγRI-γ |
| ABD | CD27 | CD28δ | FcγRIII-γ |
| ABD | CD27 | CD28δ | FcεRIβ |
| ABD | CD27 | CD28δ | FcεRIγ |
| ABD | CD27 | CD28δ | DAP10 |
| ABD | CD27 | CD28δ | DAP12 |
| ABD | CD27 | CD28δ | CD32 |
| ABD | CD27 | CD28δ | CD79a |
| ABD | CD27 | CD28δ | CD79b |
| ABD | CD27 | CD80 | CD8 |
| ABD | CD27 | CD80 | CD3ζ |
| ABD | CD27 | CD80 | CD3δ |
| ABD | CD27 | CD80 | CD3γ |
| ABD | CD27 | CD80 | CD3ε |
| ABD | CD27 | CD80 | FcγRI-γ |
| ABD | CD27 | CD80 | FcγRIII-γ |
| ABD | CD27 | CD80 | FcεRIβ |
| ABD | CD27 | CD80 | FcεRIγ |
| ABD | CD27 | CD80 | DAP10 |
| ABD | CD27 | CD80 | DAP12 |
| ABD | CD27 | CD80 | CD32 |
| ABD | CD27 | CD80 | CD79a |
| ABD | CD27 | CD80 | CD79b |
| ABD | CD27 | CD86 | CD8 |
| ABD | CD27 | CD86 | CD3ζ |
| ABD | CD27 | CD86 | CD3δ |
| ABD | CD27 | CD86 | CD3γ |
| ABD | CD27 | CD86 | CD3ε |
| ABD | CD27 | CD86 | FcγRI-γ |
| ABD | CD27 | CD86 | FcγRIII-γ |
| ABD | CD27 | CD86 | FcεRIβ |
| ABD | CD27 | CD86 | FcεRIγ |
| ABD | CD27 | CD86 | DAP10 |
| ABD | CD27 | CD86 | DAP12 |
| ABD | CD27 | CD86 | CD32 |
| ABD | CD27 | CD86 | CD79a |
| ABD | CD27 | CD86 | CD79b |
| ABD | CD27 | OX40 | CD8 |
| ABD | CD27 | OX40 | CD3ζ |
| ABD | CD27 | OX40 | CD3δ |
| ABD | CD27 | OX40 | CD3γ |
| ABD | CD27 | OX40 | CD3ε |
| ABD | CD27 | OX40 | FcγRI-γ |
| ABD | CD27 | OX40 | FcγRIII-γ |
| ABD | CD27 | OX40 | FcεRIβ |
| ABD | CD27 | OX40 | FcεRIγ |
| ABD | CD27 | OX40 | DAP10 |
| ABD | CD27 | OX40 | DAP12 |
| ABD | CD27 | OX40 | CD32 |
| ABD | CD27 | OX40 | CD79a |
| ABD | CD27 | OX40 | CD79b |
| ABD | CD27 | DAP10 | CD8 |
| ABD | CD27 | DAP10 | CD3ζ |
| ABD | CD27 | DAP10 | CD3δ |
| ABD | CD27 | DAP10 | CD3γ |
| ABD | CD27 | DAP10 | CD3ε |
| ABD | CD27 | DAP10 | FcγRI-γ |
| ABD | CD27 | DAP10 | FcγRIII-γ |
| ABD | CD27 | DAP10 | FcεRIβ |
| ABD | CD27 | DAP10 | FcεRIγ |
| ABD | CD27 | DAP10 | DAP10 |
| ABD | CD27 | DAP10 | DAP12 |
| ABD | CD27 | DAP10 | CD32 |
| ABD | CD27 | DAP10 | CD79a |
| ABD | CD27 | DAP10 | CD79b |
| ABD | CD27 | DAP12 | CD8 |
| ABD | CD27 | DAP12 | CD3ζ |
| ABD | CD27 | DAP12 | CD3δ |
| ABD | CD27 | DAP12 | CD3γ |
| ABD | CD27 | DAP12 | CD3ε |
| ABD | CD27 | DAP12 | FcγRI-γ |
| ABD | CD27 | DAP12 | FcγRIII-γ |
| ABD | CD27 | DAP12 | FcεRIβ |
| ABD | CD27 | DAP12 | FcεRIγ |
| ABD | CD27 | DAP12 | DAP10 |
| ABD | CD27 | DAP12 | DAP12 |
| ABD | CD27 | DAP12 | CD32 |
| ABD | CD27 | DAP12 | CD79a |
| ABD | CD27 | DAP12 | CD79b |
| ABD | CD27 | MyD88 | CD8 |
| ABD | CD27 | MyD88 | CD3ζ |
| ABD | CD27 | MyD88 | CD3δ |
| ABD | CD27 | MyD88 | CD3γ |
| ABD | CD27 | MyD88 | CD3ε |
| ABD | CD27 | MyD88 | FcγRI-γ |
| ABD | CD27 | MyD88 | FcγRIII-γ |
| ABD | CD27 | MyD88 | FcεRIβ |
| ABD | CD27 | MyD88 | FcεRIγ |
| ABD | CD27 | MyD88 | DAP10 |
| ABD | CD27 | MyD88 | DAP12 |
| ABD | CD27 | MyD88 | CD32 |
| ABD | CD27 | MyD88 | CD79a |
| ABD | CD27 | MyD88 | CD79b |
| ABD | CD27 | CD7 | CD8 |
| ABD | CD27 | CD7 | CD3ζ |
| ABD | CD27 | CD7 | CD3δ |
| ABD | CD27 | CD7 | CD3γ |
| ABD | CD27 | CD7 | CD3ε |
| ABD | CD27 | CD7 | FcγRI-γ |
| ABD | CD27 | CD7 | FcγRIII-γ |
| ABD | CD27 | CD7 | FcεRIβ |
| ABD | CD27 | CD7 | FcεRIγ |
| ABD | CD27 | CD7 | DAP10 |
| ABD | CD27 | CD7 | DAP12 |
| ABD | CD27 | CD7 | CD32 |
| ABD | CD27 | CD7 | CD79a |
| ABD | CD27 | CD7 | CD79b |
| ABD | CD27 | BTNL3 | CD8 |
| ABD | CD27 | BTNL3 | CD3ζ |
| ABD | CD27 | BTNL3 | CD3δ |
| ABD | CD27 | BTNL3 | CD3γ |
| ABD | CD27 | BTNL3 | CD3ε |
| ABD | CD27 | BTNL3 | FcγRI-γ |
| ABD | CD27 | BTNL3 | FcγRIII-γ |
| ABD | CD27 | BTNL3 | FcεRIβ |
| ABD | CD27 | BTNL3 | FcεRIγ |
| ABD | CD27 | BTNL3 | DAP10 |
| ABD | CD27 | BTNL3 | DAP12 |
| ABD | CD27 | BTNL3 | CD32 |
| ABD | CD27 | BTNL3 | CD79a |
| ABD | CD27 | BTNL3 | CD79b |
| ABD | CD27 | NKG2D | CD8 |
| ABD | CD27 | NKG2D | CD3ζ |
| ABD | CD27 | NKG2D | CD3δ |
| ABD | CD27 | NKG2D | CD3γ |
| ABD | CD27 | NKG2D | CD3ε |
| ABD | CD27 | NKG2D | FcγRI-γ |
| ABD | CD27 | NKG2D | FcγRIII-γ |
| ABD | CD27 | NKG2D | FcεRIβ |
| ABD | CD27 | NKG2D | FcεRIγ |
| ABD | CD27 | NKG2D | DAP10 |
| ABD | CD27 | NKG2D | DAP12 |
| ABD | CD27 | NKG2D | CD32 |
| ABD | CD27 | NKG2D | CD79a |
| ABD | CD27 | NKG2D | CD79b |
| ABD | CD28δ | CD28 | CD8 |
| ABD | CD28δ | CD28 | CD3ζ |
| ABD | CD28δ | CD28 | CD3δ |
| ABD | CD28δ | CD28 | CD3γ |
| ABD | CD28δ | CD28 | CD3ε |
| ABD | CD28δ | CD28 | FcγRI-γ |
| ABD | CD28δ | CD28 | FcγRIII-γ |
| ABD | CD28δ | CD28 | FcεRIβ |
| ABD | CD28δ | CD28 | FcεRIγ |
| ABD | CD28δ | CD28 | DAP10 |
| ABD | CD28δ | CD28 | DAP12 |
| ABD | CD28δ | CD28 | CD32 |
| ABD | CD28δ | CD28 | CD79a |
| ABD | CD28δ | CD28 | CD79b |
| ABD | CD28δ | CD8 | CD8 |
| ABD | CD28δ | CD8 | CD3ζ |
| ABD | CD28δ | CD8 | CD3δ |
| ABD | CD28δ | CD8 | CD3γ |
| ABD | CD28δ | CD8 | CD3ε |
| ABD | CD28δ | CD8 | FcγRI-γ |
| ABD | CD28δ | CD8 | FcγRIII-γ |
| ABD | CD28δ | CD8 | FcεRIβ |
| ABD | CD28δ | CD8 | FcεRIγ |
| ABD | CD28δ | CD8 | DAP10 |
| ABD | CD28δ | CD8 | DAP12 |
| ABD | CD28δ | CD8 | CD32 |
| ABD | CD28δ | CD8 | CD79a |
| ABD | CD28δ | CD8 | CD79b |
| ABD | CD28δ | CD4 | CD8 |
| ABD | CD28δ | CD4 | CD3ζ |
| ABD | CD28δ | CD4 | CD3δ |
| ABD | CD28δ | CD4 | CD3γ |
| ABD | CD28δ | CD4 | CD3ε |
| ABD | CD28δ | CD4 | FcγRI-γ |
| ABD | CD28δ | CD4 | FcγRIII-γ |
| ABD | CD28δ | CD4 | FcεRIβ |
| ABD | CD28δ | CD4 | FcεRIγ |
| ABD | CD28δ | CD4 | DAP10 |
| ABD | CD28δ | CD4 | DAP12 |
| ABD | CD28δ | CD4 | CD32 |
| ABD | CD28δ | CD4 | CD79a |
| ABD | CD28δ | CD4 | CD79b |
| ABD | CD28δ | b2c | CD8 |
| ABD | CD28δ | b2c | CD3ζ |
| ABD | CD28δ | b2c | CD3δ |
| ABD | CD28δ | b2c | CD3γ |
| ABD | CD28δ | b2c | CD3ε |
| ABD | CD28δ | b2c | FcγRI-γ |
| ABD | CD28δ | b2c | FcγRIII-γ |
| ABD | CD28δ | b2c | FcεRIβ |
| ABD | CD28δ | b2c | FcεRIγ |
| ABD | CD28δ | b2c | DAP10 |
| ABD | CD28δ | b2c | DAP12 |
| ABD | CD28δ | b2c | CD32 |
| ABD | CD28δ | b2c | CD79a |
| ABD | CD28δ | b2c | CD79b |
| ABD | CD28δ | CD137/41BB | CD8 |
| ABD | CD28δ | CD137/41BB | CD3ζ |
| ABD | CD28δ | CD137/41BB | CD3δ |
| ABD | CD28δ | CD137/41BB | CD3γ |
| ABD | CD28δ | CD137/41BB | CD3ε |
| ABD | CD28δ | CD137/41BB | FcγRI-γ |
| ABD | CD28δ | CD137/41BB | FcγRIII-γ |
| ABD | CD28δ | CD137/41BB | FcεRIβ |
| ABD | CD28δ | CD137/41BB | FcεRIγ |
| ABD | CD28δ | CD137/41BB | DAP10 |
| ABD | CD28δ | CD137/41BB | DAP12 |
| ABD | CD28δ | CD137/41BB | CD32 |
| ABD | CD28δ | CD137/41BB | CD79a |
| ABD | CD28δ | CD137/41BB | CD79b |
| ABD | CD28δ | ICOS | CD8 |
| ABD | CD28δ | ICOS | CD3ζ |
| ABD | CD28δ | ICOS | CD3δ |
| ABD | CD28δ | ICOS | CD3γ |
| ABD | CD28δ | ICOS | CD3ε |
| ABD | CD28δ | ICOS | FcγRI-γ |
| ABD | CD28δ | ICOS | FcγRIII-γ |
| ABD | CD28δ | ICOS | FcεRIβ |
| ABD | CD28δ | ICOS | FcεRIγ |
| ABD | CD28δ | ICOS | DAP10 |
| ABD | CD28δ | ICOS | DAP12 |
| ABD | CD28δ | ICOS | CD32 |
| ABD | CD28δ | ICOS | CD79a |
| ABD | CD28δ | ICOS | CD79b |
| ABD | CD28δ | CD27 | CD8 |
| ABD | CD28δ | CD27 | CD3ζ |
| ABD | CD28δ | CD27 | CD3δ |
| ABD | CD28δ | CD27 | CD3γ |
| ABD | CD28δ | CD27 | CD3ε |
| ABD | CD28δ | CD27 | FcγRI-γ |
| ABD | CD28δ | CD27 | FcγRIII-γ |
| ABD | CD28δ | CD27 | FcεRIβ |
| ABD | CD28δ | CD27 | FcεRIγ |
| ABD | CD28δ | CD27 | DAP10 |
| ABD | CD28δ | CD27 | DAP12 |
| ABD | CD28δ | CD27 | CD32 |
| ABD | CD28δ | CD27 | CD79a |
| ABD | CD28δ | CD27 | CD79b |
| ABD | CD28δ | CD28δ | CD8 |
| ABD | CD28δ | CD28δ | CD3ζ |
| ABD | CD28δ | CD28δ | CD3δ |
| ABD | CD28δ | CD28δ | CD3γ |
| ABD | CD28δ | CD28δ | CD3ε |
| ABD | CD28δ | CD28δ | FcγRI-γ |
| ABD | CD28δ | CD28δ | FcγRIII-γ |
| ABD | CD28δ | CD28δ | FcεRIβ |
| ABD | CD28δ | CD28δ | FcεRIγ |
| ABD | CD28δ | CD28δ | DAP10 |
| ABD | CD28δ | CD28δ | DAP12 |
| ABD | CD28δ | CD28δ | CD32 |
| ABD | CD28δ | CD28δ | CD79a |
| ABD | CD28δ | CD28δ | CD79b |
| ABD | CD28δ | CD80 | CD8 |
| ABD | CD28δ | CD80 | CD3ζ |
| ABD | CD28δ | CD80 | CD3δ |
| ABD | CD28δ | CD80 | CD3γ |
| ABD | CD28δ | CD80 | CD3ε |
| ABD | CD28δ | CD80 | FcγRI-γ |
| ABD | CD28δ | CD80 | FcγRIII-γ |
| ABD | CD28δ | CD80 | FcεRIβ |
| ABD | CD28δ | CD80 | FcεRIγ |
| ABD | CD28δ | CD80 | DAP10 |
| ABD | CD28δ | CD80 | DAP12 |
| ABD | CD28δ | CD80 | CD32 |
| ABD | CD28δ | CD80 | CD79a |
| ABD | CD28δ | CD80 | CD79b |
| ABD | CD28δ | CD86 | CD8 |
| ABD | CD28δ | CD86 | CD3ζ |
| ABD | CD28δ | CD86 | CD3δ |
| ABD | CD28δ | CD86 | CD3γ |
| ABD | CD28δ | CD86 | CD3ε |
| ABD | CD28δ | CD86 | FcγRI-γ |
| ABD | CD28δ | CD86 | FcγRIII-γ |
| ABD | CD28δ | CD86 | FcεRIβ |
| ABD | CD28δ | CD86 | FcεRIγ |
| ABD | CD28δ | CD86 | DAP10 |
| ABD | CD28δ | CD86 | DAP12 |
| ABD | CD28δ | CD86 | CD32 |
| ABD | CD28δ | CD86 | CD79a |
| ABD | CD28δ | CD86 | CD79b |
| ABD | CD28δ | OX40 | CD8 |
| ABD | CD28δ | OX40 | CD3ζ |
| ABD | CD28δ | OX40 | CD3δ |
| ABD | CD28δ | OX40 | CD3γ |
| ABD | CD28δ | OX40 | CD3ε |
| ABD | CD28δ | OX40 | FcγRI-γ |
| ABD | CD28δ | OX40 | FcγRIII-γ |
| ABD | CD28δ | OX40 | FcεRIβ |
| ABD | CD28δ | OX40 | FcεRIγ |
| ABD | CD28δ | OX40 | DAP10 |
| ABD | CD28δ | OX40 | DAP12 |
| ABD | CD28δ | OX40 | CD32 |
| ABD | CD28δ | OX40 | CD79a |
| ABD | CD28δ | OX40 | CD79b |
| ABD | CD28δ | DAP10 | CD8 |
| ABD | CD28δ | DAP10 | CD3ζ |
| ABD | CD28δ | DAP10 | CD3δ |
| ABD | CD28δ | DAP10 | CD3γ |
| ABD | CD28δ | DAP10 | CD3ε |
| ABD | CD28δ | DAP10 | FcγRI-γ |
| ABD | CD28δ | DAP10 | FcγRIII-γ |
| ABD | CD28δ | DAP10 | FcεRIβ |
| ABD | CD28δ | DAP10 | FcεRIγ |
| ABD | CD28δ | DAP10 | DAP10 |
| ABD | CD28δ | DAP10 | DAP12 |
| ABD | CD28δ | DAP10 | CD32 |
| ABD | CD28δ | DAP10 | CD79a |
| ABD | CD28δ | DAP10 | CD79b |
| ABD | CD28δ | DAP12 | CD8 |
| ABD | CD28δ | DAP12 | CD3ζ |
| ABD | CD28δ | DAP12 | CD3δ |
| ABD | CD28δ | DAP12 | CD3γ |
| ABD | CD28δ | DAP12 | CD3ε |
| ABD | CD28δ | DAP12 | FcγRI-γ |
| ABD | CD28δ | DAP12 | FcγRIII-γ |
| ABD | CD28δ | DAP12 | FcεRIβ |
| ABD | CD28δ | DAP12 | FcεRIγ |
| ABD | CD28δ | DAP12 | DAP10 |
| ABD | CD28δ | DAP12 | DAP12 |
| ABD | CD28δ | DAP12 | CD32 |
| ABD | CD28δ | DAP12 | CD79a |
| ABD | CD28δ | DAP12 | CD79b |
| ABD | CD28δ | MyD88 | CD8 |
| ABD | CD28δ | MyD88 | CD3ζ |
| ABD | CD28δ | MyD88 | CD3δ |
| ABD | CD28δ | MyD88 | CD3γ |
| ABD | CD28δ | MyD88 | CD3ε |
| ABD | CD28δ | MyD88 | FcγRI-γ |
| ABD | CD28δ | MyD88 | FcγRIII-γ |
| ABD | CD28δ | MyD88 | FcεRIβ |
| ABD | CD28δ | MyD88 | FcεRIγ |
| ABD | CD28δ | MyD88 | DAP10 |
| ABD | CD28δ | MyD88 | DAP12 |
| ABD | CD28δ | MyD88 | CD32 |
| ABD | CD28δ | MyD88 | CD79a |
| ABD | CD28δ | MyD88 | CD79b |
| ABD | CD28δ | CD7 | CD8 |
| ABD | CD28δ | CD7 | CD3ζ |
| ABD | CD28δ | CD7 | CD3δ |
| ABD | CD28δ | CD7 | CD3γ |
| ABD | CD28δ | CD7 | CD3ε |
| ABD | CD28δ | CD7 | FcγRI-γ |
| ABD | CD28δ | CD7 | FcγRIII-γ |
| ABD | CD28δ | CD7 | FcεRIβ |
| ABD | CD28δ | CD7 | FcεRIγ |
| ABD | CD28δ | CD7 | DAP10 |
| ABD | CD28δ | CD7 | DAP12 |
| ABD | CD28δ | CD7 | CD32 |
| ABD | CD28δ | CD7 | CD79a |
| ABD | CD28δ | CD7 | CD79b |
| ABD | CD28δ | BTNL3 | CD8 |
| ABD | CD28δ | BTNL3 | CD3ζ |
| ABD | CD28δ | BTNL3 | CD3δ |
| ABD | CD28δ | BTNL3 | CD3γ |
| ABD | CD28δ | BTNL3 | CD3ε |
| ABD | CD28δ | BTNL3 | FcγRI-γ |
| ABD | CD28δ | BTNL3 | FcγRIII-γ |
| ABD | CD28δ | BTNL3 | FcεRIβ |
| ABD | CD28δ | BTNL3 | FcεRIγ |
| ABD | CD28δ | BTNL3 | DAP10 |
| ABD | CD28δ | BTNL3 | DAP12 |
| ABD | CD28δ | BTNL3 | CD32 |
| ABD | CD28δ | BTNL3 | CD79a |
| ABD | CD28δ | BTNL3 | CD79b |
| ABD | CD28δ | NKG2D | CD8 |
| ABD | CD28δ | NKG2D | CD3ζ |
| ABD | CD28δ | NKG2D | CD3δ |
| ABD | CD28δ | NKG2D | CD3γ |
| ABD | CD28δ | NKG2D | CD3ε |
| ABD | CD28δ | NKG2D | FcγRI-γ |
| ABD | CD28δ | NKG2D | FcγRIII-γ |
| ABD | CD28δ | NKG2D | FcεRIβ |
| ABD | CD28δ | NKG2D | FcεRIγ |
| ABD | CD28δ | NKG2D | DAP10 |
| ABD | CD28δ | NKG2D | DAP12 |
| ABD | CD28δ | NKG2D | CD32 |
| ABD | CD28δ | NKG2D | CD79a |
| ABD | CD28δ | NKG2D | CD79b |
| ABD | CD80 | CD28 | CD8 |
| ABD | CD80 | CD28 | CD3ζ |
| ABD | CD80 | CD28 | CD3δ |
| ABD | CD80 | CD28 | CD3γ |
| ABD | CD80 | CD28 | CD3ε |
| ABD | CD80 | CD28 | FcγRI-γ |
| ABD | CD80 | CD28 | FcγRIII-γ |
| ABD | CD80 | CD28 | FcεRIβ |
| ABD | CD80 | CD28 | FcεRIγ |
| ABD | CD80 | CD28 | DAP10 |
| ABD | CD80 | CD28 | DAP12 |
| ABD | CD80 | CD28 | CD32 |
| ABD | CD80 | CD28 | CD79a |
| ABD | CD80 | CD28 | CD79b |
| ABD | CD80 | CD8 | CD8 |
| ABD | CD80 | CD8 | CD3ζ |
| ABD | CD80 | CD8 | CD3δ |
| ABD | CD80 | CD8 | CD3γ |
| ABD | CD80 | CD8 | CD3ε |
| ABD | CD80 | CD8 | FcγRI-γ |
| ABD | CD80 | CD8 | FcγRIII-γ |
| ABD | CD80 | CD8 | FcεRIβ |
| ABD | CD80 | CD8 | FcεRIγ |
| ABD | CD80 | CD8 | DAP10 |
| ABD | CD80 | CD8 | DAP12 |
| ABD | CD80 | CD8 | CD32 |
| ABD | CD80 | CD8 | CD79a |
| ABD | CD80 | CD8 | CD79b |
| ABD | CD80 | CD4 | CD8 |
| ABD | CD80 | CD4 | CD3ζ |
| ABD | CD80 | CD4 | CD3δ |
| ABD | CD80 | CD4 | CD3γ |
| ABD | CD80 | CD4 | CD3ε |
| ABD | CD80 | CD4 | FcγRI-γ |
| ABD | CD80 | CD4 | FcγRIII-γ |
| ABD | CD80 | CD4 | FcεRIβ |
| ABD | CD80 | CD4 | FcεRIγ |
| ABD | CD80 | CD4 | DAP10 |
| ABD | CD80 | CD4 | DAP12 |
| ABD | CD80 | CD4 | CD32 |
| ABD | CD80 | CD4 | CD79a |
| ABD | CD80 | CD4 | CD79b |
| ABD | CD80 | b2c | CD8 |
| ABD | CD80 | b2c | CD3ζ |
| ABD | CD80 | b2c | CD3δ |
| ABD | CD80 | b2c | CD3γ |
| ABD | CD80 | b2c | CD3ε |
| ABD | CD80 | b2c | FcγRI-γ |
| ABD | CD80 | b2c | FcγRIII-γ |
| ABD | CD80 | b2c | FcεRIβ |
| ABD | CD80 | b2c | FcεRIγ |
| ABD | CD80 | b2c | DAP10 |
| ABD | CD80 | b2c | DAP12 |
| ABD | CD80 | b2c | CD32 |
| ABD | CD80 | b2c | CD79a |
| ABD | CD80 | b2c | CD79b |
| ABD | CD80 | CD137/41BB | CD8 |
| ABD | CD80 | CD137/41BB | CD3ζ |
| ABD | CD80 | CD137/41BB | CD3δ |
| ABD | CD80 | CD137/41BB | CD3γ |
| ABD | CD80 | CD137/41BB | CD3ε |
| ABD | CD80 | CD137/41BB | FcγRI-γ |
| ABD | CD80 | CD137/41BB | FcγRIII-γ |
| ABD | CD80 | CD137/41BB | FcεRIβ |
| ABD | CD80 | CD137/41BB | FcεRIγ |
| ABD | CD80 | CD137/41BB | DAP10 |
| ABD | CD80 | CD137/41BB | DAP12 |
| ABD | CD80 | CD137/41BB | CD32 |
| ABD | CD80 | CD137/41BB | CD79a |
| ABD | CD80 | CD137/41BB | CD79b |
| ABD | CD80 | ICOS | CD8 |
| ABD | CD80 | ICOS | CD3ζ |
| ABD | CD80 | ICOS | CD3δ |
| ABD | CD80 | ICOS | CD3γ |
| ABD | CD80 | ICOS | CD3ε |
| ABD | CD80 | ICOS | FcγRI-γ |
| ABD | CD80 | ICOS | FcγRIII-γ |
| ABD | CD80 | ICOS | FcεRIβ |
| ABD | CD80 | ICOS | FcεRIγ |
| ABD | CD80 | ICOS | DAP10 |
| ABD | CD80 | ICOS | DAP12 |
| ABD | CD80 | ICOS | CD32 |
| ABD | CD80 | ICOS | CD79a |
| ABD | CD80 | ICOS | CD79b |
| ABD | CD80 | CD27 | CD8 |
| ABD | CD80 | CD27 | CD3ζ |
| ABD | CD80 | CD27 | CD3δ |
| ABD | CD80 | CD27 | CD3γ |
| ABD | CD80 | CD27 | CD3ε |
| ABD | CD80 | CD27 | FcγRI-γ |
| ABD | CD80 | CD27 | FcγRIII-γ |
| ABD | CD80 | CD27 | FcεRIβ |
| ABD | CD80 | CD27 | FcεRIγ |
| ABD | CD80 | CD27 | DAP10 |
| ABD | CD80 | CD27 | DAP12 |
| ABD | CD80 | CD27 | CD32 |
| ABD | CD80 | CD27 | CD79a |
| ABD | CD80 | CD27 | CD79b |
| ABD | CD80 | CD28δ | CD8 |
| ABD | CD80 | CD28δ | CD3ζ |
| ABD | CD80 | CD28δ | CD3δ |
| ABD | CD80 | CD28δ | CD3γ |
| ABD | CD80 | CD28δ | CD3ε |
| ABD | CD80 | CD28δ | FcγRI-γ |
| ABD | CD80 | CD28δ | FcγRIII-γ |
| ABD | CD80 | CD28δ | FcεRIβ |
| ABD | CD80 | CD28δ | FcεRIγ |
| ABD | CD80 | CD28δ | DAP10 |
| ABD | CD80 | CD28δ | DAP12 |
| ABD | CD80 | CD28δ | CD32 |
| ABD | CD80 | CD28δ | CD79a |
| ABD | CD80 | CD28δ | CD79b |
| ABD | CD80 | CD80 | CD8 |
| ABD | CD80 | CD80 | CD3ζ |
| ABD | CD80 | CD80 | CD3δ |
| ABD | CD80 | CD80 | CD3γ |
| ABD | CD80 | CD80 | CD3ε |
| ABD | CD80 | CD80 | FcγRI-γ |
| ABD | CD80 | CD80 | FcγRIII-γ |
| ABD | CD80 | CD80 | FcεRIβ |
| ABD | CD80 | CD80 | FcεRIγ |
| ABD | CD80 | CD80 | DAP10 |
| ABD | CD80 | CD80 | DAP12 |
| ABD | CD80 | CD80 | CD32 |
| ABD | CD80 | CD80 | CD79a |
| ABD | CD80 | CD80 | CD79b |
| ABD | CD80 | CD86 | CD8 |
| ABD | CD80 | CD86 | CD3ζ |
| ABD | CD80 | CD86 | CD3δ |
| ABD | CD80 | CD86 | CD3γ |
| ABD | CD80 | CD86 | CD3ε |
| ABD | CD80 | CD86 | FcγRI-γ |
| ABD | CD80 | CD86 | FcγRIII-γ |
| ABD | CD80 | CD86 | FcεRIβ |
| ABD | CD80 | CD86 | FcεRIγ |
| ABD | CD80 | CD86 | DAP10 |
| ABD | CD80 | CD86 | DAP12 |
| ABD | CD80 | CD86 | CD32 |
| ABD | CD80 | CD86 | CD79a |
| ABD | CD80 | CD86 | CD79b |
| ABD | CD80 | OX40 | CD8 |
| ABD | CD80 | OX40 | CD3ζ |
| ABD | CD80 | OX40 | CD3δ |
| ABD | CD80 | OX40 | CD3γ |
| ABD | CD80 | OX40 | CD3ε |
| ABD | CD80 | OX40 | FcγRI-γ |
| ABD | CD80 | OX40 | FcγRIII-γ |
| ABD | CD80 | OX40 | FcεRIβ |
| ABD | CD80 | OX40 | FcεRIγ |
| ABD | CD80 | OX40 | DAP10 |
| ABD | CD80 | OX40 | DAP12 |
| ABD | CD80 | OX40 | CD32 |
| ABD | CD80 | OX40 | CD79a |
| ABD | CD80 | OX40 | CD79b |
| ABD | CD80 | DAP10 | CD8 |
| ABD | CD80 | DAP10 | CD3ζ |
| ABD | CD80 | DAP10 | CD3δ |
| ABD | CD80 | DAP10 | CD3γ |
| ABD | CD80 | DAP10 | CD3ε |
| ABD | CD80 | DAP10 | FcγRI-γ |
| ABD | CD80 | DAP10 | FcγRIII-γ |
| ABD | CD80 | DAP10 | FcεRIβ |
| ABD | CD80 | DAP10 | FcεRIγ |
| ABD | CD80 | DAP10 | DAP10 |
| ABD | CD80 | DAP10 | DAP12 |
| ABD | CD80 | DAP10 | CD32 |
| ABD | CD80 | DAP10 | CD79a |
| ABD | CD80 | DAP10 | CD79b |
| ABD | CD80 | DAP12 | CD8 |
| ABD | CD80 | DAP12 | CD3ζ |
| ABD | CD80 | DAP12 | CD3δ |
| ABD | CD80 | DAP12 | CD3γ |
| ABD | CD80 | DAP12 | CD3ε |
| ABD | CD80 | DAP12 | FcγRI-γ |
| ABD | CD80 | DAP12 | FcγRIII-γ |
| ABD | CD80 | DAP12 | FcεRIβ |
| ABD | CD80 | DAP12 | FcεRIγ |
| ABD | CD80 | DAP12 | DAP10 |
| ABD | CD80 | DAP12 | DAP12 |
| ABD | CD80 | DAP12 | CD32 |
| ABD | CD80 | DAP12 | CD79a |
| ABD | CD80 | DAP12 | CD79b |
| ABD | CD80 | MyD88 | CD8 |
| ABD | CD80 | MyD88 | CD3ζ |
| ABD | CD80 | MyD88 | CD3δ |
| ABD | CD80 | MyD88 | CD3γ |
| ABD | CD80 | MyD88 | CD3ε |
| ABD | CD80 | MyD88 | FcγRI-γ |
| ABD | CD80 | MyD88 | FcγRIII-γ |
| ABD | CD80 | MyD88 | FcεRIβ |
| ABD | CD80 | MyD88 | FcεRIγ |
| ABD | CD80 | MyD88 | DAP10 |
| ABD | CD80 | MyD88 | DAP12 |
| ABD | CD80 | MyD88 | CD32 |
| ABD | CD80 | MyD88 | CD79a |
| ABD | CD80 | MyD88 | CD79b |
| ABD | CD80 | CD7 | CD8 |
| ABD | CD80 | CD7 | CD3ζ |
| ABD | CD80 | CD7 | CD3δ |
| ABD | CD80 | CD7 | CD3γ |
| ABD | CD80 | CD7 | CD3ε |
| ABD | CD80 | CD7 | FcγRI-γ |
| ABD | CD80 | CD7 | FcγRIII-γ |
| ABD | CD80 | CD7 | FcεRIβ |
| ABD | CD80 | CD7 | FcεRIγ |
| ABD | CD80 | CD7 | DAP10 |
| ABD | CD80 | CD7 | DAP12 |
| ABD | CD80 | CD7 | CD32 |
| ABD | CD80 | CD7 | CD79a |
| ABD | CD80 | CD7 | CD79b |
| ABD | CD80 | BTNL3 | CD8 |
| ABD | CD80 | BTNL3 | CD3ζ |
| ABD | CD80 | BTNL3 | CD3δ |
| ABD | CD80 | BTNL3 | CD3γ |
| ABD | CD80 | BTNL3 | CD3ε |
| ABD | CD80 | BTNL3 | FcγRI-γ |
| ABD | CD80 | BTNL3 | FcγRIII-γ |
| ABD | CD80 | BTNL3 | FcεRIβ |
| ABD | CD80 | BTNL3 | FcεRIγ |
| ABD | CD80 | BTNL3 | DAP10 |
| ABD | CD80 | BTNL3 | DAP12 |
| ABD | CD80 | BTNL3 | CD32 |
| ABD | CD80 | BTNL3 | CD79a |
| ABD | CD80 | BTNL3 | CD79b |
| ABD | CD80 | NKG2D | CD8 |
| ABD | CD80 | NKG2D | CD3ζ |
| ABD | CD80 | NKG2D | CD3δ |
| ABD | CD80 | NKG2D | CD3γ |
| ABD | CD80 | NKG2D | CD3ε |
| ABD | CD80 | NKG2D | FcγRI-γ |
| ABD | CD80 | NKG2D | FcγRIII-γ |
| ABD | CD80 | NKG2D | FcεRIβ |
| ABD | CD80 | NKG2D | FcεRIγ |
| ABD | CD80 | NKG2D | DAP10 |
| ABD | CD80 | NKG2D | DAP12 |
| ABD | CD80 | NKG2D | CD32 |
| ABD | CD80 | NKG2D | CD79a |
| ABD | CD80 | NKG2D | CD79b |
| ABD | CD86 | CD28 | CD8 |
| ABD | CD86 | CD28 | CD3ζ |
| ABD | CD86 | CD28 | CD3δ |
| ABD | CD86 | CD28 | CD3γ |
| ABD | CD86 | CD28 | CD3ε |
| ABD | CD86 | CD28 | FcγRI-γ |
| ABD | CD86 | CD28 | FcγRIII-γ |
| ABD | CD86 | CD28 | FcεRIβ |
| ABD | CD86 | CD28 | FcεRIγ |
| ABD | CD86 | CD28 | DAP10 |
| ABD | CD86 | CD28 | DAP12 |
| ABD | CD86 | CD28 | CD32 |
| ABD | CD86 | CD28 | CD79a |
| ABD | CD86 | CD28 | CD79b |
| ABD | CD86 | CD8 | CD8 |
| ABD | CD86 | CD8 | CD3ζ |
| ABD | CD86 | CD8 | CD3δ |
| ABD | CD86 | CD8 | CD3γ |
| ABD | CD86 | CD8 | CD3ε |
| ABD | CD86 | CD8 | FcγRI-γ |
| ABD | CD86 | CD8 | FcγRIII-γ |
| ABD | CD86 | CD8 | FcεRIβ |
| ABD | CD86 | CD8 | FcεRIγ |
| ABD | CD86 | CD8 | DAP10 |
| ABD | CD86 | CD8 | DAP12 |
| ABD | CD86 | CD8 | CD32 |
| ABD | CD86 | CD8 | CD79a |
| ABD | CD86 | CD8 | CD79b |
| ABD | CD86 | CD4 | CD8 |
| ABD | CD86 | CD4 | CD3ζ |
| ABD | CD86 | CD4 | CD3δ |
| ABD | CD86 | CD4 | CD3γ |
| ABD | CD86 | CD4 | CD3ε |
| ABD | CD86 | CD4 | FcγRI-γ |
| ABD | CD86 | CD4 | FcγRIII-γ |
| ABD | CD86 | CD4 | FcεRIβ |
| ABD | CD86 | CD4 | FcεRIγ |
| ABD | CD86 | CD4 | DAP10 |
| ABD | CD86 | CD4 | DAP12 |
| ABD | CD86 | CD4 | CD32 |
| ABD | CD86 | CD4 | CD79a |
| ABD | CD86 | CD4 | CD79b |
| ABD | CD86 | b2c | CD8 |
| ABD | CD86 | b2c | CD3ζ |
| ABD | CD86 | b2c | CD3δ |
| ABD | CD86 | b2c | CD3γ |
| ABD | CD86 | b2c | CD3ε |
| ABD | CD86 | b2c | FcγRI-γ |
| ABD | CD86 | b2c | FcγRIII-γ |
| ABD | CD86 | b2c | FcεRIβ |
| ABD | CD86 | b2c | FcεRIγ |
| ABD | CD86 | b2c | DAP10 |
| ABD | CD86 | b2c | DAP12 |
| ABD | CD86 | b2c | CD32 |
| ABD | CD86 | b2c | CD79a |
| ABD | CD86 | b2c | CD79b |
| ABD | CD86 | CD137/41BB | CD8 |
| ABD | CD86 | CD137/41BB | CD3ζ |
| ABD | CD86 | CD137/41BB | CD3δ |
| ABD | CD86 | CD137/41BB | CD3γ |
| ABD | CD86 | CD137/41BB | CD3ε |
| ABD | CD86 | CD137/41BB | FcγRI-γ |
| ABD | CD86 | CD137/41BB | FcγRIII-γ |
| ABD | CD86 | CD137/41BB | FcεRIβ |
| ABD | CD86 | CD137/41BB | FcεRIγ |
| ABD | CD86 | CD137/41BB | DAP10 |
| ABD | CD86 | CD137/41BB | DAP12 |
| ABD | CD86 | CD137/41BB | CD32 |
| ABD | CD86 | CD137/41BB | CD79a |
| ABD | CD86 | CD137/41BB | CD79b |
| ABD | CD86 | ICOS | CD8 |
| ABD | CD86 | ICOS | CD3ζ |
| ABD | CD86 | ICOS | CD3δ |
| ABD | CD86 | ICOS | CD3γ |
| ABD | CD86 | ICOS | CD3ε |
| ABD | CD86 | ICOS | FcγRI-γ |
| ABD | CD86 | ICOS | FcγRIII-γ |
| ABD | CD86 | ICOS | FcεRIβ |
| ABD | CD86 | ICOS | FcεRIγ |
| ABD | CD86 | ICOS | DAP10 |
| ABD | CD86 | ICOS | DAP12 |
| ABD | CD86 | ICOS | CD32 |
| ABD | CD86 | ICOS | CD79a |
| ABD | CD86 | ICOS | CD79b |
| ABD | CD86 | CD27 | CD8 |
| ABD | CD86 | CD27 | CD3ζ |
| ABD | CD86 | CD27 | CD3δ |
| ABD | CD86 | CD27 | CD3γ |
| ABD | CD86 | CD27 | CD3ε |
| ABD | CD86 | CD27 | FcγRI-γ |
| ABD | CD86 | CD27 | FcγRIII-γ |
| ABD | CD86 | CD27 | FcεRIβ |
| ABD | CD86 | CD27 | FcεRIγ |
| ABD | CD86 | CD27 | DAP10 |
| ABD | CD86 | CD27 | DAP12 |
| ABD | CD86 | CD27 | CD32 |
| ABD | CD86 | CD27 | CD79a |
| ABD | CD86 | CD27 | CD79b |
| ABD | CD86 | CD28δ | CD8 |
| ABD | CD86 | CD28δ | CD3ζ |
| ABD | CD86 | CD28δ | CD3δ |
| ABD | CD86 | CD28δ | CD3γ |
| ABD | CD86 | CD28δ | CD3ε |
| ABD | CD86 | CD28δ | FcγRI-γ |
| ABD | CD86 | CD28δ | FcγRIII-γ |
| ABD | CD86 | CD28δ | FcεRIβ |
| ABD | CD86 | CD28δ | FcεRIγ |
| ABD | CD86 | CD28δ | DAP10 |
| ABD | CD86 | CD28δ | DAP12 |
| ABD | CD86 | CD28δ | CD32 |
| ABD | CD86 | CD28δ | CD79a |
| ABD | CD86 | CD28δ | CD79b |
| ABD | CD86 | CD80 | CD8 |
| ABD | CD86 | CD80 | CD3ζ |
| ABD | CD86 | CD80 | CD3δ |
| ABD | CD86 | CD80 | CD3γ |
| ABD | CD86 | CD80 | CD3ε |
| ABD | CD86 | CD80 | FcγRI-γ |
| ABD | CD86 | CD80 | FcγRIII-γ |
| ABD | CD86 | CD80 | FcεRIβ |
| ABD | CD86 | CD80 | FcεRIγ |
| ABD | CD86 | CD80 | DAP10 |
| ABD | CD86 | CD80 | DAP12 |
| ABD | CD86 | CD80 | CD32 |
| ABD | CD86 | CD80 | CD79a |
| ABD | CD86 | CD80 | CD79b |
| ABD | CD86 | CD86 | CD8 |
| ABD | CD86 | CD86 | CD3ζ |
| ABD | CD86 | CD86 | CD3δ |
| ABD | CD86 | CD86 | CD3γ |
| ABD | CD86 | CD86 | CD3ε |
| ABD | CD86 | CD86 | FcγRI-γ |
| ABD | CD86 | CD86 | FcγRIII-γ |
| ABD | CD86 | CD86 | FcεRIβ |
| ABD | CD86 | CD86 | FcεRIγ |
| ABD | CD86 | CD86 | DAP10 |
| ABD | CD86 | CD86 | DAP12 |
| ABD | CD86 | CD86 | CD32 |
| ABD | CD86 | CD86 | CD79a |
| ABD | CD86 | CD86 | CD79b |
| ABD | CD86 | OX40 | CD8 |
| ABD | CD86 | OX40 | CD3ζ |
| ABD | CD86 | OX40 | CD3δ |
| ABD | CD86 | OX40 | CD3γ |
| ABD | CD86 | OX40 | CD3ε |
| ABD | CD86 | OX40 | FcγRI-γ |
| ABD | CD86 | OX40 | FcγRIII-γ |
| ABD | CD86 | OX40 | FcεRIβ |
| ABD | CD86 | OX40 | FcεRIγ |
| ABD | CD86 | OX40 | DAP10 |
| ABD | CD86 | OX40 | DAP12 |
| ABD | CD86 | OX40 | CD32 |
| ABD | CD86 | OX40 | CD79a |
| ABD | CD86 | OX40 | CD79b |
| ABD | CD86 | DAP10 | CD8 |
| ABD | CD86 | DAP10 | CD3ζ |
| ABD | CD86 | DAP10 | CD3δ |
| ABD | CD86 | DAP10 | CD3γ |
| ABD | CD86 | DAP10 | CD3ε |
| ABD | CD86 | DAP10 | FcγRI-γ |
| ABD | CD86 | DAP10 | FcγRIII-γ |
| ABD | CD86 | DAP10 | FcεRIβ |
| ABD | CD86 | DAP10 | FcεRIγ |
| ABD | CD86 | DAP10 | DAP10 |
| ABD | CD86 | DAP10 | DAP12 |
| ABD | CD86 | DAP10 | CD32 |
| ABD | CD86 | DAP10 | CD79a |
| ABD | CD86 | DAP10 | CD79b |
| ABD | CD86 | DAP12 | CD8 |
| ABD | CD86 | DAP12 | CD3ζ |
| ABD | CD86 | DAP12 | CD3δ |
| ABD | CD86 | DAP12 | CD3γ |
| ABD | CD86 | DAP12 | CD3ε |
| ABD | CD86 | DAP12 | FcγRI-γ |
| ABD | CD86 | DAP12 | FcγRIII-γ |
| ABD | CD86 | DAP12 | FcεRIβ |
| ABD | CD86 | DAP12 | FcεRIγ |
| ABD | CD86 | DAP12 | DAP10 |
| ABD | CD86 | DAP12 | DAP12 |
| ABD | CD86 | DAP12 | CD32 |
| ABD | CD86 | DAP12 | CD79a |
| ABD | CD86 | DAP12 | CD79b |
| ABD | CD86 | MyD88 | CD8 |
| ABD | CD86 | MyD88 | CD3ζ |
| ABD | CD86 | MyD88 | CD3δ |
| ABD | CD86 | MyD88 | CD3γ |
| ABD | CD86 | MyD88 | CD3ε |
| ABD | CD86 | MyD88 | FcγRI-γ |
| ABD | CD86 | MyD88 | FcγRIII-γ |
| ABD | CD86 | MyD88 | FcεRIβ |
| ABD | CD86 | MyD88 | FcεRIγ |
| ABD | CD86 | MyD88 | DAP10 |
| ABD | CD86 | MyD88 | DAP12 |
| ABD | CD86 | MyD88 | CD32 |
| ABD | CD86 | MyD88 | CD79a |
| ABD | CD86 | MyD88 | CD79b |
| ABD | CD86 | CD7 | CD8 |
| ABD | CD86 | CD7 | CD3ζ |
| ABD | CD86 | CD7 | CD3δ |
| ABD | CD86 | CD7 | CD3γ |
| ABD | CD86 | CD7 | CD3ε |
| ABD | CD86 | CD7 | FcγRI-γ |
| ABD | CD86 | CD7 | FcγRIII-γ |
| ABD | CD86 | CD7 | FcεRIβ |
| ABD | CD86 | CD7 | FcεRIγ |
| ABD | CD86 | CD7 | DAP10 |
| ABD | CD86 | CD7 | DAP12 |
| ABD | CD86 | CD7 | CD32 |
| ABD | CD86 | CD7 | CD79a |
| ABD | CD86 | CD7 | CD79b |
| ABD | CD86 | BTNL3 | CD8 |
| ABD | CD86 | BTNL3 | CD3ζ |
| ABD | CD86 | BTNL3 | CD3δ |
| ABD | CD86 | BTNL3 | CD3γ |
| ABD | CD86 | BTNL3 | CD3ε |
| ABD | CD86 | BTNL3 | FcγRI-γ |
| ABD | CD86 | BTNL3 | FcγRIII-γ |
| ABD | CD86 | BTNL3 | FcεRIβ |
| ABD | CD86 | BTNL3 | FcεRIγ |
| ABD | CD86 | BTNL3 | DAP10 |
| ABD | CD86 | BTNL3 | DAP12 |
| ABD | CD86 | BTNL3 | CD32 |
| ABD | CD86 | BTNL3 | CD79a |
| ABD | CD86 | BTNL3 | CD79b |
| ABD | CD86 | NKG2D | CD8 |
| ABD | CD86 | NKG2D | CD3ζ |
| ABD | CD86 | NKG2D | CD3δ |
| ABD | CD86 | NKG2D | CD3γ |
| ABD | CD86 | NKG2D | CD3ε |
| ABD | CD86 | NKG2D | FcγRI-γ |
| ABD | CD86 | NKG2D | FcγRIII-γ |
| ABD | CD86 | NKG2D | FcεRIβ |
| ABD | CD86 | NKG2D | FcεRIγ |
| ABD | CD86 | NKG2D | DAP10 |
| ABD | CD86 | NKG2D | DAP12 |
| ABD | CD86 | NKG2D | CD32 |
| ABD | CD86 | NKG2D | CD79a |
| ABD | CD86 | NKG2D | CD79b |
| ABD | OX40 | CD28 | CD8 |
| ABD | OX40 | CD28 | CD3ζ |
| ABD | OX40 | CD28 | CD3δ |
| ABD | OX40 | CD28 | CD3γ |
| ABD | OX40 | CD28 | CD3ε |
| ABD | OX40 | CD28 | FcγRI-γ |
| ABD | OX40 | CD28 | FcγRIII-γ |
| ABD | OX40 | CD28 | FcεRIβ |
| ABD | OX40 | CD28 | FcεRIγ |
| ABD | OX40 | CD28 | DAP10 |
| ABD | OX40 | CD28 | DAP12 |
| ABD | OX40 | CD28 | CD32 |
| ABD | OX40 | CD28 | CD79a |
| ABD | OX40 | CD28 | CD79b |
| ABD | OX40 | CD8 | CD8 |
| ABD | OX40 | CD8 | CD3ζ |
| ABD | OX40 | CD8 | CD3δ |
| ABD | OX40 | CD8 | CD3γ |
| ABD | OX40 | CD8 | CD3ε |
| ABD | OX40 | CD8 | FcγRI-γ |
| ABD | OX40 | CD8 | FcγRIII-γ |
| ABD | OX40 | CD8 | FcεRIβ |
| ABD | OX40 | CD8 | FcεRIγ |
| ABD | OX40 | CD8 | DAP10 |
| ABD | OX40 | CD8 | DAP12 |
| ABD | OX40 | CD8 | CD32 |
| ABD | OX40 | CD8 | CD79a |
| ABD | OX40 | CD8 | CD79b |
| ABD | OX40 | CD4 | CD8 |
| ABD | OX40 | CD4 | CD3ζ |
| ABD | OX40 | CD4 | CD3δ |
| ABD | OX40 | CD4 | CD3γ |
| ABD | OX40 | CD4 | CD3ε |
| ABD | OX40 | CD4 | FcγRI-γ |
| ABD | OX40 | CD4 | FcγRIII-γ |
| ABD | OX40 | CD4 | FcεRIβ |
| ABD | OX40 | CD4 | FcεRIγ |
| ABD | OX40 | CD4 | DAP10 |
| ABD | OX40 | CD4 | DAP12 |
| ABD | OX40 | CD4 | CD32 |
| ABD | OX40 | CD4 | CD79a |
| ABD | OX40 | CD4 | CD79b |
| ABD | OX40 | b2c | CD8 |
| ABD | OX40 | b2c | CD3ζ |
| ABD | OX40 | b2c | CD3δ |
| ABD | OX40 | b2c | CD3γ |
| ABD | OX40 | b2c | CD3ε |
| ABD | OX40 | b2c | FcγRI-γ |
| ABD | OX40 | b2c | FcγRIII-γ |
| ABD | OX40 | b2c | FcεRIβ |
| ABD | OX40 | b2c | FcεRIγ |
| ABD | OX40 | b2c | DAP10 |
| ABD | OX40 | b2c | DAP12 |
| ABD | OX40 | b2c | CD32 |
| ABD | OX40 | b2c | CD79a |
| ABD | OX40 | b2c | CD79b |
| ABD | OX40 | CD137/41BB | CD8 |
| ABD | OX40 | CD137/41BB | CD3ζ |
| ABD | OX40 | CD137/41BB | CD3δ |
| ABD | OX40 | CD137/41BB | CD3γ |
| ABD | OX40 | CD137/41BB | CD3ε |
| ABD | OX40 | CD137/41BB | FcγRI-γ |
| ABD | OX40 | CD137/41BB | FcγRIII-γ |
| ABD | OX40 | CD137/41BB | FcεRIβ |
| ABD | OX40 | CD137/41BB | FcεRIγ |
| ABD | OX40 | CD137/41BB | DAP10 |
| ABD | OX40 | CD137/41BB | DAP12 |
| ABD | OX40 | CD137/41BB | CD32 |
| ABD | OX40 | CD137/41BB | CD79a |
| ABD | OX40 | CD137/41BB | CD79b |
| ABD | OX40 | ICOS | CD8 |
| ABD | OX40 | ICOS | CD3ζ |
| ABD | OX40 | ICOS | CD3δ |
| ABD | OX40 | ICOS | CD3γ |
| ABD | OX40 | ICOS | CD3ε |
| ABD | OX40 | ICOS | FcγRI-γ |
| ABD | OX40 | ICOS | FcγRIII-γ |
| ABD | OX40 | ICOS | FcεRIβ |
| ABD | OX40 | ICOS | FcεRIγ |
| ABD | OX40 | ICOS | DAP10 |
| ABD | OX40 | ICOS | DAP12 |
| ABD | OX40 | ICOS | CD32 |
| ABD | OX40 | ICOS | CD79a |
| ABD | OX40 | ICOS | CD79b |
| ABD | OX40 | CD27 | CD8 |
| ABD | OX40 | CD27 | CD3ζ |
| ABD | OX40 | CD27 | CD3δ |
| ABD | OX40 | CD27 | CD3γ |
| ABD | OX40 | CD27 | CD3ε |
| ABD | OX40 | CD27 | FcγRI-γ |
| ABD | OX40 | CD27 | FcγRIII-γ |
| ABD | OX40 | CD27 | FcεRIβ |
| ABD | OX40 | CD27 | FcεRIγ |
| ABD | OX40 | CD27 | DAP10 |
| ABD | OX40 | CD27 | DAP12 |
| ABD | OX40 | CD27 | CD32 |
| ABD | OX40 | CD27 | CD79a |
| ABD | OX40 | CD27 | CD79b |
| ABD | OX40 | CD28δ | CD8 |
| ABD | OX40 | CD28δ | CD3ζ |
| ABD | OX40 | CD28δ | CD3δ |
| ABD | OX40 | CD28δ | CD3γ |
| ABD | OX40 | CD28δ | CD3ε |
| ABD | OX40 | CD28δ | FcγRI-γ |
| ABD | OX40 | CD28δ | FcγRIII-γ |
| ABD | OX40 | CD28δ | FcεRIβ |
| ABD | OX40 | CD28δ | FcεRIγ |
| ABD | OX40 | CD28δ | DAP10 |
| ABD | OX40 | CD28δ | DAP12 |
| ABD | OX40 | CD28δ | CD32 |
| ABD | OX40 | CD28δ | CD79a |
| ABD | OX40 | CD28δ | CD79b |
| ABD | OX40 | CD80 | CD8 |
| ABD | OX40 | CD80 | CD3ζ |
| ABD | OX40 | CD80 | CD3δ |
| ABD | OX40 | CD80 | CD3γ |
| ABD | OX40 | CD80 | CD3ε |
| ABD | OX40 | CD80 | FcγRI-γ |
| ABD | OX40 | CD80 | FcγRIII-γ |
| ABD | OX40 | CD80 | FcεRIβ |
| ABD | OX40 | CD80 | FcεRIγ |
| ABD | OX40 | CD80 | DAP10 |
| ABD | OX40 | CD80 | DAP12 |
| ABD | OX40 | CD80 | CD32 |
| ABD | OX40 | CD80 | CD79a |
| ABD | OX40 | CD80 | CD79b |
| ABD | OX40 | CD86 | CD8 |
| ABD | OX40 | CD86 | CD3ζ |
| ABD | OX40 | CD86 | CD3δ |
| ABD | OX40 | CD86 | CD3γ |
| ABD | OX40 | CD86 | CD3ε |
| ABD | OX40 | CD86 | FcγRI-γ |
| ABD | OX40 | CD86 | FcγRIII-γ |
| ABD | OX40 | CD86 | FcεRIβ |
| ABD | OX40 | CD86 | FcεRIγ |
| ABD | OX40 | CD86 | DAP10 |
| ABD | OX40 | CD86 | DAP12 |
| ABD | OX40 | CD86 | CD32 |
| ABD | OX40 | CD86 | CD79a |
| ABD | OX40 | CD86 | CD79b |
| ABD | OX40 | OX40 | CD8 |
| ABD | OX40 | OX40 | CD3ζ |
| ABD | OX40 | OX40 | CD3δ |
| ABD | OX40 | OX40 | CD3γ |
| ABD | OX40 | OX40 | CD3ε |
| ABD | OX40 | OX40 | FcγRI-γ |
| ABD | OX40 | OX40 | FcγRIII-γ |
| ABD | OX40 | OX40 | FcεRIβ |
| ABD | OX40 | OX40 | FcεRIγ |
| ABD | OX40 | OX40 | DAP10 |
| ABD | OX40 | OX40 | DAP12 |
| ABD | OX40 | OX40 | CD32 |
| ABD | OX40 | OX40 | CD79a |
| ABD | OX40 | OX40 | CD79b |
| ABD | OX40 | DAP10 | CD8 |
| ABD | OX40 | DAP10 | CD3ζ |
| ABD | OX40 | DAP10 | CD3δ |
| ABD | OX40 | DAP10 | CD3γ |
| ABD | OX40 | DAP10 | CD3ε |
| ABD | OX40 | DAP10 | FcγRI-γ |
| ABD | OX40 | DAP10 | FcγRIII-γ |
| ABD | OX40 | DAP10 | FcεRIβ |
| ABD | OX40 | DAP10 | FcεRIγ |
| ABD | OX40 | DAP10 | DAP10 |
| ABD | OX40 | DAP10 | DAP12 |
| ABD | OX40 | DAP10 | CD32 |
| ABD | OX40 | DAP10 | CD79a |
| ABD | OX40 | DAP10 | CD79b |
| ABD | OX40 | DAP12 | CD8 |
| ABD | OX40 | DAP12 | CD3ζ |
| ABD | OX40 | DAP12 | CD3δ |
| ABD | OX40 | DAP12 | CD3γ |
| ABD | OX40 | DAP12 | CD3ε |
| ABD | OX40 | DAP12 | FcγRI-γ |
| ABD | OX40 | DAP12 | FcγRIII-γ |
| ABD | OX40 | DAP12 | FcεRIβ |
| ABD | OX40 | DAP12 | FcεRIγ |
| ABD | OX40 | DAP12 | DAP10 |
| ABD | OX40 | DAP12 | DAP12 |
| ABD | OX40 | DAP12 | CD32 |
| ABD | OX40 | DAP12 | CD79a |
| ABD | OX40 | DAP12 | CD79b |
| ABD | OX40 | MyD88 | CD8 |
| ABD | OX40 | MyD88 | CD3ζ |
| ABD | OX40 | MyD88 | CD3δ |
| ABD | OX40 | MyD88 | CD3γ |
| ABD | OX40 | MyD88 | CD3ε |
| ABD | OX40 | MyD88 | FcγRI-γ |
| ABD | OX40 | MyD88 | FcγRIII-γ |
| ABD | OX40 | MyD88 | FcεRIβ |
| ABD | OX40 | MyD88 | FcεRIγ |
| ABD | OX40 | MyD88 | DAP10 |
| ABD | OX40 | MyD88 | DAP12 |
| ABD | OX40 | MyD88 | CD32 |
| ABD | OX40 | MyD88 | CD79a |
| ABD | OX40 | MyD88 | CD79b |
| ABD | OX40 | CD7 | CD8 |
| ABD | OX40 | CD7 | CD3ζ |
| ABD | OX40 | CD7 | CD3δ |
| ABD | OX40 | CD7 | CD3γ |
| ABD | OX40 | CD7 | CD3ε |
| ABD | OX40 | CD7 | FcγRI-γ |
| ABD | OX40 | CD7 | FcγRIII-γ |
| ABD | OX40 | CD7 | FcεRIβ |
| ABD | OX40 | CD7 | FcεRIγ |
| ABD | OX40 | CD7 | DAP10 |
| ABD | OX40 | CD7 | DAP12 |
| ABD | OX40 | CD7 | CD32 |
| ABD | OX40 | CD7 | CD79a |
| ABD | OX40 | CD7 | CD79b |
| ABD | OX40 | BTNL3 | CD8 |
| ABD | OX40 | BTNL3 | CD3ζ |
| ABD | OX40 | BTNL3 | CD3δ |
| ABD | OX40 | BTNL3 | CD3γ |
| ABD | OX40 | BTNL3 | CD3ε |
| ABD | OX40 | BTNL3 | FcγRI-γ |
| ABD | OX40 | BTNL3 | FcγRIII-γ |
| ABD | OX40 | BTNL3 | FcεRIβ |
| ABD | OX40 | BTNL3 | FcεRIγ |
| ABD | OX40 | BTNL3 | DAP10 |
| ABD | OX40 | BTNL3 | DAP12 |
| ABD | OX40 | BTNL3 | CD32 |
| ABD | OX40 | BTNL3 | CD79a |
| ABD | OX40 | BTNL3 | CD79b |
| ABD | OX40 | NKG2D | CD8 |
| ABD | OX40 | NKG2D | CD3ζ |
| ABD | OX40 | NKG2D | CD3δ |
| ABD | OX40 | NKG2D | CD3γ |
| ABD | OX40 | NKG2D | CD3ε |
| ABD | OX40 | NKG2D | FcγRI-γ |
| ABD | OX40 | NKG2D | FcγRIII-γ |
| ABD | OX40 | NKG2D | FcεRIβ |
| ABD | OX40 | NKG2D | FcεRIγ |
| ABD | OX40 | NKG2D | DAP10 |
| ABD | OX40 | NKG2D | DAP12 |
| ABD | OX40 | NKG2D | CD32 |
| ABD | OX40 | NKG2D | CD79a |
| ABD | OX40 | NKG2D | CD79b |
| ABD | DAP10 | CD28 | CD8 |
| ABD | DAP10 | CD28 | CD3ζ |
| ABD | DAP10 | CD28 | CD3δ |
| ABD | DAP10 | CD28 | CD3γ |
| ABD | DAP10 | CD28 | CD3ε |
| ABD | DAP10 | CD28 | FcγRI-γ |
| ABD | DAP10 | CD28 | FcγRIII-γ |
| ABD | DAP10 | CD28 | FcεRIβ |
| ABD | DAP10 | CD28 | FcεRIγ |
| ABD | DAP10 | CD28 | DAP10 |
| ABD | DAP10 | CD28 | DAP12 |
| ABD | DAP10 | CD28 | CD32 |
| ABD | DAP10 | CD28 | CD79a |
| ABD | DAP10 | CD28 | CD79b |
| ABD | DAP10 | CD8 | CD8 |
| ABD | DAP10 | CD8 | CD3ζ |
| ABD | DAP10 | CD8 | CD3δ |
| ABD | DAP10 | CD8 | CD3γ |
| ABD | DAP10 | CD8 | CD3ε |
| ABD | DAP10 | CD8 | FcγRI-γ |
| ABD | DAP10 | CD8 | FcγRIII-γ |
| ABD | DAP10 | CD8 | FcεRIβ |
| ABD | DAP10 | CD8 | FcεRIγ |
| ABD | DAP10 | CD8 | DAP10 |
| ABD | DAP10 | CD8 | DAP12 |
| ABD | DAP10 | CD8 | CD32 |
| ABD | DAP10 | CD8 | CD79a |
| ABD | DAP10 | CD8 | CD79b |
| ABD | DAP10 | CD4 | CD8 |
| ABD | DAP10 | CD4 | CD3ζ |
| ABD | DAP10 | CD4 | CD3δ |
| ABD | DAP10 | CD4 | CD3γ |
| ABD | DAP10 | CD4 | CD3ε |
| ABD | DAP10 | CD4 | FcγRI-γ |
| ABD | DAP10 | CD4 | FcγRIII-γ |
| ABD | DAP10 | CD4 | FcεRIβ |
| ABD | DAP10 | CD4 | FcεRIγ |
| ABD | DAP10 | CD4 | DAP10 |
| ABD | DAP10 | CD4 | DAP12 |
| ABD | DAP10 | CD4 | CD32 |
| ABD | DAP10 | CD4 | CD79a |
| ABD | DAP10 | CD4 | CD79b |
| ABD | DAP10 | b2c | CD8 |
| ABD | DAP10 | b2c | CD3ζ |
| ABD | DAP10 | b2c | CD3δ |
| ABD | DAP10 | b2c | CD3γ |
| ABD | DAP10 | b2c | CD3ε |
| ABD | DAP10 | b2c | FcγRI-γ |
| ABD | DAP10 | b2c | FcγRIII-γ |
| ABD | DAP10 | b2c | FcεRIβ |
| ABD | DAP10 | b2c | FcεRIγ |
| ABD | DAP10 | b2c | DAP10 |
| ABD | DAP10 | b2c | DAP12 |
| ABD | DAP10 | b2c | CD32 |
| ABD | DAP10 | b2c | CD79a |
| ABD | DAP10 | b2c | CD79b |
| ABD | DAP10 | CD137/41BB | CD8 |
| ABD | DAP10 | CD137/41BB | CD3ζ |
| ABD | DAP10 | CD137/41BB | CD3δ |
| ABD | DAP10 | CD137/41BB | CD3γ |
| ABD | DAP10 | CD137/41BB | CD3ε |
| ABD | DAP10 | CD137/41BB | FcγRI-γ |
| ABD | DAP10 | CD137/41BB | FcγRIII-γ |
| ABD | DAP10 | CD137/41BB | FcεRIβ |
| ABD | DAP10 | CD137/41BB | FcεRIγ |
| ABD | DAP10 | CD137/41BB | DAP10 |
| ABD | DAP10 | CD137/41BB | DAP12 |
| ABD | DAP10 | CD137/41BB | CD32 |
| ABD | DAP10 | CD137/41BB | CD79a |
| ABD | DAP10 | CD137/41BB | CD79b |
| ABD | DAP10 | ICOS | CD8 |
| ABD | DAP10 | ICOS | CD3ζ |
| ABD | DAP10 | ICOS | CD3δ |
| ABD | DAP10 | ICOS | CD3γ |
| ABD | DAP10 | ICOS | CD3ε |
| ABD | DAP10 | ICOS | FcγRI-γ |
| ABD | DAP10 | ICOS | FcγRIII-γ |
| ABD | DAP10 | ICOS | FcεRIβ |
| ABD | DAP10 | ICOS | FcεRIγ |
| ABD | DAP10 | ICOS | DAP10 |
| ABD | DAP10 | ICOS | DAP12 |
| ABD | DAP10 | ICOS | CD32 |
| ABD | DAP10 | ICOS | CD79a |
| ABD | DAP10 | ICOS | CD79b |
| ABD | DAP10 | CD27 | CD8 |
| ABD | DAP10 | CD27 | CD3ζ |
| ABD | DAP10 | CD27 | CD3δ |
| ABD | DAP10 | CD27 | CD3γ |
| ABD | DAP10 | CD27 | CD3ε |
| ABD | DAP10 | CD27 | FcγRI-γ |
| ABD | DAP10 | CD27 | FcγRIII-γ |
| ABD | DAP10 | CD27 | FcεRIβ |
| ABD | DAP10 | CD27 | FcεRIγ |
| ABD | DAP10 | CD27 | DAP10 |
| ABD | DAP10 | CD27 | DAP12 |
| ABD | DAP10 | CD27 | CD32 |
| ABD | DAP10 | CD27 | CD79a |
| ABD | DAP10 | CD27 | CD79b |
| ABD | DAP10 | CD28δ | CD8 |
| ABD | DAP10 | CD28δ | CD3ζ |
| ABD | DAP10 | CD28δ | CD3δ |
| ABD | DAP10 | CD28δ | CD3γ |
| ABD | DAP10 | CD28δ | CD3ε |
| ABD | DAP10 | CD28δ | FcγRI-γ |
| ABD | DAP10 | CD28δ | FcγRIII-γ |
| ABD | DAP10 | CD28δ | FcεRIβ |
| ABD | DAP10 | CD28δ | FcεRIγ |
| ABD | DAP10 | CD28δ | DAP10 |
| ABD | DAP10 | CD28δ | DAP12 |
| ABD | DAP10 | CD28δ | CD32 |
| ABD | DAP10 | CD28δ | CD79a |
| ABD | DAP10 | CD28δ | CD79b |
| ABD | DAP10 | CD80 | CD8 |
| ABD | DAP10 | CD80 | CD3ζ |
| ABD | DAP10 | CD80 | CD3δ |
| ABD | DAP10 | CD80 | CD3γ |
| ABD | DAP10 | CD80 | CD3ε |
| ABD | DAP10 | CD80 | FcγRI-γ |
| ABD | DAP10 | CD80 | FcγRIII-γ |
| ABD | DAP10 | CD80 | FcεRIβ |
| ABD | DAP10 | CD80 | FcεRIγ |
| ABD | DAP10 | CD80 | DAP10 |
| ABD | DAP10 | CD80 | DAP12 |
| ABD | DAP10 | CD80 | CD32 |
| ABD | DAP10 | CD80 | CD79a |
| ABD | DAP10 | CD80 | CD79b |
| ABD | DAP10 | CD86 | CD8 |
| ABD | DAP10 | CD86 | CD3ζ |
| ABD | DAP10 | CD86 | CD3δ |
| ABD | DAP10 | CD86 | CD3γ |
| ABD | DAP10 | CD86 | CD3ε |
| ABD | DAP10 | CD86 | FcγRI-γ |
| ABD | DAP10 | CD86 | FcγRIII-γ |
| ABD | DAP10 | CD86 | FcεRIβ |
| ABD | DAP10 | CD86 | FcεRIγ |
| ABD | DAP10 | CD86 | DAP10 |
| ABD | DAP10 | CD86 | DAP12 |
| ABD | DAP10 | CD86 | CD32 |
| ABD | DAP10 | CD86 | CD79a |
| ABD | DAP10 | CD86 | CD79b |
| ABD | DAP10 | OX40 | CD8 |
| ABD | DAP10 | OX40 | CD3ζ |
| ABD | DAP10 | OX40 | CD3δ |
| ABD | DAP10 | OX40 | CD3γ |
| ABD | DAP10 | OX40 | CD3ε |
| ABD | DAP10 | OX40 | FcγRI-γ |
| ABD | DAP10 | OX40 | FcγRIII-γ |
| ABD | DAP10 | OX40 | FcεRIβ |
| ABD | DAP10 | OX40 | FcεRIγ |
| ABD | DAP10 | OX40 | DAP10 |
| ABD | DAP10 | OX40 | DAP12 |
| ABD | DAP10 | OX40 | CD32 |
| ABD | DAP10 | OX40 | CD79a |
| ABD | DAP10 | OX40 | CD79b |
| ABD | DAP10 | DAP10 | CD8 |
| ABD | DAP10 | DAP10 | CD3ζ |
| ABD | DAP10 | DAP10 | CD3δ |
| ABD | DAP10 | DAP10 | CD3γ |
| ABD | DAP10 | DAP10 | CD3ε |
| ABD | DAP10 | DAP10 | FcγRI-γ |
| ABD | DAP10 | DAP10 | FcγRIII-γ |
| ABD | DAP10 | DAP10 | FcεRIβ |
| ABD | DAP10 | DAP10 | FcεRIγ |
| ABD | DAP10 | DAP10 | DAP10 |
| ABD | DAP10 | DAP10 | DAP12 |
| ABD | DAP10 | DAP10 | CD32 |
| ABD | DAP10 | DAP10 | CD79a |
| ABD | DAP10 | DAP10 | CD79b |
| ABD | DAP10 | DAP12 | CD8 |
| ABD | DAP10 | DAP12 | CD3ζ |
| ABD | DAP10 | DAP12 | CD3δ |
| ABD | DAP10 | DAP12 | CD3γ |
| ABD | DAP10 | DAP12 | CD3ε |
| ABD | DAP10 | DAP12 | FcγRI-γ |
| ABD | DAP10 | DAP12 | FcγRIII-γ |
| ABD | DAP10 | DAP12 | FcεRIβ |
| ABD | DAP10 | DAP12 | FcεRIγ |
| ABD | DAP10 | DAP12 | DAP10 |
| ABD | DAP10 | DAP12 | DAP12 |
| ABD | DAP10 | DAP12 | CD32 |
| ABD | DAP10 | DAP12 | CD79a |
| ABD | DAP10 | DAP12 | CD79b |
| ABD | DAP10 | MyD88 | CD8 |
| ABD | DAP10 | MyD88 | CD3ζ |
| ABD | DAP10 | MyD88 | CD3δ |
| ABD | DAP10 | MyD88 | CD3γ |
| ABD | DAP10 | MyD88 | CD3ε |
| ABD | DAP10 | MyD88 | FcγRI-γ |
| ABD | DAP10 | MyD88 | FcγRIII-γ |
| ABD | DAP10 | MyD88 | FcεRIβ |
| ABD | DAP10 | MyD88 | FcεRIγ |
| ABD | DAP10 | MyD88 | DAP10 |
| ABD | DAP10 | MyD88 | DAP12 |
| ABD | DAP10 | MyD88 | CD32 |
| ABD | DAP10 | MyD88 | CD79a |
| ABD | DAP10 | MyD88 | CD79b |
| ABD | DAP10 | CD7 | CD8 |
| ABD | DAP10 | CD7 | CD3ζ |
| ABD | DAP10 | CD7 | CD3δ |
| ABD | DAP10 | CD7 | CD3γ |
| ABD | DAP10 | CD7 | CD3ε |
| ABD | DAP10 | CD7 | FcγRI-γ |
| ABD | DAP10 | CD7 | FcγRIII-γ |
| ABD | DAP10 | CD7 | FcεRIβ |
| ABD | DAP10 | CD7 | FcεRIγ |
| ABD | DAP10 | CD7 | DAP10 |
| ABD | DAP10 | CD7 | DAP12 |
| ABD | DAP10 | CD7 | CD32 |
| ABD | DAP10 | CD7 | CD79a |
| ABD | DAP10 | CD7 | CD79b |
| ABD | DAP10 | BTNL3 | CD8 |
| ABD | DAP10 | BTNL3 | CD3ζ |
| ABD | DAP10 | BTNL3 | CD3δ |
| ABD | DAP10 | BTNL3 | CD3γ |
| ABD | DAP10 | BTNL3 | CD3ε |
| ABD | DAP10 | BTNL3 | FcγRI-γ |
| ABD | DAP10 | BTNL3 | FcγRIII-γ |
| ABD | DAP10 | BTNL3 | FcεRIβ |
| ABD | DAP10 | BTNL3 | FcεRIγ |
| ABD | DAP10 | BTNL3 | DAP10 |
| ABD | DAP10 | BTNL3 | DAP12 |
| ABD | DAP10 | BTNL3 | CD32 |
| ABD | DAP10 | BTNL3 | CD79a |
| ABD | DAP10 | BTNL3 | CD79b |
| ABD | DAP10 | NKG2D | CD8 |
| ABD | DAP10 | NKG2D | CD3ζ |
| ABD | DAP10 | NKG2D | CD3δ |
| ABD | DAP10 | NKG2D | CD3γ |
| ABD | DAP10 | NKG2D | CD3ε |
| ABD | DAP10 | NKG2D | FcγRI-γ |
| ABD | DAP10 | NKG2D | FcγRIII-γ |
| ABD | DAP10 | NKG2D | FcεRIβ |
| ABD | DAP10 | NKG2D | FcεRIγ |
| ABD | DAP10 | NKG2D | DAP10 |
| ABD | DAP10 | NKG2D | DAP12 |
| ABD | DAP10 | NKG2D | CD32 |
| ABD | DAP10 | NKG2D | CD79a |
| ABD | DAP10 | NKG2D | CD79b |
| ABD | DAP12 | CD28 | CD8 |
| ABD | DAP12 | CD28 | CD3ζ |
| ABD | DAP12 | CD28 | CD3δ |
| ABD | DAP12 | CD28 | CD3γ |
| ABD | DAP12 | CD28 | CD3ε |
| ABD | DAP12 | CD28 | FcγRI-γ |
| ABD | DAP12 | CD28 | FcγRIII-γ |
| ABD | DAP12 | CD28 | FcεRIβ |
| ABD | DAP12 | CD28 | FcεRIγ |
| ABD | DAP12 | CD28 | DAP10 |
| ABD | DAP12 | CD28 | DAP12 |
| ABD | DAP12 | CD28 | CD32 |
| ABD | DAP12 | CD28 | CD79a |
| ABD | DAP12 | CD28 | CD79b |
| ABD | DAP12 | CD8 | CD8 |
| ABD | DAP12 | CD8 | CD3ζ |
| ABD | DAP12 | CD8 | CD3δ |
| ABD | DAP12 | CD8 | CD3γ |
| ABD | DAP12 | CD8 | CD3ε |
| ABD | DAP12 | CD8 | FcγRI-γ |
| ABD | DAP12 | CD8 | FcγRIII-γ |
| ABD | DAP12 | CD8 | FcεRIβ |
| ABD | DAP12 | CD8 | FcεRIγ |
| ABD | DAP12 | CD8 | DAP10 |
| ABD | DAP12 | CD8 | DAP12 |
| ABD | DAP12 | CD8 | CD32 |
| ABD | DAP12 | CD8 | CD79a |
| ABD | DAP12 | CD8 | CD79b |
| ABD | DAP12 | CD4 | CD8 |
| ABD | DAP12 | CD4 | CD3ζ |
| ABD | DAP12 | CD4 | CD3δ |
| ABD | DAP12 | CD4 | CD3γ |
| ABD | DAP12 | CD4 | CD3ε |
| ABD | DAP12 | CD4 | FcγRI-γ |
| ABD | DAP12 | CD4 | FcγRIII-γ |
| ABD | DAP12 | CD4 | FcεRIβ |
| ABD | DAP12 | CD4 | FcεRIγ |
| ABD | DAP12 | CD4 | DAP10 |
| ABD | DAP12 | CD4 | DAP12 |
| ABD | DAP12 | CD4 | CD32 |
| ABD | DAP12 | CD4 | CD79a |
| ABD | DAP12 | CD4 | CD79b |
| ABD | DAP12 | b2c | CD8 |
| ABD | DAP12 | b2c | CD3ζ |
| ABD | DAP12 | b2c | CD3δ |
| ABD | DAP12 | b2c | CD3γ |
| ABD | DAP12 | b2c | CD3ε |
| ABD | DAP12 | b2c | FcγRI-γ |
| ABD | DAP12 | b2c | FcγRIII-γ |
| ABD | DAP12 | b2c | FcεRIβ |
| ABD | DAP12 | b2c | FcεRIγ |
| ABD | DAP12 | b2c | DAP10 |
| ABD | DAP12 | b2c | DAP12 |
| ABD | DAP12 | b2c | CD32 |
| ABD | DAP12 | b2c | CD79a |
| ABD | DAP12 | b2c | CD79b |
| ABD | DAP12 | CD137/41BB | CD8 |
| ABD | DAP12 | CD137/41BB | CD3ζ |
| ABD | DAP12 | CD137/41BB | CD3δ |
| ABD | DAP12 | CD137/41BB | CD3γ |
| ABD | DAP12 | CD137/41BB | CD3ε |
| ABD | DAP12 | CD137/41BB | FcγRI-γ |
| ABD | DAP12 | CD137/41BB | FcγRIII-γ |
| ABD | DAP12 | CD137/41BB | FcεRIβ |
| ABD | DAP12 | CD137/41BB | FcεRIγ |
| ABD | DAP12 | CD137/41BB | DAP10 |
| ABD | DAP12 | CD137/41BB | DAP12 |
| ABD | DAP12 | CD137/41BB | CD32 |
| ABD | DAP12 | CD137/41BB | CD79a |
| ABD | DAP12 | CD137/41BB | CD79b |
| ABD | DAP12 | ICOS | CD8 |
| ABD | DAP12 | ICOS | CD3ζ |
| ABD | DAP12 | ICOS | CD3δ |
| ABD | DAP12 | ICOS | CD3γ |
| ABD | DAP12 | ICOS | CD3ε |
| ABD | DAP12 | ICOS | FcγRI-γ |
| ABD | DAP12 | ICOS | FcγRIII-γ |
| ABD | DAP12 | ICOS | FcεRIβ |
| ABD | DAP12 | ICOS | FcεRIγ |
| ABD | DAP12 | ICOS | DAP10 |
| ABD | DAP12 | ICOS | DAP12 |
| ABD | DAP12 | ICOS | CD32 |
| ABD | DAP12 | ICOS | CD79a |
| ABD | DAP12 | ICOS | CD79b |
| ABD | DAP12 | CD27 | CD8 |
| ABD | DAP12 | CD27 | CD3ζ |
| ABD | DAP12 | CD27 | CD3δ |
| ABD | DAP12 | CD27 | CD3γ |
| ABD | DAP12 | CD27 | CD3ε |
| ABD | DAP12 | CD27 | FcγRI-γ |
| ABD | DAP12 | CD27 | FcγRIII-γ |
| ABD | DAP12 | CD27 | FcεRIβ |
| ABD | DAP12 | CD27 | FcεRIγ |
| ABD | DAP12 | CD27 | DAP10 |
| ABD | DAP12 | CD27 | DAP12 |
| ABD | DAP12 | CD27 | CD32 |
| ABD | DAP12 | CD27 | CD79a |
| ABD | DAP12 | CD27 | CD79b |
| ABD | DAP12 | CD28δ | CD8 |
| ABD | DAP12 | CD28δ | CD3ζ |
| ABD | DAP12 | CD28δ | CD3δ |
| ABD | DAP12 | CD28δ | CD3γ |
| ABD | DAP12 | CD28δ | CD3ε |
| ABD | DAP12 | CD28δ | FcγRI-γ |
| ABD | DAP12 | CD28δ | FcγRIII-γ |
| ABD | DAP12 | CD28δ | FcεRIβ |
| ABD | DAP12 | CD28δ | FcεRIγ |
| ABD | DAP12 | CD28δ | DAP10 |
| ABD | DAP12 | CD28δ | DAP12 |
| ABD | DAP12 | CD28δ | CD32 |
| ABD | DAP12 | CD28δ | CD79a |
| ABD | DAP12 | CD28δ | CD79b |
| ABD | DAP12 | CD80 | CD8 |
| ABD | DAP12 | CD80 | CD3ζ |
| ABD | DAP12 | CD80 | CD3δ |
| ABD | DAP12 | CD80 | CD3γ |
| ABD | DAP12 | CD80 | CD3ε |
| ABD | DAP12 | CD80 | FcγRI-γ |
| ABD | DAP12 | CD80 | FcγRIII-γ |
| ABD | DAP12 | CD80 | FcεRIβ |
| ABD | DAP12 | CD80 | FcεRIγ |
| ABD | DAP12 | CD80 | DAP10 |
| ABD | DAP12 | CD80 | DAP12 |
| ABD | DAP12 | CD80 | CD32 |
| ABD | DAP12 | CD80 | CD79a |
| ABD | DAP12 | CD80 | CD79b |
| ABD | DAP12 | CD86 | CD8 |
| ABD | DAP12 | CD86 | CD3ζ |
| ABD | DAP12 | CD86 | CD3δ |
| ABD | DAP12 | CD86 | CD3γ |
| ABD | DAP12 | CD86 | CD3ε |
| ABD | DAP12 | CD86 | FcγRI-γ |
| ABD | DAP12 | CD86 | FcγRIII-γ |
| ABD | DAP12 | CD86 | FcεRIβ |
| ABD | DAP12 | CD86 | FcεRIγ |
| ABD | DAP12 | CD86 | DAP10 |
| ABD | DAP12 | CD86 | DAP12 |
| ABD | DAP12 | CD86 | CD32 |
| ABD | DAP12 | CD86 | CD79a |
| ABD | DAP12 | CD86 | CD79b |
| ABD | DAP12 | OX40 | CD8 |
| ABD | DAP12 | OX40 | CD3ζ |
| ABD | DAP12 | OX40 | CD3δ |
| ABD | DAP12 | OX40 | CD3γ |
| ABD | DAP12 | OX40 | CD3ε |
| ABD | DAP12 | OX40 | FcγRI-γ |
| ABD | DAP12 | OX40 | FcγRIII-γ |
| ABD | DAP12 | OX40 | FcεRIβ |
| ABD | DAP12 | OX40 | FcεRIγ |
| ABD | DAP12 | OX40 | DAP10 |
| ABD | DAP12 | OX40 | DAP12 |
| ABD | DAP12 | OX40 | CD32 |
| ABD | DAP12 | OX40 | CD79a |
| ABD | DAP12 | OX40 | CD79b |
| ABD | DAP12 | DAP10 | CD8 |
| ABD | DAP12 | DAP10 | CD3ζ |
| ABD | DAP12 | DAP10 | CD3δ |
| ABD | DAP12 | DAP10 | CD3γ |
| ABD | DAP12 | DAP10 | CD3ε |
| ABD | DAP12 | DAP10 | FcγRI-γ |
| ABD | DAP12 | DAP10 | FcγRIII-γ |
| ABD | DAP12 | DAP10 | FcεRIβ |
| ABD | DAP12 | DAP10 | FcεRIγ |
| ABD | DAP12 | DAP10 | DAP10 |
| ABD | DAP12 | DAP10 | DAP12 |
| ABD | DAP12 | DAP10 | CD32 |
| ABD | DAP12 | DAP10 | CD79a |
| ABD | DAP12 | DAP10 | CD79b |
| ABD | DAP12 | DAP12 | CD8 |
| ABD | DAP12 | DAP12 | CD3ζ |
| ABD | DAP12 | DAP12 | CD3δ |
| ABD | DAP12 | DAP12 | CD3γ |
| ABD | DAP12 | DAP12 | CD3ε |
| ABD | DAP12 | DAP12 | FcγRI-γ |
| ABD | DAP12 | DAP12 | FcγRIII-γ |
| ABD | DAP12 | DAP12 | FcεRIβ |
| ABD | DAP12 | DAP12 | FcεRIγ |
| ABD | DAP12 | DAP12 | DAP10 |
| ABD | DAP12 | DAP12 | DAP12 |
| ABD | DAP12 | DAP12 | CD32 |
| ABD | DAP12 | DAP12 | CD79a |
| ABD | DAP12 | DAP12 | CD79b |
| ABD | DAP12 | MyD88 | CD8 |
| ABD | DAP12 | MyD88 | CD3ζ |
| ABD | DAP12 | MyD88 | CD3δ |
| ABD | DAP12 | MyD88 | CD3γ |
| ABD | DAP12 | MyD88 | CD3ε |
| ABD | DAP12 | MyD88 | FcγRI-γ |
| ABD | DAP12 | MyD88 | FcγRIII-γ |
| ABD | DAP12 | MyD88 | FcεRIβ |
| ABD | DAP12 | MyD88 | FcεRIγ |
| ABD | DAP12 | MyD88 | DAP10 |
| ABD | DAP12 | MyD88 | DAP12 |
| ABD | DAP12 | MyD88 | CD32 |
| ABD | DAP12 | MyD88 | CD79a |
| ABD | DAP12 | MyD88 | CD79b |
| ABD | DAP12 | CD7 | CD8 |
| ABD | DAP12 | CD7 | CD3ζ |
| ABD | DAP12 | CD7 | CD3δ |
| ABD | DAP12 | CD7 | CD3γ |
| ABD | DAP12 | CD7 | CD3ε |
| ABD | DAP12 | CD7 | FcγRI-γ |
| ABD | DAP12 | CD7 | FcγRIII-γ |
| ABD | DAP12 | CD7 | FcεRIβ |
| ABD | DAP12 | CD7 | FcεRIγ |
| ABD | DAP12 | CD7 | DAP10 |
| ABD | DAP12 | CD7 | DAP12 |
| ABD | DAP12 | CD7 | CD32 |
| ABD | DAP12 | CD7 | CD79a |
| ABD | DAP12 | CD7 | CD79b |
| ABD | DAP12 | BTNL3 | CD8 |
| ABD | DAP12 | BTNL3 | CD3ζ |
| ABD | DAP12 | BTNL3 | CD3δ |
| ABD | DAP12 | BTNL3 | CD3γ |
| ABD | DAP12 | BTNL3 | CD3ε |
| ABD | DAP12 | BTNL3 | FcγRI-γ |
| ABD | DAP12 | BTNL3 | FcγRIII-γ |
| ABD | DAP12 | BTNL3 | FcεRIβ |
| ABD | DAP12 | BTNL3 | FcεRIγ |
| ABD | DAP12 | BTNL3 | DAP10 |
| ABD | DAP12 | BTNL3 | DAP12 |
| ABD | DAP12 | BTNL3 | CD32 |
| ABD | DAP12 | BTNL3 | CD79a |
| ABD | DAP12 | BTNL3 | CD79b |
| ABD | DAP12 | NKG2D | CD8 |
| ABD | DAP12 | NKG2D | CD3ζ |
| ABD | DAP12 | NKG2D | CD3δ |
| ABD | DAP12 | NKG2D | CD3γ |
| ABD | DAP12 | NKG2D | CD3ε |
| ABD | DAP12 | NKG2D | FcγRI-γ |
| ABD | DAP12 | NKG2D | FcγRIII-γ |
| ABD | DAP12 | NKG2D | FcεRIβ |
| ABD | DAP12 | NKG2D | FcεRIγ |
| ABD | DAP12 | NKG2D | DAP10 |
| ABD | DAP12 | NKG2D | DAP12 |
| ABD | DAP12 | NKG2D | CD32 |
| ABD | DAP12 | NKG2D | CD79a |
| ABD | DAP12 | NKG2D | CD79b |
| ABD | MyD88 | CD28 | CD8 |
| ABD | MyD88 | CD28 | CD3ζ |
| ABD | MyD88 | CD28 | CD3δ |
| ABD | MyD88 | CD28 | CD3γ |
| ABD | MyD88 | CD28 | CD3ε |
| ABD | MyD88 | CD28 | FcγRI-γ |
| ABD | MyD88 | CD28 | FcγRIII-γ |
| ABD | MyD88 | CD28 | FcεRIβ |
| ABD | MyD88 | CD28 | FcεRIγ |
| ABD | MyD88 | CD28 | DAP10 |
| ABD | MyD88 | CD28 | DAP12 |
| ABD | MyD88 | CD28 | CD32 |
| ABD | MyD88 | CD28 | CD79a |
| ABD | MyD88 | CD28 | CD79b |
| ABD | MyD88 | CD8 | CD8 |
| ABD | MyD88 | CD8 | CD3ζ |
| ABD | MyD88 | CD8 | CD3δ |
| ABD | MyD88 | CD8 | CD3γ |
| ABD | MyD88 | CD8 | CD3ε |
| ABD | MyD88 | CD8 | FcγRI-γ |
| ABD | MyD88 | CD8 | FcγRIII-γ |
| ABD | MyD88 | CD8 | FcεRIβ |
| ABD | MyD88 | CD8 | FcεRIγ |
| ABD | MyD88 | CD8 | DAP10 |
| ABD | MyD88 | CD8 | DAP12 |
| ABD | MyD88 | CD8 | CD32 |
| ABD | MyD88 | CD8 | CD79a |
| ABD | MyD88 | CD8 | CD79b |
| ABD | MyD88 | CD4 | CD8 |
| ABD | MyD88 | CD4 | CD3ζ |
| ABD | MyD88 | CD4 | CD3δ |
| ABD | MyD88 | CD4 | CD3γ |
| ABD | MyD88 | CD4 | CD3ε |
| ABD | MyD88 | CD4 | FcγRI-γ |
| ABD | MyD88 | CD4 | FcγRIII-γ |
| ABD | MyD88 | CD4 | FcεRIβ |
| ABD | MyD88 | CD4 | FcεRIγ |
| ABD | MyD88 | CD4 | DAP10 |
| ABD | MyD88 | CD4 | DAP12 |
| ABD | MyD88 | CD4 | CD32 |
| ABD | MyD88 | CD4 | CD79a |
| ABD | MyD88 | CD4 | CD79b |
| ABD | MyD88 | b2c | CD8 |
| ABD | MyD88 | b2c | CD3ζ |
| ABD | MyD88 | b2c | CD3δ |
| ABD | MyD88 | b2c | CD3γ |
| ABD | MyD88 | b2c | CD3ε |
| ABD | MyD88 | b2c | FcγRI-γ |
| ABD | MyD88 | b2c | FcγRIII-γ |
| ABD | MyD88 | b2c | FcεRIβ |
| ABD | MyD88 | b2c | FcεRIγ |
| ABD | MyD88 | b2c | DAP10 |
| ABD | MyD88 | b2c | DAP12 |
| ABD | MyD88 | b2c | CD32 |
| ABD | MyD88 | b2c | CD79a |
| ABD | MyD88 | b2c | CD79b |
| ABD | MyD88 | CD137/41BB | CD8 |
| ABD | MyD88 | CD137/41BB | CD3ζ |
| ABD | MyD88 | CD137/41BB | CD3δ |
| ABD | MyD88 | CD137/41BB | CD3γ |
| ABD | MyD88 | CD137/41BB | CD3ε |
| ABD | MyD88 | CD137/41BB | FcγRI-γ |
| ABD | MyD88 | CD137/41BB | FcγRIII-γ |
| ABD | MyD88 | CD137/41BB | FcεRIβ |
| ABD | MyD88 | CD137/41BB | FcεRIγ |
| ABD | MyD88 | CD137/41BB | DAP10 |
| ABD | MyD88 | CD137/41BB | DAP12 |
| ABD | MyD88 | CD137/41BB | CD32 |
| ABD | MyD88 | CD137/41BB | CD79a |
| ABD | MyD88 | CD137/41BB | CD79b |
| ABD | MyD88 | ICOS | CD8 |
| ABD | MyD88 | ICOS | CD3ζ |
| ABD | MyD88 | ICOS | CD3δ |
| ABD | MyD88 | ICOS | CD3γ |
| ABD | MyD88 | ICOS | CD3ε |
| ABD | MyD88 | ICOS | FcγRI-γ |
| ABD | MyD88 | ICOS | FcγRIII-γ |
| ABD | MyD88 | ICOS | FcεRIβ |
| ABD | MyD88 | ICOS | FcεRIγ |
| ABD | MyD88 | ICOS | DAP10 |
| ABD | MyD88 | ICOS | DAP12 |
| ABD | MyD88 | ICOS | CD32 |
| ABD | MyD88 | ICOS | CD79a |
| ABD | MyD88 | ICOS | CD79b |
| ABD | MyD88 | CD27 | CD8 |
| ABD | MyD88 | CD27 | CD3ζ |
| ABD | MyD88 | CD27 | CD3δ |
| ABD | MyD88 | CD27 | CD3γ |
| ABD | MyD88 | CD27 | CD3ε |
| ABD | MyD88 | CD27 | FcγRI-γ |
| ABD | MyD88 | CD27 | FcγRIII-γ |
| ABD | MyD88 | CD27 | FcεRIβ |
| ABD | MyD88 | CD27 | FcεRIγ |
| ABD | MyD88 | CD27 | DAP10 |
| ABD | MyD88 | CD27 | DAP12 |
| ABD | MyD88 | CD27 | CD32 |
| ABD | MyD88 | CD27 | CD79a |
| ABD | MyD88 | CD27 | CD79b |
| ABD | MyD88 | CD28δ | CD8 |
| ABD | MyD88 | CD28δ | CD3ζ |
| ABD | MyD88 | CD28δ | CD3δ |
| ABD | MyD88 | CD28δ | CD3γ |
| ABD | MyD88 | CD28δ | CD3ε |
| ABD | MyD88 | CD28δ | FcγRI-γ |
| ABD | MyD88 | CD28δ | FcγRIII-γ |
| ABD | MyD88 | CD28δ | FcεRIβ |
| ABD | MyD88 | CD28δ | FcεRIγ |
| ABD | MyD88 | CD28δ | DAP10 |
| ABD | MyD88 | CD28δ | DAP12 |
| ABD | MyD88 | CD28δ | CD32 |
| ABD | MyD88 | CD28δ | CD79a |
| ABD | MyD88 | CD28δ | CD79b |
| ABD | MyD88 | CD80 | CD8 |
| ABD | MyD88 | CD80 | CD3ζ |
| ABD | MyD88 | CD80 | CD3δ |
| ABD | MyD88 | CD80 | CD3γ |
| ABD | MyD88 | CD80 | CD3ε |
| ABD | MyD88 | CD80 | FcγRI-γ |
| ABD | MyD88 | CD80 | FcγRIII-γ |
| ABD | MyD88 | CD80 | FcεRIβ |
| ABD | MyD88 | CD80 | FcεRIγ |
| ABD | MyD88 | CD80 | DAP10 |
| ABD | MyD88 | CD80 | DAP12 |
| ABD | MyD88 | CD80 | CD32 |
| ABD | MyD88 | CD80 | CD79a |
| ABD | MyD88 | CD80 | CD79b |
| ABD | MyD88 | CD86 | CD8 |
| ABD | MyD88 | CD86 | CD3ζ |
| ABD | MyD88 | CD86 | CD3δ |
| ABD | MyD88 | CD86 | CD3γ |
| ABD | MyD88 | CD86 | CD3ε |
| ABD | MyD88 | CD86 | FcγRI-γ |
| ABD | MyD88 | CD86 | FcγRIII-γ |
| ABD | MyD88 | CD86 | FcεRIβ |
| ABD | MyD88 | CD86 | FcεRIγ |
| ABD | MyD88 | CD86 | DAP10 |
| ABD | MyD88 | CD86 | DAP12 |
| ABD | MyD88 | CD86 | CD32 |
| ABD | MyD88 | CD86 | CD79a |
| ABD | MyD88 | CD86 | CD79b |
| ABD | MyD88 | OX40 | CD8 |
| ABD | MyD88 | OX40 | CD3ζ |
| ABD | MyD88 | OX40 | CD3δ |
| ABD | MyD88 | OX40 | CD3γ |
| ABD | MyD88 | OX40 | CD3ε |
| ABD | MyD88 | OX40 | FcγRI-γ |
| ABD | MyD88 | OX40 | FcγRIII-γ |
| ABD | MyD88 | OX40 | FcεRIβ |
| ABD | MyD88 | OX40 | FcεRIγ |
| ABD | MyD88 | OX40 | DAP10 |
| ABD | MyD88 | OX40 | DAP12 |
| ABD | MyD88 | OX40 | CD32 |
| ABD | MyD88 | OX40 | CD79a |
| ABD | MyD88 | OX40 | CD79b |
| ABD | MyD88 | DAP10 | CD8 |
| ABD | MyD88 | DAP10 | CD3ζ |
| ABD | MyD88 | DAP10 | CD3δ |
| ABD | MyD88 | DAP10 | CD3γ |
| ABD | MyD88 | DAP10 | CD3ε |
| ABD | MyD88 | DAP10 | FcγRI-γ |
| ABD | MyD88 | DAP10 | FcγRIII-γ |
| ABD | MyD88 | DAP10 | FcεRIβ |
| ABD | MyD88 | DAP10 | FcεRIγ |
| ABD | MyD88 | DAP10 | DAP10 |
| ABD | MyD88 | DAP10 | DAP12 |
| ABD | MyD88 | DAP10 | CD32 |
| ABD | MyD88 | DAP10 | CD79a |
| ABD | MyD88 | DAP10 | CD79b |
| ABD | MyD88 | DAP12 | CD8 |
| ABD | MyD88 | DAP12 | CD3ζ |
| ABD | MyD88 | DAP12 | CD3δ |
| ABD | MyD88 | DAP12 | CD3γ |
| ABD | MyD88 | DAP12 | CD3ε |
| ABD | MyD88 | DAP12 | FcγRI-γ |
| ABD | MyD88 | DAP12 | FcγRIII-γ |
| ABD | MyD88 | DAP12 | FcεRIβ |
| ABD | MyD88 | DAP12 | FcεRIγ |
| ABD | MyD88 | DAP12 | DAP10 |
| ABD | MyD88 | DAP12 | DAP12 |
| ABD | MyD88 | DAP12 | CD32 |
| ABD | MyD88 | DAP12 | CD79a |
| ABD | MyD88 | DAP12 | CD79b |
| ABD | MyD88 | MyD88 | CD8 |
| ABD | MyD88 | MyD88 | CD3ζ |
| ABD | MyD88 | MyD88 | CD3δ |
| ABD | MyD88 | MyD88 | CD3γ |
| ABD | MyD88 | MyD88 | CD3ε |
| ABD | MyD88 | MyD88 | FcγRI-γ |
| ABD | MyD88 | MyD88 | FcγRIII-γ |
| ABD | MyD88 | MyD88 | FcεRIβ |
| ABD | MyD88 | MyD88 | FcεRIγ |
| ABD | MyD88 | MyD88 | DAP10 |
| ABD | MyD88 | MyD88 | DAP12 |
| ABD | MyD88 | MyD88 | CD32 |
| ABD | MyD88 | MyD88 | CD79a |
| ABD | MyD88 | MyD88 | CD79b |
| ABD | MyD88 | CD7 | CD8 |
| ABD | MyD88 | CD7 | CD3ζ |
| ABD | MyD88 | CD7 | CD3δ |
| ABD | MyD88 | CD7 | CD3γ |
| ABD | MyD88 | CD7 | CD3ε |
| ABD | MyD88 | CD7 | FcγRI-γ |
| ABD | MyD88 | CD7 | FcγRIII-γ |
| ABD | MyD88 | CD7 | FcεRIβ |
| ABD | MyD88 | CD7 | FcεRIγ |
| ABD | MyD88 | CD7 | DAP10 |
| ABD | MyD88 | CD7 | DAP12 |
| ABD | MyD88 | CD7 | CD32 |
| ABD | MyD88 | CD7 | CD79a |
| ABD | MyD88 | CD7 | CD79b |
| ABD | MyD88 | BTNL3 | CD8 |
| ABD | MyD88 | BTNL3 | CD3ζ |
| ABD | MyD88 | BTNL3 | CD3δ |
| ABD | MyD88 | BTNL3 | CD3γ |
| ABD | MyD88 | BTNL3 | CD3ε |
| ABD | MyD88 | BTNL3 | FcγRI-γ |
| ABD | MyD88 | BTNL3 | FcγRIII-γ |
| ABD | MyD88 | BTNL3 | FcεRIβ |
| ABD | MyD88 | BTNL3 | FcεRIγ |
| ABD | MyD88 | BTNL3 | DAP10 |
| ABD | MyD88 | BTNL3 | DAP12 |
| ABD | MyD88 | BTNL3 | CD32 |
| ABD | MyD88 | BTNL3 | CD79a |
| ABD | MyD88 | BTNL3 | CD79b |
| ABD | MyD88 | NKG2D | CD8 |
| ABD | MyD88 | NKG2D | CD3ζ |
| ABD | MyD88 | NKG2D | CD3δ |
| ABD | MyD88 | NKG2D | CD3γ |
| ABD | MyD88 | NKG2D | CD3ε |
| ABD | MyD88 | NKG2D | FcγRI-γ |
| ABD | MyD88 | NKG2D | FcγRIII-γ |
| ABD | MyD88 | NKG2D | FcεRIβ |
| ABD | MyD88 | NKG2D | FcεRIγ |
| ABD | MyD88 | NKG2D | DAP10 |
| ABD | MyD88 | NKG2D | DAP12 |
| ABD | MyD88 | NKG2D | CD32 |
| ABD | MyD88 | NKG2D | CD79a |
| ABD | MyD88 | NKG2D | CD79b |
| ABD | CD7 | CD28 | CD8 |
| ABD | CD7 | CD28 | CD3ζ |
| ABD | CD7 | CD28 | CD3δ |
| ABD | CD7 | CD28 | CD3γ |
| ABD | CD7 | CD28 | CD3ε |
| ABD | CD7 | CD28 | FcγRI-γ |
| ABD | CD7 | CD28 | FcγRIII-γ |
| ABD | CD7 | CD28 | FcεRIβ |
| ABD | CD7 | CD28 | FcεRIγ |
| ABD | CD7 | CD28 | DAP10 |
| ABD | CD7 | CD28 | DAP12 |
| ABD | CD7 | CD28 | CD32 |
| ABD | CD7 | CD28 | CD79a |
| ABD | CD7 | CD28 | CD79b |
| ABD | CD7 | CD8 | CD8 |
| ABD | CD7 | CD8 | CD3ζ |
| ABD | CD7 | CD8 | CD3δ |
| ABD | CD7 | CD8 | CD3γ |
| ABD | CD7 | CD8 | CD3ε |
| ABD | CD7 | CD8 | FcγRI-γ |
| ABD | CD7 | CD8 | FcγRIII-γ |
| ABD | CD7 | CD8 | FcεRIβ |
| ABD | CD7 | CD8 | FcεRIγ |
| ABD | CD7 | CD8 | DAP10 |
| ABD | CD7 | CD8 | DAP12 |
| ABD | CD7 | CD8 | CD32 |
| ABD | CD7 | CD8 | CD79a |
| ABD | CD7 | CD8 | CD79b |
| ABD | CD7 | CD4 | CD8 |
| ABD | CD7 | CD4 | CD3ζ |
| ABD | CD7 | CD4 | CD3δ |
| ABD | CD7 | CD4 | CD3γ |
| ABD | CD7 | CD4 | CD3ε |
| ABD | CD7 | CD4 | FcγRI-γ |
| ABD | CD7 | CD4 | FcγRIII-γ |
| ABD | CD7 | CD4 | FcεRIβ |
| ABD | CD7 | CD4 | FcεRIγ |
| ABD | CD7 | CD4 | DAP10 |
| ABD | CD7 | CD4 | DAP12 |
| ABD | CD7 | CD4 | CD32 |
| ABD | CD7 | CD4 | CD79a |
| ABD | CD7 | CD4 | CD79b |
| ABD | CD7 | b2c | CD8 |
| ABD | CD7 | b2c | CD3ζ |
| ABD | CD7 | b2c | CD3δ |
| ABD | CD7 | b2c | CD3γ |
| ABD | CD7 | b2c | CD3ε |
| ABD | CD7 | b2c | FcγRI-γ |
| ABD | CD7 | b2c | FcγRIII-γ |
| ABD | CD7 | b2c | FcεRIβ |
| ABD | CD7 | b2c | FcεRIγ |
| ABD | CD7 | b2c | DAP10 |
| ABD | CD7 | b2c | DAP12 |
| ABD | CD7 | b2c | CD32 |
| ABD | CD7 | b2c | CD79a |
| ABD | CD7 | b2c | CD79b |
| ABD | CD7 | CD137/41BB | CD8 |
| ABD | CD7 | CD137/41BB | CD3ζ |
| ABD | CD7 | CD137/41BB | CD3δ |
| ABD | CD7 | CD137/41BB | CD3γ |
| ABD | CD7 | CD137/41BB | CD3ε |
| ABD | CD7 | CD137/41BB | FcγRI-γ |
| ABD | CD7 | CD137/41BB | FcγRIII-γ |
| ABD | CD7 | CD137/41BB | FcεRIβ |
| ABD | CD7 | CD137/41BB | FcεRIγ |
| ABD | CD7 | CD137/41BB | DAP10 |
| ABD | CD7 | CD137/41BB | DAP12 |
| ABD | CD7 | CD137/41BB | CD32 |
| ABD | CD7 | CD137/41BB | CD79a |
| ABD | CD7 | CD137/41BB | CD79b |
| ABD | CD7 | ICOS | CD8 |
| ABD | CD7 | ICOS | CD3ζ |
| ABD | CD7 | ICOS | CD3δ |
| ABD | CD7 | ICOS | CD3γ |
| ABD | CD7 | ICOS | CD3ε |
| ABD | CD7 | ICOS | FcγRI-γ |
| ABD | CD7 | ICOS | FcγRIII-γ |
| ABD | CD7 | ICOS | FcεRIβ |
| ABD | CD7 | ICOS | FcεRIγ |
| ABD | CD7 | ICOS | DAP10 |
| ABD | CD7 | ICOS | DAP12 |
| ABD | CD7 | ICOS | CD32 |
| ABD | CD7 | ICOS | CD79a |
| ABD | CD7 | ICOS | CD79b |
| ABD | CD7 | CD27 | CD8 |
| ABD | CD7 | CD27 | CD3ζ |
| ABD | CD7 | CD27 | CD3δ |
| ABD | CD7 | CD27 | CD3γ |
| ABD | CD7 | CD27 | CD3ε |
| ABD | CD7 | CD27 | FcγRI-γ |
| ABD | CD7 | CD27 | FcγRIII-γ |
| ABD | CD7 | CD27 | FcεRIβ |
| ABD | CD7 | CD27 | FcεRIγ |
| ABD | CD7 | CD27 | DAP10 |
| ABD | CD7 | CD27 | DAP12 |
| ABD | CD7 | CD27 | CD32 |
| ABD | CD7 | CD27 | CD79a |
| ABD | CD7 | CD27 | CD79b |
| ABD | CD7 | CD28δ | CD8 |
| ABD | CD7 | CD28δ | CD3ζ |
| ABD | CD7 | CD28δ | CD3δ |
| ABD | CD7 | CD28δ | CD3γ |
| ABD | CD7 | CD28δ | CD3ε |
| ABD | CD7 | CD28δ | FcγRI-γ |
| ABD | CD7 | CD28δ | FcγRIII-γ |
| ABD | CD7 | CD28δ | FcεRIβ |
| ABD | CD7 | CD28δ | FcεRIγ |
| ABD | CD7 | CD28δ | DAP10 |
| ABD | CD7 | CD28δ | DAP12 |
| ABD | CD7 | CD28δ | CD32 |
| ABD | CD7 | CD28δ | CD79a |
| ABD | CD7 | CD28δ | CD79b |
| ABD | CD7 | CD80 | CD8 |
| ABD | CD7 | CD80 | CD3ζ |
| ABD | CD7 | CD80 | CD3δ |
| ABD | CD7 | CD80 | CD3γ |
| ABD | CD7 | CD80 | CD3ε |
| ABD | CD7 | CD80 | FcγRI-γ |
| ABD | CD7 | CD80 | FcγRIII-γ |
| ABD | CD7 | CD80 | FcεRIβ |
| ABD | CD7 | CD80 | FcεRIγ |
| ABD | CD7 | CD80 | DAP10 |
| ABD | CD7 | CD80 | DAP12 |
| ABD | CD7 | CD80 | CD32 |
| ABD | CD7 | CD80 | CD79a |
| ABD | CD7 | CD80 | CD79b |
| ABD | CD7 | CD86 | CD8 |
| ABD | CD7 | CD86 | CD3ζ |
| ABD | CD7 | CD86 | CD3δ |
| ABD | CD7 | CD86 | CD3γ |
| ABD | CD7 | CD86 | CD3ε |
| ABD | CD7 | CD86 | FcγRI-γ |
| ABD | CD7 | CD86 | FcγRIII-γ |
| ABD | CD7 | CD86 | FcεRIβ |
| ABD | CD7 | CD86 | FcεRIγ |
| ABD | CD7 | CD86 | DAP10 |
| ABD | CD7 | CD86 | DAP12 |
| ABD | CD7 | CD86 | CD32 |
| ABD | CD7 | CD86 | CD79a |
| ABD | CD7 | CD86 | CD79b |
| ABD | CD7 | OX40 | CD8 |
| ABD | CD7 | OX40 | CD3ζ |
| ABD | CD7 | OX40 | CD3δ |
| ABD | CD7 | OX40 | CD3γ |
| ABD | CD7 | OX40 | CD3ε |
| ABD | CD7 | OX40 | FcγRI-γ |
| ABD | CD7 | OX40 | FcγRIII-γ |
| ABD | CD7 | OX40 | FcεRIβ |
| ABD | CD7 | OX40 | FcεRIγ |
| ABD | CD7 | OX40 | DAP10 |
| ABD | CD7 | OX40 | DAP12 |
| ABD | CD7 | OX40 | CD32 |
| ABD | CD7 | OX40 | CD79a |
| ABD | CD7 | OX40 | CD79b |
| ABD | CD7 | DAP10 | CD8 |
| ABD | CD7 | DAP10 | CD3ζ |
| ABD | CD7 | DAP10 | CD3δ |
| ABD | CD7 | DAP10 | CD3γ |
| ABD | CD7 | DAP10 | CD3ε |
| ABD | CD7 | DAP10 | FcγRI-γ |
| ABD | CD7 | DAP10 | FcγRIII-γ |
| ABD | CD7 | DAP10 | FcεRIβ |
| ABD | CD7 | DAP10 | FcεRIγ |
| ABD | CD7 | DAP10 | DAP10 |
| ABD | CD7 | DAP10 | DAP12 |
| ABD | CD7 | DAP10 | CD32 |
| ABD | CD7 | DAP10 | CD79a |
| ABD | CD7 | DAP10 | CD79b |
| ABD | CD7 | DAP12 | CD8 |
| ABD | CD7 | DAP12 | CD3ζ |
| ABD | CD7 | DAP12 | CD3δ |
| ABD | CD7 | DAP12 | CD3γ |
| ABD | CD7 | DAP12 | CD3ε |
| ABD | CD7 | DAP12 | FcγRI-γ |
| ABD | CD7 | DAP12 | FcγRIII-γ |
| ABD | CD7 | DAP12 | FcεRIβ |
| ABD | CD7 | DAP12 | FcεRIγ |
| ABD | CD7 | DAP12 | DAP10 |
| ABD | CD7 | DAP12 | DAP12 |
| ABD | CD7 | DAP12 | CD32 |
| ABD | CD7 | DAP12 | CD79a |
| ABD | CD7 | DAP12 | CD79b |
| ABD | CD7 | MyD88 | CD8 |
| ABD | CD7 | MyD88 | CD3ζ |
| ABD | CD7 | MyD88 | CD3δ |
| ABD | CD7 | MyD88 | CD3γ |
| ABD | CD7 | MyD88 | CD3ε |
| ABD | CD7 | MyD88 | FcγRI-γ |
| ABD | CD7 | MyD88 | FcγRIII-γ |
| ABD | CD7 | MyD88 | FcεRIβ |
| ABD | CD7 | MyD88 | FcεRIγ |
| ABD | CD7 | MyD88 | DAP10 |
| ABD | CD7 | MyD88 | DAP12 |
| ABD | CD7 | MyD88 | CD32 |
| ABD | CD7 | MyD88 | CD79a |
| ABD | CD7 | MyD88 | CD79b |
| ABD | CD7 | CD7 | CD8 |
| ABD | CD7 | CD7 | CD3ζ |
| ABD | CD7 | CD7 | CD3δ |
| ABD | CD7 | CD7 | CD3γ |
| ABD | CD7 | CD7 | CD3ε |
| ABD | CD7 | CD7 | FcγRI-γ |
| ABD | CD7 | CD7 | FcγRIII-γ |
| ABD | CD7 | CD7 | FcεRIβ |
| ABD | CD7 | CD7 | FcεRIγ |
| ABD | CD7 | CD7 | DAP10 |
| ABD | CD7 | CD7 | DAP12 |
| ABD | CD7 | CD7 | CD32 |
| ABD | CD7 | CD7 | CD79a |
| ABD | CD7 | CD7 | CD79b |
| ABD | CD7 | BTNL3 | CD8 |
| ABD | CD7 | BTNL3 | CD3ζ |
| ABD | CD7 | BTNL3 | CD3δ |
| ABD | CD7 | BTNL3 | CD3γ |
| ABD | CD7 | BTNL3 | CD3ε |
| ABD | CD7 | BTNL3 | FcγRI-γ |
| ABD | CD7 | BTNL3 | FcγRIII-γ |
| ABD | CD7 | BTNL3 | FcεRIβ |
| ABD | CD7 | BTNL3 | FcεRIγ |
| ABD | CD7 | BTNL3 | DAP10 |
| ABD | CD7 | BTNL3 | DAP12 |
| ABD | CD7 | BTNL3 | CD32 |
| ABD | CD7 | BTNL3 | CD79a |
| ABD | CD7 | BTNL3 | CD79b |
| ABD | CD7 | NKG2D | CD8 |
| ABD | CD7 | NKG2D | CD3ζ |
| ABD | CD7 | NKG2D | CD3δ |
| ABD | CD7 | NKG2D | CD3γ |
| ABD | CD7 | NKG2D | CD3ε |
| ABD | CD7 | NKG2D | FcγRI-γ |
| ABD | CD7 | NKG2D | FcγRIII-γ |
| ABD | CD7 | NKG2D | FcεRIβ |
| ABD | CD7 | NKG2D | FcεRIγ |
| ABD | CD7 | NKG2D | DAP10 |
| ABD | CD7 | NKG2D | DAP12 |
| ABD | CD7 | NKG2D | CD32 |
| ABD | CD7 | NKG2D | CD79a |
| ABD | CD7 | NKG2D | CD79b |
| ABD | BTNL3 | CD28 | CD8 |
| ABD | BTNL3 | CD28 | CD3ζ |
| ABD | BTNL3 | CD28 | CD3δ |
| ABD | BTNL3 | CD28 | CD3γ |
| ABD | BTNL3 | CD28 | CD3ε |
| ABD | BTNL3 | CD28 | FcγRI-γ |
| ABD | BTNL3 | CD28 | FcγRIII-γ |
| ABD | BTNL3 | CD28 | FcεRIβ |
| ABD | BTNL3 | CD28 | FcεRIγ |
| ABD | BTNL3 | CD28 | DAP10 |
| ABD | BTNL3 | CD28 | DAP12 |
| ABD | BTNL3 | CD28 | CD32 |
| ABD | BTNL3 | CD28 | CD79a |
| ABD | BTNL3 | CD28 | CD79b |
| ABD | BTNL3 | CD8 | CD8 |
| ABD | BTNL3 | CD8 | CD3ζ |
| ABD | BTNL3 | CD8 | CD3δ |
| ABD | BTNL3 | CD8 | CD3γ |
| ABD | BTNL3 | CD8 | CD3ε |
| ABD | BTNL3 | CD8 | FcγRI-γ |
| ABD | BTNL3 | CD8 | FcγRIII-γ |
| ABD | BTNL3 | CD8 | FcεRIβ |
| ABD | BTNL3 | CD8 | FcεRIγ |
| ABD | BTNL3 | CD8 | DAP10 |
| ABD | BTNL3 | CD8 | DAP12 |
| ABD | BTNL3 | CD8 | CD32 |
| ABD | BTNL3 | CD8 | CD79a |
| ABD | BTNL3 | CD8 | CD79b |
| ABD | BTNL3 | CD4 | CD8 |
| ABD | BTNL3 | CD4 | CD3ζ |
| ABD | BTNL3 | CD4 | CD3δ |
| ABD | BTNL3 | CD4 | CD3γ |
| ABD | BTNL3 | CD4 | CD3ε |
| ABD | BTNL3 | CD4 | FcγRI-γ |
| ABD | BTNL3 | CD4 | FcγRIII-γ |
| ABD | BTNL3 | CD4 | FcεRIβ |
| ABD | BTNL3 | CD4 | FcεRIγ |
| ABD | BTNL3 | CD4 | DAP10 |
| ABD | BTNL3 | CD4 | DAP12 |
| ABD | BTNL3 | CD4 | CD32 |
| ABD | BTNL3 | CD4 | CD79a |
| ABD | BTNL3 | CD4 | CD79b |
| ABD | BTNL3 | b2c | CD8 |
| ABD | BTNL3 | b2c | CD3ζ |
| ABD | BTNL3 | b2c | CD3δ |
| ABD | BTNL3 | b2c | CD3γ |
| ABD | BTNL3 | b2c | CD3ε |
| ABD | BTNL3 | b2c | FcγRI-γ |
| ABD | BTNL3 | b2c | FcγRIII-γ |
| ABD | BTNL3 | b2c | FcεRIβ |
| ABD | BTNL3 | b2c | FcεRIγ |
| ABD | BTNL3 | b2c | DAP10 |
| ABD | BTNL3 | b2c | DAP12 |
| ABD | BTNL3 | b2c | CD32 |
| ABD | BTNL3 | b2c | CD79a |
| ABD | BTNL3 | b2c | CD79b |
| ABD | BTNL3 | CD137/41BB | CD8 |
| ABD | BTNL3 | CD137/41BB | CD3ζ |
| ABD | BTNL3 | CD137/41BB | CD3δ |
| ABD | BTNL3 | CD137/41BB | CD3γ |
| ABD | BTNL3 | CD137/41BB | CD3ε |
| ABD | BTNL3 | CD137/41BB | FcγRI-γ |
| ABD | BTNL3 | CD137/41BB | FcγRIII-γ |
| ABD | BTNL3 | CD137/41BB | FcεRIβ |
| ABD | BTNL3 | CD137/41BB | FcεRIγ |
| ABD | BTNL3 | CD137/41BB | DAP10 |
| ABD | BTNL3 | CD137/41BB | DAP12 |
| ABD | BTNL3 | CD137/41BB | CD32 |
| ABD | BTNL3 | CD137/41BB | CD79a |
| ABD | BTNL3 | CD137/41BB | CD79b |
| ABD | BTNL3 | ICOS | CD8 |
| ABD | BTNL3 | ICOS | CD3ζ |
| ABD | BTNL3 | ICOS | CD3δ |
| ABD | BTNL3 | ICOS | CD3γ |
| ABD | BTNL3 | ICOS | CD3ε |
| ABD | BTNL3 | ICOS | FcγRI-γ |
| ABD | BTNL3 | ICOS | FcγRIII-γ |
| ABD | BTNL3 | ICOS | FcεRIβ |
| ABD | BTNL3 | ICOS | FcεRIγ |
| ABD | BTNL3 | ICOS | DAP10 |
| ABD | BTNL3 | ICOS | DAP12 |
| ABD | BTNL3 | ICOS | CD32 |
| ABD | BTNL3 | ICOS | CD79a |
| ABD | BTNL3 | ICOS | CD79b |
| ABD | BTNL3 | CD27 | CD8 |
| ABD | BTNL3 | CD27 | CD3ζ |
| ABD | BTNL3 | CD27 | CD3δ |
| ABD | BTNL3 | CD27 | CD3γ |
| ABD | BTNL3 | CD27 | CD3ε |
| ABD | BTNL3 | CD27 | FcγRI-γ |
| ABD | BTNL3 | CD27 | FcγRIII-γ |
| ABD | BTNL3 | CD27 | FcεRIβ |
| ABD | BTNL3 | CD27 | FcεRIγ |
| ABD | BTNL3 | CD27 | DAP10 |
| ABD | BTNL3 | CD27 | DAP12 |
| ABD | BTNL3 | CD27 | CD32 |
| ABD | BTNL3 | CD27 | CD79a |
| ABD | BTNL3 | CD27 | CD79b |
| ABD | BTNL3 | CD28δ | CD8 |
| ABD | BTNL3 | CD28δ | CD3ζ |
| ABD | BTNL3 | CD28δ | CD3δ |
| ABD | BTNL3 | CD28δ | CD3γ |
| ABD | BTNL3 | CD28δ | CD3ε |
| ABD | BTNL3 | CD28δ | FcγRI-γ |
| ABD | BTNL3 | CD28δ | FcγRIII-γ |
| ABD | BTNL3 | CD28δ | FcεRIβ |
| ABD | BTNL3 | CD28δ | FcεRIγ |
| ABD | BTNL3 | CD28δ | DAP10 |
| ABD | BTNL3 | CD28δ | DAP12 |
| ABD | BTNL3 | CD28δ | CD32 |
| ABD | BTNL3 | CD28δ | CD79a |
| ABD | BTNL3 | CD28δ | CD79b |
| ABD | BTNL3 | CD80 | CD8 |
| ABD | BTNL3 | CD80 | CD3ζ |
| ABD | BTNL3 | CD80 | CD3δ |
| ABD | BTNL3 | CD80 | CD3γ |
| ABD | BTNL3 | CD80 | CD3ε |
| ABD | BTNL3 | CD80 | FcγRI-γ |
| ABD | BTNL3 | CD80 | FcγRIII-γ |
| ABD | BTNL3 | CD80 | FcεRIβ |
| ABD | BTNL3 | CD80 | FcεRIγ |
| ABD | BTNL3 | CD80 | DAP10 |
| ABD | BTNL3 | CD80 | DAP12 |
| ABD | BTNL3 | CD80 | CD32 |
| ABD | BTNL3 | CD80 | CD79a |
| ABD | BTNL3 | CD80 | CD79b |
| ABD | BTNL3 | CD86 | CD8 |
| ABD | BTNL3 | CD86 | CD3ζ |
| ABD | BTNL3 | CD86 | CD3δ |
| ABD | BTNL3 | CD86 | CD3γ |
| ABD | BTNL3 | CD86 | CD3ε |
| ABD | BTNL3 | CD86 | FcγRI-γ |
| ABD | BTNL3 | CD86 | FcγRIII-γ |
| ABD | BTNL3 | CD86 | FcεRIβ |
| ABD | BTNL3 | CD86 | FcεRIγ |
| ABD | BTNL3 | CD86 | DAP10 |
| ABD | BTNL3 | CD86 | DAP12 |
| ABD | BTNL3 | CD86 | CD32 |
| ABD | BTNL3 | CD86 | CD79a |
| ABD | BTNL3 | CD86 | CD79b |
| ABD | BTNL3 | OX40 | CD8 |
| ABD | BTNL3 | OX40 | CD3ζ |
| ABD | BTNL3 | OX40 | CD3δ |
| ABD | BTNL3 | OX40 | CD3γ |
| ABD | BTNL3 | OX40 | CD3ε |
| ABD | BTNL3 | OX40 | FcγRI-γ |
| ABD | BTNL3 | OX40 | FcγRIII-γ |
| ABD | BTNL3 | OX40 | FcεRIβ |
| ABD | BTNL3 | OX40 | FcεRIγ |
| ABD | BTNL3 | OX40 | DAP10 |
| ABD | BTNL3 | OX40 | DAP12 |
| ABD | BTNL3 | OX40 | CD32 |
| ABD | BTNL3 | OX40 | CD79a |
| ABD | BTNL3 | OX40 | CD79b |
| ABD | BTNL3 | DAP10 | CD8 |
| ABD | BTNL3 | DAP10 | CD3ζ |
| ABD | BTNL3 | DAP10 | CD3δ |
| ABD | BTNL3 | DAP10 | CD3γ |
| ABD | BTNL3 | DAP10 | CD3ε |
| ABD | BTNL3 | DAP10 | FcγRI-γ |
| ABD | BTNL3 | DAP10 | FcγRIII-γ |
| ABD | BTNL3 | DAP10 | FcεRIβ |
| ABD | BTNL3 | DAP10 | FcεRIγ |
| ABD | BTNL3 | DAP10 | DAP10 |
| ABD | BTNL3 | DAP10 | DAP12 |
| ABD | BTNL3 | DAP10 | CD32 |
| ABD | BTNL3 | DAP10 | CD79a |
| ABD | BTNL3 | DAP10 | CD79b |
| ABD | BTNL3 | DAP12 | CD8 |
| ABD | BTNL3 | DAP12 | CD3ζ |
| ABD | BTNL3 | DAP12 | CD3δ |
| ABD | BTNL3 | DAP12 | CD3γ |
| ABD | BTNL3 | DAP12 | CD3ε |
| ABD | BTNL3 | DAP12 | FcγRI-γ |
| ABD | BTNL3 | DAP12 | FcγRIII-γ |
| ABD | BTNL3 | DAP12 | FcεRIβ |
| ABD | BTNL3 | DAP12 | FcεRIγ |
| ABD | BTNL3 | DAP12 | DAP10 |
| ABD | BTNL3 | DAP12 | DAP12 |
| ABD | BTNL3 | DAP12 | CD32 |
| ABD | BTNL3 | DAP12 | CD79a |
| ABD | BTNL3 | DAP12 | CD79b |
| ABD | BTNL3 | MyD88 | CD8 |
| ABD | BTNL3 | MyD88 | CD3ζ |
| ABD | BTNL3 | MyD88 | CD3δ |
| ABD | BTNL3 | MyD88 | CD3γ |
| ABD | BTNL3 | MyD88 | CD3ε |
| ABD | BTNL3 | MyD88 | FcγRI-γ |
| ABD | BTNL3 | MyD88 | FcγRIII-γ |
| ABD | BTNL3 | MyD88 | FcεRIβ |
| ABD | BTNL3 | MyD88 | FcεRIγ |
| ABD | BTNL3 | MyD88 | DAP10 |
| ABD | BTNL3 | MyD88 | DAP12 |
| ABD | BTNL3 | MyD88 | CD32 |
| ABD | BTNL3 | MyD88 | CD79a |
| ABD | BTNL3 | MyD88 | CD79b |
| ABD | BTNL3 | CD7 | CD8 |
| ABD | BTNL3 | CD7 | CD3ζ |
| ABD | BTNL3 | CD7 | CD3δ |
| ABD | BTNL3 | CD7 | CD3γ |
| ABD | BTNL3 | CD7 | CD3ε |
| ABD | BTNL3 | CD7 | FcγRI-γ |
| ABD | BTNL3 | CD7 | FcγRIII-γ |
| ABD | BTNL3 | CD7 | FcεRIβ |
| ABD | BTNL3 | CD7 | FcεRIγ |
| ABD | BTNL3 | CD7 | DAP10 |
| ABD | BTNL3 | CD7 | DAP12 |
| ABD | BTNL3 | CD7 | CD32 |
| ABD | BTNL3 | CD7 | CD79a |
| ABD | BTNL3 | CD7 | CD79b |
| ABD | BTNL3 | BTNL3 | CD8 |
| ABD | BTNL3 | BTNL3 | CD3ζ |
| ABD | BTNL3 | BTNL3 | CD3δ |
| ABD | BTNL3 | BTNL3 | CD3γ |
| ABD | BTNL3 | BTNL3 | CD3ε |
| ABD | BTNL3 | BTNL3 | FcγRI-γ |
| ABD | BTNL3 | BTNL3 | FcγRIII-γ |
| ABD | BTNL3 | BTNL3 | FcεRIβ |
| ABD | BTNL3 | BTNL3 | FcεRIγ |
| ABD | BTNL3 | BTNL3 | DAP10 |
| ABD | BTNL3 | BTNL3 | DAP12 |
| ABD | BTNL3 | BTNL3 | CD32 |
| ABD | BTNL3 | BTNL3 | CD79a |
| ABD | BTNL3 | BTNL3 | CD79b |
| ABD | BTNL3 | NKG2D | CD8 |
| ABD | BTNL3 | NKG2D | CD3ζ |
| ABD | BTNL3 | NKG2D | CD3δ |
| ABD | BTNL3 | NKG2D | CD3γ |
| ABD | BTNL3 | NKG2D | CD3ε |
| ABD | BTNL3 | NKG2D | FcγRI-γ |
| ABD | BTNL3 | NKG2D | FcγRIII-γ |
| ABD | BTNL3 | NKG2D | FcεRIβ |
| ABD | BTNL3 | NKG2D | FcεRIγ |
| ABD | BTNL3 | NKG2D | DAP10 |
| ABD | BTNL3 | NKG2D | DAP12 |
| ABD | BTNL3 | NKG2D | CD32 |
| ABD | BTNL3 | NKG2D | CD79a |
| ABD | BTNL3 | NKG2D | CD79b |
| ABD | NKG2D | CD28 | CD8 |
| ABD | NKG2D | CD28 | CD3ζ |
| ABD | NKG2D | CD28 | CD3δ |
| ABD | NKG2D | CD28 | CD3γ |
| ABD | NKG2D | CD28 | CD3ε |
| ABD | NKG2D | CD28 | FcγRI-γ |
| ABD | NKG2D | CD28 | FcγRIII-γ |
| ABD | NKG2D | CD28 | FcεRIβ |
| ABD | NKG2D | CD28 | FcεRIγ |
| ABD | NKG2D | CD28 | DAP10 |
| ABD | NKG2D | CD28 | DAP12 |
| ABD | NKG2D | CD28 | CD32 |
| ABD | NKG2D | CD28 | CD79a |
| ABD | NKG2D | CD28 | CD79b |
| ABD | NKG2D | CD8 | CD8 |
| ABD | NKG2D | CD8 | CD3ζ |
| ABD | NKG2D | CD8 | CD3δ |
| ABD | NKG2D | CD8 | CD3γ |
| ABD | NKG2D | CD8 | CD3ε |
| ABD | NKG2D | CD8 | FcγRI-γ |
| ABD | NKG2D | CD8 | FcγRIII-γ |
| ABD | NKG2D | CD8 | FcεRIβ |
| ABD | NKG2D | CD8 | FcεRIγ |
| ABD | NKG2D | CD8 | DAP10 |
| ABD | NKG2D | CD8 | DAP12 |
| ABD | NKG2D | CD8 | CD32 |
| ABD | NKG2D | CD8 | CD79a |
| ABD | NKG2D | CD8 | CD79b |
| ABD | NKG2D | CD4 | CD8 |
| ABD | NKG2D | CD4 | CD3ζ |
| ABD | NKG2D | CD4 | CD3δ |
| ABD | NKG2D | CD4 | CD3γ |
| ABD | NKG2D | CD4 | CD3ε |
| ABD | NKG2D | CD4 | FcγRI-γ |
| ABD | NKG2D | CD4 | FcγRIII-γ |
| ABD | NKG2D | CD4 | FcεRIβ |
| ABD | NKG2D | CD4 | FcεRIγ |
| ABD | NKG2D | CD4 | DAP10 |
| ABD | NKG2D | CD4 | DAP12 |
| ABD | NKG2D | CD4 | CD32 |
| ABD | NKG2D | CD4 | CD79a |
| ABD | NKG2D | CD4 | CD79b |
| ABD | NKG2D | b2c | CD8 |
| ABD | NKG2D | b2c | CD3ζ |
| ABD | NKG2D | b2c | CD3δ |
| ABD | NKG2D | b2c | CD3γ |
| ABD | NKG2D | b2c | CD3ε |
| ABD | NKG2D | b2c | FcγRI-γ |
| ABD | NKG2D | b2c | FcγRIII-γ |
| ABD | NKG2D | b2c | FcεRIβ |
| ABD | NKG2D | b2c | FcεRIγ |
| ABD | NKG2D | b2c | DAP10 |
| ABD | NKG2D | b2c | DAP12 |
| ABD | NKG2D | b2c | CD32 |
| ABD | NKG2D | b2c | CD79a |
| ABD | NKG2D | b2c | CD79b |
| ABD | NKG2D | CD137/41BB | CD8 |
| ABD | NKG2D | CD137/41BB | CD3ζ |
| ABD | NKG2D | CD137/41BB | CD3δ |
| ABD | NKG2D | CD137/41BB | CD3γ |
| ABD | NKG2D | CD137/41BB | CD3ε |
| ABD | NKG2D | CD137/41BB | FcγRI-γ |
| ABD | NKG2D | CD137/41BB | FcγRIII-γ |
| ABD | NKG2D | CD137/41BB | FcεRIβ |
| ABD | NKG2D | CD137/41BB | FcεRIγ |
| ABD | NKG2D | CD137/41BB | DAP10 |
| ABD | NKG2D | CD137/41BB | DAP12 |
| ABD | NKG2D | CD137/41BB | CD32 |
| ABD | NKG2D | CD137/41BB | CD79a |
| ABD | NKG2D | CD137/41BB | CD79b |
| ABD | NKG2D | ICOS | CD8 |
| ABD | NKG2D | ICOS | CD3ζ |
| ABD | NKG2D | ICOS | CD3δ |
| ABD | NKG2D | ICOS | CD3γ |
| ABD | NKG2D | ICOS | CD3ε |
| ABD | NKG2D | ICOS | FcγRI-γ |
| ABD | NKG2D | ICOS | FcγRIII-γ |
| ABD | NKG2D | ICOS | FcεRIβ |
| ABD | NKG2D | ICOS | FcεRIγ |
| ABD | NKG2D | ICOS | DAP10 |
| ABD | NKG2D | ICOS | DAP12 |
| ABD | NKG2D | ICOS | CD32 |
| ABD | NKG2D | ICOS | CD79a |
| ABD | NKG2D | ICOS | CD79b |
| ABD | NKG2D | CD27 | CD8 |
| ABD | NKG2D | CD27 | CD3ζ |
| ABD | NKG2D | CD27 | CD3δ |
| ABD | NKG2D | CD27 | CD3γ |
| ABD | NKG2D | CD27 | CD3ε |
| ABD | NKG2D | CD27 | FcγRI-γ |
| ABD | NKG2D | CD27 | FcγRIII-γ |
| ABD | NKG2D | CD27 | FcεRIβ |
| ABD | NKG2D | CD27 | FcεRIγ |
| ABD | NKG2D | CD27 | DAP10 |
| ABD | NKG2D | CD27 | DAP12 |
| ABD | NKG2D | CD27 | CD32 |
| ABD | NKG2D | CD27 | CD79a |
| ABD | NKG2D | CD27 | CD79b |
| ABD | NKG2D | CD28δ | CD8 |
| ABD | NKG2D | CD28δ | CD3ζ |
| ABD | NKG2D | CD28δ | CD3δ |
| ABD | NKG2D | CD28δ | CD3γ |
| ABD | NKG2D | CD28δ | CD3ε |
| ABD | NKG2D | CD28δ | FcγRI-γ |
| ABD | NKG2D | CD28δ | FcγRIII-γ |
| ABD | NKG2D | CD28δ | FcεRIβ |
| ABD | NKG2D | CD28δ | FcεRIγ |
| ABD | NKG2D | CD28δ | DAP10 |
| ABD | NKG2D | CD28δ | DAP12 |
| ABD | NKG2D | CD28δ | CD32 |
| ABD | NKG2D | CD28δ | CD79a |
| ABD | NKG2D | CD28δ | CD79b |
| ABD | NKG2D | CD80 | CD8 |
| ABD | NKG2D | CD80 | CD3ζ |
| ABD | NKG2D | CD80 | CD3δ |
| ABD | NKG2D | CD80 | CD3γ |
| ABD | NKG2D | CD80 | CD3ε |
| ABD | NKG2D | CD80 | FcγRI-γ |
| ABD | NKG2D | CD80 | FcγRIII-γ |
| ABD | NKG2D | CD80 | FcεRIβ |
| ABD | NKG2D | CD80 | FcεRIγ |
| ABD | NKG2D | CD80 | DAP10 |
| ABD | NKG2D | CD80 | DAP12 |
| ABD | NKG2D | CD80 | CD32 |
| ABD | NKG2D | CD80 | CD79a |
| ABD | NKG2D | CD80 | CD79b |
| ABD | NKG2D | CD86 | CD8 |
| ABD | NKG2D | CD86 | CD3ζ |
| ABD | NKG2D | CD86 | CD3δ |
| ABD | NKG2D | CD86 | CD3γ |
| ABD | NKG2D | CD86 | CD3ε |
| ABD | NKG2D | CD86 | FcγRI-γ |
| ABD | NKG2D | CD86 | FcγRIII-γ |
| ABD | NKG2D | CD86 | FcεRIβ |
| ABD | NKG2D | CD86 | FcεRIγ |
| ABD | NKG2D | CD86 | DAP10 |
| ABD | NKG2D | CD86 | DAP12 |
| ABD | NKG2D | CD86 | CD32 |
| ABD | NKG2D | CD86 | CD79a |
| ABD | NKG2D | CD86 | CD79b |
| ABD | NKG2D | OX40 | CD8 |
| ABD | NKG2D | OX40 | CD3ζ |
| ABD | NKG2D | OX40 | CD3δ |
| ABD | NKG2D | OX40 | CD3γ |
| ABD | NKG2D | OX40 | CD3ε |
| ABD | NKG2D | OX40 | FcγRI-γ |
| ABD | NKG2D | OX40 | FcγRIII-γ |
| ABD | NKG2D | OX40 | FcεRIβ |
| ABD | NKG2D | OX40 | FcεRIγ |
| ABD | NKG2D | OX40 | DAP10 |
| ABD | NKG2D | OX40 | DAP12 |
| ABD | NKG2D | OX40 | CD32 |
| ABD | NKG2D | OX40 | CD79a |
| ABD | NKG2D | OX40 | CD79b |
| ABD | NKG2D | DAP10 | CD8 |
| ABD | NKG2D | DAP10 | CD3ζ |
| ABD | NKG2D | DAP10 | CD3δ |
| ABD | NKG2D | DAP10 | CD3γ |
| ABD | NKG2D | DAP10 | CD3ε |
| ABD | NKG2D | DAP10 | FcγRI-γ |
| ABD | NKG2D | DAP10 | FcγRIII-γ |
| ABD | NKG2D | DAP10 | FcεRIβ |
| ABD | NKG2D | DAP10 | FcεRIγ |
| ABD | NKG2D | DAP10 | DAP10 |
| ABD | NKG2D | DAP10 | DAP12 |
| ABD | NKG2D | DAP10 | CD32 |
| ABD | NKG2D | DAP10 | CD79a |
| ABD | NKG2D | DAP10 | CD79b |
| ABD | NKG2D | DAP12 | CD8 |
| ABD | NKG2D | DAP12 | CD3ζ |
| ABD | NKG2D | DAP12 | CD3δ |
| ABD | NKG2D | DAP12 | CD3γ |
| ABD | NKG2D | DAP12 | CD3ε |
| ABD | NKG2D | DAP12 | FcγRI-γ |
| ABD | NKG2D | DAP12 | FcγRIII-γ |
| ABD | NKG2D | DAP12 | FcεRIβ |
| ABD | NKG2D | DAP12 | FcεRIγ |
| ABD | NKG2D | DAP12 | DAP10 |
| ABD | NKG2D | DAP12 | DAP12 |
| ABD | NKG2D | DAP12 | CD32 |
| ABD | NKG2D | DAP12 | CD79a |
| ABD | NKG2D | DAP12 | CD79b |
| ABD | NKG2D | MyD88 | CD8 |
| ABD | NKG2D | MyD88 | CD3ζ |
| ABD | NKG2D | MyD88 | CD3δ |
| ABD | NKG2D | MyD88 | CD3γ |
| ABD | NKG2D | MyD88 | CD3ε |
| ABD | NKG2D | MyD88 | FcγRI-γ |
| ABD | NKG2D | MyD88 | FcγRIII-γ |
| ABD | NKG2D | MyD88 | FcεRIβ |
| ABD | NKG2D | MyD88 | FcεRIγ |
| ABD | NKG2D | MyD88 | DAP10 |
| ABD | NKG2D | MyD88 | DAP12 |
| ABD | NKG2D | MyD88 | CD32 |
| ABD | NKG2D | MyD88 | CD79a |
| ABD | NKG2D | MyD88 | CD79b |
| ABD | NKG2D | CD7 | CD8 |
| ABD | NKG2D | CD7 | CD3ζ |
| ABD | NKG2D | CD7 | CD3δ |
| ABD | NKG2D | CD7 | CD3γ |
| ABD | NKG2D | CD7 | CD3ε |
| ABD | NKG2D | CD7 | FcγRI-γ |
| ABD | NKG2D | CD7 | FcγRIII-γ |
| ABD | NKG2D | CD7 | FcεRIβ |
| ABD | NKG2D | CD7 | FcεRIγ |
| ABD | NKG2D | CD7 | DAP10 |
| ABD | NKG2D | CD7 | DAP12 |
| ABD | NKG2D | CD7 | CD32 |
| ABD | NKG2D | CD7 | CD79a |
| ABD | NKG2D | CD7 | CD79b |
| ABD | NKG2D | BTNL3 | CD8 |
| ABD | NKG2D | BTNL3 | CD3ζ |
| ABD | NKG2D | BTNL3 | CD3δ |
| ABD | NKG2D | BTNL3 | CD3γ |
| ABD | NKG2D | BTNL3 | CD3ε |
| ABD | NKG2D | BTNL3 | FcγRI-γ |
| ABD | NKG2D | BTNL3 | FcγRIII-γ |
| ABD | NKG2D | BTNL3 | FcεRIβ |
| ABD | NKG2D | BTNL3 | FcεRIγ |
| ABD | NKG2D | BTNL3 | DAP10 |
| ABD | NKG2D | BTNL3 | DAP12 |
| ABD | NKG2D | BTNL3 | CD32 |
| ABD | NKG2D | BTNL3 | CD79a |
| ABD | NKG2D | BTNL3 | CD79b |
| ABD | NKG2D | NKG2D | CD8 |
| ABD | NKG2D | NKG2D | CD3ζ |
| ABD | NKG2D | NKG2D | CD3δ |
| ABD | NKG2D | NKG2D | CD3γ |
| ABD | NKG2D | NKG2D | CD3ε |
| ABD | NKG2D | NKG2D | FcγRI-γ |
| ABD | NKG2D | NKG2D | FcγRIII-γ |
| ABD | NKG2D | NKG2D | FcεRIβ |
| ABD | NKG2D | NKG2D | FcεRIγ |
| ABD | NKG2D | NKG2D | DAP10 |
| ABD | NKG2D | NKG2D | DAP12 |
| ABD | NKG2D | NKG2D | CD32 |
| ABD | NKG2D | NKG2D | CD79a |
| ABD | NKG2D | NKG2D | CD79b |

TABLE 4
CARs lacking Co-Simulatory Signal (for dual CAR approach)

| ScFv | Co-stimulatory Signal | Signal Domain |
|---|---|---|
| ABD | none | CD8 |
| ABD | none | CD3ζ |
| ABD | none | CD3δ |
| ABD | none | CD3γ |
| ABD | none | CD3ε |
| ABD | none | FcγRI-γ |
| ABD | none | FcγRIII-γ |
| ABD | none | FcεRIβ |
| ABD | none | FcεRIγ |
| ABD | none | DAP10 |
| ABD | none | DAP12 |
| ABD | none | CD32 |
| ABD | none | CD79a |
| ABD | none | CD8 |
| ABD | none | CD3ζ |
| ABD | none | CD3δ |
| ABD | none | CD3γ |
| ABD | none | CD3ε |
| ABD | none | FcγRI-γ |

TABLE 5
CARs lacking Signal Domain (for dual CAR approach)

| ScFv | Co-stimulatory Signal | Signal Domain |
|---|---|---|
| ABD | CD28 | none |
| ABD | CD8 | none |
| ABD | CD4 | none |
| ABD | b2c | none |
| ABD | CD137/41BB | none |
| ABD | ICOS | none |
| ABD | CD27 | none |
| ABD | CD28δ | none |
| ABD | CD80 | none |
| ABD | CD86 | none |
| ABD | OX40 | none |
| ABD | DAP10 | none |
| ABD | MyD88 | none |
| ABD | CD7 | none |
| ABD | DAP12 | none |
| ABD | MyD88 | none |
| ABD | CD7 | none |
| ABD | BTNL3 | none |
| ABD | NKG2D | none |

TABLE 6
Third Generation CARs lacking Signal
Domain (for dual CAR approach)

| | Co-stimulatory | Co-stimulatory | Signal |
|---|---|---|---|
| ScFv | Signal | Signal | Domain |
| ABD | CD28 | CD28 | none |
| ABD | CD28 | CD8 | none |
| ABD | CD28 | CD4 | none |
| ABD | CD28 | b2c | none |
| ABD | CD28 | CD137/41BB | none |
| ABD | CD28 | ICOS | none |
| ABD | CD28 | CD27 | none |
| ABD | CD28 | CD28δ | none |
| ABD | CD28 | CD80 | none |
| ABD | CD28 | CD86 | none |
| ABD | CD28 | OX40 | none |
| ABD | CD28 | DAP10 | none |
| ABD | CD28 | MyD88 | none |
| ABD | CD28 | CD7 | none |
| ABD | CD28 | DAP12 | none |
| ABD | CD28 | MyD88 | none |
| ABD | CD28 | CD7 | none |
| ABD | CD8 | CD28 | none |
| ABD | CD8 | CD8 | none |
| ABD | CD8 | CD4 | none |
| ABD | CD8 | b2c | none |
| ABD | CD8 | CD137/41BB | none |
| ABD | CD8 | ICOS | none |
| ABD | CD8 | CD27 | none |
| ABD | CD8 | CD28δ | none |
| ABD | CD8 | CD80 | none |
| ABD | CD8 | CD86 | none |
| ABD | CD8 | OX40 | none |
| ABD | CD8 | DAP10 | none |
| ABD | CD8 | MyD88 | none |
| ABD | CD8 | CD7 | none |
| ABD | CD8 | DAP12 | none |
| ABD | CD8 | MyD88 | none |
| ABD | CD8 | CD7 | none |
| ABD | CD4 | CD28 | none |
| ABD | CD4 | CD8 | none |
| ABD | CD4 | CD4 | none |
| ABD | CD4 | b2c | none |
| ABD | CD4 | CD137/41BB | none |
| ABD | CD4 | ICOS | none |
| ABD | CD4 | CD27 | none |
| ABD | CD4 | CD28δ | none |
| ABD | CD4 | CD80 | none |
| ABD | CD4 | CD86 | none |
| ABD | CD4 | OX40 | none |
| ABD | CD4 | DAP10 | none |
| ABD | CD4 | MyD88 | none |
| ABD | CD4 | CD7 | none |
| ABD | CD4 | DAP12 | none |
| ABD | CD4 | MyD88 | none |
| ABD | CD4 | CD7 | none |
| ABD | b2c | CD28 | none |
| ABD | b2c | CD8 | none |
| ABD | b2c | CD4 | none |
| ABD | b2c | b2c | none |
| ABD | b2c | CD137/41BB | none |
| ABD | b2c | ICOS | none |
| ABD | b2c | CD27 | none |
| ABD | b2c | CD28δ | none |
| ABD | b2c | CD80 | none |
| ABD | b2c | CD86 | none |
| ABD | b2c | OX40 | none |
| ABD | b2c | DAP10 | none |
| ABD | b2c | MyD88 | none |
| ABD | b2c | CD7 | none |
| ABD | b2c | DAP12 | none |
| ABD | b2c | MyD88 | none |
| ABD | b2c | CD7 | none |
| ABD | CD137/41BB | CD28 | none |
| ABD | CD137/41BB | CD8 | none |
| ABD | CD137/41BB | CD4 | none |
| ABD | CD137/41BB | b2c | none |
| ABD | CD137/41BB | CD137/41BB | none |
| ABD | CD137/41BB | ICOS | none |
| ABD | CD137/41BB | CD27 | none |
| ABD | CD137/41BB | CD28δ | none |
| ABD | CD137/41BB | CD80 | none |
| ABD | CD137/41BB | CD86 | none |
| ABD | CD137/41BB | OX40 | none |
| ABD | CD137/41BB | DAP10 | none |
| ABD | CD137/41BB | MyD88 | none |
| ABD | CD137/41BB | CD7 | none |
| ABD | CD137/41BB | DAP12 | none |
| ABD | CD137/41BB | MyD88 | none |
| ABD | CD137/41BB | CD7 | none |
| ABD | ICOS | CD28 | none |
| ABD | ICOS | CD8 | none |
| ABD | ICOS | CD4 | none |
| ABD | ICOS | b2c | none |
| ABD | ICOS | CD137/41BB | none |
| ABD | ICOS | ICOS | none |
| ABD | ICOS | CD27 | none |
| ABD | ICOS | CD28δ | none |
| ABD | ICOS | CD80 | none |
| ABD | ICOS | CD86 | none |
| ABD | ICOS | OX40 | none |
| ABD | ICOS | DAP10 | none |
| ABD | ICOS | MyD88 | none |
| ABD | ICOS | CD7 | none |
| ABD | ICOS | DAP12 | none |
| ABD | ICOS | MyD88 | none |
| ABD | ICOS | CD7 | none |
| ABD | ICOS | CD28 | none |
| ABD | ICOS | CD8 | none |
| ABD | ICOS | CD4 | none |
| ABD | ICOS | b2c | none |
| ABD | ICOS | CD137/41BB | none |
| ABD | ICOS | ICOS | none |
| ABD | ICOS | CD27 | none |
| ABD | ICOS | CD28δ | none |
| ABD | ICOS | CD80 | none |
| ABD | ICOS | CD86 | none |
| ABD | ICOS | OX40 | none |
| ABD | ICOS | DAP10 | none |
| ABD | ICOS | MyD88 | none |
| ABD | ICOS | CD7 | none |
| ABD | ICOS | DAP12 | none |
| ABD | ICOS | MyD88 | none |
| ABD | ICOS | CD7 | none |
| ABD | CD27 | CD28 | none |
| ABD | CD27 | CD8 | none |
| ABD | CD27 | CD4 | none |
| ABD | CD27 | b2c | none |
| ABD | CD27 | CD137/41BB | none |
| ABD | CD27 | ICOS | none |
| ABD | CD27 | CD27 | none |
| ABD | CD27 | CD28δ | none |
| ABD | CD27 | CD80 | none |
| ABD | CD27 | CD86 | none |
| ABD | CD27 | OX40 | none |
| ABD | CD27 | DAP10 | none |
| ABD | CD27 | MyD88 | none |
| ABD | CD27 | CD7 | none |
| ABD | CD27 | DAP12 | none |
| ABD | CD27 | MyD88 | none |
| ABD | CD27 | CD7 | none |
| ABD | CD28δ | CD28 | none |
| ABD | CD28δ | CD8 | none |
| ABD | CD28δ | CD4 | none |
| ABD | CD28δ | b2c | none |
| ABD | CD28δ | CD137/41BB | none |
| ABD | CD28δ | ICOS | none |
| ABD | CD28δ | CD27 | none |
| ABD | CD28δ | CD28δ | none |
| ABD | CD28δ | CD80 | none |
| ABD | CD28δ | CD86 | none |
| ABD | CD28δ | OX40 | none |
| ABD | CD28δ | DAP10 | none |
| ABD | CD28δ | MyD88 | none |
| ABD | CD28δ | CD7 | none |
| ABD | CD28δ | DAP12 | none |
| ABD | CD28δ | MyD88 | none |
| ABD | CD28δ | CD7 | none |
| ABD | CD80 | CD28 | none |
| ABD | CD80 | CD8 | none |
| ABD | CD80 | CD4 | none |
| ABD | CD80 | b2c | none |
| ABD | CD80 | CD137/41BB | none |
| ABD | CD80 | ICOS | none |
| ABD | CD80 | CD27 | none |
| ABD | CD80 | CD28δ | none |
| ABD | CD80 | CD80 | none |
| ABD | CD80 | CD86 | none |
| ABD | CD80 | OX40 | none |
| ABD | CD80 | DAP10 | none |
| ABD | CD80 | MyD88 | none |
| ABD | CD80 | CD7 | none |
| ABD | CD80 | DAP12 | none |
| ABD | CD80 | MyD88 | none |
| ABD | CD80 | CD7 | none |
| ABD | CD86 | CD28 | none |
| ABD | CD86 | CD8 | none |
| ABD | CD86 | CD4 | none |
| ABD | CD86 | b2c | none |
| ABD | CD86 | CD137/41BB | none |
| ABD | CD86 | ICOS | none |
| ABD | CD86 | CD27 | none |
| ABD | CD86 | CD28δ | none |
| ABD | CD86 | CD80 | none |
| ABD | CD86 | CD86 | none |
| ABD | CD86 | OX40 | none |
| ABD | CD86 | DAP10 | none |
| ABD | CD86 | MyD88 | none |
| ABD | CD86 | CD7 | none |
| ABD | CD86 | DAP12 | none |
| ABD | CD86 | MyD88 | none |
| ABD | CD86 | CD7 | none |
| ABD | OX40 | CD28 | none |
| ABD | OX40 | CD8 | none |
| ABD | OX40 | CD4 | none |
| ABD | OX40 | b2c | none |
| ABD | OX40 | CD137/41BB | none |
| ABD | OX40 | ICOS | none |
| ABD | OX40 | CD27 | none |
| ABD | OX40 | CD28δ | none |
| ABD | OX40 | CD80 | none |
| ABD | OX40 | CD86 | none |
| ABD | OX40 | OX40 | none |
| ABD | OX40 | DAP10 | none |
| ABD | OX40 | MyD88 | none |
| ABD | OX40 | CD7 | none |
| ABD | OX40 | DAP12 | none |
| ABD | OX40 | MyD88 | none |
| ABD | OX40 | CD7 | none |
| ABD | DAP10 | CD28 | none |
| ABD | DAP10 | CD8 | none |
| ABD | DAP10 | CD4 | none |
| ABD | DAP10 | b2c | none |
| ABD | DAP10 | CD137/41BB | none |
| ABD | DAP10 | ICOS | none |
| ABD | DAP10 | CD27 | none |
| ABD | DAP10 | CD28δ | none |
| ABD | DAP10 | CD80 | none |
| ABD | DAP10 | CD86 | none |
| ABD | DAP10 | OX40 | none |
| ABD | DAP10 | DAP10 | none |
| ABD | DAP10 | MyD88 | none |
| ABD | DAP10 | CD7 | none |
| ABD | DAP10 | DAP12 | none |
| ABD | DAP10 | MyD88 | none |
| ABD | DAP10 | CD7 | none |
| ABD | DAP12 | CD28 | none |
| ABD | DAP12 | CD8 | none |
| ABD | DAP12 | CD4 | none |
| ABD | DAP12 | b2c | none |
| ABD | DAP12 | CD137/41BB | none |
| ABD | DAP12 | ICOS | none |
| ABD | DAP12 | CD27 | none |
| ABD | DAP12 | CD28δ | none |
| ABD | DAP12 | CD80 | none |
| ABD | DAP12 | CD86 | none |
| ABD | DAP12 | OX40 | none |
| ABD | DAP12 | DAP10 | none |
| ABD | DAP12 | MyD88 | none |
| ABD | DAP12 | CD7 | none |
| ABD | DAP12 | DAP12 | none |
| ABD | DAP12 | MyD88 | none |
| ABD | DAP12 | CD7 | none |
| ABD | MyD88 | CD28 | none |
| ABD | MyD88 | CD8 | none |
| ABD | MyD88 | CD4 | none |
| ABD | MyD88 | b2c | none |
| ABD | MyD88 | CD137/41BB | none |
| ABD | MyD88 | ICOS | none |
| ABD | MyD88 | CD27 | none |
| ABD | MyD88 | CD28δ | none |
| ABD | MyD88 | CD80 | none |
| ABD | MyD88 | CD86 | none |
| ABD | MyD88 | OX40 | none |
| ABD | MyD88 | DAP10 | none |
| ABD | MyD88 | MyD88 | none |
| ABD | MyD88 | CD7 | none |
| ABD | MyD88 | DAP12 | none |
| ABD | MyD88 | MyD88 | none |
| ABD | MyD88 | CD7 | none |
| ABD | CD7 | CD28 | none |
| ABD | CD7 | CD8 | none |
| ABD | CD7 | CD4 | none |
| ABD | CD7 | b2c | none |
| ABD | CD7 | CD137/41BB | none |
| ABD | CD7 | ICOS | none |
| ABD | CD7 | CD27 | none |
| ABD | CD7 | CD28δ | none |
| ABD | CD7 | CD80 | none |
| ABD | CD7 | CD86 | none |
| ABD | CD7 | OX40 | none |
| ABD | CD7 | DAP10 | none |
| ABD | CD7 | MyD88 | none |
| ABD | CD7 | CD7 | none |
| ABD | CD7 | DAP12 | none |
| ABD | CD7 | MyD88 | none |
| ABD | CD7 | CD7 | none |
| ABD | BTNL3 | CD28 | none |
| ABD | BTNL3 | CD8 | none |
| ABD | BTNL3 | CD4 | none |
| ABD | BTNL3 | b2c | none |
| ABD | BTNL3 | CD137/41BB | none |
| ABD | BTNL3 | ICOS | none |
| ABD | BTNL3 | CD27 | none |
| ABD | BTNL3 | CD28δ | none |
| ABD | BTNL3 | CD80 | none |
| ABD | BTNL3 | CD86 | none |
| ABD | BTNL3 | OX40 | none |
| ABD | BTNL3 | DAP10 | none |
| ABD | BTNL3 | MyD88 | none |
| ABD | BTNL3 | CD7 | none |
| ABD | BTNL3 | DAP12 | none |
| ABD | BTNL3 | MyD88 | none |
| ABD | BTNL3 | CD7 | none |
| ABD | NKG2D | CD28 | none |
| ABD | NKG2D | CD8 | none |
| ABD | NKG2D | CD4 | none |
| ABD | NKG2D | b2c | none |
| ABD | NKG2D | CD137/41BB | none |
| ABD | NKG2D | ICOS | none |
| ABD | NKG2D | CD27 | none |
| ABD | NKG2D | CD28δ | none |
| ABD | NKG2D | CD80 | none |
| ABD | NKG2D | CD86 | none |
| ABD | NKG2D | OX40 | none |
| ABD | NKG2D | DAP10 | none |
| ABD | NKG2D | MyD88 | none |
| ABD | NKG2D | CD7 | none |
| ABD | NKG2D | DAP12 | none |
| ABD | NKG2D | MyD88 | none |
| ABD | NKG2D | CD7 | none |

In some embodiments, the anti-CD33 or anti-CD123 binding agent is single chain variable fragment (scFv) antibody. The affinity/specificity of an anti-CD33 scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the anti-CD33 or anti-CD123 binding agent is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

The disclosed immune effector cells contain at least one addition CAR that binds a different antigen, such as a tumor antigen. Tumor antigens are proteins that are produced by tumor cells that elicit an immune response, particularly T-cell mediated immune responses. The additional antigen binding domain can be an antibody or a natural ligand of the tumor antigen. The selection of the additional antigen binding domain will depend on the particular type of cancer to be treated. Tumor antigens are well known in the art and include, for example, a glioma-associated antigen, carcinoembryonic antigen (CEA), EGFRvIII, IL-IIRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, CD123, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-Ia, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6, E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, TIM3, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvIII, IL-13Ra, CD123, CD19, TIM3, BCMA, GD2, CLL-1, CA-IX, MUCI, HER2, and any combination thereof.

Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG1 6, TA-90\Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

### CD99 CAR

The anti-CD99 binding agent is in some embodiments an antibody fragment that specifically binds CD99. For example, the antigen binding domain can be a Fab or a single-chain variable fragment (scFv) of an antibody that specifically binds CD99. The anti-CD99 binding agent is in some embodiments an aptamer that specifically binds CD99. For example, the anti-CD99 binding agent can be a peptide aptamer selected from a random sequence pool based on its ability to bind CD99. The anti-CD99 binding agent can also be a natural ligand of CD99, or a variant and/or fragment thereof capable of binding CD99.

In some embodiments, the anti-CD99 region of the disclosed antibody or CAR is derived from hybridoma 1H3, 4C5, 9G12, 3C7, 2F11, 4D5, 4F4, 6A10, or combinations thereof. In some embodiments, the anti-CD99 region (e.g. scFv) can comprise a variable heavy (V_{H}) domain having CDR1, CDR2 and CDR3 sequences and a variable light (V_{L}) domain having CDR1, CDR2 and CDR3 sequences.

In some embodiments, the CDR1 sequence of the V_{H} domain comprises the amino acid sequence GFDIKDTY (SEQ ID NO:86), TYAMY (SEQ ID NO:87), TFWM (SEQ ID NO:88), or TFWMQ (SEQ ID NO:89); the CDR2 sequence of the V_{H} domain comprises the amino acid sequence IDPANGDT (SEQ ID NO:90), RIRSKVNNYATYYADSVKDRFT (SEQ ID NO:91), or TIYPGDDDTRYTQKFKGRAT (SEQ ID NO:92); the CDR3 sequence of the V_{H} domain comprises the amino acid sequence ARRGGLS (SEQ ID NO:93), DPMDY (SEQ ID NO:94), or SGYERGPYYFDS (SEQ ID NO:95), or SGYERGPYYF (SEQ ID NO:96); the CDR1 sequence of the V_{L} comprises the amino acid sequence GNIHNY (SEQ ID NO:97), GSSKSLLHSNGNTYLY (SEQ ID NO:98), KSSQSLLCRSNQKNYLA (SEQ ID NO:99), or KSSQSLLYRSNQKNYLA (SEQ ID NO:100); the CDR2 sequence of the V_{L} domain comprises the amino acid sequence NAK, RVSNLAS (SEQ ID NO:101), or WASTRES (SEQ ID NO:102); and the CDR3 sequence of the V_{L} domain comprises the amino acid sequence QHFWSTPWT (SEQ ID NO: 103), MQHLEYPYT (SEQ ID NO:104), or QQYYSYPLT (SEQ ID NO:105).

Therefore, in some embodiments, the anti-CD99 V_{H} domain comprises the amino acid sequence

(SEQ ID NO: 106, 1H3H7, 1H3H9)
EVQLQQSGAELVKPGASVKLSCTASGFDIKDTYIHWVKQRPEQGLEWIG
RIDPANGDTRYDPEFQGKASLTADTSSNTAYLQFSNLTSEDTAVYYCAR
RGGLSWGQGTTLTVSS.

Therefore, in some embodiments, the anti-CD99 V_{H} domain comprises the amino acid sequence:

(SEQ ID NO: 107, 4C5E2)
EVQLEESGGGLVQPKGSLKLSCAASGFTFNTYAMYWVCQAPGKGLKWVA
RIRSKVNNYATYYADSVKDRFTISRDDSQNMLFLHMNNLKTEDTAIYFC
VRDPMDYWGQGISVTVSS.

Therefore, in some embodiments, the anti-CD99 V_{H} domain comprises the amino acid sequence:

(SEQ ID NO: 108, 4C5H10)
EVQLVESGGGLVQPKGSLKLSCAASGFTFNTYAMYWVCQAPGKGLKWVA
RIRSKVNNYATYYADSVKDRFTISRDDSQNMLFLHMNNLKTEDTAIYFC
VRDPMDYWGQGISVTVSS.

Therefore, in some embodiments, the anti-CD99 V_{H} domain comprises the amino acid sequence:

(SEQ ID NO: 109, 9G12C9)
QVQLQQSGAELARPGASVKLSCKASGYTFTTFWMQWVKQRPGQGLEWIG
TIYPGDDDTRYTQKFKGRATLTADKSSTTAYMQLSNLSSEDSAVYYCAR
SGYERGPYYFDSWGQGTTLTVSS.

Therefore, in some embodiments, the anti-CD99 V_{H} domain comprises the amino acid sequence:

(SEQ ID NO: 110, 9G12G6 HB1)
DVKLQESGAELARPGASVKLSCKASGYTFTTFWMQRVKQRPGQGLEWIG
TIYPGDDDTRYTQKFKGRATLTADKSSTTAYMQLSNLSSEDSAVYYCAR
SGYERGPYYFDSWGQGTTLTVSS.

In some embodiments, the anti-CD99 V_{H} domain comprises the amino acid sequence:

(SEQ ID NO: 111, 9G12G6 HB3)
QVQLKESGAELARPGASVKLSCKASGYTFTTFWMQWAKQRPGQGLEWIG
TIYPGDDDTRYTQKFKGRATLTADKSSTTAYMQLSNLSSEDSAVYYCAR
SGYERGPYYFDSWGQGTTLTVSS.

In some embodiments, the anti-CD99 V_{L} domain comprises the amino acid sequence:

(SEQ ID NO: 112, 1H3H9)
DIQMTQSPASLSASVGETVTITCRASGNIHNYLAWYQQKQGKSPQLLVY
NAKTLADGVPSRFSGSGSGTQYSLKINSLQPEDFGSYYCQHFWSTPWTF
GGGTKLEIK.

In some embodiments, the anti-CD99 V_{L} domain comprises the amino acid sequence:

(SEQ ID NO: 113, 1H3H7 LC1)
GNSWSHSLRSLSVTIGQPASISCKSSQSLLDGNGKTYLNWLLQRPGQSP
KRLLYLVSKLDSGVPDRFTGSGSGTDFTLKISRVEAEDLGIYYCWQGTH
FPRTFGGGTKLEIK.

In some embodiments, the anti-CD99 V_{L} domain comprises the amino acid sequence:

(SEQ ID NO: 114, 1H3H7 LC2)
GNSWRHSPRSLSVTIGQPASISCKSSQSLLDGNGKTYLNWLLQRPGQSP
KRLLYLVSKLDSGVPDRFTGSGSGTDFTLKISRVEAEDLGIYYCWQGTH
FPRTFGGGTKLEIK.

In some embodiments, the anti-CD99 V_{L} domain comprises the amino acid sequence:

(SEQ ID NO: 115, 4C5E2)
DIVMTQAAPSVPVTPGESVSISCGSSKSLLHSNGNTYLYWFLQRPGQSP
QLLIYRVSNLASGVPDRFSGSGSGTAFTLRISRVEAEDVGVYYCMQHLE
YPYTFGGGTRLEIK.

In some embodiments, the anti-CD99 V_{L} domain comprises the amino acid sequence:

(SEQ ID NO: 116, 4C5H10)
DIVMTQAAPSVPVTPGESVSISCGSSKSLLHSNGNTYLYWFLQRPGQSP
QLLIYRVSNLASGVPDRFSGSGSGTAFTLRISRVEAEDVGVYYCMQHLE
YPYTFGGGTRLEIK.

In some embodiments, the anti-CD99 V_{L} domain comprises the amino acid sequence:

(SEQ ID NO: 117, 9G12C9)
DTVMSQSPSSLAVSVGEKITMSCKSSQSLLCRSNQKNYLAWYQQKPGQS
PKQLIYWASTRESGVPDRFTGSGSGTDFTLTISSVKAEDLAVYYCQQYY
SYPLTFGAGTKLELK.

In some embodiments, the anti-CD99 V_{L} domain comprises the amino acid sequence:

(SEQ ID NO: 118, 9G12G6)
DTVMSQSPSSLAVSVGEKITMSCKSSQSLLYRSNQKNYLAWYQQK
PGQSPKQLIYWASTRESGVPDRFTGSGSGTDFTLTISSVKAEDLA
VYYCQQYYSYPLTFGAGTKLELK.

In some embodiments, the anti-CD99 scFv comprises an amino acid sequence:

(SEQ ID NO: 119, 1H3H9 v1)
EVQLQQSGAELVKPGASVKLSCTASGFDIKDTYIHWVKQRPEQGL
EWIGRIDPANGDTRYDPEFQGKASLTADTSSNTAYLQFSNLTSED
TAVYYCARRGGLSWGQGTTLTVSSGGGGSGGGGSGGGGSDIQMTQ
SPASLSASVGETVTITCRASGNIHNYLAWYQQKQGKSPQLLVYNA
KTLADGVPSRFSGSGSGTQYSLKINSLQPEDFGSYYCQHFWSTPW
TFGGGTKLEIK.

In some embodiments, the anti-CD99 scFv comprises an amino acid sequence:

(SEQ ID NO: 120)
EVQLQQSGAELVKPGASVKLSCTASGFDIKDTYIHWKQRPEQGLE
WIGRIDPANGDTRYDPEFQGKASLTADTSSNTAYLQFSNLTSEDT
AVYYCARRGGLSWGQGTTLTVSSGGGGSGGGGSGGGGSGNSWSHS
LRSLSVTIGQPASISCKSSQSLLDGNGKTYLNWLLQRPGQSPKRL
LYLVSKLDSGVPDRFTGSGSGTDFTLKISRVEAEDLGIYYCWQGT
HFPRTFGGGTKLEIK.

In some embodiments, the anti-CD99 scFv comprises an amino acid sequence:

(SEQ ID NO: 121)
EVQLQQSGAELVKPGASVKLSCTASGFDIKDTYIHWVKQRPEQGL
EWIGRIDPANGDTRYDPEFQGKASLTADTSSNTAYLQFSNLTSED
TAVYYCARRGGLSWGQGTTLTVSSGGGGSGGGGSGGGGSGNSWRH
SPRSLSVTIGQPASISCKSSQSLLDGNGKTYLNWLLQRPGQSPKR
LLYLVSKLDSGVPDRFTGSGSGTDFTLKISRVEAEDLGIYYCWQG
THFPRTFGGGTKLEIK.

In some embodiments, the anti-CD99 scFv comprises an amino acid sequence:

(SEQ ID NO: 122)
EVQLQQSGAELVKPGASVKLSCTASGFDIKDTYIHWVKQRPEQGL
EWIGRIDPANGDTRYDPEFQGKASLTADTSSNTAYLQFSNLTSED
TAVYYCARRGGLSWGQGTTLTVSSGGGGSGGGGSGGGGSDIVMTQ
AAPSVPVTPGESVSISCGSSKSLLHSNGNTYLYWFLQRPGQSPQL
LIYRVSNLASGVPDRFSGSGSGTAFTLRISRVEAEDVGVYYCMQH
LEYPYTFGGGTRLEIK.

In some embodiments, the anti-CD99 scFv comprises an amino acid sequence:

(SEQ ID NO: 123)
EVQLQQSGAELVKPGASVKLSCTASGFDIKDTYIHWVKQRPEQGL
EWIGRIDPANGDTRYDPEFQGKASLTADTSSNTAYLQFSNLTSED
TAVYYCARRGGLSWGQGTTLTVSSGGGGSGGGGSGGGGSDTVMSQ
SPSSLAVSVGEKITMSCKSSQSLLCRSNQKNYLAWYQQKPGQSPK
QLIYWASTRESGVPDRFTGSGSGTDFTLTISSVKAEDLAVYYCQQ
YYSYPLTFGAGTKLELK.

In some embodiments, the anti-CD99 scFv comprises an amino acid sequence:

(SEQ ID NO: 124)
EVQLQQSGAELVKPGASVKLSCTASGFDIKDTYIHWVKQRPEQGL
EWIGRIDPANGDTRYDPEFQGKASLTADTSSNTAYLQFSNLTSED
TAVYYCARRGGLSWGQGTTLTVSSGGGGSGGGGSGGGGSDTVMSQ
SPSSLAVSVGEKITMSCKSSQSLLYRSNQKNYLAWYQQKPGQSPK
QLIYWASTRESGVPDRFTGSGSGTDFTLTISSVKAEDLAVYYCQQ
YYSYPLTFGAGTKLELK.

In some embodiments, the anti-CD99 scFv comprises an amino acid sequence:

(SEQ ID NO: 125)
EVQLEESGGGLVQPKGSLKLSCAASGFTFNTYAMYWVCQAPGKGL
KWVARIRSKVNNYATYYADSVKDRFTISRDDSQNMLFLHMNNLKT
EDTAIYFCVRDPMDYWGQGISVTVSSGGGGSGGGGSGGGGSDIQM
TQSPASLSASVGETVTITCRASGNIHNYLAWYQQKQGKSPQLLVY
NAKTLADGVPSRFSGSGSGTQYSLKINSLQPEDFGSYYCQHFWST
PWTFGGGTKLEIK.

In some embodiments, the anti-CD99 scFv comprises an amino acid sequence:

(SEQ ID NO: 126)
EVQLEESGGGLVQPKGSLKLSCAASGFTFNTYAMYWVCQAPGKGL
KWVARIRSKVNNYATYYADSVKDRFTISRDDSQNMLFLHMNNLKT
EDTAIYFCVRDPMDYWGQGISVTVSSGGGGSGGGGSGGGGSGNSW
SHSLRSLSVTIGQPASISCKSSQSLLDGNGKTYLNWLLQRPGQSP
KRLLYLVSKLDSGVPDRFTGSGSGTDFTLKISRVEAEDLGIYYCW
QGTHFPRTFGGGTKLEIK.

In some embodiments, the anti-CD99 scFv comprises an amino acid sequence:

(SEQ ID NO: 127)
EVQLEESGGGLVQPKGSLKLSCAASGFTFNTYAMYWVCQAPGKGL
KWVARIRSKVNNYATYYADSVKDRFTISRDDSQNMLFLHMNNLKT
EDTAIYFCVRDPMDYWGQGISVTVSSGGGGSGGGGSGGGGSGNSW
RHSPRSLSVTIGQPASISCKSSQSLLDGNGKTYLNWLLQRPGQSP
KRLLYLVSKLDSGVPDRFTGSGSGTDFTLKISRVEAEDLGIYYCW
QGTHFPRTFGGGTKLEIK.

In some embodiments, the anti-CD99 scFv comprises an amino acid sequence:

(SEQ ID NO: 128, 4C5E2 v1)
EVQLEESGGGLVQPKGSLKLSCAASGFTFNTYAMYWVCQAPGKGL
KWVARIRSKVNNYATYYADSVKDRFTISRDDSQNMLFLHMNNLKT
EDTAIYFCVRDPMDYWGQGISVTVSSGGGGSGGGGSGGGGSDIVM
TQAAPSVPVTPGESVSISCGSSKSLLHSNGNTYLYWFLQRPGQSP
QLLIYRVSNLASGVPDRFSGSGSGTAFTLRISRVEAEDVGVYYCM
QHLEYPYTFGGGTRLEIK.

In some embodiments, the anti-CD99 scFv comprises an amino acid sequence:

(SEQ ID NO: 129)
EVQLEESGGGLVQPKGSLKLSCAASGFTFNTYAMYWVCQAPGKGL
KWVARIRSKVNNYATYYADSVKDRFTISRDDSQNMLFLHMNNLKT
EDTAIYFCVRDPMDYWGQGISVTVSSGGGGSGGGGSGGGGSDTVM
SQSPSSLAVSVGEKITMSCKSSQSLLCRSNQKNYLAWYQQKPGQS
PKQLIYWASTRESGVPDRFTGSGSGTDFTLTISSVKAEDLAVYYC
QQYYSYPLTFGAGTKLELK.

In some embodiments, the anti-CD99 scFv comprises an amino acid sequence:

(SEQ ID NO: 130)
EVQLEESGGGLVQPKGSLKLSCAASGFTFNTYAMYWVCQAPGKGL
KWVARIRSKVNNYATYYADSVKDRFTISRDDSQNMLFLHMNNLKT
EDTAIYFCVRDPMDYWGQGISVTVSSGGGGSGGGGSGGGGSDTVM
SQSPSSLAVSVGEKITMSCKSSQSLLYRSNQKNYLAWYQQKPGQS
PKQLIYWASTRESGVPDRFTGSGSGTDFTLTISSVKAEDLAVYYC
QQYYSYPLTFGAGTKLELK.

In some embodiments, the anti-CD99 scFv comprises an amino acid sequence:

(SEQ ID NO: 131)
EVQLVESGGGLVQPKGSLKLSCAASGFTFNTYAMYWVCQAPGKGL
KWVARIRSKVNNYATYYADSVKDRFTISRDDSQNMLFLHMNNLKT
EDTAIYFCVRDPMDYWGQGISVTVSSGGGGSGGGGSGGGGSDIQM
TQSPASLSASVGETVTITCRASGNIHNYLAWYQQKQGKSPQLLVY
NAKTLADGVPSRFSGSGSGTQYSLKINSLQPEDFGSYYCQHFWST
PWTFGGGTKLEIK.

In some embodiments, the anti-CD99 scFv comprises an amino acid sequence:

(SEQ ID NO: 132)
EVQLVESGGGLVQPKGSLKLSCAASGFTFNTYAMYWVCQAPGKGL
KWVARIRSKVNNYATYYADSVKDRFTISRDDSQNMLFLHMNNLKT
EDTAIYFCVRDPMDYWGQGISVTVSSGGGGSGGGGSGGGGSGNSW
SHSLRSLSVTIGQPASISCKSSQSLLDGNGKTYLNWLLQRPGQSP
KRLLYLVSKLDSGVPDRFTGSGSGTDFTLKISRVEAEDLGIYYCW
QGTHFPRTFGGGTKLEIK.

In some embodiments, the anti-CD99 scFv comprises an amino acid sequence:

(SEQ ID NO: 133)
EVQLVESGGGLVQPKGSLKLSCAASGFTFNTYAMYWVCQAPGKGL
KWVARIRSKVNNYATYYADSVKDRFTISRDDSQNMLFLHMNNLKT
EDTAIYFCVRDPMDYWGQGISVTVSSGGGGSGGGGSGGGGSGNSW
RHSPRSLSVTIGQPASISCKSSQSLLDGNGKTYLNWLLQRPGQSP
KRLLYLVSKLDSGVPDRFTGSGSGTDFTLKISRVEAEDLGIYYCW
QGTHFPRTFGGGTKLEIK.

In some embodiments, the anti-CD99 scFv comprises an amino acid sequence:

(SEQ ID NO: 134, 4C5H10 v1)
EVQLVESGGGLVQPKGSLKLSCAASGFTFNTYAMYWVCQAPGKGL
KWVARIRSKVNNYATYYADSVKDRFTISRDDSQNMLFLHMNNLKT
EDTAIYFCVRDPMDYWGQGISVTVSSGGGGSGGGGSGGGGSDIVM
TQAAPSVPVTPGESVSISCGSSKSLLHSNGNTYLYWFLQRPGQSP
QLLIYRVSNLASGVPDRFSGSGSGTAFTLRISRVEAEDVGVYYCM
QHLEYPYTFGGGTRLEIK.

In some embodiments, the anti-CD99 scFv comprises an amino acid sequence:

(SEQ ID NO: 135)
EVQLVESGGGLVQPKGSLKLSCAASGFTFNTYAMYWVCQAPGKGL
KWVARIRSKVNNYATYYADSVKDRFTISRDDSQNMLFLHMNNLKT
EDTAIYFCVRDPMDYWGQGISVTVSSGGGGSGGGGSGGGGSDTVM
SQSPSSLAVSVGEKITMSCKSSQSLLCRSNQKNYLAWYQQKPGQS
PKQLIYWASTRESGVPDRFTGSGSGTDFTLTISSVKAEDLAVYYC
QQYYSYPLTFGAGTKLELK.

In some embodiments, the anti-CD99 scFv comprises an amino acid sequence:

(SEQ ID NO: 136)
EVQLVESGGGLVQPKGSLKLSCAASGFTFNTYAMYWVCQAPGKGL
KWVARIRSKVNNYATYYADSVKDRFTISRDDSQNMLFLHMNNLKT
EDTAIYFCVRDPMDYWGQGISVTVSSGGGGSGGGGSGGGGSDTVM
SQSPSSLAVSVGEKITMSCKSSQSLLYRSNQKNYLAWYQQKPGQS
PKQLIYWASTRESGVPDRFTGSGSGTDFTLTISSVKAEDLAVYYC
QQYYSYPLTFGAGTKLELK.

In some embodiments, the anti-CD99 scFv comprises an amino acid sequence:

(SEQ ID NO: 137)
QVQLQQSGAELARPGASVKLSCKASGYTFTTFWMQWVKQRPGQGL
EWIGTIYPGDDDTRYTQKFKGRATLTADKSSTTAYMQLSNLSSED
SAVYYCARSGYERGPYYFDSWGQGTTLTVSSGGGGGGGGSGGGGS
DIQMTQSPASLSASVGETVTITCRASGNIHNYLAWYQQKQGKSPQ
LLVYNAKTLADGVPSRFSGSGSGTQYSLKINSLQPEDFGSYYCQH
FWSTPWTFGGGTKLEIK.

In some embodiments, the anti-CD99 scFv comprises an amino acid sequence:

(SEQ ID NO: 138)
QVQLQQSGAELARPGASVKLSCKASGYTFTTFWMQWVKQRPGQGL
EWIGTIYPGDDDTRYTQKFKGRATLTADKSSTTAYMQLSNLSSED
SAVYYCARSGYERGPYYFDSWGQGTTLTVSSGGGGSGGGGSGGGG
SGNSWSHSLRSLSVTIGQPASISCKSSQSLLDGNGKTYLNWLLQR
PGQSPKRLLYLVSKLDSGVPDRFTGSGSGTDFTLKISRVEAEDLG
IYYCWQGTHFPRTFGGGTKLEIK.

In some embodiments, the anti-CD99 scFv comprises an amino acid sequence:

(SEQ ID NO: 139)
QVQLQQSGAELARPGASVKLSCKASGYTFTTFWMQWVKQRPGQGL
EWIGTIYPGDDDTRYTQKFKGRATLTADKSSTTAYMQLSNLSSED
SAVYYCARSGYERGPYYFDSWGQGTTLTVSSGGGGSGGGGSGGGG
SGNSWRHSPRSLSVTIGQPASISCKSSQSLLDGNGKTYLNWLLQR
PGQSPKRLLYLVSKLDSGVPDRFTGSGSGTDFTLKISRVEAEDLG
IYYCWQGTHFPRTFGGGTKLEIK.

In some embodiments, the anti-CD99 scFv comprises an amino acid sequence:

(SEQ ID NO: 140)
QVQLQQSGAELARPGASVKLSCKASGYTFTTFWMQWVKQRPGQGL
EWIGTIYPGDDDTRYTQKFKGRATLTADKSSTTAYMQLSNLSSED
SAVYYCARSGYERGPYYFDSWGQGTTLTVSSGGGGSGGGGSGGGG
SDIVMTQAAPSVPVTPGESVSISCGSSKSLLHSNGNTYLYWFLQR
PGQSPQLLIYRVSNLASGVPDRFSGSGSGTAFTLRISRVEAEDVG
VYYCMQHLEYPYTFGGGTRLEIK.

In some embodiments, the anti-CD99 scFv comprises an amino acid sequence:

(SEQ ID NO: 141, 9G12C9 v1)
QVQLQQSGAELARPGASVKLSCKASGYTFTTFWMQWVKQRPGQGL
EWIGTIYPGDDDTRYTQKFKGRATLTADKSSTTAYMQLSNLSSED
SAVYYCARSGYERGPYYFDSWGQGTTLTVSSGGGGSGGGGSGGGG
SDTVMSQSPSSLAVSVGEKITMSCKSSQSLLCRSNQKNYLAWYQQ
KPGQSPKQLIYWASTRESGVPDRFTGSGSGTDFTLTISSVKAEDL
AVYYCQQYYSYPLTFGAGTKLELK.

In some embodiments, the anti-CD99 scFv comprises an amino acid sequence:

(SEQ ID NO: 142)
QVQLQQSGAELARPGASVKLSCKASGYTFTTFWMQWVKQRPGQGL
EWIGTIYPGDDDTRYTQKFKGRATLTADKSSTTAYMQLSNLSSED
SAVYYCARSGYERGPYYFDSWGQGTTLTVSSGGGGSGGGGSGGGG
SDTVMSQSPSSLAVSVGEKITMSCKSSQSLLYRSNQKNYLAWYQQ
KPGQSPKQLIYWASTRESGVPDRFTGSGSGTDFTLTISSVKAEDL
AVYYCQQYYSYPLTFGAGTKLELK.

In some embodiments, the anti-CD99 scFv comprises an amino acid sequence:

(SEQ ID NO: 143)
DVKLQESGAELARPGASVKLSCKASGYTFTTFWMQRVKQRPGQGL
EWIGTIYPGDDDTRYTQKFKGRATLTADKSSTTAYMQLSNLSSED
SAVYYCARSGYERGPYYFDSWGQGTTLTVSSGGGGSGGGGSGGGG
SDIQMTQSPASLSASVGETVTITCRASGNIHNYLAWYQQKQGKSP
QLLVYNAKTLADGVPSRFSGSGSGTQYSLKINSLQPEDFGSYYCQ
HFWSTPWTFGGGTKLEIK.

In some embodiments, the anti-CD99 scFv comprises an amino acid sequence:

(SEQ ID NO: 144)
DVKLQESGAELARPGASVKLSCKASGYTFTTFWMQRVKQRPGQGL
EWIGTIYPGDDDTRYTQKFKGRATLTADKSSTTAYMQLSNLSSED
SAVYYCARSGYERGPYYFDSWGQGTTLTVSSGGGGSGGGGSGGGG
SGNSWSHSLRSLSVTIGQPASISCKSSQSLLDGNGKTYLNWLLQR
PGQSPKRLLYLVSKLDSGVPDRFTGSGSGTDFTLKISRVEAEDLG
IYYCWQGTHFPRTFGGGTKLEIK.

In some embodiments, the anti-CD99 scFv comprises an amino acid sequence:

(SEQ ID NO: 145)
DVKLQESGAELARPGASVKLSCKASGYTFTTFWMQRVKQRPGQGL
EWIGTIYPGDDDTRYTQKFKGRATLTADKSSTTAYMQLSNLSSED
SAVYYCARSGYERGPYYFDSWGQGTTLTVSSGGGGSGGGGSGGGG
SGNSWRHSPRSLSVTIGQPASISCKSSQSLLDGNGKTYLNWLLQR
PGQSPKRLLYLVSKLDSGVPDRFTGSGSGTDFTLKISRVEAEDLG
IYYCWQGTHFPRTFGGGTKLEIK.

In some embodiments, the anti-CD99 scFv comprises an amino acid sequence:

(SEQ ID NO: 146)
DVKLQESGAELARPGASVKLSCKASGYTFTTFWMQRVKQRPGQGL
EWIGTIYPGDDDTRYTQKFKGRATLTADKSSTTAYMQLSNLSSED
SAVYYCARSGYERGPYYFDSWGQGTTLTVSSGGGGSGGGGSGGGG
SDIVMTQAAPSVPVTPGESVSISCGSSKSLLHSNGNTYLYWFLQR
PGQSPQLLIYRVSNLASGVPDRFSGSGSGTAFTLRISRVEAEDVG
VYYCMQHLEYPYTFGGGTRLEIK.

In some embodiments, the anti-CD99 scFv comprises an amino acid sequence:

(SEQ ID NO: 147)
DVKLQESGAELARPGASVKLSCKASGYTFTTFWMQRVKQRPGQGL
EWIGTIYPGDDDTRYTQKFKGRATLTADKSSTTAYMQLSNLSSED
SAVYYCARSGYERGPYYFDSWGQGTTLTVSSGGGGSGGGGSGGGG
SDIVMTQAAPSVPVTPGESVSISCGSSKSLLHSNGNTYLYWFLQR
PGQSPQLLIYRVSNLASGVPDRFSGSGSGTAFTLRISRVEAEDVG
VYYCMQHLEYPYTFGGGTRLEIK.

In some embodiments, the anti-CD99 scFv comprises an amino acid sequence:

(SEQ ID NO: 148)
DVKLQESGAELARPGASVKLSCKASGYTFTTFWMQRVKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDTVMSQSPSSLAVS
VGEKITMSCKSSQSLLCRSNQKNYLAWYQQKPGQSPKQLIYWASTRESGV
PDRFTGSGSGTDFTLTISSVKAEDLAVYYCQQYYSYPLTFGAGTKLELK.

In some embodiments, the anti-CD99 scFv comprises an amino acid sequence:

(SEQ ID NO: 149)
DVKLQESGAELARPGASVKLSCKASGYTFTTFWMQRVKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDTVMSQSPSSLAVS
VGEKITMSCKSSQSLLYRSNQKNYLAWYQQKPGQSPKQLIYWASTRESGV
PDRFTGSGSGTDFTLTISSVKAEDLAVYYCQQYYSYPLTFGAGTKLELK.

In some embodiments, the anti-CD99 scFv comprises an amino acid sequence:

(SEQ ID NO: 150)
QVQLKESGAELARPGASVKLSCKASGYTFTTFWMQWAKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDIQMTQSPASLSAS
VGETVTITCRASGNIHNYLAWYQQKQGKSPQLLVYNAKTLADGVPSRFSG
SGSGTQYSLKINSLQPEDFGSYYCQHFWSTPWTFGGGTKLEIK.

In some embodiments, the anti-CD99 scFv comprises an amino acid sequence:

(SEQ ID NO: 151)
QVQLKESGAELARPGASVKLSCKASGYTFTTFWMQWAKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSGNSWSHSLRSLSVT
IGQPASISCKSSQSLLDGNGKTYLNWLLQRPGQSPKRLLYLVSKLDSGVP
DRFTGSGSGTDFTLKISRVEAEDLGIYYCWQGTHFPRTFGGGTKLEIK.

In some embodiments, the anti-CD99 scFv comprises an amino acid sequence:

(SEQ ID NO: 152)
QVQLKESGAELARPGASVKLSCKASGYTFTTFWMQWAKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSGNSWRHSPRSLSVT
IGQPASISCKSSQSLLDGNGKTYLNWLLQRPGQSPKRLLYLVSKLDSGVP
DRFTGSGSGTDFTLKISRVEAEDLGIYYCWQGTHFPRTFGGGTKLEIK.

In some embodiments, the anti-CD99 scFv comprises an amino acid sequence:

(SEQ ID NO: 153)
QVQLKESGAELARPGASVKLSCKASGYTFTTFWMQWAKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDIVMTQAAPSVPVT
PGESVSISCGSSKSLLHSNGNTYLYWFLQRPGQSPQLLIYRVSNLASGVP
DRFSGSGSGTAFTLRISRVEAEDVGVYYCMQHLEYPYTFGGGTRLEIK.

In some embodiments, the anti-CD99 scFv comprises an amino acid sequence:

(SEQ ID NO: 154)
QVQLKESGAELARPGASVKLSCKASGYTFTTFWMQWAKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDIVMTQAAPSVPVT
PGESVSISCGSSKSLLHSNGNTYLYWFLQRPGQSPQLLIYRVSNLASGVP
DRFSGSGSGTAFTLRISRVEAEDVGVYYCMQHLEYPYTFGGGTRLEIK.

In some embodiments, the anti-CD99 scFv comprises an amino acid sequence:

(SEQ ID NO: 155)
QVQLKESGAELARPGASVKLSCKASGYTFTTFWMQWAKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDTVMSQSPSSLAVS
VGEKITMSCKSSQSLLCRSNQKNYLAWYQQKPGQSPKQLIYWASTRESGV
PDRFTGSGSGTDFTLTISSVKAEDLAVYYCQQYYSYPLTFGAGTKLELK.

In some embodiments, the anti-CD99 scFv comprises an amino acid sequence:

(SEQ ID NO: 156)
QVQLKESGAELARPGASVKLSCKASGYTFTTFWMQWAKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDTVMSQSPSSLAVS
VGEKITMSCKSSQSLLYRSNQKNYLAWYQQKPGQSPKQLIYWASTRESGV
PDRFTGSGSGTDFTLTISSVKAEDLAVYYCQQYYSYPLTFGAGTKLELK.

In some embodiments, the anti-CD99 scFv comprises an amino acid sequence:

(SEQ ID NO: 157, 1H3H9 v2)
DIQMTQSPASLSASVGETVTITCRASGNIHNYLAWYQQKQGKSPQLLVYN
AKTLADGVPSRFSGSGSGTQYSLKINSLQPEDFGSYYCQHFWSTPWTFGG
GTKLEIKGGGGSGGGGSGGGGSEVQLQQSGAELVKPGASVKLSCTASGFD
IKDTYIHWVKQRPEQGLEWIGRIDPANGDTRYDPEFQGKASLTADTSSNT
AYLQFSNLTSEDTAVYYCARRGGLSWGQGTTLTVSS.

In some embodiments, the anti-CD99 scFv comprises an amino acid sequence:

(SEQ ID NO: 158)
DIQMTQSPASLSASVGETVTITCRASGNIHNYLAWYQQKQGKSPQLLVYN
AKTLADGVPSRFSGSGSGTQYSLKINSLQPEDFGSYYCQHFWSTPWTFGG
GTKLEIKGGGGSGGGGSGGGGSEVQLEESGGGLVQPKGSLKLSCAASGFT
FNTYAMYWVCQAPGKGLKWVARIRSKVNNYATYYADSVKDRFTISRDDSQ
NMLFLHMNNLKTEDTAIYFCVRDPMDYWGQGISVTVSS.

In some embodiments, the anti-CD99 scFv comprises an amino acid sequence:

(SEQ ID NO: 159)
DIQMTQSPASLSASVGETVTITCRASGNIHNYLAWYQQKQGKSPQLLVYN
AKTLADGVPSRFSGSGSGTQYSLKINSLQPEDFGSYYCQHFWSTPWTFGG
GTKLEIKGGGGSGGGGSGGGGSEVQLVESGGGLVQPKGSLKLSCAASGFT
FNTYAMYWVCQAPGKGLKWVARIRSKVNNYATYYADSVKDRFTISRDDSQ
NMLFLHMNNLKTEDTAIYFCVRDPMDYWGQGISVTVSS.

In some embodiments, the anti-CD99 scFv comprises an amino acid sequence:

(SEQ ID NO: 160)
DIQMTQSPASLSASVGETVTITCRASGNIHNYLAWYQQKQGKSPQLLVYN
AKTLADGVPSRFSGSGSGTQYSLKINSLQPEDFGSYYCQHFWSTPWTFGG
GTKLEIKGGGGSGGGGSGGGGSQVQLQQSGAELARPGASVKLSCKASGYT
FTTFWMQWVKQRPGQGLEWIGTIYPGDDDTRYTQKFKGRATLTADKSSTT
AYMQLSNLSSEDSAVYYCARSGYERGPYYFDSWGQGTTLTVSS.

In some embodiments, the anti-CD99 scFv comprises an amino acid sequence:

(SEQ ID NO: 161)
DIQMTQSPASLSASVGETVTITCRASGNIHNYLAWYQQKQGKSPQLLVYN
AKTLADGVPSRFSGSGSGTQYSLKINSLQPEDFGSYYCQHFWSTPWTFGG
GTKLEIKGGGGSGGGGSGGGGSDVKLQESGAELARPGASVKLSCKASGYT
FTTFWMQRVKQRPGQGLEWIGTIYPGDDDTRYTQKFKGRATLTADKSSTT
AYMQLSNLSSEDSAVYYCARSGYERGPYYFDSWGQGTTLTVSS.

In some embodiments, the anti-CD99 scFv comprises an amino acid sequence:

(SEQ ID NO: 162)
DIQMTQSPASLSASVGETVTITCRASGNIHNYLAWYQQKQGKSPQLLVYN
AKTLADGVPSRFSGSGSGTQYSLKINSLQPEDFGSYYCQHFWSTPWTFGG
GTKLEIKGGGGSGGGGSGGGGSQVOLKESGAELARPGASVKLSCKASGYT
FTTFWMQWAKQRPGQGLEWIGTIYPGDDDTRYTQKFKGRATLTADKSSTT
AYMQLSNLSSEDSAVYYCARSGYERGPYYFDSWGQGTTLTVSS.

In some embodiments, the anti-CD99 scFv comprises an amino acid sequence:

(SEQ ID NO: 163)
DIVMTQAAPSVPVTPGESVSISCGSSKSLLHSNGNTYLYWFLQRPGQSP
QLLIYRVSNLASGVPDRFSGSGSGTAFTLRISRVEAEDVGVYYCMQHLE
YPYTFGGGTRLEIKGGGGSGGGGSGGGGSEVQLQQSGAELVKPGASVKL
SCTASGFDIKDTYIHWVKQRPEQGLEWIGRIDPANGDTRYDPEFQGKAS
LTADTSSNTAYLQFSNLTSEDTAVYYCARRGGLSWGQGTTLTVSS.

In some embodiments, the anti-CD99 scFv comprises an amino acid sequence:

(SEQ ID NO: 164, 4C5E2 v2)
DIVMTQAAPSVPVTPGESVSISCGSSKSLLHSNGNTYLYWFLQRPGQSP
QLLIYRVSNLASGVPDRFSGSGSGTAFTLRISRVEAEDVGVYYCMQHLE
YPYTFGGGTRLEIKGGGGSGGGGSGGGGSEVQLEESGGGLVQPKGSLKL
SCAASGFTFNTYAMYWVCQAPGKGLKWVARIRSKVNNYATYYADSVKDR
FTISRDDSQNMLFLHMNNLKTEDTAIYFCVRDPMDYWGQGISVTVSS.

In some embodiments, the anti-CD99 scFv comprises an amino acid sequence:

(SEQ ID NO: 165, 4C5H10 v2)
DIVMTQAAPSVPVTPGESVSISCGSSKSLLHSNGNTYLYWFLQRPGQSP
QLLIYRVSNLASGVPDRFSGSGSGTAFTLRISRVEAEDVGVYYCMQHLE
YPYTFGGGTRLEIKGGGGSGGGGGGGGSEVQLVESGGGLVQPKGSLKLS
CAASGFTFNTYAMYWVCQAPGKGLKWARIRSKVNNYATYYADSVKDRFT
ISRDDSQNMLFLHMNNLKTEDTAIYFCVRDPMDYWGQGISVTVSS.

In some embodiments, the anti-CD99 scFv comprises an amino acid sequence:

(SEQ ID NO: 166)
DIVMTQAAPSVPVTPGESVSISCGSSKSLLHSNGNTYLYWFLQRPGQSP
QLLIYRVSNLASGVPDRFSGSGSGTAFTLRISRVEAEDVGVYYCMQHLE
YPYTFGGGTRLEIKGGGGSGGGGSGGGGSQVQLQQSGAELARPGASVKL
SCKASGYTFTTFWMQWVKQRPGQGLEWIGTIYPGDDDTRYTQKFKGRAT
LTADKSSTTAYMQLSNLSSEDSAVYYCARSGYERGPYYFDSWGQGTTLT
VSS.

In some embodiments, the anti-CD99 scFv comprises an amino acid sequence:

(SEQ ID NO: 167)
DIVMTQAAPSVPVTPGESVSISCGSSKSLLHSNGNTYLYWFLQRPGQSP
QLLIYRVSNLASGVPDRFSGSGSGTAFTLRISRVEAEDVGVYYCMQHLE
YPYTFGGGTRLEIKGGGGSGGGGSGGGGSDVKLQESGAELARPGASVKL
SCKASGYTFTTFWMQRVKQRPGQGLEWIGTIYPGDDDTRYTQKFKGRAT
LTADKSSTTAYMQLSNLSSEDSAVYYCARSGYERGPYYFDSWGQGTTLT
VSS.

In some embodiments, the anti-CD99 scFv comprises an amino acid sequence:

(SEQ ID NO: 168)
DIVMTQAAPSVPVTPGESVSISCGSSKSLLHSNGNTYLYWFLQRPGQSP
QLLIYRVSNLASGVPDRFSGSGSGTAFTLRISRVEAEDVGVYYCMQHLE
YPYTFGGGTRLEIKGGGGSGGGGSGGGGSQVQLKESGAELARPGASVKL
SCKASGYTFTTFWMQWAKQRPGQGLEWIGTIYPGDDDTRYTQKFKGRAT
LTADKSSTTAYMQLSNLSSEDSAVYYCARSGYERGPYYFDSWGQGTTLT
VSS.

In some embodiments, the anti-CD99 scFv comprises an amino acid sequence:

(SEQ ID NO: 169)
DTVMSQSPSSLAVSVGEKITMSCKSSQSLLCRSNQKNYLAWYQQKPGQS
PKQLIYWASTRESGVPDRFTGSGSGTDFTLTISSVKAEDLAVYYCQQYY
SYPLTFGAGTKLELKGGGGSGGGGSGGGGSEVQLQQSGAELVKPGASVK
LSCTASGFDIKDTYIHWVKQRPEQGLEWIGRIDPANGDTRYDPEFQGKA
SLTADTSSNTAYLQFSNLTSEDTAVYYCARRGGLSWGQGTTLTVSS.

In some embodiments, the anti-CD99 scFv comprises an amino acid sequence:

(SEQ ID NO: 170)
DTVMSQSPSSLAVSVGEKITMSCKSSQSLLCRSNQKNYLAWYQQKPGQS
PKQLIYWASTRESGVPDRFTGSGSGTDFTLTISSVKAEDLAVYYCQQYY
SYPLTFGAGTKLELKGGGGSGGGGGGGGSEVQLEESGGGLVQPKGSLKL
SCAASGFTFNTYAMYWVCQAPGKGLKWVARIRSKVNNYATYYADSVKDR
FTISRDDSQNMLFLHMNNLKTEDTAIYFCVRDPMDYWGQGISVTVSS.

In some embodiments, the anti-CD99 scFv comprises an amino acid sequence:

(SEQ ID NO: 171)
DTVMSQSPSSLAVSVGEKITMSCKSSQSLLCRSNQKNYLAWYQQKPGQS
PKQLIYWASTRESGVPDRFTGSGSGTDFTLTISSVKAEDLAVYYCQQYY
SYPLTFGAGTKLELKGGGGSGGGGSGGGGSEVQLVESGGGLVQPKGSLK
LSCAASGFTFNTYAMYWVCQAPGKGLKWVARIRSKVNNYATYYADSVKD
RFTISRDDSQNMLFLHMNNLKTEDTAIYFCVRDPMDYWGQGISVTVSS.

In some embodiments, the anti-CD99 scFv comprises an amino acid sequence:

(SEQ ID NO: 172, 9G12C9 v2)
DTVMSQSPSSLAVSVGEKITMSCKSSQSLLCRSNQKNYLAWYQQKPGQS
PKQLIYWASTRESGVPDRFTGSGSGTDFTLTISSVKAEDLAVYYCQQYY
SYPLTFGAGTKLELKGGGGSGGGGSGGGGSQVQLQQSGAELARPGASVK
LSCKASGYTFTTFWMQWVKQRPGQGLEWIGTIYPGDDDTRYTQKFKGRA
TLTADKSSTTAYMQLSNLSSEDSAVYYCARSGYERGPYYFDSWGQGTTL
TVSS.

In some embodiments, the anti-CD99 scFv comprises an amino acid sequence:

(SEQ ID NO: 173)
DTVMSQSPSSLAVSVGEKITMSCKSSQSLLCRSNQKNYLAWYQQKPGQS
PKQLIYWASTRESGVPDRFTGSGSGTDFTLTISSVKAEDLAVYYCQQYY
SYPLTFGAGTKLELKGGGGSGGGGSGGGGSDVKLQESGAELARPGASVK
LSCKASGYTFTTFWMQRVKQRPGQGLEWIGTIYPGDDDTRYTQKFKGRA
TLTADKSSTTAYMQLSNLSSEDSAVYYCARSGYERGPYYFDSWGQGTTL
TVSS.

In some embodiments, the anti-CD99 scFv comprises an amino acid sequence:

(SEQ ID NO: 174)
DTVMSQSPSSLAVSVGEKITMSCKSSQSLLCRSNQKNYLAWYQQKPGQS
PKQLIYWASTRESGVPDRFTGSGSGTDFTLTISSVKAEDLAVYYCQQYY
SYPLTFGAGTKLELKGGGGSGGGGSGGGGSQVQLKESGAELARPGASVK
LSCKASGYTFTTFWMQWAKQRPGQGLEWIGTIYPGDDDTRYTQKFKGRA
TLTADKSSTTAYMQLSNLSSEDSAVYYCARSGYERGPYYFDSWGQGTTL
TVSS.

In some embodiments, the anti-CD99 scFv comprises an amino acid sequence:

(SEQ ID NO: 175)
DTVMSQSPSSLAVSVGEKITMSCKSSQSLLYRSNQKNYLAWYQQKPGQS
PKQLIYWASTRESGVPDRFTGSGSGTDFTLTISSVKAEDLAVYYCQQYY
SYPLTFGAGTKLELKGGGGSGGGGSGGGGSEVQLQQSGAELVKPGASVK
LSCTASGFDIKDTYIHWVKQRPEQGLEWIGRIDPANGDTRYDPEFQGKA
SLTADTSSNTAYLQFSNLTSEDTAVYYCARRGGLSWGQGTTLTVSS.

In some embodiments, the anti-CD99 scFv comprises an amino acid sequence:

(SEQ ID NO: 176)
DTVMSQSPSSLAVSVGEKITMSCKSSQSLLYRSNQKNYLAWYQQKPGQS
PKQLIYWASTRESGVPDRFTGSGSGTDFTLTISSVKAEDLAVYYCQQYY
SYPLTFGAGTKLELKGGGGSGGGGGGGGSEVQLEESGGGLVQPKGSLKL
SCAASGFTFNTYAMYWVCQAPGKGLKWVARIRSKVNNYATYYADSVKDR
FTISRDDSQNMLFLHMNNLKTEDTAIYFCVRDPMDYWGQGISVTVSS.

In some embodiments, the anti-CD99 scFv comprises an amino acid sequence:

(SEQ ID NO: 177)
DTVMSQSPSSLAVSVGEKITMSCKSSQSLLYRSNQKNYLAWYQQKPGQS
PKQLIYWASTRESGVPDRFTGSGSGTDFTLTISSVKAEDLAVYYCQQYY
SYPLTFGAGTKLELKGGGGSGGGGSGGGGSEVQLVESGGGLVQPKGSLK
LSCAASGFTFNTYAMYWVCQAPGKGLKWVARIRSKVNNYATYYADSVKD
RFTISRDDSQNMLFLHMNNLKTEDTAIYFCVRDPMDYWGQGISVTVSS.

In some embodiments, the anti-CD99 scFv comprises an amino acid sequence:

(SEQ ID NO: 178)
DTVMSQSPSSLAVSVGEKITMSCKSSQSLLYRSNQKNYLAWYQQKPGQS
PKQLIYWASTRESGVPDRFTGSGSGTDFTLTISSVKAEDLAVYYCQQYY
SYPLTFGAGTKLELKGGGGSGGGGSGGGGSQVQLQQSGAELARPGASVK
LSCKASGYTFTTFWMQWVKQRPGQGLEWIGTIYPGDDDTRYTQKFKGRA
TLTADKSSTTAYMQLSNLSSEDSAVYYCARSGYERGPYYFDSWGQGTTL
TVSS.

In some embodiments, the anti-CD99 scFv comprises an amino acid sequence:

(SEQ ID NO: 179)
DTVMSQSPSSLAVSVGEKITMSCKSSQSLLYRSNQKNYLAWYQQKPGQS
PKQLIYWASTRESGVPDRFTGSGSGTDFTLTISSVKAEDLAVYYCQQYY
SYPLTFGAGTKLELKGGGGSGGGGSGGGGSDVKLQESGAELARPGASVK
LSCKASGYTFTTFWMQRVKQRPGQGLEWIGTIYPGDDDTRYTQKFKGRA
TLTADKSSTTAYMQLSNLSSEDSAVYYCARSGYERGPYYFDSWGQGTTL
TVSS.

In some embodiments, the anti-CD99 scFv comprises an amino acid sequence:

(SEQ ID NO: 180)
DTVMSQSPSSLAVSVGEKITMSCKSSQSLLYRSNQKNYLAWYQQKPGQS
PKQLIYWASTRESGVPDRFTGSGSGTDFTLTISSVKAEDLAVYYCQQYY
SYPLTFGAGTKLELKGGGGSGGGGSGGGGSQVQLKESGAELARPGASVK
LSCKASGYTFTTFWMQWAKQRPGQGLEWIGTIYPGDDDTRYTQKFKGRA
TLTADKSSTTAYMQLSNLSSEDSAVYYCARSGYERGPYYFDSWGQGTTL
TVSS.

### CLEC12A CAR

In some embodiments, the anti-CLEC12A region of the disclosed antibody or CAR is derived from hybridoma 1F3, 1F8, 1G3, 2A10, 3F12, 4E3, 4E10, 5B2, 5F10, 6C7, 9A2, 11C7, 11H1, 12D6, or combinations thereof. In some embodiments, the anti-CLEC12A region (e.g. scFv) can comprise a variable heavy (V_{H}) domain having CDR1, CDR2 and CDR3 sequences and a variable light (V_{L}) domain having CDR1, CDR2 and CDR3 sequences.

In some embodiments, the CDR1 sequence of the V_{H} domain comprises the amino acid sequence GFTFSSFA (SEQ ID NO:181) SFAVS (SEQ ID NO:182), or SHDMS (SEQ ID NO:183); the CDR2 sequence of the V_{H} domain comprises the amino acid sequence ISSGGAYT (SEQ ID NO:184) or TISSGGAYTFYKDSVKGRFT (SEQ ID NO: 185), or YISGGGTNIYYSDTVKGRFT (SEQ ID NO: 186); the CDR3 sequence of the V_{H} domain comprises the amino acid sequence ARHSGYDGYYLYAMDY (SEQ ID NO: 187), HSGYDGYYLYAMDY (SEQ ID NO: 188), or PNYNYGGSWFAY (SEQ ID NO: 189); the CDR1 sequence of the V_{L} comprises the amino acid sequence SSVHY (SEQ ID NO:190), ASSSVHYMH (SEQ ID NO: 191), or SASSSVHYMH (SEQ ID NO: 192); the CDR2 sequence of the V_{L} domain comprises the amino acid sequence DTS or DTSKLAS (SEQ ID NO:193); and the CDR3 sequence of the V_{L} domain comprises the amino acid sequence QQWTSNPPT (SEQ ID NO: 194).

In some embodiments, the anti-CLEC12A V_{H} domain comprises the amino acid sequence:

(SEQ ID NO: 195, 1F3H8)
ELILVESGGGLVKPGGSLKLSCAVSGFTFSSFAMSWVRQTPEKRLEWVA
TISSGGAYTFYKDSVKGRFTISRDNAKNTLYLQMSSLRSEDSAMYYCAR
HSGYDGYYLYAMDYWGQGTSVTVSS.

In some embodiments, the anti-CLEC12A V_{H} domain comprises the amino acid sequence:

(SEQ ID NO: 196, 1F3A10)
GVQCELILVESGGGLVKPGGSLKLSCAVSGFTFSSFAVSWVRQTPEKRL
EWVATISSGGAYTFYKDSVKGRFTISRDNAKNTLYLQMSSLRSEDSAMY
YCARHSGYDGYYLYAMDYWGQGTSVTVSS.

In some embodiments, the anti-CLEC12A V_{H} domain comprises the amino acid sequence:

(SEQ ID NO: 197, 1F3F3)
EVQLEESGGGLVQPGGSLKVSCAVSGLAFSSHDMSWVRQTPEKRLEWVA
YISGGGTNIYYSDTVKGRFTISRDNAKNTLYLQMSSLKSEDTAIYYCAR
PNYNYGGSWFAYWGQGTLVTVSA.

In some embodiments, the anti-CLEC12A V_{L} domain comprises the amino acid sequence:

(SEQ ID NO: 198, 1F3H8, 1F3F3, 1F3A10)
QIVLTQSPEIMSASPGEKVTMTCSASSSVHYMHWYQQKSGTSPKRWIYD
TSKLASGVPGRFSGSGSGTSYSLTISSMESEDAATYYCQQWTSNPPTFG
GGTKLEIK.

In some embodiments, the anti-CLEC12A scFv comprises an amino acid sequence:

(SEQ ID NO: 199, 1F3H8 v1)
ELILVESGGGLVKPGGSLKLSCAVSGFTFSSFAMSWVRQTPEKRLEWVA
TISSGGAYTFYKDSVKGRFTISRDNAKNTLYLQMSSLRSEDSAMYYCAR
HSGYDGYYLYAMDYWGQGTSVTVSSGGGGGGGGSGGGGSQIVLTQSPEI
MSASPGEKVTMTCSASSSVHYMHWYQQKSGTSPKRWIYDTSKLASGVPG
RFSGSGSGTSYSLTISSMESEDAATYYCQQWTSNPPTFGGGTKLEIK.

In some embodiments, the anti-CLEC12A scFv comprises an amino acid sequence:

(SEQ ID NO: 200, 1F3A10 v1)
GVQCELILVESGGGLVKPGGSLKLSCAVSGFTFSSFAVSWVRQTPEKRL
EWVATISSGGAYTFYKDSVKGRFTISRDNAKNTLYLQMSSLRSEDSAMY
YCARHSGYDGYYLYAMDYWGQGTSVTVSSGGGGSGGGGSGGGGSQIVLT
QSPEIMSASPGEKVTMTCSASSSVHYMHWYQQKSGTSPKRWIYDTSKLA
SGVPGRFSGSGSGTSYSLTISSMESEDAATYYCQQWTSNPPTFGGGTKL
EIK.

In some embodiments, the anti-CLEC12A scFv comprises an amino acid sequence:

(SEQ ID NO: 201, 1F3F3 v1)
EVQLEESGGGLVQPGGSLKVSCAVSGLAFSSHDMSWVRQTPEKRLEWVA
YISGGGTNIYYSDTVKGRFTISRDNAKNTLYLQMSSLKSEDTAIYYCAR
PNYNYGGSWFAYWGQGTLVTVSAGGGGSGGGGSGGGGSQIVLTQSPEIM
SASPGEKVTMTCSASSSVHYMHWYQQKSGTSPKRWIYDTSKLASGVPGR
FSGSGSGTSYSLTISSMESEDAATYYCQQWTSNPPTFGGGTKLEIK.

In some embodiments, the anti-CLEC12A scFv comprises an amino acid sequence:

(SEQ ID NO: 202, 1F3H8 v2)
QIVLTQSPEIMSASPGEKVTMTCSASSSVHYMHWYQQKSGTSPKRWIYD
TSKLASGVPGRFSGSGSGTSYSLTISSMESEDAATYYCQQWTSNPPTFG
GGTKLEIKGGGGSGGGGSGGGGSELILVESGGGLVKPGGSLKLSCAVSG
FTFSSFAMSWVRQTPEKRLEWVATISSGGAYTFYKDSVKGRFTISRDNA
KNTLYLQMSSLRSEDSAMYYCARHSGYDGYYLYAMDYWGQGTSVTVSS.

In some embodiments, the anti-CLEC12A scFv comprises an amino acid sequence:

(SEQ ID NO: 203, 1F3A10 v2)
QIVLTQSPEIMSASPGEKVTMTCSASSSVHYMHWYQQKSGTSPKRWIYD
TSKLASGVPGRFSGSGSGTSYSLTISSMESEDAATYYCQQWTSNPPTFG
GGTKLEIKGGGGSGGGGSGGGGSGVQCELILVESGGGLVKPGGSLKLSC
AVSGFTFSSFAVSWVRQTPEKRLEWVATISSGGAYTFYKDSVKGRFTIS
RDNAKNTLYLQMSSLRSEDSAMYYCARHSGYDGYYLYAMDYWGQGTSVT
VSS.

In some embodiments, the anti-CLEC12A scFv comprises an amino acid sequence:

(SEQ ID NO: 204, 1F3F3 v2)
QIVLTQSPEIMSASPGEKVTMTCSASSSVHYMHWYQQKSGTSPKRWIYD
TSKLASGVPGRFSGSGSGTSYSLTISSMESEDAATYYCQQWTSNPPTFG
GGTKLEIKGGGGSGGGGSGGGGSEVQLEESGGGLVQPGGSLKVSCAVSG
LAFSSHDMSWVRQTPEKRLEWVAYISGGGTNIYYSDTVKGRFTISRDNA
KNTLYLQMSSLKSEDTAIYYCARPNYNYGGSWFAYWGQGTLVTVSA.

A dual CAR T cell expresses two separate CARs with different ligand binding targets; one CAR includes only the CD3Z domain and the other CAR includes only the co-stimulatory domain(s). Dual CAR T cell activation requires co-expression of both targets on the tumor. In some embodiments, the two CARs are expressed separately. In some embodiments, the two CARs are co-expressed by a single expression construct. In some embodiments, the two CARs are co-expressed in a single fusion protein separated by a self-cleavable peptide.

Therefore, in some embodiments, the disclosed a dual CAR fusion protein is defined by the formula:

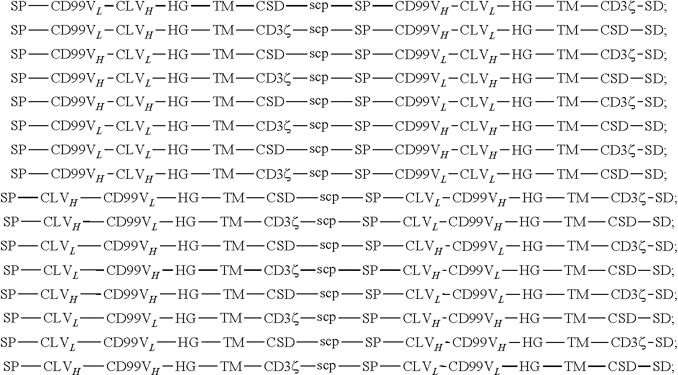

wherein “SP” represents an optional signal peptide,
wherein “CD99V_{H}” represents a CD99 variable heavy domain,
wherein “CD99V_{L}” represents a CD99 variable light domain,
wherein “CLVH” represents a CLEC12A variable heavy domain,
wherein “CLVL” represents a CLEC12A variable light domain,
wherein “HG” represents an optional hinge domain,
wherein “TM” represents a transmembrane domain,
wherein “scp” represents a self-cleaving peptide domain,
wherein “CD34” represents a CD3Z domain,
wherein “CSD” represents a costimulatory-domain, and
wherein “-” represents a peptide bond or linker.

### EGFR/MUC-1 CAR

Also disclosed herein is a bi-specific CAR polypeptide that includes a EGFR antigen binding domain, a MUC1 antigen binding domain, a transmembrane domain, an intracellular signaling domain, and a co-stimulatory signaling region. In some embodiments, the EGFR antigen binding domain is a single-chain variable fragment (scFv) of an antibody comprising a variable heavy (V_{H}) domain and a variable light (V_{L}) domain, and wherein the MUC1 antigen binding domain is a scFv comprising a V_{H} domain and a V_{L} domain.

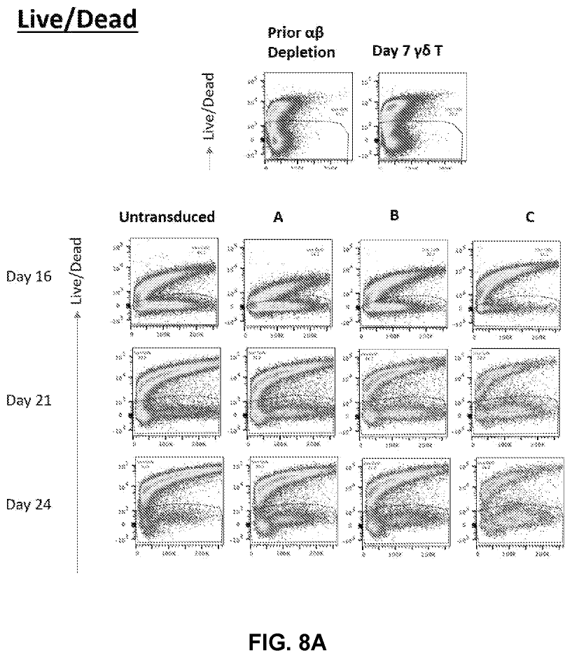

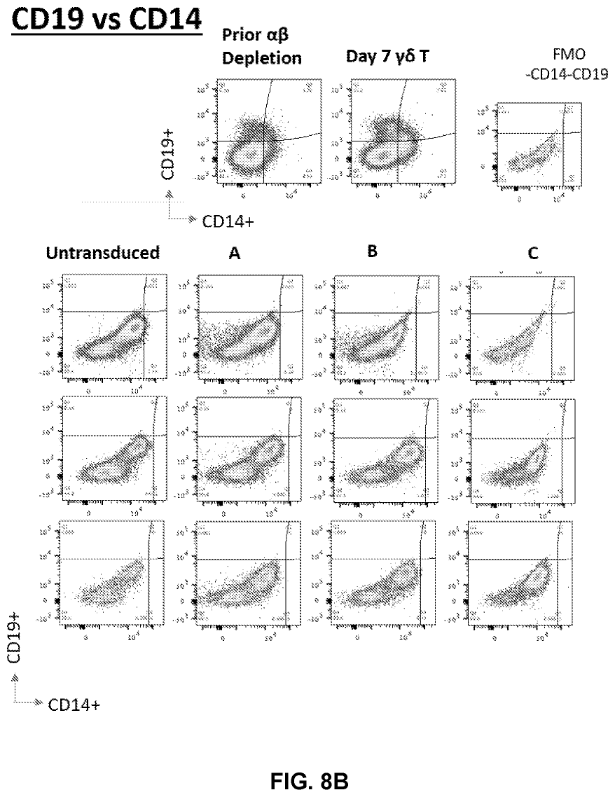

As shown in FIG. 8, the bi-specific CAR polypeptide can have a tandem format and therefore be defined by the formula:

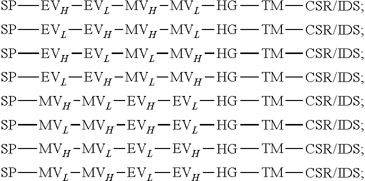

wherein “SP” represents a signal peptide,
wherein “EV_{H}” represents the EGFR scFv V_{H} domain,
wherein “EV_{L}” represents the EGFR scFv V_{L} domain,
wherein “MV_{H}” represents the MUC1 scFv V_{H} domain,
wherein “MV_{L}” represents the MUC1 scFv V_{L} domain,
wherein “HG” represents and optional hinge domain,
wherein “TM” represents a transmembrane domain,
wherein “CSR/IDS” represents a co-stimulatory signaling region and an intracellular signaling domain,
wherein “-” represents a bivalent linker.

As shown in FIG. 8, the bi-specific CAR polypeptide can have a loop format and therefore be defined by the formula:

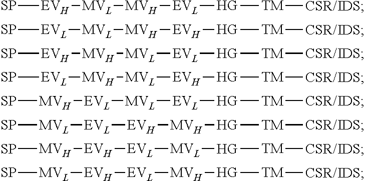

wherein “SP” represents a signal peptide,
wherein “EV_{H}” represents the EGFR scFv V_{H} domain,
wherein “EV_{L}” represents the EGFR scFv V_{L} domain,
wherein “MV_{H}” represents the MUC1 scFv V_{H} domain,
wherein “MV_{L}” represents the MUC1 scFv V_{L} domain,
wherein “HG” represents and optional hinge domain,
wherein “TM” represents a transmembrane domain,
wherein “CSR/IDS” represents a co-stimulatory signaling region and an intracellular signaling domain,
wherein “-” represents a bivalent linker.

Anti-EGFR antibodies are disclosed in U.S. Pat. No. 8,580,263, which is incorporated by reference for the these antibodies, including sequences for use in preparing scFVs.

For example, in some embodiments of the anti-EGFR scFv, the CDR1 sequence of the V_{H} domain comprises the amino acid sequence KASGGTFSSYAIS (SEQ ID NO: 205); CDR2 sequence of the V_{H} domain comprises the amino acid sequence GIIPIFGTANYAQKFQG (SEQ ID NO:206); CDR3 sequence of the V_{H} domain comprises the amino acid sequence AREEGPYCSSTSCYGAFDI (SEQ ID NO:207); CDR1 sequence of the V_{L} comprises the amino acid sequence QGDSLRSYFAS (SEQ ID NO: 208); CDR2 sequence of the V_{L} domain comprises the amino acid sequence YARNDRPA (SEQ ID NO:209); and CDR3 sequence of the V_{L} domain comprises the amino acid sequence AAWDDSLNGYL (SEQ ID NO:210).

In some embodiments, the anti-EGFR scFv V_{H} domain comprises the amino acid sequence:

(SEQ ID NO: 211)
QVQLKQSGPGLVQPSQSLSITCTVSGFSLTNYGVHWVRQSPGKGLEWLG
VIWSGGNTDYNTPFTSRLSINKDNSKSQVFFKMNSLQSNDTAIYYCARA
LTYYDYEFAYWGQGTLVTV.

In some embodiments, the anti-EGFR scFv V_{H} domain comprises the amino acid sequence:

(SEQ ID NO: 212)
EVQLVQSGAEVKKPGSSVKVSCKASGGTFSSYAISWVRQAPGQGLEWMG
GIIPIFGTANYAQKFQGRVTITADESTSTAYMELSSLRSEDTAVYYCAR
EEGPYCSSTSCYGAFDIWGQGTLVTVSS.

In some embodiments, the anti-EGFR scFv V_{L} domain comprises the amino acid sequence:

(SEQ ID NO: 213)
LLTQSPVILSVSPGERVSFSCRASQSIGTNIHWYQQRTNGSPRLLI
KYASESISGIPSRFSGSGSGTDFTLSINSVESEDIADYYCQQNNNW
PTTFGAGTKLELKRTVA.

In some embodiments, the anti-EGFR scFv V_{L} domain comprises the amino acid sequence:

(SEQ ID NO: 214)
QSVLTQDPAVSVALGQTVKITCQGDSLRSYFASWYQQKPGQAPTLV
MYGVPDRFSGSKSGTSASLAISGLQSEDEADYYCAAWDDSLNGYLF
GAGTKLTVL.

In some embodiments, the anti-EGFR comprises an amino acid sequence:

(SEQ ID NO: 215)
EVQLVQSGAEVKKPGSSVKVSCKASGGTFSSYAISWVRQAPGQGLE
WMGGIIPIFGTANYAQKFQGRVTITADESTSTAYMELSSLRSEDTA
VYYCAREEGPYCSSTSCYGAFDIWGQGTLVTVSSGGGGSGGGGSGG
GGSQSVLTQDPAVSVALGQTVKITCQGDSLRSYFASWYQQKPGQAP
TLVMYGVPDRFSGSKSGTSASLAISGLQSEDEADYYCAAWDDSLNG
YLFGAGTKLTVL.

In some embodiments, the anti-EGFR comprises an amino acid sequence:

(SEQ ID NO: 216)
QSVLTQDPAVSVALGQTVKITCQGDSLRSYFASWYQQKPGQAPTLV
MYGVPDRFSGSKSGTSASLAISGLQSEDEADYYCAAWDDSLNGYLF
GAGTKLTVLGGGGSGGGGSGGGGSEVQLVQSGAEVKKPGSSVKVSC
KASGGTFSSYAISWVRQAPGQGLEWMGGIIPIFGTANYAQKFQGRV
TITADESTSTAYMELSSLRSEDTAVYYCAREEGPYCSSTSCYGAFD
IWGQGTLVTVSS.

Anti-MUC1* antibodies are disclosed in U.S. Patent Publication 2017/0204191A1, which is incorporated by reference for these antibodies, including sequences for use in preparing scFVs.

In some embodiments of the anti-MUC1 scFv, the CDR1 sequence of the V_{H} domain comprises the amino acid sequence NYGMN (SEQ ID NO:217), GYAMS (SEQ ID NO: 218), or R/GYA/GMS; CDR2 sequence of the V_{H} domain comprises the amino acid sequence WINTYTGEPTYA/VG/DDFKG (SEQ ID NO:219) or TISSGGTYIYYPDSVKG (SEQ ID NO:220); CDR3 sequence of the V_{H} domain comprises the amino acid sequence S/TGT/DT/AXXY/FYA, TGTTAILNG (SEQ ID NO: 221), SGDGYWYYA (SEQ ID NO:222) or DNYGXXYDYG/A (SEQ ID NO:223); CDR1 sequence of the V_{L} comprises the amino acid sequence SASSSV/ISYM/IH/Y (SEQ ID NO:224) or RASKSVSTSGYSYMH (SEQ ID NO:225); CDR2 sequence of the V_{L} domain comprises the amino acid sequence S/GTSNLAS (SEQ ID NO:226) or LASNLES (SEQ ID NO:227); and CDR3 sequence of the V_{L} domain comprises the amino acid sequence QQRSS/NYPS/FT (SEQ ID NO:228) or QHSRELPFT (SEQ ID NO: 229).

In some embodiments, the anti-MUC1 scFv V_{H} domain comprises the amino acid sequence:

(SEQ ID NO: 230)
VQLQESGGGLVQPGGSMKLSCVASGFTFSNYWMNWVRQSPEKGLEW
VAEIRLKSNNYATHYAESVKGRFTISRDDSKSSVYLQMNNLRAEDT
GIYYCTGVGQFAYWGQGTTVTVSS.

In some embodiments, the anti-MUC1 scFv V_{H} domain comprises the amino acid sequence:

(SEQ ID NO: 231)
DIELTQESALTTSPGETVTLTCRSSTGAVTTSNYANWVQEKPDHLF
TGLIGGTNNRAPGVPARFSGSLIGDKAALTITGAQTEDEAIYFCAL
WYSNHWVFGGGTKL.

In some embodiments, the anti-MUC1 scFv V_{L} domain comprises the amino acid sequence:

(SEQ ID NO: 232)
DIELTQESALTTSPGETVTLTCRSSTGAVTTSNYANWVQEKPDHLF
TGLIGGTNNRAPGVPARFSGSLIGDKAALTITGAQTEDEAIYFCAL
WYSNHWVFGGGTKL.

In some embodiments, the anti-MUC1 scFv V_{L} domain comprises the amino acid sequence:

(SEQ ID NO: 233)
GGGGSVQLQESGGGLVQPGGSMKLSCVASGFTFSNYWMNWVRQSPE
KGLEWVAEIRLKSNNYATHYAESVKGRFTISRDDSKSSVYLQMNNL
RAEDTGIYYCTGVGQFAYWGQGTTVTVSS.

In some embodiments, the anti-MUC1 comprises an amino acid sequence:

(SEQ ID NO: 234)
VQLQESGGGLVQPGGSMKLSCVASGFTFSNYWMNWVRQSPEKGLEW
VAEIRLKSNNYATHYAESVKGRFTISRDDSKSSVYLQMNNLRAEDT
GIYYCTGVGQFAYWGQGTTVTVSSGGGGSGGGGSGGGGSDIELTQE
SALTTSPGETVTLTCRSSTGAVTTSNYANWVQEKPDHLFTGLIGGT
NNRAPGVPARFSGSLIGDKAALTITGAQTEDEAIYFCALWYSNHWV
FGGGTKL.

In some embodiments, the anti-MUC1 comprises an amino acid sequence:

(SEQ ID NO: 235)
EIVLTQSPATLSLSPGERATLTCSATSSVSYIHWYQQRPGQSPRLL
IYSTSNLASGIPARFSGSGSGSDYTLTISSLEPEDFAVYYCQQRSS
SPFTFGSGTKVEIKGGGGSGGGGSGGGGSEVQLVESGGGLVKPGGS
LRLSCAASGFTFSRYGMSWVRQAPGKRLEWVSTISGGGTYIYYPDS
VKGRFTISRDNAKNTLYLQMNSLRAEDTAVYYCTRDNYGRNYDYGM
DYWGQGTLVTVSS.

Membrane Bound IL-15 and/or IL-21

In some embodiments, the membrane bound IL-15 and/or IL-21 comprises a CD8 hinge and transmembrane domain having the amino acid sequence:

(SEQ ID NO: 236)
TTTPAPRPPTPAPTIASQPLSLRPEACRPAAGGAVHTRGLDFACDI
YIWAPLAGTCGVLLLSLVITLYC.

In some embodiments, the IL-15 comprises the amino acid sequence:

(SEQ ID NO: 237)
NWVNVISDLKKIEDLIQSMHIDATLYTESDVHPSCKVTAMKCFLLE
LQVISLESGDASIHDTVENLIILANNSLSSNGNVTESGCKECEELE
EKNIKEFLQSFVHIVQMFINTS.

In some embodiments, the membrane bound IL-15 comprises the amino acid sequence:

(SEQ ID NO: 238)
NWVNVISDLKKIEDLIQSMHIDATLYTESDVHPSCKVTAMKCFLLE
LQVISLESGDASIHDTVENLIILANNSLSSNGNVTESGCKECEELE
EKNIKEFLQSFVHIVQMFINTSTTTPAPRPPTPAPTIASQPLSLRP
EACRPAAGGAVHTRGLDFACDIYIWAPLAGTCGVLLLSLVITLYC.

In some embodiments, the IL-21 comprises the amino acid sequence:

(SEQ ID NO: 239)
HKSSSQGQDRHMIRMRQLIDIVDQLKNYVNDLVPEFLPAPEDVETN
CEWSAFSCFQKAQLKSANTGNNERIINVSIKKLKRKPPSTNAGRRQ
KHRLTCPSCDSYEKKPPKEFLERFKSLLQKMIHQHLSSRTHGSEDS.

In some embodiments, the membrane bound IL-21 comprises the amino acid sequence:

(SEQ ID NO: 240)
HKSSSQGQDRHMIRMRQLIDIVDQLKNYVNDLVPEFLPAPEDVETN
CEWSAFSCFQKAQLKSANTGNNERIINVSIKKLKRKPPSTNAGRRQ
KHRLTCPSCDSYEKKPPKEFLERFKSLLQKMIHQHLSSRTHGSEDS
TTTPAPRPPTPAPTIASQPLSLRPEACRPAAGGAVHTRGLDFACDI
YIWAPLAGTCGVLLLSLVITLYC.

### Nucleic Acids and Vectors

Also disclosed are polynucleotides and polynucleotide vectors encoding the disclosed CD33-specific and CD123-specific CARs that allow expression of the CD33-specific and CD123-specific CARs in the disclosed immune effector cells.

Nucleic acid sequences encoding the disclosed CARs, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Expression of nucleic acids encoding CARs is typically achieved by operably linking a nucleic acid encoding the CAR polypeptide to a promoter, and incorporating the construct into an expression vector. Typical cloning vectors contain transcription and translation terminators, initiation sequences, and promoters useful for regulation of the expression of the desired nucleic acid sequence.

The disclosed nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some embodimens, the polynucleotide vectors are lentiviral or retroviral vectors.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1a (EF-1a). However, other constitutive promoter sequences may also be used, including, but not limited to the simian virus 40 (SV40) early promoter, MND (myeloproliferative sarcoma virus) promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The promoter can alternatively be an inducible promoter. Examples of inducible promoters include, but are not limited to a metallothionine promoter, a glucocorticoid promoter, a progesterone promoter, and a tetracycline promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.).

### Immune Effector Cells

Also disclosed are immune effector cells that are engineered to express the disclosed CARs (also referred to herein as “CAR-T cells.” These cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials that expresses endogenous NKG2D.

In some embodiments, the T cells comprise γδ T cells, which possess a distinct T-cell receptor (TCR) having one γ chain and one δ chain instead of α and β chains.

In some embodiments, the T cells comprise Natural-killer (NK) cells, which are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can also eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects. Although NK cells have a well-known role as killers of cancer cells, and NK cell impairment has been extensively documented as crucial for progression of MM (Godfrey J, et al. Leuk Lymphoma 2012 53:1666-1676; Fauriat C, et al. Leukemia 2006 20:732-733), the means by which one might enhance NK cell-mediated anti-MM activity has been largely unexplored prior to the disclosed CARs.

In some embodiments, the immune effector cells are derived from stem cells, such as induced pluripotent stem cells (IPSCs). For example, in some embodiments, the immune effector cells are γδ T cells or NK cells derived from IPSCs.

### Therapeutic Methods

Immune effector cells expressing the disclosed CARs can elicit an anti-tumor immune response against CD33-expressing and/or CD123-expressing cancer cells. The anti-tumor immune response elicited by the disclosed CAR-modified immune effector cells may be an active or a passive immune response. In addition, the CAR-mediated immune response may be part of an adoptive immunotherapy approach in which CAR-modified immune effector cells induce an immune response specific to CD33 and/or CD123.

Adoptive transfer of immune effector cells expressing chimeric antigen receptors is a promising anti-cancer therapeutic. Following the collection of a patient's immune effector cells, the cells may be genetically engineered to express the disclosed CD33-specific and/or CD123-specific CARs, then infused back into the patient.

The disclosed CAR-modified immune effector cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat MM. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.)

injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed CAR-modified immune effector cells are administered to a patient in conjunction with (e.g., before, simultaneously or following) any number of relevant treatment modalities, including but not limited to thalidomide, dexamethasone, bortezomib, and lenalidomide. In further embodiments, the CAR-modified immune effector cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The cancer of the disclosed methods can be any CD33-expressing and/or CD123-expressing cell in a subject undergoing unregulated growth, invasion, or metastasis. Cancers that express CD33 and/or CD123 include prostate cancer, ovarian cancer, adenocarcinoma of the lung, breast cancer, endometrial cancer, gastric cancer, colon cancer, and pancreatic cancer. CD33 has also been found on Jurkat cells. In some aspects, the cancer is a gallbladder cancer, exocrine adenocarcinoma, or apocrine adenocarcinomas. In some cases, the cancer comprises myelodysplastic syndrome, acute myeloid leukemia, or bi-phenotypic leukemia.

In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed CARs can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed CARs can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHlgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MEDI4736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed CARs can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent is a targeted agent, such as ibrutinib or idelalisib.

In some embodiments, such an additional therapeutic agent is an epigenetic modifier such as azacitdine or vidaza.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec STI571) or lapatinib. Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with a CARs for treating the disorders as described above may be an anti-cancer cytokine, chemokine, or combination thereof. Examples of suitable cytokines and growth factors include IFNy, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-Ia from the human CXC and C-C chemokine families. Suitable cytokines include cytokine derivatives, cytokine variants, cytokine fragments, and cytokine fusion proteins.

In some embodiments, a therapeutic agent for use in combination with a CARs for treating the disorders as described above may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent for use in combination with a CARs for treating the disorders as described above may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use in combination with an CARs for treating the disorders as described above may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed CARs is administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed CARs is administered in combination with surgery.

CAR-T cells may be designed in several ways that enhance tumor cytotoxicity and specificity, evade tumor immunosuppression, avoid host rejection, and prolong their therapeutic half-life. TRUCK (T-cells Redirected for Universal Cytokine Killing) T cells for example, possess a CAR but are also engineered to release cytokines such as IL-12 that promote tumor killing. Because these cells are designed to release a molecular payload upon activation of the CAR once localized to the tumor environment, these CAR-T cells are sometimes also referred to as ‘armored CARs’. Several cytokines as cancer therapies are being investigated both pre-clinically and clinically, and may also prove useful when similarly incorporated into a TRUCK form of CAR-T therapy. Among these include IL-2, IL-3. IL-4, IL-5, IL-6, IL-7, IL-10, IL-12, IL-13, IL-15, IL-18, M-CSF, GM-CSF, IFN-α, IFN-γ, TNF-α, TRAIL, FLT3 ligand, Lymphotactin, and TGF-β (Dranoff 2004). “Self-driving” or “homing” CAR-T cells are engineered to express a chemokine receptor in addition to their CAR. As certain chemokines can be upregulated in tumors, incorporation of a chemokine receptor aids in tumor trafficking to and infiltration by the adoptive T-cell, thereby enhancing both specificity and functionality of the CAR-T (Moon 2011). Universal CAR-T cells also possess a CAR, but are engineered such that they do not express endogenous TCR (T-cell receptor) or MHC (major histocompatibility complex) proteins. Removal of these two proteins from the signaling repertoire of the adoptive T-cell therapy prevents graft-versus-host-disease and rejection, respectively. Armored CAR-T cells are additionally so named for their ability to evade tumor immunosuppression and tumor-induced CAR-T hypofunction. These particular CAR-Ts possess a CAR, and may be engineered to not express checkpoint inhibitors. Alternatively, these CAR-Ts can be co-administered with a monoclonal antibody (mAb) that blocks checkpoint signaling. Administration of an anti-PDL1 antibody significantly restored the killing ability of CAR TILs (tumor infiltrating lymphocytes). While PD1-PDL1 and CTLA-4-CD80/CD86 signaling pathways have been investigated, it is possible to target other immune checkpoint signaling molecules in the design of an armored CAR-T including LAG-3, Tim-3, IDO-1, 2B4, and KIR. Other intracellular inhibitors of TILs include phosphatases (SHP1), ubiquitin-ligases (i.e., cbl-b), and kinases (i.e., diacylglycerol kinase). Armored CAR-Ts may also be engineered to express proteins or receptors that protect them against or make them resistant to the effects of tumor-secreted cytokines. For example, CTLs (cytotoxic T lymphocytes) transduced with the double negative form of the TGF-β receptor are resistant to the immunosuppression by lymphoma secreted TGF-β. These transduced cells showed notably increased antitumor activity in vivo when compared to their control counterparts.

Tandem and dual CAR-T cells are unique in that they possess two distinct antigen binding domains. A tandem CAR contains two sequential antigen binding domains facing the extracellular environment connected to the intracellular costimulatory and stimulatory domains. A dual CAR is engineered such that one extracellular antigen binding domain is connected to the intracellular costimulatory domain and a second, distinct extracellular antigen binding domain is connected to the intracellular stimulatory domain. Because the stimulatory and costimulatory domains are split between two separate antigen binding domains, dual CARs are also referred to as “split CARs”. In both tandem and dual CAR designs, binding of both antigen binding domains is necessary to allow signaling of the CAR circuit in the T-cell. Because these two CAR designs have binding affinities for different, distinct antigens, they are also referred to as “bi-specific” CARs.

One primary concern with CAR-T cells as a form of “living therapeutic” is their manipulability in vivo and their potential immune-stimulating side effects. To better control CAR-T therapy and prevent against unwanted side effects, a variety of features have been engineered including off-switches, safety mechanisms, and conditional control mechanisms. Both self-destruct and marked/tagged CAR-T cells for example, are engineered to have an “off-switch” that promotes clearance of the CAR-expressing T-cell. A self-destruct CAR-T contains a CAR, but is also engineered to express a pro-apoptotic suicide gene or “elimination gene” inducible upon administration of an exogenous molecule. A variety of suicide genes may be employed for this purpose, including HSV-TK (herpes simplex virus thymidine kinase), Fas, iCasp9 (inducible caspase 9), CD20, MYC TAG, and truncated EGFR (endothelial growth factor receptor). HSK for example, will convert the prodrug ganciclovir (GCV) into GCV-triphosphate that incorporates itself into replicating DNA, ultimately leading to cell death. iCasp9 is a chimeric protein containing components of FK506-binding protein that binds the small molecule AP1903, leading to caspase 9 dimerization and apoptosis. A marked/tagged CAR-T cell however, is one that possesses a CAR but also is engineered to express a selection marker. Administration of a mAb against this selection marker will promote clearance of the CAR-T cell. Truncated EGFR is one such targetable antigen by the anti-EGFR mAb, and administration of cetuximab works to promotes elimination of the CAR-T cell. CARs created to have these features are also referred to as sCARs for ‘switchable CARs’, and RCARs for ‘regulatable CARs’. A “safety CAR”, also known as an “inhibitory CAR” (iCAR), is engineered to express two antigen binding domains. One of these extracellular domains is directed against a tumor related antigen and bound to an intracellular costimulatory and stimulatory domain. The second extracellular antigen binding domain however is specific for normal tissue and bound to an intracellular checkpoint domain such as CTLA4, PD1, or CD45. Incorporation of multiple intracellular inhibitory domains to the iCAR is also possible. Some inhibitory molecules that may provide these inhibitory domains include B7-H1, B7-1, CD160, PIH, 2B4, CEACAM (CEACAM⁻. CEACAM-3, and/or CEACAM-5), LAG-3, TIGIT, BTLA, LAIR1, and TGFB-R. In the presence of normal tissue, stimulation of this second antigen binding domain will work to inhibit the CAR. It should be noted that due to this dual antigen specificity, iCARs are also a form of bi-specific CAR-T cells. The safety CAR-T engineering enhances specificity of the CAR-T cell for tumor tissue, and is advantageous in situations where certain normal tissues may express very low levels of a tumor associated antigen that would lead to off target effects with a standard CAR (Morgan 2010). A conditional CAR-T cell expresses an extracellular antigen binding domain connected to an intracellular costimulatory domain and a separate, intracellular costimulator. The costimulatory and stimulatory domain sequences are engineered in such a way that upon administration of an exogenous molecule the resultant proteins will come together intracellularly to complete the CAR circuit. In this way, CAR-T activation can be modulated, and possibly even ‘fine-tuned’ or personalized to a specific patient. Similar to a dual CAR design, the stimulatory and costimulatory domains are physically separated when inactive in the conditional CAR; for this reason these too are also referred to as a “split CAR”.

In some embodiments, two or more of these engineered features may be combined to create an enhanced, multifunctional CAR-T. For example, it is possible to create a CAR-T cell with either dual- or conditional-CAR design that also releases cytokines like a TRUCK. In some embodiments, a dual-conditional CAR-T cell could be made such that it expresses two CARs with two separate antigen binding domains against two distinct cancer antigens, each bound to their respective costimulatory domains. The costimulatory domain would only become functional with the stimulatory domain after the activating molecule is administered. For this CAR-T cell to be effective the cancer must express both cancer antigens and the activating molecule must be administered to the patient; this design thereby incorporating features of both dual and conditional CAR-T cells.

Typically, CAR-T cells are created using a-B T cells, however y-o T cells may also be used. In some embodiments, the described CAR constructs, domains, and engineered features used to generate CAR-T cells could similarly be employed in the generation of other types of CAR-expressing immune cells including NK (natural killer) cells, B cells, mast cells, myeloid-derived phagocytes, and NKT cells. Alternatively, a CAR-expressing cell may be created to have properties of both T-cell and NK cells. In an additional embodiment, the transduced with CARs may be autologous or allogeneic.

Several different methods for CAR expression may be used including retroviral transduction (including y-retroviral), lentiviral transduction, transposon/transposases (Sleeping Beauty and PiggyBac systems), and messenger RNA transfer-mediated gene expression. Gene editing (gene insertion or gene deletion/disruption) has become of increasing importance with respect to the possibility for engineering CAR-T cells as well. CRISPR-Cas9, ZFN (zinc finger nuclease), and TALEN (transcription activator like effector nuclease) systems are three potential methods through which CAR-T cells may be generated.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Large Scale Ex Vivo Expansion of γδ T Cells Using Artificial Antigen Presenting Cells for the Treatment of Acute Myeloid Leukemia

### Materials and Methods

Cells: Healthy donor apheresis was purchased from All Cells (Emeryville, CA USA). K-562 and CHO (Chinese hamster ovary) cells were purchased from ATCC (Manassas, VA USA). CHO cells were transduced with CD33 to create target cells. Cell lines were authenticated by using a cell line authentication kit (ATCC). CHO media F-12K is ATCC-formulated media supplemented with 10% fetal bovine serum, L-glutamine, and penicillin/streptomycin. Certified bovine spongiform encephalitis free fetal bovine serum was purchased from Atlanta Biologicals (Atlanta, GA USA), and all other media and reagents were obtained from ThermoFisher (Waltham, MA USA).

Genetic constructs and cell-based aAPCs: All constructs used the SFG retroviral backbone. The SFG plasmid was modified to include an anti-human CD3 scFv, a P2A self-cleaving sequence, and human CD137L. The second SFG-based construct included an anti-human CD28 scFv, a P2A self-cleaving sequence, and human IL15RA. Both SFG constructs were transfected into H29 cells using a Calcium Phosphate Transfection Kit (Prometa, Madison, WI USA). K-562 cells were transduced with H29 retroviral supernatant expressing CD3scFv/CD137L and cultured in RPMI complete media for 4-5 days. K-562 CD137L positive cells were flow-sorted with a 5-laser FACSAria (BD Biosciences, San Jose, CA USA) and expanded in RPMI media. K-562 CD3/137L cells were then transduced with H29 retroviral supernatant expressing CD28scFv/IL15RA. CD137L and IL15RA double-positive cells were flow-sorted with a 5-laser FACSAria. K-562 CD3/137L/28/IL15RA cells were expanded, collected, and cryopreserved.

Enrichment and expansion of γδ T Cells: Healthy donor apheresis was elutriated using the ELUTRA and the lymphocyte fraction (fraction 2) was cultured in AIM-V supplemented with 10% human AB serum, 3 μM/L zol, and 300 IU/mL IL-2 for 7 days. αβ T cells were depleted from zol expanded γδ T cells. γδ T cells were further expanded with aAPCs in supplemented AIM-V1 media for 10 days.

Flow cytometry: γδ T cells were defined by gating on live CD45⁺CD3⁺ TCRγδ⁺ CD20⁻ TCRαβ⁻ cells. The percentage natural killer (NK) cells (live CD45⁺CD16⁺ CD56⁺ CD3⁻) was also assessed. All the other biomarkers were gated on γδ T cells including γδ T cell memory subtypes: central memory (CM) defined as CD45RO⁺ CD45RA⁻ CCR7⁺, effector memory (EM) as CD45RO⁺ CD45RA⁻ CCR7⁻, terminally differentiated effector memory RA (EMRA) cells as CD45RO⁻ CD45RA⁻, and naïve cells as CD45RO⁻ CD45RA⁺.

γδ T Cell Cytotoxicity: Cytotoxicity assays were performed on an xCelligence RTCA (real-time cell analysis) instrument (ACEA Biosciences, San Diego, CA USA) according to the manufacturer's instructions. Briefly, γδ T cells were stimulated with CD3/CD28 Dynabeads (ThermoFisher) for 7 days. Target CHO cells were plated at 1×10⁴ per well on an E-Plate 96. The next day γδ T cells were resuspended in fresh complete medium without IL-2 and added onto target cells at various E/T ratios, and growth was monitored.

### Results

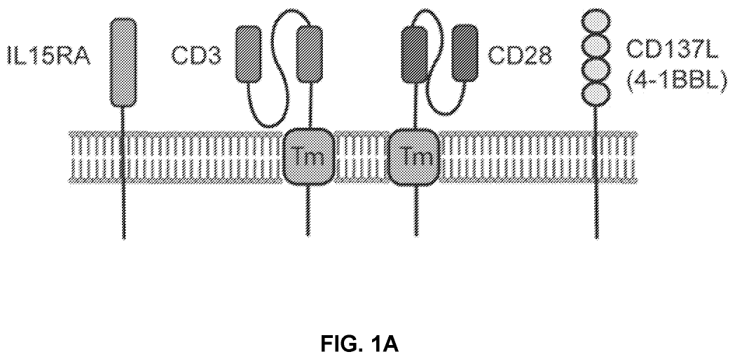

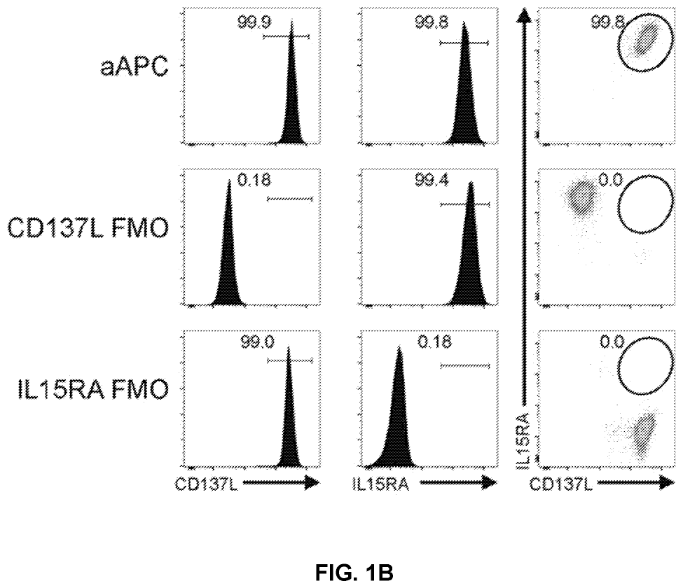

K-562 aAPCs enhance γδ T cell expansion: Cell-based aAPCs can be an economical way to generate a large number of antineoplastic T cells (Maus M V, et al. Clin Immunol. 2003 106(1):16-22; Butler M O, et al. Clin Cancer Res. 2007 13(6):1857-67; Hasan A N, et al. J Immunol. 2009 183(4):2837-50; Maus M V, et al. Nature Biotechnol. 2002 20(2):143-8). To expand γδ T cells, a new quadruple aAPC, K-562 CD3/CD137L/CD28/IL15RA, was created by transducing 2 vectors into K-562 cells (FIG. 1A). The first vector contained anti-human CD3 scFv, a P2A self-cleaving sequence, and human CD137L. The second encoded for an anti-human CD28 scFv, a P2A self-cleaving sequence, and human IL15RA. After transduction, K-562 cells were FACS sorted, and only cells that were positive for both CD137L and IL15RA were collected and used for subsequent experiments (FIG. 1B).

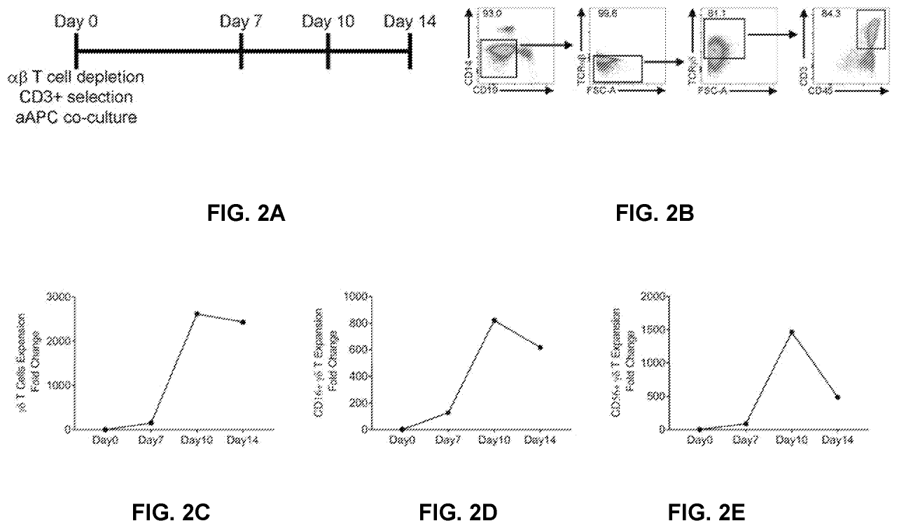

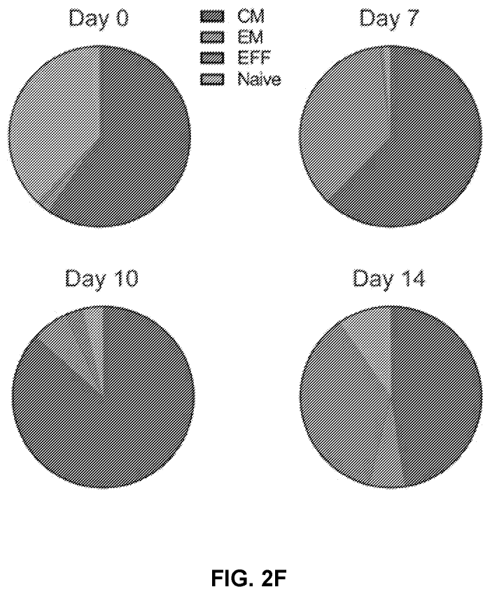

To investigate the ability of aAPCs to support γδ T cell expansion, γδ T cells were isolated from healthy donor PBMCs by αβ T cell depletion followed by CD3 positive selection. aAPCs were cultured with the enriched γδ T cells at a 100:1 aAPC:γδ T cell ratio for up to 14 days (FIG. 2A). At days 7, 10, and 14 after aAPC addition, cells were counted, and γδ T cell percentage was determined by flow cytometry (FIG. 2B). There was a 156-fold γδ T cell expansion at day 7 but by days 10 and 14, and there was a 2612- and a 2429-fold increase in γδ T cells respectively from day 0 (FIG. 2C). An 820-fold expansion of CD16⁺ (FIG. 2D) was also observed, resulting in a 1461-fold increase in CD56⁺ (FIG. 2E) γδ T cells after day 10 of aAPC co-culture. The fold increase of these fell by day 14. These data are representative of 4 independent donors and demonstrate that γδ T cells can rapidly and significantly increase in numbers after aAPC co-culture.

To examine these γδ T cell phenotypes, flow cytometry was used showing that at all days examined the CM γδ T cells constituted the most abundant phenotype (FIG. 2F). At day 10, there were 86% CM cells, while at day 14, there was a reduction of CM cells to 47%, and effector γδ T cells had increased to 36%. Additionally, no viable K562 aAPC were found in the final expanded γδ T cell product. Based on γδ T cell fold increase and memory phenotype, 10 days was determined to be the optimal co-culture period.

γδ T cell enrichment and expansion by zoledronic acid and IL-2: Data demonstrate that γδ T cell co-culture with aAPCs enhances γδ T cell expansion and memory phenotypes. To further enhance γδ T cell expansion, a pre-culture of PBMCs was incorporated with 5 μM zol and 300 IU/mL IL-2, as previously reported (Xiao L, et al. Cytotherapy. 2018 20(3):420-35), prior to αβ T cell depletion and co-culture with K-562 quadruplet aAPCs. By using this method, an enrichment of γδ T cells from 1.98% to 54.58% was achieve while reducing the αβ T cell component from 67.40% to 26.83% after 7 days of culture. γδ T cells were further enriched by αβ T cell depletion, which increased the average percentage of γδ T cells to 74.80% and decreased the αβ T cells to 0.05% (Table 1).

TABLE 1
αβ T cell depletion enhances γδ T cell purity.

| | γδ T cell Average (%) | Standard Deviation |
|---|---|---|
| PBMC Isolation (Day −7) | 1.98 | 0.54 |
| Pre αβ depletion (Day 0) | 54.58 | 58.80 |
| Post αβ depletion (Day 0) | 74.80 | 26.80 |
| Harvest (Day 10) | 75.23 | 32.43 |

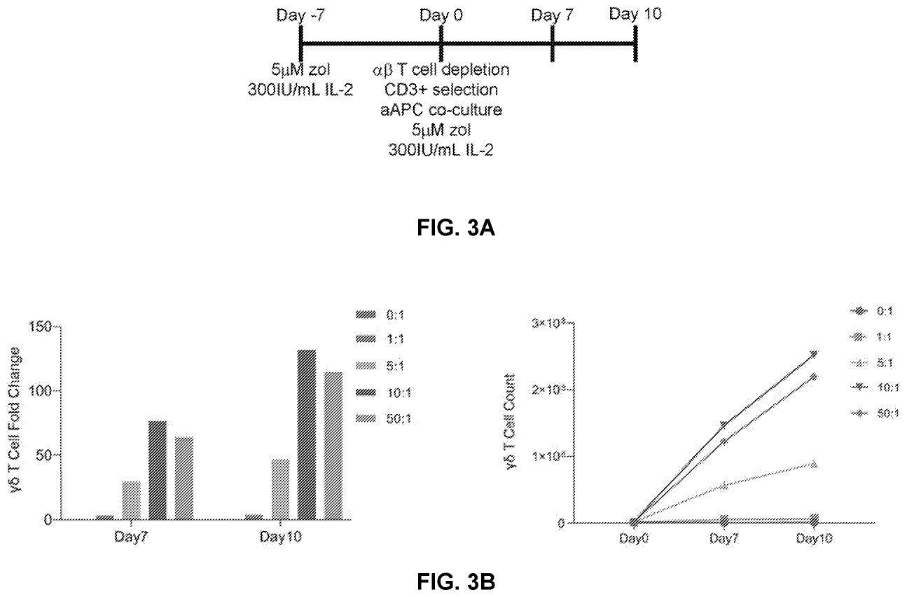

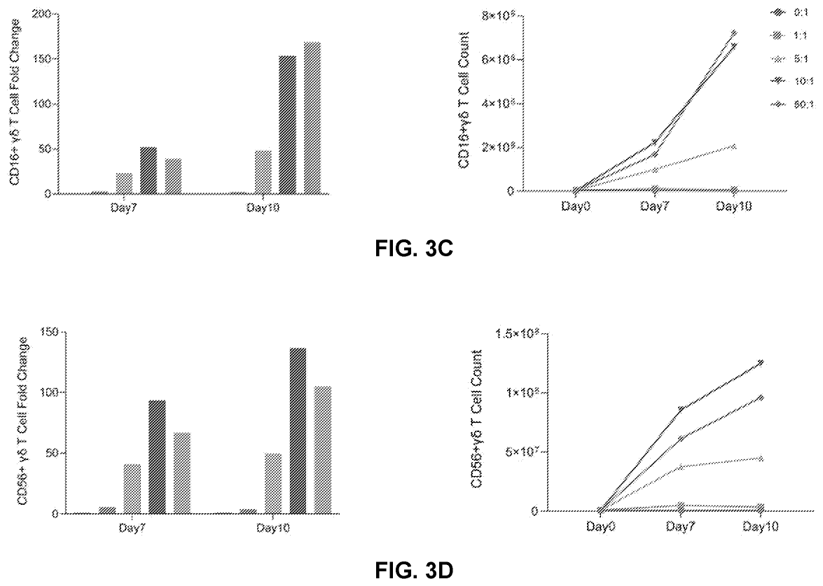

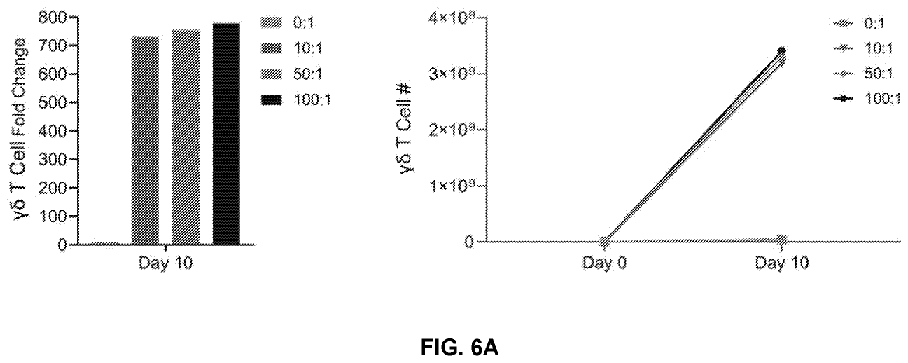

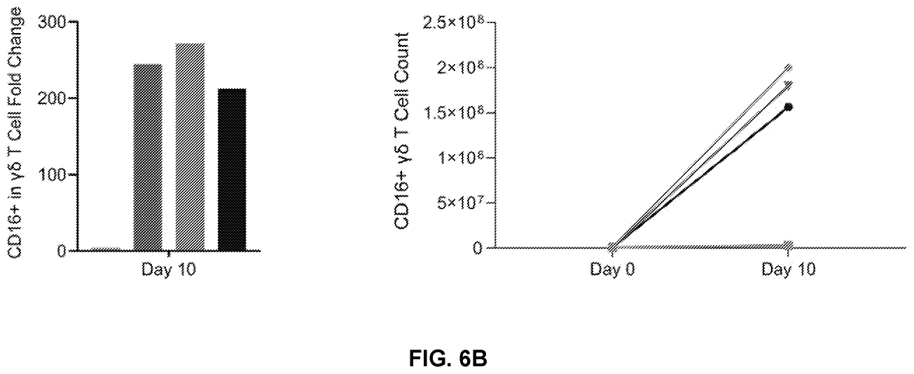

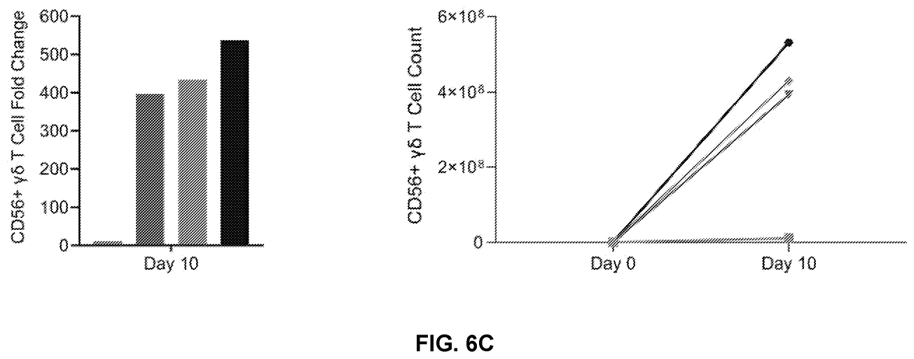

10:1 is the optimal aAPC:γδ T cell ratio for expansion: Previous experiments (FIG. 2) were performed at aAPC:γδ T cell ratios of 100:1. To determine if the total number of aAPCs could be reduced, thus reducing the culture volume and facilitating scale-up for clinical use, aAPC:γδ T cell ratios were examined. Enriched γδ T cells were co-cultured with various numbers of aAPCs in fresh media containing the same concentration of zol and IL-2 that had been used from day −7 to day 0 (FIG. 3A). no substantial differences between 100:1, 50:1, and 10:1 aAPC:γδ T cell ratios was observed in γδ T cell percentage, fold change or absolute count (FIG. 6A). Lower aAPC:γδ T cell ratios (0:1, 1:1, and 5:1) were evaluated in subsequent experiments and it was determined that γδ T cells had the greatest fold change and increased in absolute count at a 10:1 ratio at both days 7 and 10 (FIG. 3B). CD16⁺ γδ T cells (FIG. 3C and FIG. 6B) and CD56⁺ γδ T cells (FIGS. 3D and 6C) were also optimally expanded at ratios of 10:1 and 50:1. Therefore, all subsequent experiments were performed at 10:1 aAPC:γδ T cell. Contamination of αβ T cells in post-expansion γδ T cell product was reproducibly <1%.

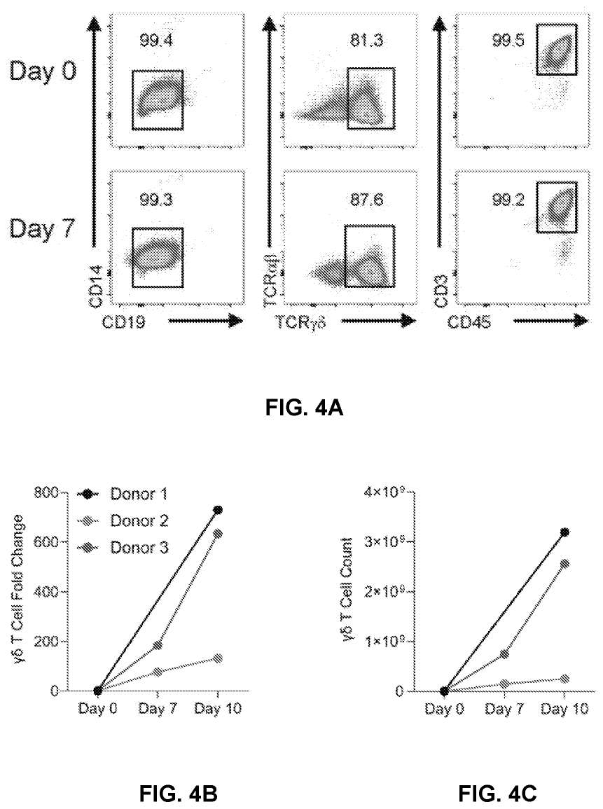

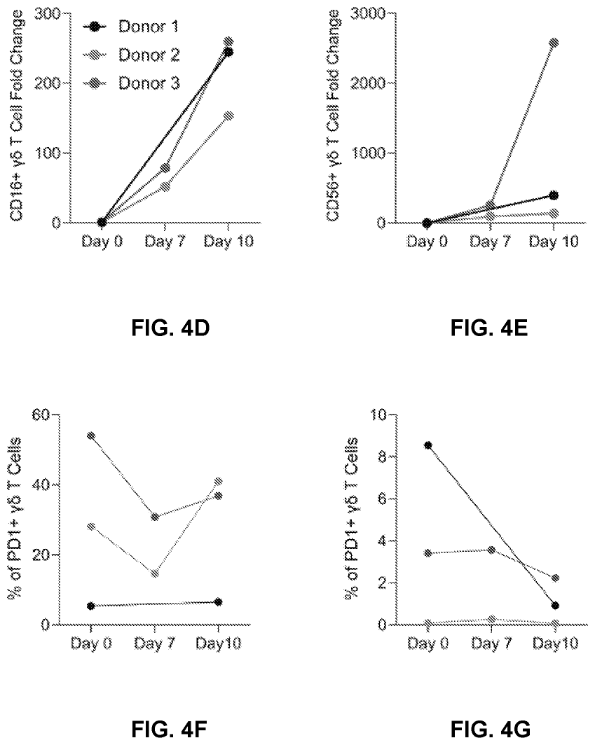

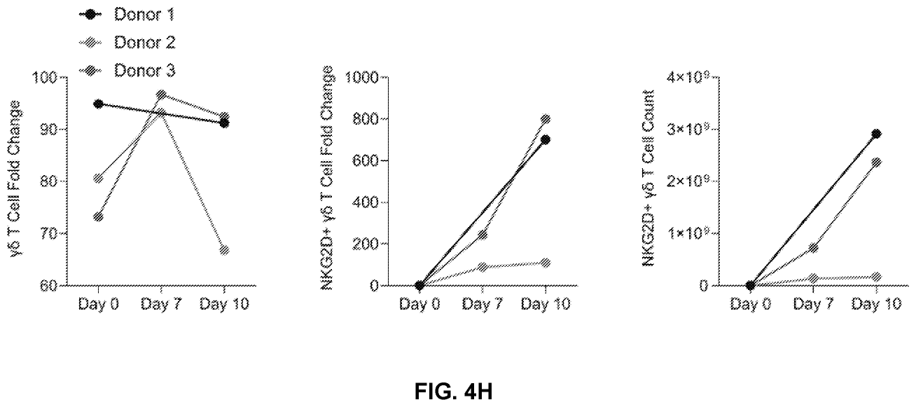

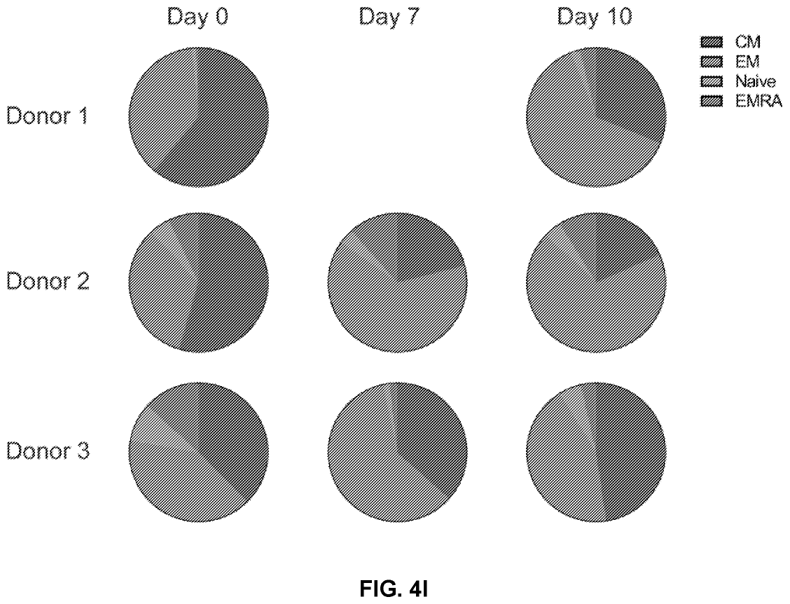

Zol/IL-2 enriched γδ T cells have increased expansion after aAPC co-culture: Flow cytometry was performed to determine whether pre-culture with zol affects subsequent γδ T cell expansion with aAPC and their memory phenotype (FIG. 4A). Representative of 3 healthy donors, γδ T cells co-cultured with aAPCs resulted in a 184-fold increase at day 7 and a 633-fold increase by day 10 (FIG. 4B). Absolute numbers of γδ T cells also increased from 4.0×10⁶ at day 0 to 7.4×10⁸ at day 7 and 2.5×10⁹ by day 10 (FIG. 4C). A fold increase of 259 was also observed in CD16⁺ (FIG. 4D) and 2578 in CD56⁺ (FIG. 4E) γδ T cells on day 10.

The expression of inhibitory or cytotoxic markers on γδ T cells can affect function. After aAPC co-culture, there was a decrease over time in PD1 (FIG. 4F) and LAG3 (FIG. 4G). There was an increase in the percentage of γδ T cells expressing NKG2D from 73.2% on day 0 to 92.4% on day 10 (FIG. 4H). In addition, there was a 799-fold increase in γδ T cells expressing NKG2D by day 10.

To assess γδ T cell differentiation naïve, CM, EM, and EMRA γδ T cells were analyzed. A high percentage of CM, 36% and 47%, and EM, 60% and 43%, γδ T cells was found at days 7 and 10 respectively (FIG. 4I). A low percentage of EMRA cells (1.8% day 7 and 3.5% day 10) was also observed. Similar to data from FIG. 2 this suggests that γδ T cells retain a favorable memory phenotype after 10 days of co-culture with quadruple aAPCs.

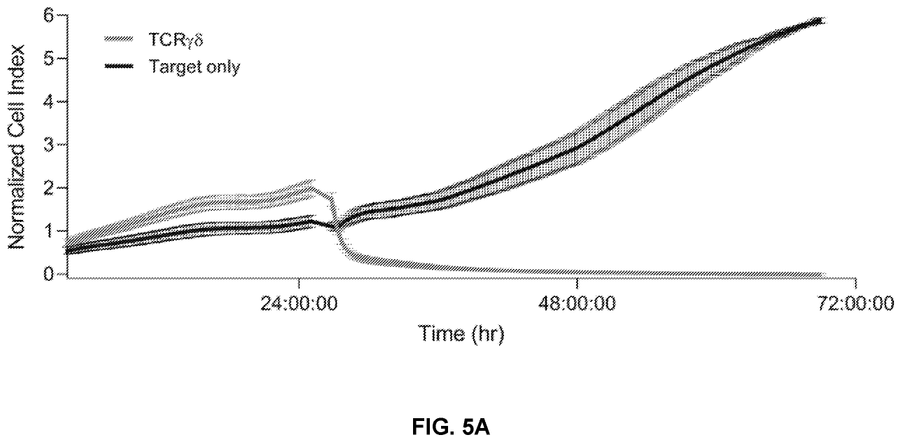

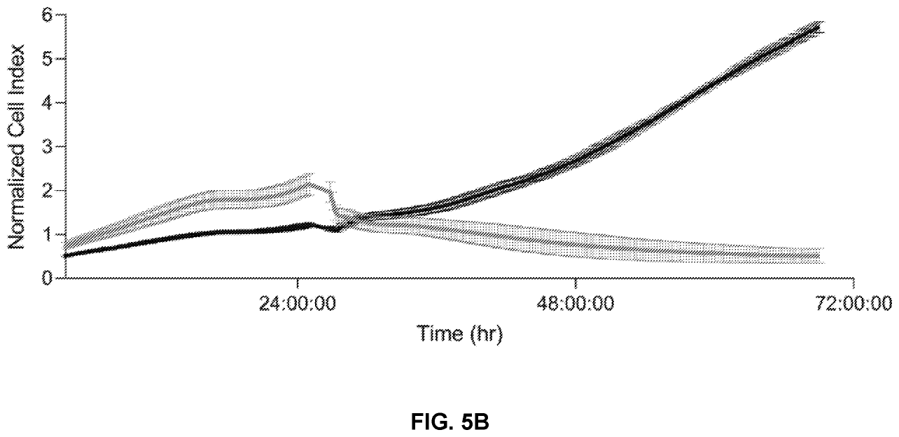

γδ T cells are cytotoxic after aAPC expansion: To demonstrate that culture with zol/IL-2 and quadruple aAPCs results in functional γδ T cells their cytotoxicity was examined in vitro using a real-time cell killing assay. To better approximate use in a clinical setting γδ T cells that were cryopreserved were used. When the cytotoxic ability of these cells from 2 healthy donors was examined it was found that they were able to effectively kill the target cells (FIG. 5). These results demonstrate that zol enriched γδT cells after 10 days of aAPC co-culture retain their cytotoxic abilities.

### Discussion

Healthy donor γδ T cell infusion have therapeutic potential for high-risk AML. However, low numbers of circulating peripheral blood γδ T cells limit their clinical use. Here it is demonstrated that γδ T cells can significantly expand ex vivo in co-culture with genetically engineered K-562 CD3/137L/28/IL15RA aAPC using a scaled-up production system suitable for clinical-grade cells. Thus, this methodology provides an opportunity to use ex vivo expanded healthy donor-derived γδ T cells for clinical application as antineoplastic immunotherapy.

Although the process builds upon the γδ T cell expansion protocol reported by Xiao and colleagues, there are several critical differences between the methodologies used (Xiao L, et al. Cytotherapy. 2018 20(3):420-35). Following the initial step of zol and IL-2 treatment and subsequent αβ T cell depletion, co-culture was done using K-562 quadruplet aAPC and zol/IL-2 without need to use antihuman CD3 monoclonal antibody OKT3 since the aAPCs already express CD3. Moreover, K-562 CD3/137L/28/IL15RA was used, which is the first aAPC expressing 4 different antigens for γδ T cell expansion. As previously described, these aAPCs support efficacious pan-T cell expansion and exhibit lower exhaustion compared to bead expanded T cells (Shrestha B, et al. J Immunother. 2020 43(3):79-88). CD137L is shown to be the dominant co-stimulatory proliferative signal on aAPCs for the expansion of γδ T cells (Deniger D C, et al. Clin Cancer Res. 2014 20(22):5708-19). While anti-CD28 and IL-15RA were the additional markers expressed on aAPCs, Xiao et al. used aAPCs expressing CD64, CD86, and CD137L. CD28-mediated costimulation is necessary for the activation of γδ T cells (Sperling A I, et al. J Immunol. 1993 151(11):6043-50), and IL-15 is important for in vivo expansion of γδ T cells (Izumi T, et al. Cytotherapy. 2013 15(4):481-91). This suggests that the expression of both CD28 and IL-15RA on aAPCs could further optimize the protocol for the clinical application. It was also determined that a 10:1 aAPC to γδ T cell ratio was optimal for expansion. This ratio is markedly less than the 100:1 ratio used by Xiao et al. which also included zol and IL-2 in their aAPC and γδ T cell co-culture (Xiao L, et al. Cytotherapy. 2018 20(3):420-35). The reduced ratio in the system can reduce costs by needing fewer aAPCs for a sufficient number of γδ T cell expansion to be used in a clinical trial setting.

While several studies report the effective expansion of γδ T cells with in vivo use of zol in patients with malignancies (Siegers G M, et al. PLOS One. 2011 6(2):e16700; Tosolini M, et al. Oncoimmunology. 2017 6(3):e1284723; Godder K T, et al. Bone marrow Transplant. 2007 39(12):751-7; Kunzmann V, et al. J Immunother. 2012 35(2):205-13; Wilhelm M, et al. J Transl Med. 2014 12:45), the experience of ex vivo γδ T cell expansion is still limited (Xiao L, et al. Cytotherapy. 2018 20(3):420-35; Silva-Santos B, et al. Nature Rev Immunol. 2015 15(11):683-91; Legut M, et al. Cellular & Molecular Immunol. 2015 12(6):656-68). Initial treatment of PBMCs with zol and IL-2 is an important phase that yields >90% γδ T cell enrichment. These γδ T cells preferentially express NKG2D that can further enhance the cytotoxicity of γδ T cells as previously reported (Niu C, et al. Oncotarget. 2017 8(4):5954-64; Ang W X, et al. Mol Ther Oncolytics. 2020 17:421-30). NKG2D is an activating receptor expressed on γδ T cells, CD8 T cells, and natural killer cells that can provide potent co-stimulatory and activation signals (Zhang J, et al. Frontiers in Immunology. 2015 6:97; Rincon-Orozco B, et al. J Immunol. 2005 175(4):2144-51) and mediate antineoplastic cytotoxicity (Deniger D C, et al. Clin Cancer Res. 2014 20(22):5708-19; Ang W X, et al. Mol Ther Oncolytics. 2020 17:421-30; Bauer S, et al. Science. 1999 285(5428):727-9). NKG2D expression with the use of quadruplet aAPCs in the protocol further increased to >90% after day 10 of expansion. These cells were found to exhibit potent cytotoxic activity against neoplastic cell targets. These findings suggest that aAPC expanded γδ T cells can enhance tumor killing by NKG2D expression in addition to γδ T cell expansion. This is particularly important in AML therapy since NKG2D ligand expression in leukemic blasts is a determinant of susceptibility to γδ T cell cytotoxicity (Lanca T, et al. Blood. 2010 115(12):2407-11). There was also a significant fold increase in CD16+ and CD56+ expressing γδ T cells following aAPC co-culture, which can further enhance γδ T cell cytotoxicity by mechanisms that also includes antibody-dependent cell-mediated cytotoxicity via CD16 (Alexander A A, et al. Clin Cancer Res. 2008 14(13):4232-40; Tokuyama H, et al. Int J Cancer. 2008 122(11):2526-34; Seidel U J, et al. Frontiers in immunology. 2014 5:618; Fisher J P, et al. Oncoimmunology. 2014 3(1):e27572).

The phenotype of γδ T cells after day 10 of expansion with aAPCs was different between zol treated and untreated cells. While a higher frequency of EM and CM cells were present in a final γδ T cell product of zol treated patients, zol untreated cells had a higher proportion of more EMRA cells. This is an informative observation since T cells that maintain a less differentiated state are critical for therapeutic efficacy (Xiao L, et al. Cytotherapy. 2018 20(3):420-35; Abate G, et al. J Infectious diseases. 2005 192(8):1362-71). Ex vivo stimulation and expansion of T cells can cause a transition through progressive stages of differentiation, which is characterized by a loss of effector function and therapeutic potential (Abate G, et al. J Infectious diseases. 2005 192(8):1362-71; de Witte M A, et al. Biol Blood Marrow Transplant. 2018 24(6):1152-62). Thus, observation further highlights the importance of incorporating zol in ex vivo expansion of γδ T cells with the use of aAPCs in order to maintain their antineoplastic efficacy. There was also decreased expression of checkpoint receptor PD1 and LAG3 on γδ T cells after 10-day expansion with aAPCs and zol treatment. These findings are consistent with the experience by Xiao and colleagues (Xiao L, et al. Cytotherapy. 2018 20(3):420-35). Thus, downregulation of immune checkpoint receptors can potentially promote effective antineoplastic activity (Lopez R D. Blood. 2013 122(6):857-8).

Although the scarcity of γδ T cells circulating in patients with malignancies is a significant obstacle for γδ T cell adoptive transfer (Ribeiro S T, et al. Frontiers in immunology. 2015 6:15), the robust production system results in >600-fold increase in γδ T cells, making ex vivo expanded γδ T cell immunotherapy feasible in patients with malignancies. The effective reduction of αβ T cells to <1% in a final expansion product makes γδ T cells an attractive allogeneic donor-derived immunotherapy that is not associated with increased risk of GVHD (Blazar B R, et al. Nat Rev Immunol. 2012 12(6):443-58; Xiao L, et al. Cytotherapy. 2018 20(3):420-35). Such therapy can potentially benefit patients with various cancer types but particularly those with relapsed AML after allogeneic HCT, who generally have no further effective GVHD risk free treatment options (Bejanyan N, et al. Biol Blood Marrow Transplant. 2015 21(3):454-9; Bejanyan N, et al. Bone Marrow Transplant. 2014 49(8):1029-35). Moreover, T cells in patients with malignancies can exhibit increased exhaustion phenotype (Catakovic K, et al. Cell Commun Signal. 2017 15(1):1), thus using allogeneic donor-derived γδ T cells can provide an additional advantage over the use of autologous cells as an anticancer immunotherapy. The next step is to conduct a clinical trial to study the safety and effectiveness of ex vivo aAPC expanded donor-derived γδ T cells for the treatment of patients with high-risk AML.

### Example 2: Gamma Delta CAR T Expansion

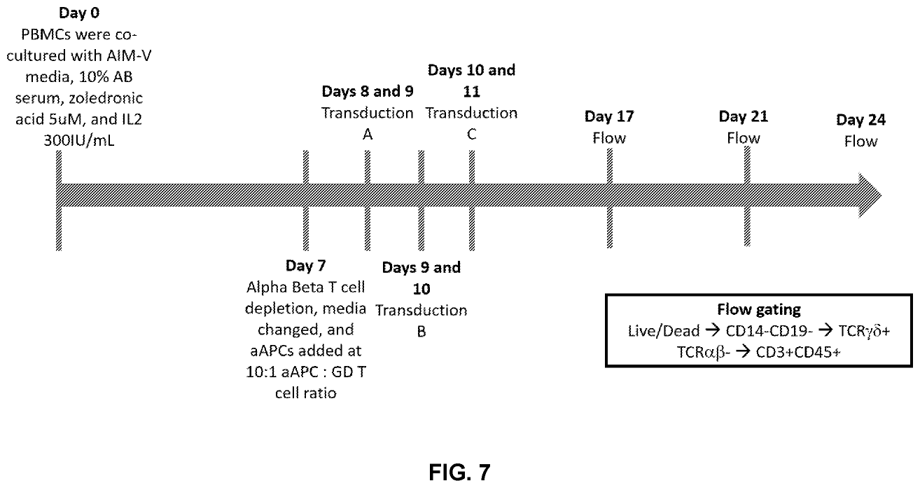

FIG. 7 is a schematic of gamma delta CAR T cell enrichment and CAR transduction at timepoints A, B, and C.

FIG. 8A shows live dead staining of cells at indicated days. FIG. 8B shows live cells stained for CD19 and CD14. Live cells that were double negative for CD19 and CD14 were possible gamma delta cells.

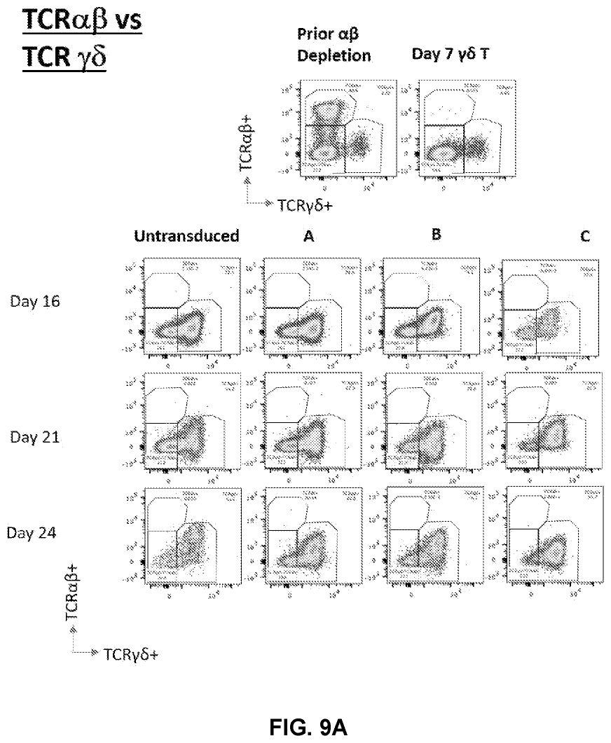

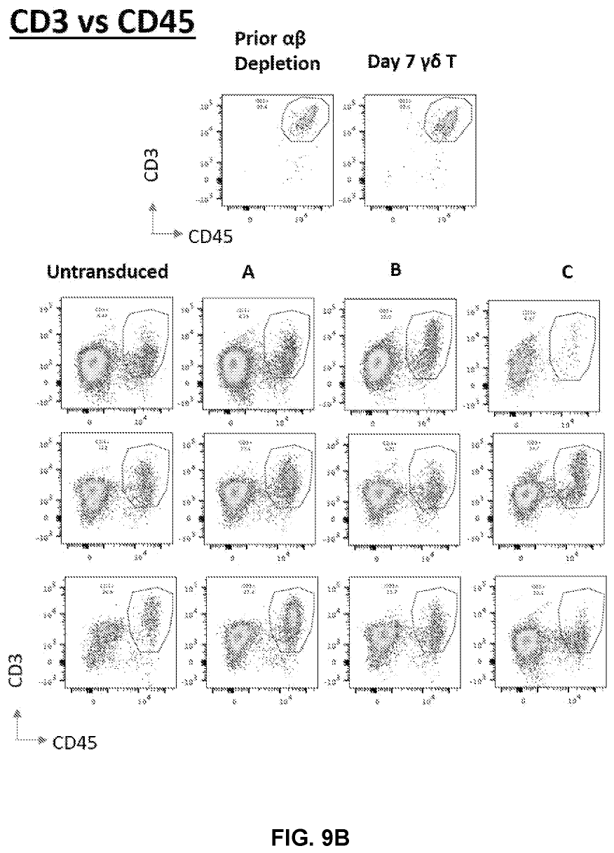

FIG. 9A shows live CD14− CD19− cells stained for TCRab and TCRgd. FIG. 9B shows live CD14− CD19− TCRgd+ cells stained for CD3 and CD45. Cells which were double positive for CD3 and CD45 were considered true gamma delta cells for further figures.

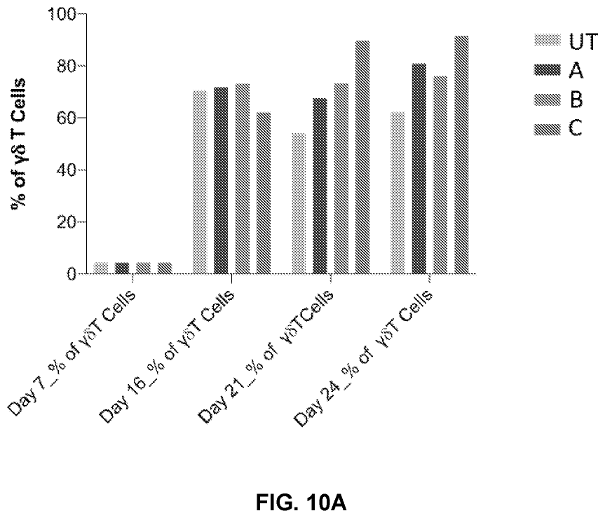

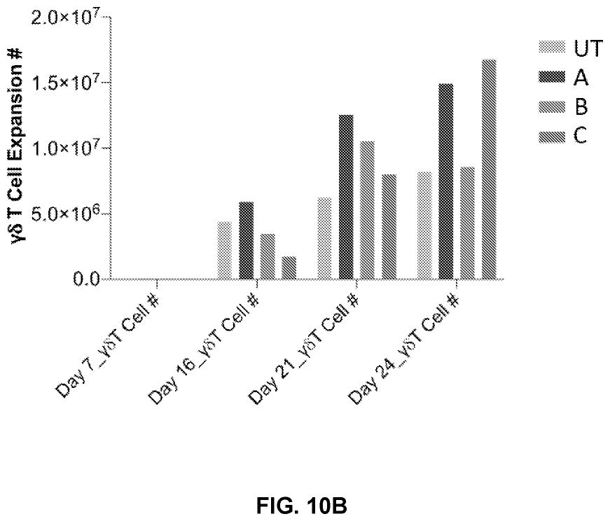

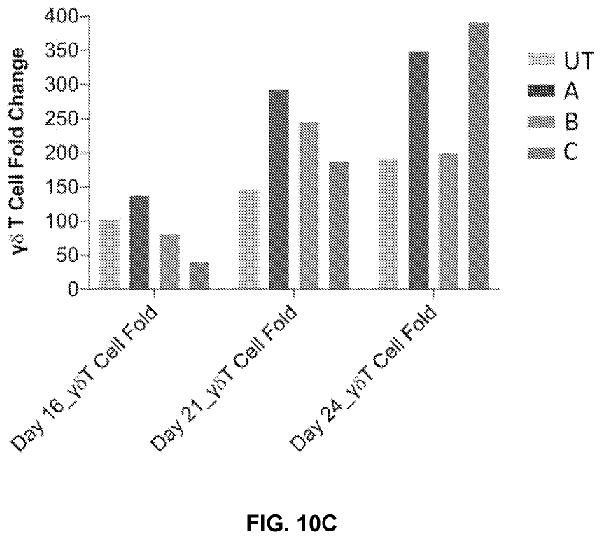

FIG. 10A shows gamma delta T cell percentage. FIG. 10B shows absolute counts of gamma delta T cells. FIG. 10C shows fold increase of gamma delta T cells from day 7.

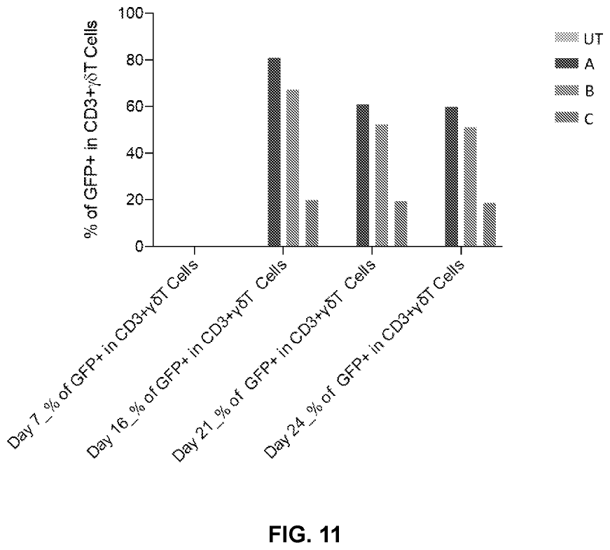

FIG. 11 shows percentage of GFP (CAR) positive gamma delta T cells.

### Example 3: Gamma Delta CAR Killing and Cytokines after Alpha Beta Depletion

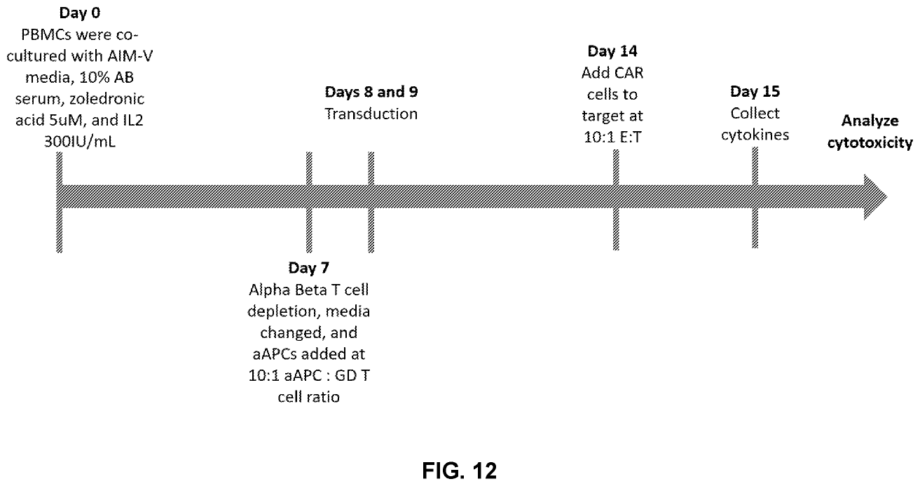

FIG. 12 is a schematic of an experimental design.

### Transduction Efficiency:

$$UT = 0.3$$

$$33 z = 43.3$$

$$33 bb = 74.2$$

$$33 bbz = 68.1$$

$$123 z = 38.6$$

$$123 bb = 74.8$$

$$123 bbz = 58.7$$

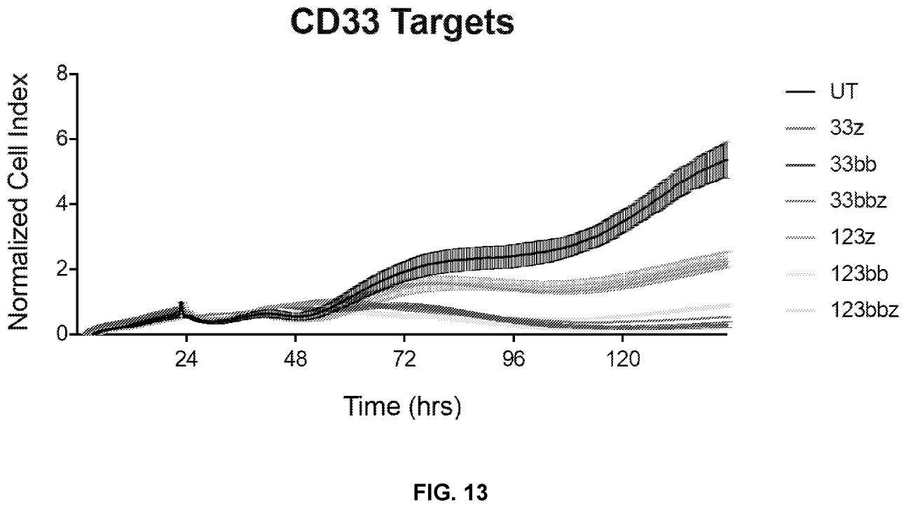

FIG. 13 shows CAR cytotoxicity against CD33 expressing targets. 10:1 effector:target ratio. Lower the line equals more killing.

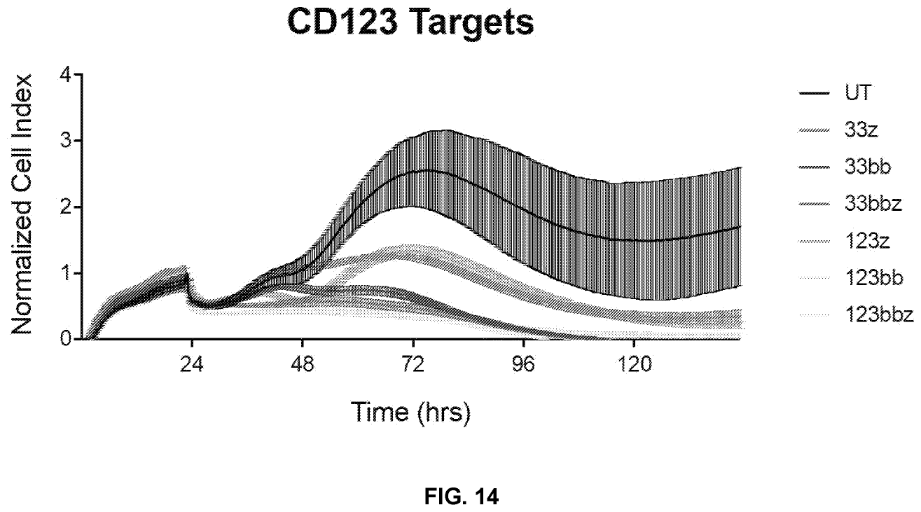

FIG. 14 shows CAR cytotoxicity against CD123 expressing targets. 10:1 effector:target ratio. Lower the line equals more killing.

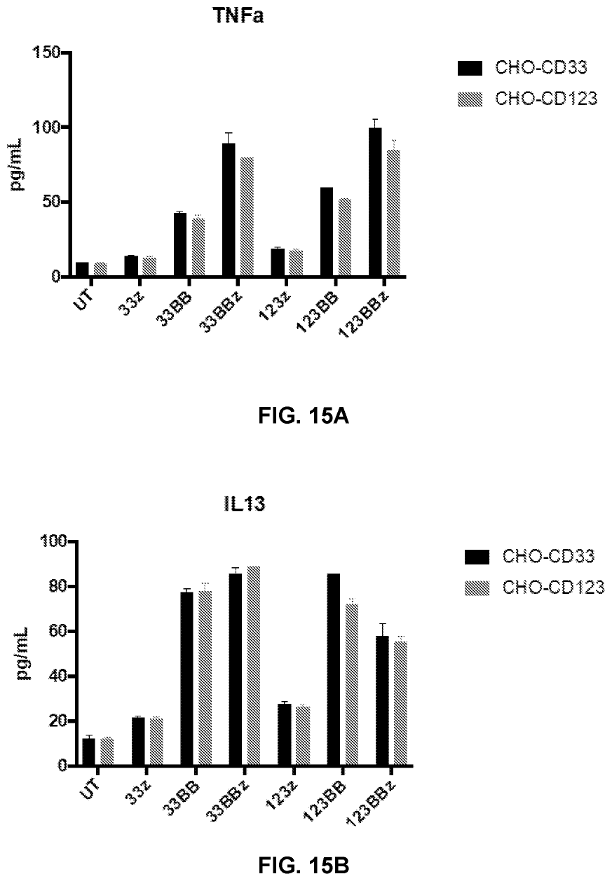

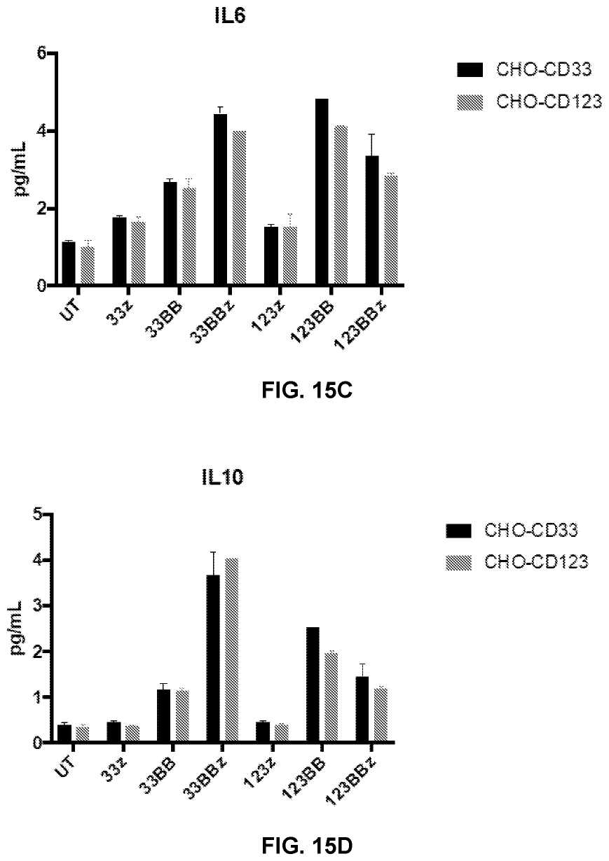

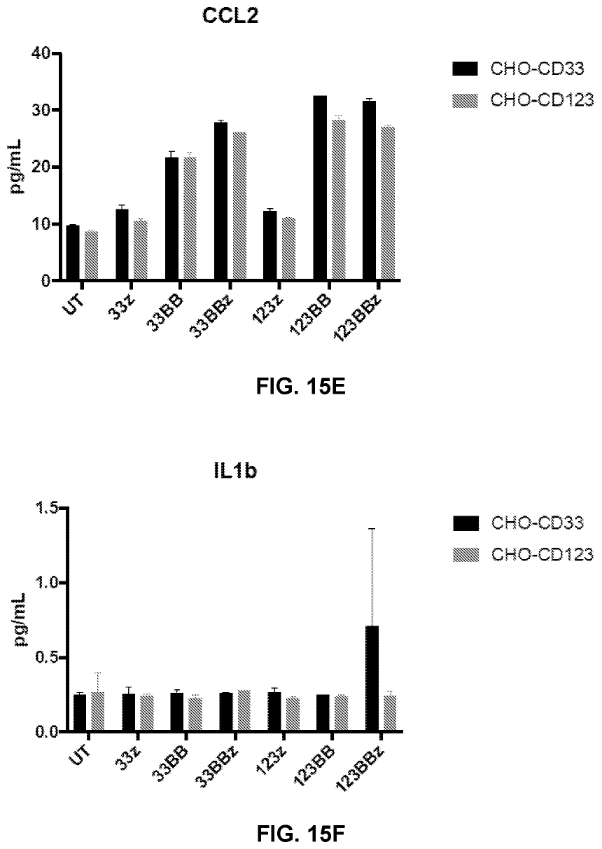

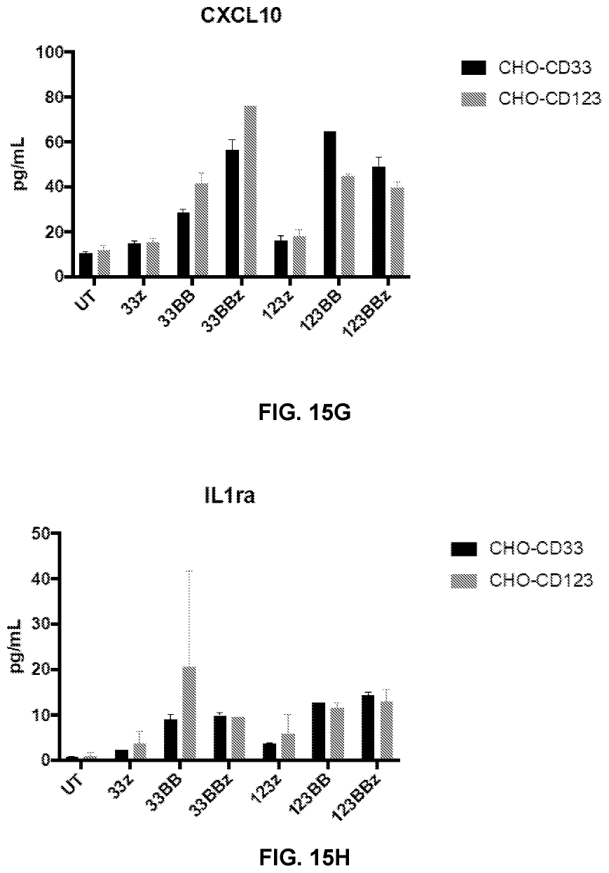

FIGS. 15A to 15H show cytokine secretion from gamma delta CAR T cells.

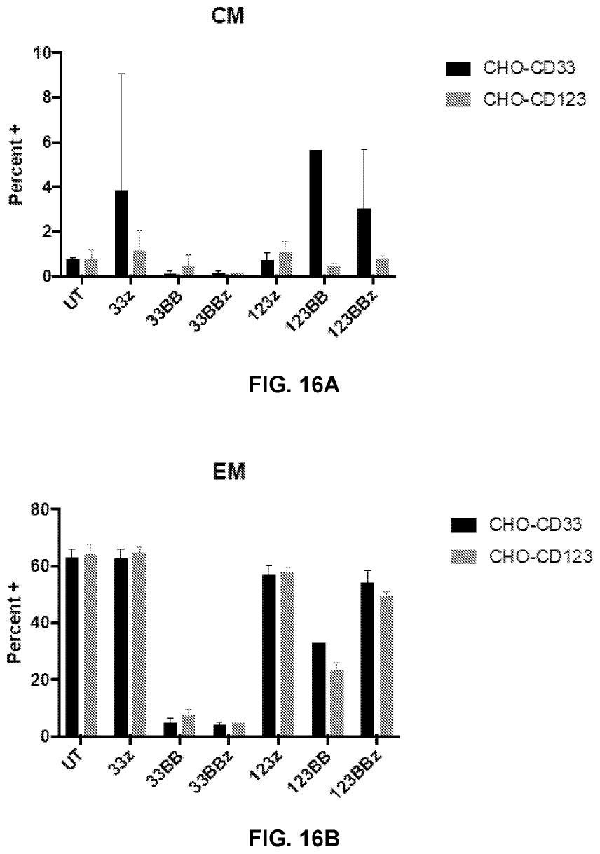

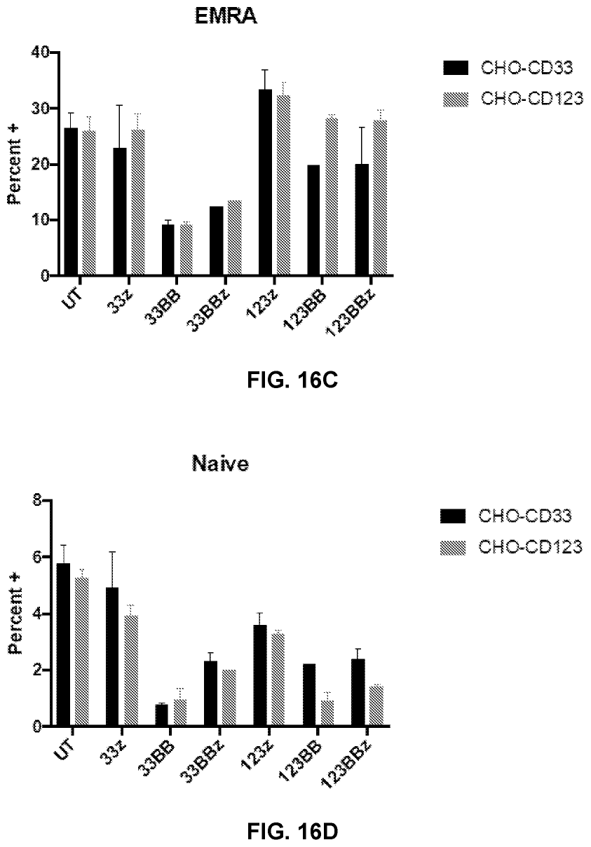

FIGS. 16A to 16D show immune phenotype of gamma delta CAR T cells. CM=central memory. EM=effector memory. EMRA=effector memory RA (most exhausted).

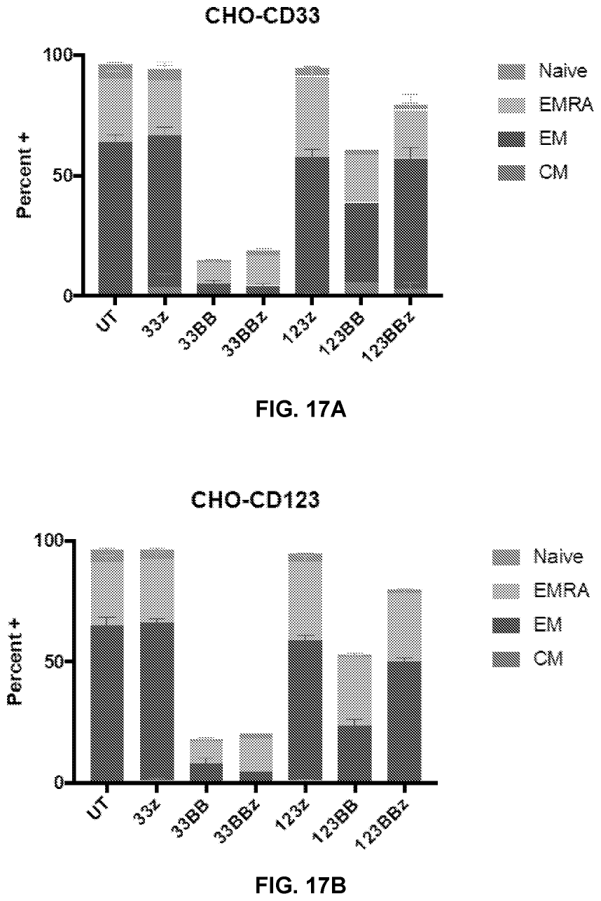

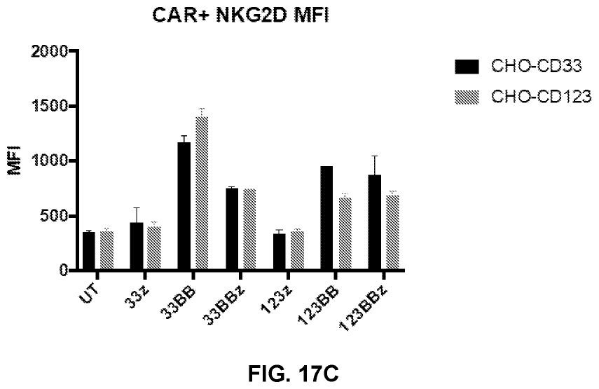

FIGS. 17A and 17B show immune phenotype after stimulation with CD33 targets (FIG. 17A) or CD123 targets (FIG. 17B). FIG. 17C shows NKG2D expression on gamma delta CAR T cells.

### Example 4: CAR-NK Cell Production

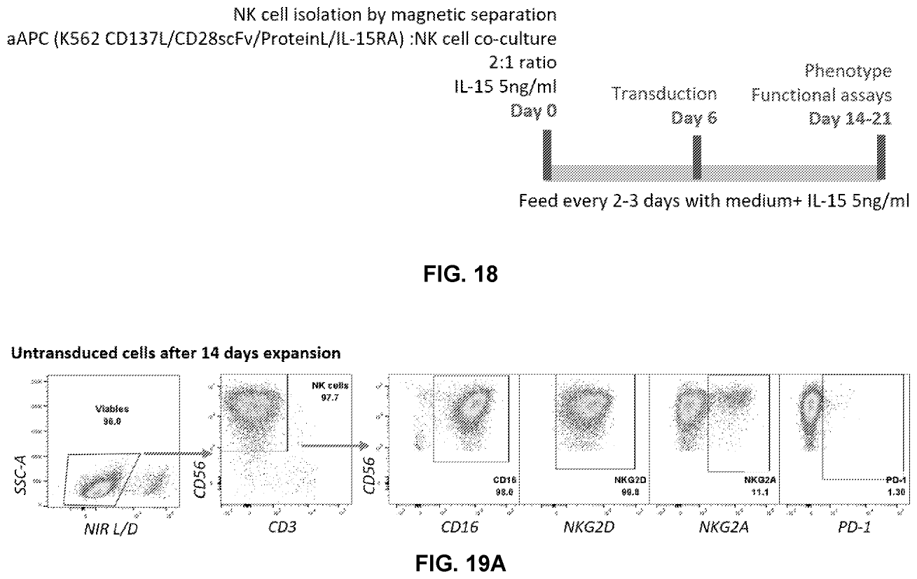

FIG. 18 is a schematic of a NK cell expansion and transduction protocol. NK cells were isolated from healthy donor PBMC and cultured with 30Gy-irradiated aAPC (K562 cells expressing 4-1BBL, IL-15RA, anti-CD28 scFv and ProteinL (aAPC:NK 2:1 ratio) in the presence of IL-15 5 ng/ml (Day 0). After 6 days, NK cells were transduced with SFG retrovirus containing hCD33BBz CAR with different anti-CD33 scFvs sequences (6A11-HC1 LC, 6A11-HC2 LC, 27A3-HC1 LC1, 27A3-HC1 LC2 or 27A3-HC1 LC3) or mock transduced (UT). Between day 14 and 21, CAR-NK cell were harvest and characterized by flow cytometry and functional assays.

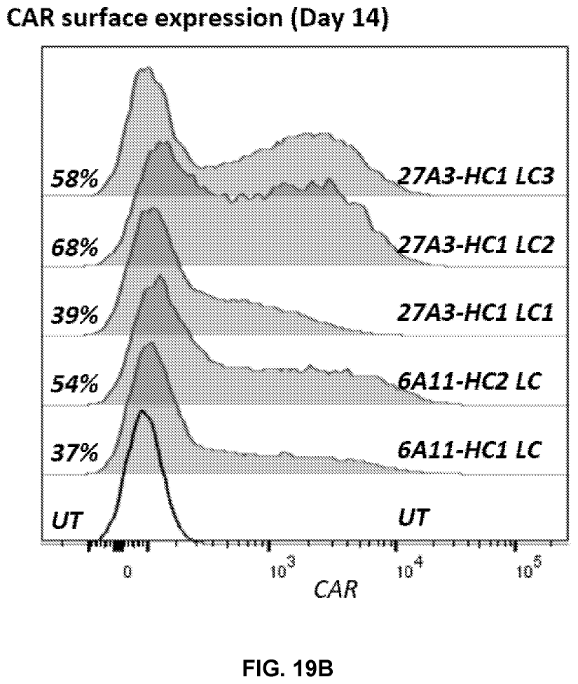

FIG. 19A shows NK cells obtained after expansion were characterized by flow cytometry. FIG. 2A shows representative plots for UT cells: NK cells, gated on live cells based on their expression of CD56 and lack of CD3, represented more than 97% of the product after 14 days. NK cells highly expressed CD16 and NKG2D with variable levels of NKG2A and low PD-1. FIG. 19B shows transduction efficiency estimated by flow cytometry after staining with biotinylated Protein L followed by fluorophore-labeled streptavidin. Percentage of Protein L positive cells was calculated after gating on CD3-CD56+ live cells. At least 37% of the NK cells expressed CD33BBz CAR on the surface, with different expression levels for each anti-CD33 scFv construct.

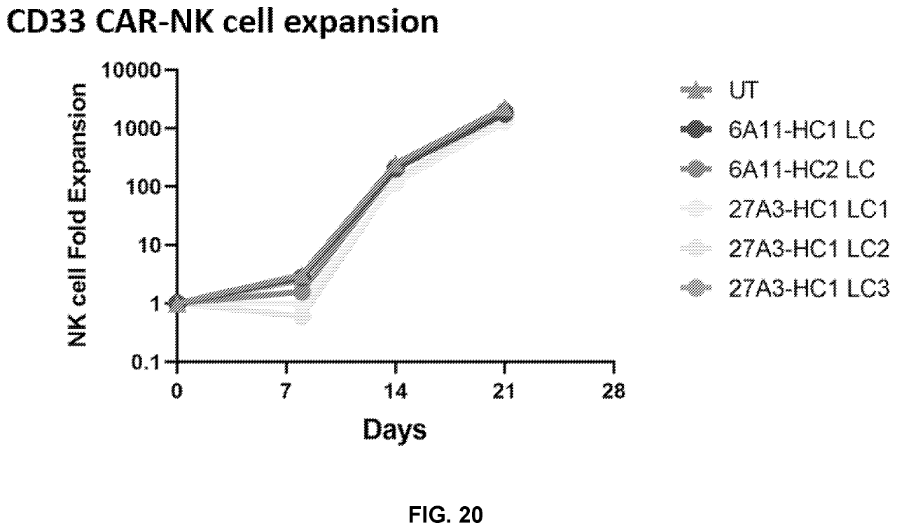

FIG. 20 shows NK cells counted every week by flow cytometry using CountBright absolute counting beads. Co-culture with aAPC resulted in a fold increase of around 2000 for UT and CD33BBz CAR-NK cells on D21.

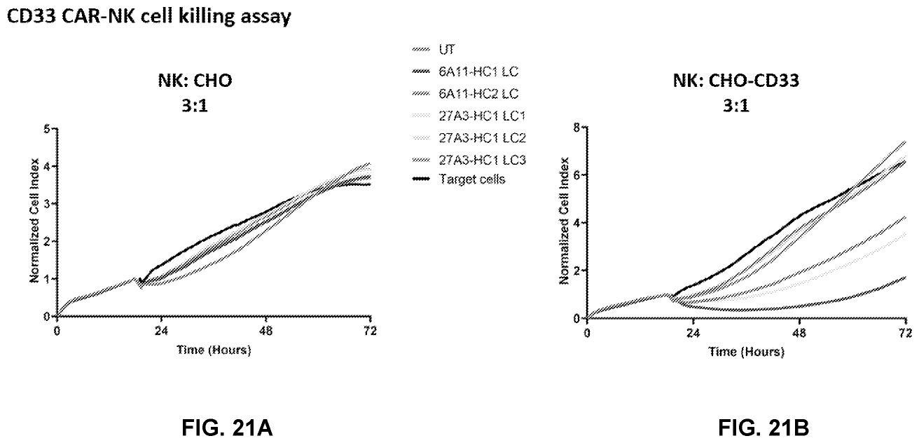

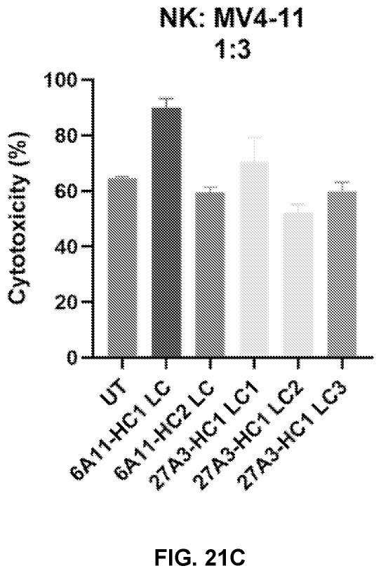

FIGS. 21A and 21B show cytotoxicity evaluated by xCelligence real-time cell analysis (RTCA) using CHO (FIG. 21A) or CHO-CD33 (FIG. 21B) target cells at 3:1 E:T ratio. CD33BBz CAR-NK cells expressing 6A11-HC1 LC scFv showed the highest cytotoxicity against CHO-CD33 cell line. FIG. 21C shows cytotoxicity evaluated by a luminescence assay using MV4-11 AML cell line (expressing luciferase) at 1:3 E:T ratio. CD33BBz CAR-NK cells expressing 6A11-HC1 LC scFv showed the highest cytotoxicity.

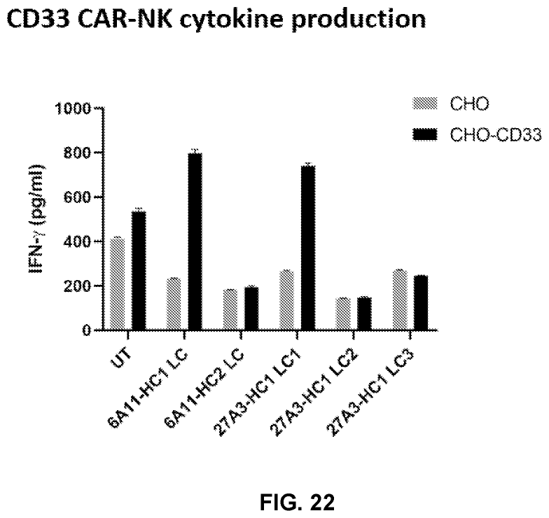

FIG. 22 shows IFN-γ production by CD33 CAR-NK cells evaluated in the supernatant of a co-culture with CHO or CHO-CD33 target cells at 1:1 E:T ratio by a Simple Plex assay on ELLA platform. CD33BBz CAR-NK cells expressing 6A11-HC1 LC scFv showed the highest IFN-γ production against CHO-CD33 cell line.

### Example 5: CAR-NK Cells with Membrane Bound IL-14 and IL-21

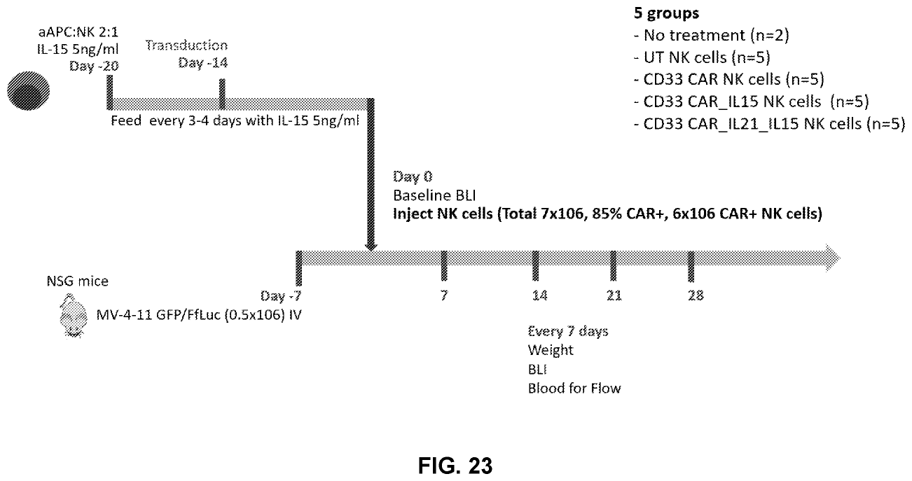

FIG. 23 illustrates an experiment to study the ability of CD33 CAR-NK cells expressing membrane bound IL-15 (mb-IL15) to survive and kill tumor cells in vivo and to compare the activity of CAR-NK cells expressing mb-IL15 vs mb-IL15+membrane bound IL-21 (mb-IL-21).

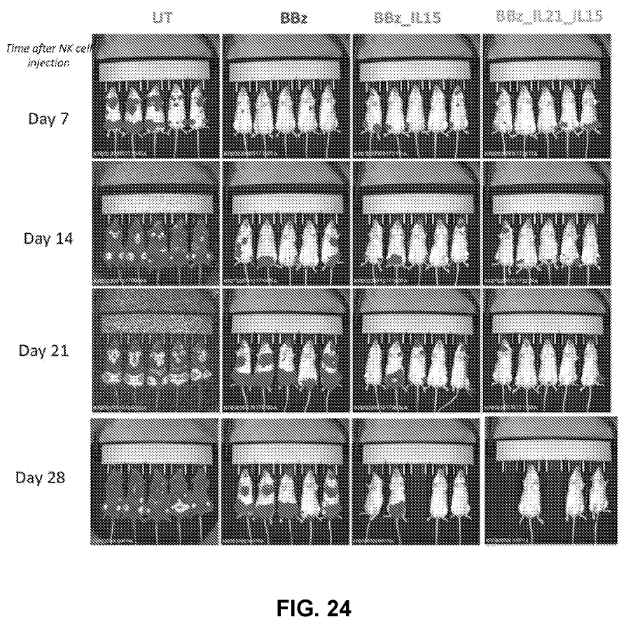

FIG. 24 shows tumors 7, 14, 21, and 28 days after treatment with UT, CD33 CAR-NK cells, CD33 CAR-NK cells with mb-IL5, and CD33 CAR-NK cells with mb-IL5 and mb-IL21.

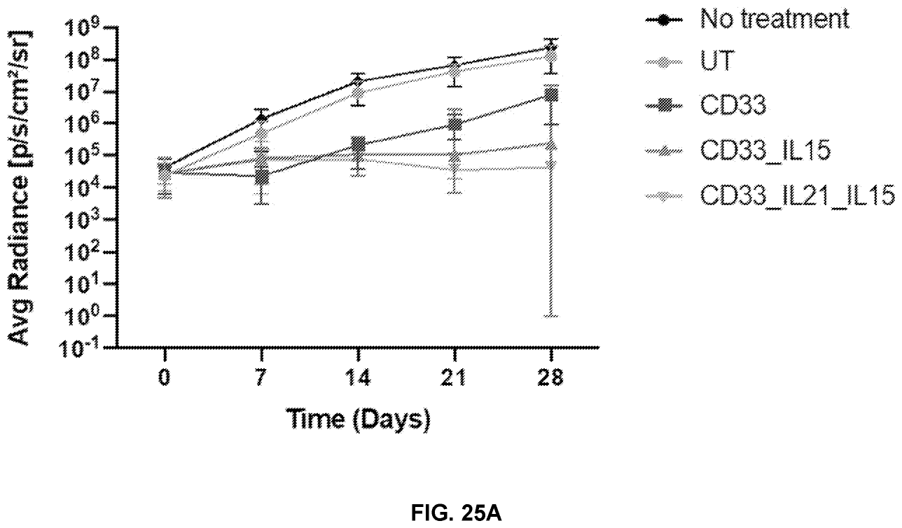

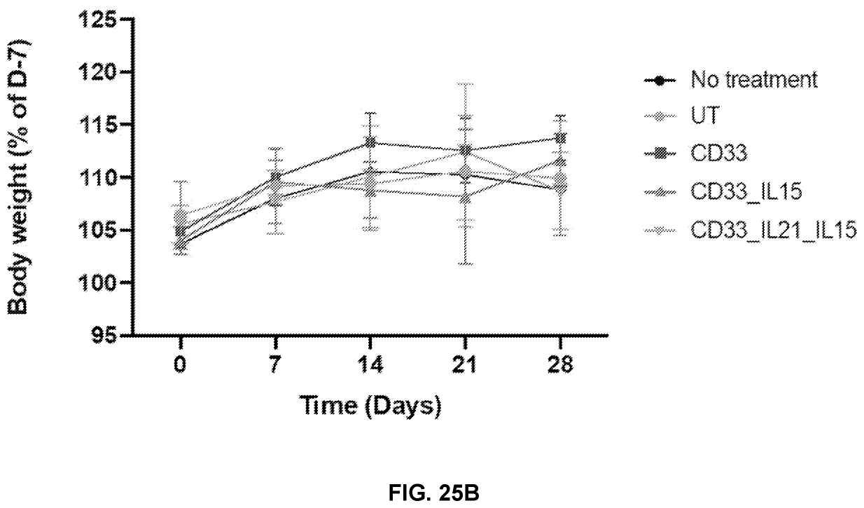

FIGS. 25A and 25B show BLI (FIG. 25A) and body weight (FIG. 25B) 7, 14, 21, and 28 days after treatment with UT, CD33 CAR-NK cells, CD33 CAR-NK cells with mb-IL5, and CD33 CAR-NK cells with mb-IL5 and mb-IL21.

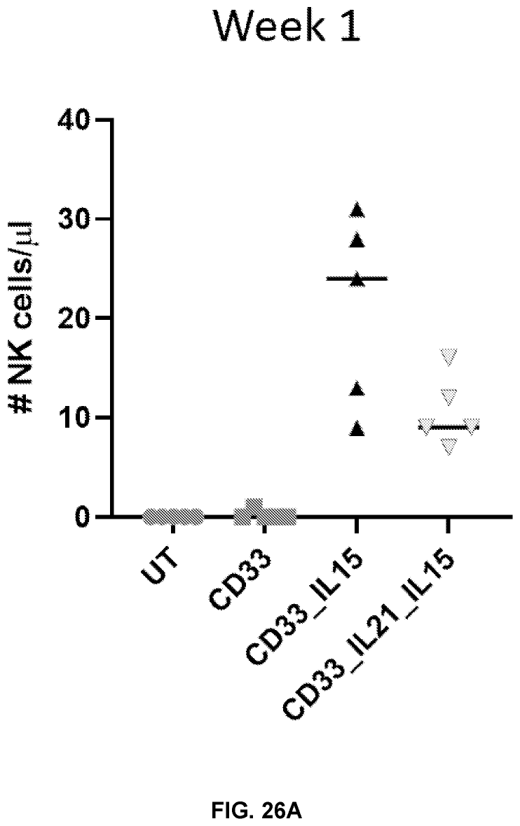

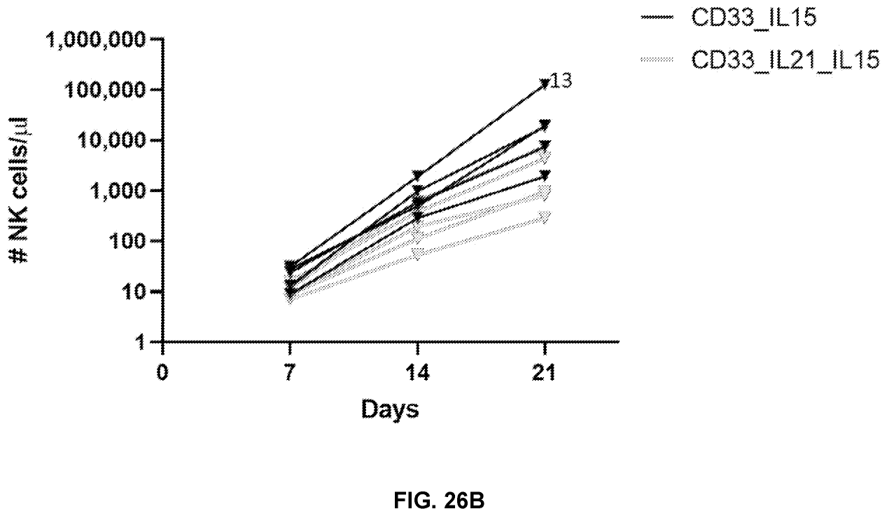

FIGS. 26A and 26B show number of NK cells per microliter one week (FIG. 26A) or 7 to 21 days (FIG. 26B) after treatment with UT, CD33 CAR-NK cells, CD33 CAR-NK cells with mb-IL5, and CD33 CAR-NK cells with mb-IL5 and mb-IL21.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of providing anti-cancer immunity in a subject, the method comprising administering to the subject an effective amount of an NKG2D-expressing immune effector cell genetically modified to express an anti-CD33 CAR polypeptide, an anti-CD123 CAR polypeptide, an anti-CD99 CAR polypeptide, an anti-CLEC12A CAR polypeptide, an anti-EGFR CAR polypeptide, an anti-MUC1 CAR polypeptide, or a combination thereof, thereby providing an anti-tumor immunity in the mammal, wherein the NKG2D-expressing immune effector cell is further genetically modified to express a membrane-bound IL-15 molecule, a membrane-bound IL-21 molecule, or a combination thereof.

2. The method of claim 1, wherein at least 70% of the immune effector cells express detectable NKG2D.

3. The method of claim 1, wherein at least 70% of the immune effector cells are selected from the group consisting of a γδT cell, a Natural Killer (NK) cell, or a combination thereof.

4. The method of claim 3, wherein the immune effector cells have been expanded with artificial antigen presenting cells (aAPCs).

5. The method of claim 1, wherein the membrane-bound IL-15 molecule comprises the amino acid sequence SEQ ID NO:230.

6. The method of claim 1, wherein the membrane-bound IL-21 molecule comprises the amino acid sequence SEQ ID NO:240.

7. The method of claim 1, further comprising administering to the subject a checkpoint inhibitor.

8. The method of claim 7, wherein the checkpoint inhibitor comprises an anti-PD-1 antibody, anti-PD-L1 antibody, anti-CTLA-4 antibody, or a combination thereof.

9. The method of claim 1, wherein the cancer comprises myelodysplastic syndromes, acute myeloid leukemia, or bi-phenotypic leukemia.

10. The method of claim 1, wherein the cancer comprises non-small-cell lung carcinoma (NSCLC).

11. An expression vector comprising a gene encoding a CAR polypeptide, a gene encoding a membrane-bound IL-15 molecule, a gene encoding a membrane-bound IL-21 molecule, or a combination thereof, operably linked to a single expression control sequence.

12. The expression vector of claim 11, wherein the gene encoding the CAR polypeptide, the gene encoding the membrane-bound IL-15 molecule, the gene encoding the membrane-bound IL-21 molecule, or combination thereof are separated by a nucleic acid sequence encoding a self-cleaving peptide, such as P2A.

13. The expression vector of claim 11, wherein the CAR polypeptide is an anti-CD33 CAR polypeptide, an anti-CD123 CAR polypeptide, or a combination thereof.

14. The expression vector of claim 11, wherein the CAR polypeptide is an anti-CD99 CAR polypeptide.

15. The expression vector of claim 11, wherein the CAR polypeptide is an anti-CLEC12A CAR polypeptide.

16. The expression vector of claim 11, wherein the CAR polypeptide is an anti-EGFR CAR polypeptide, an anti-MUC1 CAR polypeptide, or a combination thereof.
